SEQUENCE LISTING (223 sequences)

SEQ ID NO: 1 - DNA, 1270 bp, Rahnella aquatilis
  gene 1..1270: note = 16S
  source 1..1270: mol_type = genomic DNA
attgaagagtttgatcatggctcagattgaacgctggcggcaggcctaacacatgcaagtcgagcggcagcgggaagtagcttgctactttgccggcgagcggcggacgggtgagtaatgtctgggaaactgcctgatggagggggataactactggaaacggtagctaataccgcatgacctcgnaagagcaaagtgggggatcttcggacctcacgccatcggatgtgcccagatgggattagctagtaggtgaggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttagggttgtaaagcactttcagcgaggaggaaggcatcanacttaatacgtgtggtgattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgcgcttaacgtgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccacggaattcgccagagatggcttagtgccttcgggaaccgtganacaggtgctgcatggctgtcgtcagctcgtgttgtgaaatgttgggttaagtcccgcaacgagcgcaacccttatcctttgttgccagcnngtnatgnngggaactcaaaggagactgccggtgataaaccggaggaaggtggggatgacgtcaagtcatcatggcccttacgagtagggctacacacgtgctacaatggcatatacaaagagaagcgaactcgcgagg

SEQ ID NO: 2 - DNA, 876 bp, Rahnella aquatilis
  gene 1..876: note = nifH
  source 1..876: mol_type = genomic DNA
atggcaatgcgtcaatgcgcaatctacgggaaagggggtattgggaaatccaccactacccaaaaccttgtagcggctctggccgaaatgaataagaaggtcatgatcgtcggctgtgaccctaaggctgattcaacccgcctcattctgcatgcgaaagcacagaacaccatcatggaaatggccgctgaagtgggctccgtggaagatctggagctggaagatgtgatgcaaatcggctatggcggcgtgcgctgtgcggaatcaggcggccctgagcctggtgtgggttgtgccggacgcggggtgatcaccgccatcaacttcctcgaagaagaaggcgcgtatgtgccggatctggattttgtgttttacgacgtattgggcgatgtggtctgtggcggtttcgcgatgccaattcgcgaaaacaaagcgcaggaaatctacatcgtgtgctccggtgaaatgatggcgatgtatgccgccaacaacatttccaaaggcatcgtgaaatacgcgaaatcgggcaaagttcgcctggccgggctgatctgtaactcccgccagacggatcgcgaagatgaactgatcatcgcgctggctgaaaaacttggcacgcaaatgatccacttcgtgccgcgtgacaacattgtgcaacgcgctgaaatccgccgcatgacggtcatcgaatacgacccgacttgtgcgcaggcagatcagtatcgtgcactggcgaacaaaatcgtcaacaacaccaaaatggtggtgccgacaccggtcaccatggatgagctggaagccctgttaatggaatttggcattatggaagaagaagacctggccatcgtcggtcgtaccgccgccgaagaggcgtga

SEQ ID NO: 3 - DNA, 1374 bp, Rahnella aquatilis
  gene 1..1374: note = nifD1
  source 1..1374: mol_type = genomic DNA
atgaaggcaaaagagattctggcgctgattgatgagccagcctgtgagcataaccacaagcagaagtcgggttgcagcctgccgaaaccgggcgcgacggcaggcggttgtgcgtttgatggcgcgcagattgcgctgctgccggtcgcggacgtcgcgcatctggtgcacggcccgattggctgtaccggcagttcatgggacaaccgtggcagccgcagttccgggccttccatcaaccgcatgggcttcaccaccgacatgagcgagcaggatgtgattatggggcgcggcgagcgacgcttatttcacgccgtgcagcacatcgtcagccattaccatccggtggcggtctttatttacaacacctgcgtacccgcgatggaaggggatgacgttgaagccgtgtgtcgcgccgcatcggccgctgccggtgtgccggttatttcagtcgatgccgccggtttctacggcagcaaaaatctcggtaaccgcattgccggggacgtgatggtcaaaaaggtgatcggccagcgcgaacccgcgccgtggccggaaaactcaccgatccccgccggacaccgccacagcatcagcctgattggcgaattcaatattgccggcgagttctggcacgttctgccgctgctcgatgagctcgggatccgcgtgctgtgcagcctttccggggattcccgttttgctgaaatccagactatgcaccgtggcgaagccaacatgctggtgtgctcgcgggcgctgatcaacgtcgcccgaaaaatggaagagcgttaccagatcccatggtttgaaggcagtttttatggcctgcgttccatggctgattccctgcgcacgatcgccgtgctgctcaaagacccggatttacaggcgcgcacagaacgtctgattgagcgcgaggaggcggcgacacatcttgcgcttgcgccttaccgtgcgcggctcagcgggcgcaaggcgctgctgtataccggtggcgtgaaatcctggtcggtggtctcggcgttacaggatttaggcatcacggtggtggcgaccggcacccgaaaatcaaccgaagaagacaagcagcgtattcgcgaactgatgggtgaagacgtgctgatgctcgacgaaggcaatgccagaaccttgctcgacaccctctatcgtttcggcggcgacatcatgatcgccgggggccgcaacatgtataccgcgtacaaagcccgcctgccgttcctggatatcaatcaggagcgcgagcatgcgtttgccggatatcacgggctggtaaatctggccgaacagttgtgtatcaccctggaaagcccggtctgggcgcaggtcaaccgtctggcgccgtggcgctaa

SEQ ID NO: 4 - DNA, 1449 bp, Rahnella aquatilis
  gene 1..1449: note = nifD2
  source 1..1449: mol_type = genomic DNA
atgaccagtgaaacacgcgaacgtaacgaggcattgatccaggaagtgctggagatcttccccgagaaggcgcttaaagatcgtaagaaacacatgatgaccaccgacccggcgatggaatctgtcggcaagtgtattgtctcaaaccgcaaatcacagccgggcgtgatgaccgtgcgaggctgcgcttacgccggttccaaaggcgtggtctttggcccgatcaaagacatggcgcatatctcccacggcccggttggttgcggccagtattcccgtgccggacgccgtaactattacaccggctggagcggcgtgaacagctttggcaccctcaacttcaccagtgattttcaggaacgggacatcgtatttggcggcgataaaaagctcgacaaattgatcgatgaactggagatgttgttcccgctgagcaaaggcatttcggtgcagtcggaatgtccggtcggtctgatcggcgatgacatttctgccgtcgccaaagccagcagcgccaaaatcggtaagccggtcgtgccggtacgctgcgaggggttccgcggtgtgtcgcaatcgctcggccatcacattgctaacgatgtcatccgcgactgggtgctggataaccgcgaaggcaatgaatttgaaaccacgccttacgacgtggcgattatcggcgactacaacatcggcggtgacgcctgggcctcacgtattctgctcgaagaaatggggctgcgcgtggtggcgcagtggtccggcgacggcacgctggtggagatggaaaacaccccgaaagtcgcgctcaatctggtgcactgctaccgctcgatgaactacatctcccgtcatatggaagaaaaacacggcattccgtggatggaatacaacttctttggcccgaccaaaattgcggaatctctgcgcgaaatcgcggcgcgttttgacgataccatccggaaaaacgccgaagcggtgattgaaaaatatcaggcgcaaacgcaggcggtgatcgacaaataccgtccgcgtctggaaggcaaaaaggtgctgttgtatctcggcggtttacgtccgcgccacatcatcggggcgtatgaagatctgggaatggaaatcatcggtaccggctatgaattcggtcataacgatgattacgaccgcaccttaccgatgctcaaagaaggcacgttgctgttcgatgacctgagcagttatgagctggaagcgttcgttaaagcgctgaaaccggatcttgtcgggtcaggtatcaaagaaaaatacattttccagaaaatgggcgtgccgttccgccagatgcactcctgggattattccggcccttatcacggctacgacggtttcggcatttttgcccgtgacatggacatgacgctgaacaatccgggctggagtcagctgaccgccccctggttgaaaacggcctga

SEQ ID NO: 5 - DNA, 1386 bp, Rahnella aquatilis
  gene 1..1386: note = nifK1
  source 1..1386: mol_type = genomic DNA
atggctcaaattctgcgtaatgccaagccgcttgccaccacgcctgtcaaaagcgggcaaccgctcggggcgatcctggccagtcaggggctggaaaattgcatcccgctggttcacggcgcgcaaggttgtagcgcgttcgccaaagttttcttcatccagcattttcacgatccgatcccgttgcagtccacggcgatggaatcgaccacgactatcatgggctcggatggcaacgtcagtactgcgttgaccacgttgtgtcagcgcagtaatccaaaagccattgtgattttgagcaccggactgtcagaagcgcagggcagtgatttgtcgatggcgctgcgtgagtttcgcgacaaagaaccgcgctttaatgccatcgctattctgaccgttaacacgccggatttttacggctcgctggaaaacggctacagcgcgctgatggaaagcgtgatcactcagtgggtgccggaaaagccgccgaccggcatgcgtaacaagcgcgtgaacctgctggtgagccatctgctgacgccgggggatctggaattactgcgcagctatgtcgaagcctttggcctgcaaccggtgatcctgccggatttatcacagtcgctggacggacatctggcgaatggcgatttcaatccggtcacgcagggcggcacgtcgcaacgccagattgaacaaatggggcagagcctgaccaccattaccattggcagttcgctcaactgcgccgccagtctgatggcgatgcgcagccgtggcatggcgctgaacctgccgcacctgatgacgctggaaaacatggacagtctgatccgccatctgcatcaggtgtcaggccgcgaggtaccggcatggattgagcgccagcgcgggcaactgctggacgccatgatcgactgccatacctggctgcagtcacagcgtattgcgctggcggcagaagcggatttgctggtggcgtggtgcgatttcgctcagagccagggaatgcgcgtcgggccggtgattgcgccggttaatcagcagtcactggccgggctgccggtcgaacaggtggtgatcggcgatctggaagatttacaaacccggctcgacagctacccggtttcactgctggtggcgaactcccacgctgcaccactggcggaaaaaaacggtatcgcgctggtacgtgccggtttcccgctttacgaccgtctcggggaatttcgccgcgtgcggcagggctatgcgggtattcgcgacaccttgttcgaactcgcgaacctgatgcaggcgcgccatcacatgctgacggcgtatcactcaccgcttaggcaggtgttcggcctgagcccggtaccggaggccagtcatgaggcgcgctaa

SEQ ID NO: 6 - DNA, 1569 bp, Rahnella aquatilis
  gene 1..1569: note = nifK2
  source 1..1569: mol_type = genomic DNA
atgagtcaagatcttggcaccccaaaatcctgtttcccgctgttcgagcaggatgaataccagaatatgtttacccacaaacgcgcgctggaagaagcacacggcgaggcgaaagtgcgggaagtgtttgaatggaccaccacgcaggaatatcaggatctgaacttctcgcgtgaagcgctgaccgtcgacccggcgaaagcctgccagccgttaggcgcggtactttgcgcgctgggttttaccaacacgttgccgtatgtccatggttcacaaggctgtgtggcgtatttccgtacctattttaatcgtcatttcaaagagccggtggcctgtgtttccgactcaatgaccgaagatgccgccgtttttggcggaaataacaacatgaatgtcggtctggaaaacgccagcgcgctgtacaagccggaaattattgcggtctccaccacctgtatggcggaagtgatcggtgatgacctgcaggcttttatcgccaacgccaaaaaagacggatttgtggatgccggtatgccaatcccgtatgcccatacaccgagttttctgggcagtcatgtcaccggctgggacaacatgtttgaaggcttcgcccgtacctttaccaccgacgccacgcgggaatatcagccgggcaaacttgccaaactgaacgtggtgaccggttttgaaacttatctcggcaactaccgggttattcaccgcatgatgagccagatgggggtcgaatgcagcgtcttgtccgatccgtctgaagtgctcgacaccccggctgacggccaataccgcatgtatgccggcggcaccacgcaaaccgaaatgcgtgatgcaccggatgccatcgacaccttgctgctgcaaccgtggcaattacagaaaaccaaaaaggtggtgcagggcgactggaatcagccgggcaccgaagtcagtgtaccgattggcctggcggcgaccgatgccttgctgatgacggtaagcgaactgaccggcaaaccgatagctgacgcgctggcgactgaacgtggccgtctggtggacatgatgctcgattctcacacctggctgcacggcaagcgtttcggtctctacggtgacccggattttgtgatgggcatgaccgcattcctgctggaactgggctgtgaaccgaccaccattctcagccataacggcaacaaacgctggcagaaagccatgaagaaaatgctggctgattcgccttacggacaggacagcgaagtgtatgtgaactgcgatctgtggcatttccgctcgctgatgtttacccgtaaaccggactttatgatcggcaactcttacggaaaattcattcagcgtgacacgctggccaaaggcgaacagttcgaagtgccgctgatccgtatcggattcccgatttttgaccggcaccatttgcaccgtcagaccacctggggatacgagggcgcgatgagcatcctgacgcaactggtgaatgcggtgctcgaacagctggatcgcgaaaccatgaagctcggcaaaaccgactacaacttcgatctgatccgctaa

SEQ ID NO: 7 - DNA, 1488 bp, Rahnella aquatilis
  gene 1..1488: note = nifL
  source 1..1488: mol_type = genomic DNA
atgagcatcacggcgttatcagcatcatttcctgaggggaatatcgccagccgcttgtcgctgcaacatccttcactgttttataccgtggttgaacaatcatcggtggcgatttcgctgaccgatccgcaggcgcgcatttgttatgccaatccggcattctgccgccagacgggttttgcacttgagacacttttgggcgagaaccaccgtctgctggccagccagcagacgccgaaacatatctatgacgaaatgtggcgcactttgttgcagggcaaatcctggaacggccaactgatcaaccggcgtaataaccgttcgctttatctggcggatgtcactatcacgcctgttttaggcgcggacgggcaggtggagcattacctcggcatgcacaaagatatcagcgagaaatacgcgctggagcagcggttgcgcaaccacatcaccttgttcacggaggtgctgaacaatattcccgccgccgtggtggtggtggatgagcaggacaatgtggtgatggacaatctggcctacaaaaccctgtgcgctgactgcggcggaaaagagctgttggccgaaatgggctatccgcaactcaaagagatgctcaacagtggcgaaccggtgccggtttccatgcgcggcaacgtacgctggttttctttcggtcagtggtcattgcagggcgttaatgaagaggccagccgcttttttaccggcattaccgcgccgggaaaactgattgttctcaccgactgcaccgatcagcatcaccggcagcagcagggttatcttgaccggctcaagcaaaaacttaccaacggcaaattgctggcagccatccgcgagtcgcttgatgccgcgctgattcagctcaacgggccaattaatatgctggcggctgcgcgtcgtcttaacggcgaagaaggcaacaacatggcgctggaattcgcctggcgcgaaggcgagcaggcggtgagtcgcttacaggcctgccgtccgtcgctggattttgagccgcaggcagaatggccggtcagtgaattcttcgacgatctgagcgcgctgtacgacagccattttctcagtgacggtgaattgcgttacgtggtcatgccatctgatctgcacgctgtcgggcaacgaacgcaaatccttaccgcgctgagcttatggattgatcacacgctgtcacaggcgcaggccatgccgtctctgaagctctcggtgaacattgttgcgaagcaggatgcgagctggttgtgttttgacattaccgataatgtgccgcgtgaacgggtgcgttatgcccgcccggaagcggcgttttcccgtccggggaatggcatggagctgcgccttatccagacgctgatcgcccatcatcgcggttctttagatctctcggtccgccctgatggcggcaccttgctgacgttacgcctgccggtacagcaggttatcaccggaggcttaaaatga

SEQ ID NO: 8 - DNA, 1557 bp, Rahnella aquatilis
  gene 1..1557: note = nifA
  source 1..1557: mol_type = genomic DNA
atgacccagttacctaccgcgggcccggttatccggcgctttgatatgtctgcccagtttacggcgctttatcgcatcagcgtggcgctgagtcaggaaagcaataccgcgcgcgcactggcggcgatcctcgaagtgcttcacgatcatgcatttatgcaatacggcatggtgtgtctgttcgataaagaacgcaatgcactgtttgtggaatccctgcatggcatcgacggcgaaaggaaaaaagagacccgccatgtccgttaccgcatgggggaaggcgtgatcggcgcggtgatgagccagcgtcaggcgctggtgttaccgcgcatttcagacgatcagcgttttctcgaccgcctgaatatttacgattacagcctgccgctgattggtgtgccgatccccggtgcggataatcagcctgcgggtgtgctggtggcacagccgatggcgttgcacgaagaccggctggctgccagtacgcggtttttagaaatggtcgccaatctcatcagccagccactgcgttctgccacgcccccggaatcattgcctgctcaaacgccggtccggtgcagtgttccgcgccagtttggttttgagcagatggtcgggaaaagtcaggcgatgcgccagacgatggacattttacggcaggtttccaaatgggataccacggttctggtgcgtggtgaaagcggcaccggcaaggaacttatcgccaatgccattcattacaactcaccccgtgcggccgcgccatttgtgaaattcaactgcgccgcgctgccggataacctgctggaaagcgaactgttcggtcatgaaaaaggggccttcaccggcgctatacgcacccgaaaaggccgctttgaactggcggacgggggcacgttattcctcgatgaaatcggcgaatcgagcgcgtcgtttcaggccaaattgctgcgcattttgcaggaaggtgaaatggaacgggtcggcggcgataccacgctgaaagttgatgtgcgcattattgctgccaccaaccgtaatcttgaggaggaagtgcgtgccgggaattttcgcgaagacctgtattatcgcctgaacgtgatgccggtttcgctgcctgcactgcgtgaaaggctggatgatatcgccgatctggcgccgtttctggtcaaaaagattgcgctgcgtcaggggcgggaactgcgcatcagtgatggtgcggtgcgtctgctgatgacctacagctggccaggcaacgtgcgtgaactggaaaactgcctcgaacgggcgtcggtaatgaccgatgaagggctgatcgaccgcgacgtgatcctgttcaatcaccatgagtccccggcgctgtccgtcaaacccggtctgccgctggcgacagatgaaagctggctggatcaggaactcgacgaacgccagcgggtgattgctgcactggagaaaaccggctgggtgcaggccaaagcggcccgactgctgggcatgacaccgcgccagattgcctaccgtatccagattatggacatcaacatgcaccgtatctga

SEQ ID NO: 9 - DNA, 2838 bp, Rahnella aquatilis
  gene 1..2838: note = glnE
  source 1..2838: mol_type = genomic DNA
atgttgccactttcttctgttttgcaaagccacgcgcagagtttgcctgaacgctggcatgaacatcctgaaaacctgcccctccccgatgatgaacagctggctgtgctgagcagcagtgaattcatgacggacagtttgctggcttttccgcagtggtggcatgaaattgtccaaaatccccctcaggcgcaggagtggcaactttaccgtcagtggctggatgaatcgctgacgcaggtgactgacgaagccgggttaatgaaagctttgcgtctgttccgccgccgtattctgacccgcattgcgtggtcacagtccgcgcaaaccagcgaagcaaaagatacgcttcaccagctgagtgaactggcggaattattgattgtcagcgcccgtgactggctgtatgccgcttgctgtcgcgagttcggtacgccggtcaatgccgcaggggaaccgcagagaatgctgatcctcgggatgggcaaactcggcggtggcgagctgaatttctcatcggacatcgacctgatttttgcttatccggaaaatggccagacacgcggcggtcggcgtgaactggataacgcacaatttttcacccggctcggccagcgtctgatcaaagcgctggatcagcccactatcgacggttttgtctatcgcgtggacatgcgtttgcgtccgttcggcgacagtggcccgctggtgatgagcttcccggcactggaagattattatcaggaacaggggcgcgactgggaacgctacgcaatggtgaaagcgcgtctgatgggcggcgcggaggacatcagcagtcaggaattgcgtaaaatgctgatgccttttgtcttccgccgttatatcgatttcagtgtgatccagtccctgcgtaacatgaaaggcatgatcgcccgcgaagtacgccgccgtggtctgaaagacaacatcaaactcggcgcaggcggtattcgtgaaattgaatttatcgtgcaggtatttcagctgatccgtggcggtcgtgaaccggcattgcagcagcgtgcgttgttgccaacgcttcaggcgctggaaaatctggggctgctgccggtagagcaggtgttgcagttgcgtaacagctatctgttcctgcgacgtctggaaaacctgttgcaggccattgctgacgagcaaacgcaaaccttaccgtccgatgagctgaatcaggcgcgtctggcgtgggggatgaattacgctggctggccgcagcttctggatgcagtgaatgctcacatgcaggccgtacgcgcggtatttaacgatctgattggcgatgacacgccagatgccgaagatgacgtgcaactctcccggttcagcagtttatggattgatacgcttgagcctgacgagctggctccgctggtgccgcaacttgacgaaaatgcgcaacggcatgttttacatcagattgctgattttcgccgtgacgtggataaacgcacgatagggccacgtgggcgtgatcagttggatttgctgatgccgcgtttactggcccaggtctgcacctataaaaatgcggatgtgacgttacagcgcctgatgcagttgctgctcaatatcgtcacgcgcacgacgtatatcgagctgctggtggaatatcccggtgcgctcaaacagttaatacgtctgtgcgctgcctcgccgatggtggcgacgcaacttgcgcgtcatcctttattgctcgacgaactgctcgacccgcgcacgctttaccagccgattgagccgggcgcgtaccgtgatgaactgcggcaatatctgatgcgggtgccaaccgaagacgaagaacaacagcttgaagccgtgcgccagttcaaacaggcacagcatttacgtattgcggccggggatatttccggtgcgttgccggtgatgaaagtcagtgaccatttaacctaccttgcggaggccattctcgacgttgtggtgcaacaggcgtgggaacaaatggtcgtaaaatacggtcagccaacccatcttcagcaccgtaaagggcgcggttttgccgtggtgggttacggaaaactcggtggctgggagctgggttacagctcggatctggatctggtcttcctgctcgattgcgcgccggaagtcatgaccgacggcgaacgcagcattgacgggcgtcagttttatctgcggctggcgcagcgcatcatgcatttattcagcacccgtacgtcgtcaggcattctttatgaggttgacccgcgtctgcggccttccggtgcttccggcatgctggtcagcaccatcgaagcttttgcggattatcaggccaacgaagcctggacatgggagcatcaggcgctggttcgcgcgcgtgtggtttatggtgatccgcaactgacgcagcaatttaatgccacgcgtcgcgacattctttgccgccagcgcgatgccgacggcttgcgtaaggaagtccgtgaaatgcgcgagaaaatgtatgcccatctgggcagcaaaagagccgacgagtttgatctgaaagccgatccgggtggcataacggatattgaattcatcgcacaatatctggttctgcgtttcgcgcatgatgagccgaagctgacccgctggtctgataacgtgcggattttcgaactgatggcgcgacatgacatcatgccggaagaggaagcacgccatctgacgcaggcttacgtgacattgcgcgatgaaattcatcatctggcgttgcaggaacacagcgggaaagtggccgcagacagctttgccactgagcgcgcgcaaatccgcgccagctgggcaaactggcttggctga

SEQ ID NO: 10 - DNA, 1290 bp, Rahnella aquatilis
  gene 1..1290: note = amtB
  source 1..1290: mol_type = genomic DNA
atgaaaaaacttttatccatgatggggcttggtgcagtggctttgctaccttcgcttgccatggcagcagcaccagcagcggcaaacggtgctgataacgcctttatgatgatttgtaccgcgctggtattgttcatgaccgtacccggtgtggcgttgttctacggcggcttactgcgttctaaaaacgttttgtccatgctgactcaggttattgttacctttgctctggtctgcgtcctgtggatcctctacggttacagccttgccttcagtgaaggtaacgcgttcttcggtggtttcagcaacgtaatgatgaaaggcattggcctggattctgtgactggcaccttctcgcagatgatccacgttgcattccagtgttcatttgcctgcatcactgtagcgctgatcgtaggtggtattgctgaacgtgtgcgtttctcagcagttctgattttcactgtgatctggctgactttctcttatattccgatggctcacatggtatgggcaggcggtttcctggctgctgacggtgcgctggactttgccggtggtaccgttgttcatatcaatgccgcaattgctggcctggtaggggcttatctgctgggtaaacgcgccggttttggcaaagaagctttcaaaccacacaacctgccaatggtcttcactggcgcctcaatcctgtatgtgggctggttcggcttcaatgcgggttcagcaagtgccgcaagctctgttgccgcgctggctttcctgaacactgtcattgctactgctggcgcaatcctgtcctggacgctggttgagtggatggtgcgcggtaagccctcactgctgggcgcaagctccggtgctatcgcaggtctggtggctatcacgcctgcatgtggtacggtcggcgtaggtggtgctctgattatcggtctggtaggcggtatcactggtctgtggggggttgttaccctgaaaaaatggctgcgtgttgatgacacctgtgatgtgttcggtgttcatggcgtgtgcggtatcgtaggttgtctgctgacgggtgtattcacgtccagttcacttggcggcgtgggctacgcagaaggcgtgaccatgggccatcaggtttgggtgcagttcttcagcgtgtgcgtaacattggtctggtcaggcgttgttgccttcatcggttacaaagtggctgacatgatcgtaggtctgcgtgttcctgaagaacaagaacgcgaaggtctggacgttaacagccacggcgaaaacgcttacaaccaataa

SEQ ID NO: 11 - DNA, 500 bp, Rahnella aquatilis
  gene 1..500: note = PinfC
  source 1..500: mol_type = genomic DNA
tgaatatcactgactcacaagctacctatgtcgaagaattaactaaaaaactgcaagatgcaggcattcgcgttaaagccgacttgagaaatgagaagattggctttaaaattcgcgaacacacgctacgccgtgttccttatatgttagtttgtggcgataaagaggtcgaagcaggcaaagttgctgttcgtactcgtcgcggcaaagacttaggaagcatggatgttagcgaagtcgttgacaaactgctggcggaaatccgcagcagaagtcatcatcaactggaggaataaagtattaaaggcggaaaacgagttcaaccggcgcgtcctaatcgcattaacaaagagattcgcgcgcaagaagttcgcctcaccggcgtcgatggcgagcagattggtattgtcagtctgaatgaagctcttgaaaaagctgaggaagcgggcgtcgatttagtagaaatcagtccgaatgccgagccgccagtttgtcgaatc

SEQ ID NO: 12 - DNA, 1085 bp, Klebsiella variicola
  gene 1..1085: note = 16S
  source 1..1085: mol_type = genomic DNA
attgaagagtttgatcatggctcagattgaacgctggcggcaggcctaacacatgcaagtcgagcggtagcacagagagcttgctctcgggtgacgagcggcggacgggtgagtaatgtctgggaaactgcctgatggagggggataactactggaaacggtagctaataccgcataacgtcgcaagaccaaagtgggggaccttcgggcctcatgccatcagatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttcgggttgtaaagcactttcagcggggaggaaggcggtgaggttaataacctcaccgattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgatttggaggttgtgcccttgaggcgtggcttccggagctaacgcgttaaatcgaccgcctggggagtacggccgcaaggttaaaactcaaatgaattgacgggggcccgcacaagcggtggagcatgtggtttaattcgatgcaacgcgaagaaccttacctggtcttgacatccacagaactttccagagatggattggtgccttcgggaactgtgagacaggtgctgcatggctgtcgtcagctcgtgttgtgaaatgttggg

SEQ ID NO: 13 - DNA, 882 bp, Klebsiella variicola
  gene 1..882: note = nifH1
  source 1..882: mol_type = genomic DNA
atgaccatgcgtcaatgcgctatctacggtaaaggcggtatcggtaaatccaccaccacccagaatctcgtcgcggccctcgccgagatgggtaagaaagtgatgatcgtcggctgcgatccgaaagcggattccacccgtctgatcctccacgctaaagcccagaacaccatcatggagatggcggcggaagtgggctcggtcgaggatctggagctcgaagacgttctgcaaatcggctatggcgatgtccgttgcgccgaatccggcggcccggagccaggcgtcggctgcgccggacgcggggtgatcaccgccatcaacttcctcgaggaagaaggcgcctatgaagaagatttggatttcgtcttctatgacgtcctcggcgacgtggtctgcggcggcttcgctatgccgatccgcgaaaacaaagcccaggagatctacatcgtctgctccggcgagatgatggcgatgtatgccgccaacaatatctccaaagggatcgtgaagtacgccaaatccggcaaggtgcgcctcggcggcctgatctgtaactcgcgcaaaaccgaccgggaagacgaactgatcatcgccctggcggagaagcttggcacgcagatgatccacttcgttccccgcgacaacattgtgcagcgcgcggagatccgccggatgacggtgatcgagtacgacccgacctgtcagcaggcgaatgaatatcgtcaactggcgcagaagatcgtcaataacaccaaaaaagtggtgccgacgccgtgcaccatggacgagctggaatcgctgctgatggagttcggcatcatggaagaagaagacaccagcatcattggtaaaaccgccgctgaagaaaacgcggcctga

SEQ ID NO: 14 - DNA, 1113 bp, Klebsiella variicola
  gene 1..1113: note = nifH2
  source 1..1113: mol_type = genomic DNA
atggttaggaaaagtagaagtaaaaatacaaatatagaactaactgaacatgaccatttattaataagtcaaataaaaaagcttaaaacacaaaccacttgcttttttaataataaaggaggggttgggaagactacattagtagcaaatttaggagcagagctatcaataaactttagtgcaaaagttcttattgtggatgccgaccctcaatgtaatctcacgcagtatgtattaagtgatgaagaaactcaggacttatatgggcaagaaaatccagatagtatttatacagtaataagaccactatcctttggtaaaggatatgaaagtgacctccctataaggcatgtagagaatttcggttttgacataattgtcggtgaccctagacttgctttacaggaagaccttttagctggagactggcgagatgccaaaggcggtgggatgcgaggaattaggacaacttttgtatttgcagagttaattaagaaagctcgtgagctaaattatgattttgttttctttgacatgggaccatcattaggcgcaatcaacagggcagtattactggcaatggaattctttgtcgtcccaatgtcaatcgatgtattttcactatgggctattaaaaatattggctccacggtttcaatatggaaaaaagaattagacacagggattcggctctcagaggaacctagcgaattatcacaattatcacctcaaggaaaactaaagtttctcggttacgtcacccaacaacataaagaacgctctggatacgatacaattcagcttgagaatactgaggaagaaataaaatcgaaacgtcgggtaaaggcgtatgaagacattggagaggtgtttccttctaaaattactgagcatctttctaaactttatgcatcaaaagatatgaacccacaccttggagatatacgtcatttaggtagtttagctccgaaatcacaatcacaacacgttccgatgatatcagtgtctggtacaggaaattacaccagacttagaaaaagcgcgcgtgaactttatcgagatattgcaagaagatacttagagaacattcagactgctaatggcgagaaatag

SEQ ID NO: 15 - DNA, 1374 bp, Klebsiella variicola
  gene 1..1374: note = nifD1
  source 1..1374: mol_type = genomic DNA
atgaagggaaaggaaattctggcgctgctggacgaacccgcctgcgagcacaaccagaagcaaaaatccggctgcagcgcccctaagcccggcgctaccgccggcggttgcgccttcgacggcgcgcagataacgctcctgcccatcgccgacgtcgcgcacctggtgcacggccccatcggctgcgcgggcagctcgtgggataaccgcggcagcgtcagcgccggcccggccctcaaccggctcggctttaccaccgatcttaacgaacaggatgtgattatgggccgcggcgaacgccgcctgttccacgccgtgcgtcacatcgtcgaccgctatcatccggcggcggtctttatctacaacacctgcgtaccggcgatggagggcgatgacatcgaggcggtctgccaggccgcacagaccgccaccggcgtcccggtcatcgctattgacgccgccggtttctacggcagtaaaaatcttggcaaccgaatggcgggcgacgtgatgctcaggcaggtgattggccagcgcgaaccggccccgtggccagacaacacgccctttgccccggcccagcgccacgatatcggcctgattggcgaattcaatatcgccggcgagttctggcaggtccagccgctgctcgacgagctggggatccgcgtcctcggcagcctctccggcgacggccgctttgccgagatccagaccctgcaccgggcgcaggccaatatgctggtgtgctcgcgcgcgctgatcaacgtcgcccgggggctggagctgcgctacggcacgccgtggtttgaaggcagcttctacgggatccgcgccacctccgacgccttgcgccagctggcgacgctgctgggggatgacgacctgcgccgccgcaccgaggcgctgatcgcccgcgaagagcaggcggcggagcaggctcttgcgccgtggcgtgagcagctccgcgggcgcaaagtgctgctctataccggcggcgtgaaatcctggtcggtggtatcggccctgcaggatctcggcatgaccgtggtggccaccggcacgcgcaaatccaccgaggaggacaaacagcggatccgtgagctgatgggcgacgaggcggtgatgcttgaggagggcaatgcccgcaccctgctcgacgtggtgtaccgctatcaggccgacctgatgatcgccggcggacgcaatatgtacaccgcctggaaagcccggctgccgtttctcgatatcaatcaggagcgcgagcacgcctacgccggctatcagggcatcatcaccctcgcccgccagctctgtctgaccctcgccagccccgtctggccgcaaacgcatacccgcgccccgtggcgctag

SEQ ID NO: 16 - DNA, 1449 bp, Klebsiella variicola
  gene 1..1449: note = nifD2
  source 1..1449: mol_type = genomic DNA
atgaccaacgcaacaggcgaacgtaaccttgcgctcatccaggaagtcctggaggtgtttcccgaaaccgcgcgcaaagagcgcagaaagcacatgatgatcagcgatccgcagatggagagcgtcggcaagtgcattatctcgaaccgtaaatcgcagcccggggtgatgaccgtgcgcggctgcgcctatgcgggctcgaaaggggtggtgtttgggccaatcaaagacatggcccatatctcgcacggccccatcggctgcggccagtattcccgcgccggacggcgcaactactataccggcgtcagcggtgtcgacagcttcggcaccctgaacttcacctctgattttcaggagcgcgatattgttttcggcggcgataaaaagctgaccaaactgatcgaagagatggagctgctgttcccgctgaccaaagggatcaccatccagtcggagtgcccggtgggcctgatcggcgatgacatcagcgccgtagccaacgccagcagcaaggcgctggataaaccggtgatcccggtgcgctgcgaaggctttcgcggcgtatcgcaatcgctgggccaccatatcgccaacgacgtggtgcgcgactgggtgctgaacaatcgcgaagggcagccgtttgccagcaccccgtacgatgttgccatcattggcgattacaacatcggcggcgacgcctgggcctcgcgcattctgctggaagagatggggctgcgcgtagtggcgcagtggtccggcgacggcaccctggtggagatggagaacaccccattcgttaagcttaacctcgtccactgctaccgttcgatgaactatatcgcccgccatatggaggagaaacatcagatcccatggatggaatataacttcttcggcccgaccaaaatcgccgaatcgctgcgcaagatcgccgatcaatttgatgacaccattcgcgccaatgcggaagcggtgatcgccaaatatgaggggcagatggcggccatcatcgccaaatatcgcccgcggctggaggggcgcaaagtgctgctgtacatgggggggctgcggccgcgccacgtcatcggcgcctatgaggatctcgggatggagatcatcgccgccggctacgagtttgcccataacgatgattacgaccgcaccctgccggacctgaaagagggcaccctgctgtttgacgatgccagcagctatgagctggaggccttcgtcaaagcgctgaaacctgacctcatcggctccgggatcaaagagaaatatatcttccagaaaatgggggtgccgttccgccagatgcactcctgggactattccggcccctatcacggctatgacggcttcgccatctttgcccgcgatatggatatgaccctgaacaatccggcgtggaacgaactgactgccccgtggctgaagtctgcgtga

SEQ ID NO: 17 - DNA, 1386 bp, Klebsiella variicola
  gene 1..1386: note = nifK1
  source 1..1386: mol_type = genomic DNA
atggcagatattatccgcagtgaaaaaccgctggcggtgagcccgattaaaaccgggcaaccgctcggggcgatcctcgccagcctcgggctggcccaggccatcccgctggtccacggcgcccagggctgcagcgccttcgccaaagttttctttattcagcatttccatgacccggtgccgctgcagtcgacggccatggatccgaccgccacgatcatgggggccgacggcaatatcttcaccgcgctcgacaccctctgccagcgccacagcccgcaggccatcgtgctgctcagcaccggtctggcggaagcgcagggcagcgatatcgcccgggtggtgcgccagtttcgcgaggcgcatccgcgccataacggcgtggcgatcctcaccgtcaataccccggatttttttggctctatggaaaacggctacagcgcggtgatcgagagcgtgatcgagcagtgggtcgcgccgacgccgcgtccggggcagcggccccggcgggtcaacctgctggtcagccacctctgttcgccaggggatatcgaatggctgggccgctgcgtggaggcctttggcctgcagccggtgatcctgccggacctctcgcagtcaatggatggccacctcggtgaaggggattttacgcccctgacccagggcggcgcctcgctgcgccagattgcccagatgggccagagtctgggcagcttcgccattggcgtgtcgctccagcgggcggcatcgctcctgacccaacgcagccgcggcgacgtgatcgccctgccgcatctgatgaccctcgaccattgcgatacctttatccatcagctggcgaagatgtccggacgccgcgtaccggcctggattgagcgccagcgtggccagctgcaggatgcgatgatcgactgccatatgtggcttcagggccagcgcatggcgatggcggcggagggcgacctgctggcggcgtggtgtgatttcgcccgcagccaggggatgcagcccggcccgctggtcgcccccaccagccaccccagcctgcgccagctgccggtcgagcaagtcgtgccgggggatcttgaggatctgcagcagctgctgagccaccaacccgccgatctgctggtggctaactctcacgcccgcgatctggcggagcagtttgccctgccgctgatccgcgtcggttttcccctcttcgaccggctcggtgagtttcgtcgcgtccgccaggggtacgccggtatgcgagatacgctgtttgaactggccaatctgctgcgcgaccgccatcaccacaccgccctctaccgctcgccgcttcgccagggcgccgacccccagccggcttcaggagacgcttatgccgcccattaa

SEQ ID NO: 18 - DNA, 1563 bp, Klebsiella variicola
  gene 1..1563: note = nifK2
  source 1..1563: mol_type = genomic DNA
atgagccaaacgatcgataaaattcacagctgttatccgctgtttgaacaggatgaataccagaccctgttccagaataaaaagacccttgaagaggcgcacgacgcgcagcgtgtgcaggaggtttttgcctggaccaccaccgccgagtatgaagcgctgaacttccagcgcgaggcgctgaccgtcgacccggccaaagcctgccagccgctcggcgccgtactctgcgcgctggggttcgccggcaccctgccctacgtgcacggctcccagggctgcgtcgcctattttcgcacctactttaaccgccattttaaagagccggtcgcctgcgtctccgactccatgaccgaggacgcggcggtgttcggcggcaacaacaacatgaatctgggcctgcagaatgccagcgcgctgtataaacccgagattatcgccgtctccaccacctgtatggccgaggtgatcggcgacgatctgcaggcgtttatcgccaacgccaaaaaagagggatttgttgacgaccgcatcgccattccttacgcccatacccccagctttatcggcagccatgtcaccggctgggacaatatgttcgaagggttcgcgaagacctttaccgctgactacgccgggcagccgggcaaacagcaaaagctcaatctggtgaccggatttgagacctatctcggcaacttccgcgtgctgaagcggatgatggcgcagatggatgtcccgtgcagcctgctctccgacccatcagaggtgctcgacacccccgccgacggccattaccggatgtacgccggcggcaccagccagcaggagatcaaaaccgcgccggacgccattgacaccctgctgctgcagccgtggcagctggtgaaaagcaaaaaggtggttcaggagatgtggaaccagcccgccaccgaggtggccgttccgctgggcctggccgccaccgacgcgctgctgatgaccgtcagtcagctgaccggcaaaccgatcgccgacgctctgaccctggagcgcggccggctggtcgacatgatgctggattcccacacctggctgcatggcaaaaaattcggcctctacggcgatccggatttcgtgatggggctgacgcgcttcctgctggagctgggctgcgagccgacggtgatcctcagtcataacgccaataaacgctggcaaaaagcgatgaagaaaatgctcgatgcctcgccgtacggtcaggaaagcgaagtgttcatcaactgcgacctgtggcacttccggtcgctgatgttcacccgtcagccggactttatgatcggtaactcctacggcaagtttatccagcgcgataccctggcaaagggcaaagccttcgaagtgccgctgatccgtctgggctttccgctgttcgaccgccatcatctgcaccgccagaccacctggggctatgaaggcgcaatgaacatcgtcacgacgctggtgaacgccgtgctggaaaaactggaccacgacaccagccagttgggcaaaaccgattacagcttcgacctcgttcgttaa

SEQ ID NO: 19 - DNA, 1485 bp, Klebsiella variicola
  gene 1..1485: note = nifL
  source 1..1485: mol_type = genomic DNA
atgaccctgaatatgatgctcgataacgccgcgccggaggccatcgccggcgcgctgactcaacaacatccggggctgttttttaccatggtggaacaggcctcggtggccatctccctcaccgatgccagcgccaggatcatttacgccaacccggcgttttgccgccagaccggctattcgctggcgcaattgttaaaccagaacccgcgcctgctggccagcagccagacgccgcgcgagatctatcaggagatgtggcataccctgctccagcgtcagccctggcgcggtcagctgattaatcagcgtcgggacggcggcctgtacctggtggagattgacatcaccccggtgcttagcccgcaaggggaactggagcattatctggcgatgcagcgggatatcagcgtcagctacaccctcgaacagcggctgcgcaaccatatgaccctgatggaggcggtgctgaataatatccccgccgccgtggtagtggtggacgagcaggatcgggtggtgatggacaacctcgcctacaaaaccttctgcgctgactgcggcggccgggagctgctcaccgagctgcaggtctcccctggccggatgacgcccggcgtggaggcgatcctgccggtggcgctgcgcggggccgcgcgctggctgtcggtaacctgctggccgttgcccggcgtcagtgaagaggccagccgctactttatcgacagcgcgctggcgcggaccctggtggtgatcgccgactgtacccagcagcgtcagcagcaggagcaagggcgccttgaccggctgaagcagcaaatgaccgccggcaagctgctggcggcgatccgcgagtcgctggacgccgcgctgatccagctgaactgcccgattaatatgctggcggcagcccgtcggctgaacggcgagggaagcgggaatgtggcgctggaggccgcctggcgtgaaggggaagaggcgatggcgcggctccagcgctgtcgcccatcgctggaactcgaaaaccccgccgtctggccgctgcagccctttttcgacgatctgtgcgccctctaccgtacacgcttcgatcccgacgggctgcaggtcgacatggcctcaccgcatctgatcggctttggccagcgcaccccactgctggcgtgcttaagcctgtggctcgatcgcaccctggccctcgccgccgaactcccctccgtgccgctggcgatgcagctctacgccgaggagaacgacggctggctgtcgctgtatctgactgacaacgtaccgctgctgcaggtgcgctacgctcactcccccgacgcgctgaactcgccgggcaaaggcatggagctgcggctgatccagaccctggtggcgcaccatcgcggggccattgagctggcttcccgaccgcagggcggcacctgcctgaccctgcgtttcccgctgtttaacaccctgaccggaggtgaagcatga

SEQ ID NO: 20 - DNA, 1575 bp, Klebsiella variicola
  gene 1..1575: note = nifA
  source 1..1575: mol_type = genomic DNA
atgatccctgaatccgacccggacaccaccgtcagacgcttcgacctctctcagcagttcaccgccatgcagcggataagcgtggtgctgagccgggccaccgaggccagcaaaacgctgcaggaggtgctcagcgtattacacaacgatgcctttatgcagcacgggatgatctgcctgtacgacagcgagcaggagatcctcagtatcgaagcgctgcagcaaaccggccagcagcccctccccggcagcacgcagatccgctatcgccccggcgagggactggtggggaccgtgctggcccaggggcagtcgctggtgctgccccgggtcgccgacgatcagcgttttctcgaccgcctgagcctctacgattacgatctgccgtttatcgccgtaccgttgatggggcccaacgcccggccaataggggtgctggcggcccagccgatggcgcgccaggaagagcggctgccggcctgcacccgttttctcgaaaccgtcgccaacctcgtcgcccagaccatccggctgatgatccttccggcctcacccgccctgtcgagccgccagccgccgaaggtggaacggccgccggcctgctcgtcgtcgcgcggcgtgggccttgacaatatggtcggcaagagcccggcgatgcgccagatcgtggaggtgatccgtcaggtttcgcgctgggacaccaccgtgctggtacgcggcgaaagcggcaccgggaaagagctgatcgccaacgccatccatcaccattcgccacgggctggcgccgccttcgtcaaatttaactgcgcggcgctgccggacaccctgctggaaagcgaactgttcggccatgagaaaggcgcctttaccggggcggtgcgtcagcgtaaaggacgttttgagctggcggatggcggcaccctgttcctcgatgagattggtgaaagcagcgcctcgttccaggccaagctgctgcgtatcctccaggagggggagatggagcgggtcggcggcgatgagaccctgcgggtgaatgtccgcatcatcgccgccaccaaccgtcacctggaggaggaggtccggctgggccatttccgcgaggatctctactatcgtctgaacgtgatgcccatcgccctgcccccgctgcgcgagcgtcaggaggacatcgccgagctggcgcacttcctggtgcgcaaaatcggccagcatcaggggcgcacgctgcggatcagcgagggcgcgatccgcctgctgatggagtacagctggccgggtaacgttcgcgaactggagaactgcctcgaacgatcggcggtgatgtcggagagtggcctgatcgatcgcgacgtgatcctcttcactcaccaggatcgtcccgccaaagccctgcctgccagcgggccagcggaagacagctggctggacaacagcctggacgaacgtcagcgactgatcgccgcgctggaaaaagccggctgggtgcaggccaaggcggcacggctgctggggatgacgccgcgccaggtcgcttatcggatccagatcatggatatcaccctgccgcgtctgtag

SEQ ID NO: 21 - DNA, 2838 bp, Klebsiella variicola
  gene 1..2838: note = glnE
  source 1..2838: mol_type = genomic DNA
atgatgccgctttctccgcaattacagcagcactggcagacggtcgctgaccgtctgccagcggattttcccattgccgaactgagcccacaggccaggtcggtcatggcgttcagcgattttgtcgaacagagtgtgatcgcccagccgggctggctgaatgagcttgcggactcctcgccggaggcggaagagtggcggcattacgaggcctggctgcaggatcgcctgcaggccgtcactgacgaagcggggttgatgcgagagctgcgtctcttccgccgccagatgatggtccgcatcgcctgggcgcaggcgctgtcgctggtgagcgaagaagagactctgcagcagctgagcgtcctggcggagaccctgattgtcgccgcccgcgactggctgtacgccgcctgctgtaaggagtggggaacgccatgcaatgccgagggccagccgcagccgctgctgatcctcgggatgggaaagctgggcggcggcgagctgaacttctcttccgatatcgatctgatctttgcctggcctgagcatggcgccacccgcggcggccgccgcgagctggataacgcccagttctttacccgtctggggcagcggctgatcaaggcccttgaccagccgacgcaggacggctttgtctatcgggttgacatgcgcctgcggccgtttggcgacagtgggccgctggtactcagttttgcggcgctggaagattattaccaggagcagggtcgggactgggaacgctatgcgatggtgaaagcgcggatcatgggcgataacgacggcgtgtacgccagcgagttgcgcgcgatgctccgtcctttcgtcttccgccgttatatcgacttcagcgtgatccagtcgctgcgtaacatgaaaggcatgatcgcccgcgaagtgcggcgtcgcgggctgaaagacaacatcaagctcggcgccggcgggatccgtgaaattgagtttatcgttcaggtctttcaactgatccgcggtggtcgcgaacctgcactgcagcagcgcgccctgctgccgacgctggcggcgattgatgagctacatctgctgccggaaggcgacgcggcgctgctgcgcgaggcctatctgttcctgcgccggctggaaaacctgctgcaaagcatcaacgatgagcagacccagaccctgccgcaggatgaacttaaccgcgccaggctggcgtgggggatgcataccgaagactgggagacgctgagcgcgcagctggcgagccagatggccaacgtgcggcgagtgtttaatgaactgatcggcgatgatgaggatcagtccccggatgagcaactggccgagtactggcgcgagctgtggcaggatgcgctggaagaagatgacgccagcccggcgctggcgcatttaaacgataccgaccgccgtagcgtgctggcgctgattgccgattttcgtaaagagctggatcggcgcaccatcggcccgcgcggccgccaggtgctggatcagctgatgccgcatctgctgagcgaaatctgctcgcgcgccgatgcgccgctgcctctggcgcggatcacgccgctgttgaccgggatcgtcacccgtaccacctatcttgagctgctgagcgaattccccggcgcgctgaagcacctgatcacgctctgcgcggcgtcgccgatggtcgccagccagctggcgcgccacccgctgctgctggatgagctgctggatcccaacaccctctatcagccgacggcgaccgatgcctatcgcgacgagctgcgccagtacctgctgcgcgtgccggaagaggatgaagagcagcagctggaggcgttgcgccagtttaagcaggcgcagcagctgcatatcgcggcggcggatatcgctggtaccctgccggtgatgaaggtcagcgatcacttaacctggcttgccgaagcgatcctcgacgcggtggtgcagcaggcatgggggcagatggtcgctcgctacggccagccgacccacctgcacgatcgccagggtcgcggcttcgccgtcgtcggctacggtaagcttggcggctgggagctgggctacagctccgatctcgatctggtgttcctccatgactgcccggcggaggtgatgaccgacggcgagcgggagattgacggccgtcagttctacctgcggctggcccagcggatcatgcacctgttcagcacccgcacctcgtccggtattctctacgaagtggacgcccggctgcgtccttctggcgcggcggggatgctggtcaccaccgccgacgcgtttgctgactatcagcagaacgaagcctggacgtgggaacatcaggcgctggtgcgcgcccgcgtggtctatggcgacccggcgctgcaggcgcgctttgacgccattcgtcgcgatatcctgaccaccccgcgggaggggatgaccctgcagaccgaggttcgcgagatgcgcgagaagatgcgcgcccaccttggcaacaaacatcccgatcgttttgatatcaaagccgatgccggcgggatcaccgatattgaatttattactcagtatctggtcctacgctatgccagtgacaagccgaagctgacccgctggtctgacaacgtgcgtattcttgagctgctggcgcagaacgacatcatggacgaggaggaggcgcgcgccttaacgcatgcgtacaccaccttgcgtgatgcgctccatcacctggccctgcaggagcagccgggacacgtggcgccagaggccttcagccgggagcgtcagcaggtcagcgccagctggcagaagtggctgatggcttaa

SEQ ID NO: 22 - DNA, 500 bp, Klebsiella variicola
  gene 1..500: note = PinfC
  source 1..500: mol_type = genomic DNA
agcgtcaggtaccggtcatgattcaccgtgcgattctcggttccctggagcgcttcattggcatcctgaccgaagagttcgctggcttcttcccaacctggattgcaccagtgcaggtagtggtcatgaatattaccgattctcaggctgaatacgttaacgaattgacgcgtaaactacaaaatgcgggcattcgtgtaaaagcagacttgagaaatgagaagattggctttaaaatccgcgagcacactttacgtcgtgtcccgtatatgttggtctgtggcgacaaagaagtcgaagccggcaaagtggccgtgcgcacccgtcgcgggaaagacctcggcagcatggacgtaagtgaagtgattgagaagctgcaacaagagattcgcagccgcagtcttcaacaactggaggaataaggtattaaaggcggaaaacgagttcaaacggcacgtccgaatcgtatcaatggcgagattcgcgccctggaagttcgc

SEQ ID NO: 23 - DNA, 1287 bp, Klebsiella variicola
  gene 1..1287: note = amtB
  source 1..1287: mol_type = genomic DNA
atgaaaatggcaacaatgaaatcgggtctgggggcattagcccttcttccgggactggcaatggccgcgcccgcagtggcggacaaagccgataacgcgtttatgatgatttgcaccgcgctggttctgtttatgaccatcccggggatcgcgctgttttacggcggcctgatccgcggcaaaaacgtcctttccatgctgactcaggtgattgtgacctttggcctggtctgcgtactgtgggtgatttatggctataccctggccttcggcaccggcggcagcttcttcggtagttttgactgggtgatgctgaaaaatattgaactgaaagcgctgatgggcaccttctatcagtacatccacgtggccttccagggctcgttcgcctgtatcaccgtcgggctgatcgtgggggcgctggctgagcgtattcgtttctccgccgtgctgatttttgtggtggtgtggatgacgctctcttatgttccgattgcgcacatggtctggggcggcggtctgctggcgacccacggcgcgctggacttcgcgggcggcaccgttgtacacatcaacgccgcggttgccgggctggtgggtgcgtacatgatgggcaaacgtgtgggcttcggcaaagaagcgttcaaaccgcacaatctgccgatggtgttcaccggaaccgccatcctctacgtgggctggttcggcttcaacgccggctccgccagcgcagcgaacgaaattgccgcattggctttcgtcaacaccgtcgtcgccacagcggctgccatcctggcgtggacctttggcgaatgggccctgcgcggtaaaccttcactgctgggcgcctgctccggggcgattgccggtctggttggcgtcacaccagcctgtgggtatatcggtgtcggtggggcgttgattgtgggtatcgcatctggtctggcgggcatctggggcgtaacggcgctgaaacgctggctgcgggttgatgacccttgcgacgtcttcggcgtccacggcgtctgcggcatcgtcggctgtatcctgaccggtatcttcgcggccacctctctgggcggcgtgggttatgcagaaggcgtcaccatgggccatcagctgctggtgcaactcgagagtatcgcgattaccatcgtctggtcgggcgttgtcgctttcattggctacaaagtggcggacatgaccgtggggctgcgcgtaccagaagagcaggagcgcgaaggactggacgtcaacagccatggcgaaaacgcctacaacgcctga

SEQ ID NO: 24 - DNA, 299 bp, Klebsiella variicola
  gene 1..299: note = Prm8.2
  source 1..299: mol_type = genomic DNA
cgccgtcctcgcagtaccattgcaaccgactttacagcaagaagtgattctggcacgcatggaacaaattcttgccagtcgggctttatccgatgacgaacgcgcacagcttttatatgagcgcggagtgttgtatgatagtctcggtctgagggcattagcgcgaaatgatttttcacaagcgctggcaatccgacccgatatgcctgaagtattcaattacttaggcatttacttaacgcaggcaggcaattttgatgctgcctatgaagcgtttgattctgtacttgagcttgatc

SEQ ID NO: 25 - DNA, 300 bp, Klebsiella variicola
  gene 1..300: note = Prm6.2
  source 1..300: mol_type = genomic DNA
gctaaagttctcggctaatcgctgataacatttgacgcaatgcgcaataaaagggcatcatttgatgccctttttgcacgctttcataccagaacctggctcatcagtgattttttttgtcataatcattgctgagacaggctctgaagagggcgtttatacaccaaaccattcgagcggtagcgcgacggcaagtcagcgttctcctttgcaatagcagggaagaggcgccagaaccgccagcgttgaagcagtttgaacgcgttcagtgtataatccgaaacttaatttcggtttgga

SEQ ID NO: 26 - DNA, 400 bp, Klebsiella variicola
  gene 1..400: note = Prm1.2
  source 1..400: mol_type = genomic DNA
gcccgctgaccgaccagaacttccaccttggactcggctatacccttggcgtgacggcgcgcgataactgggactacatccccattccggtgatcttaccattggcgtcaataggttacggtccggcgactttccagatgacctatattcccggcacctacaataacggtaacgtttacttcgcctgggctcgtatacagttttaattcgctaagtcttagcaataaatgagataagcggtgtgtcttgtggaaaaacaaggactaaagcgttacccactaaaaaagatagcgacttttatcactttttagcaaagttgcactggacaaaaggtaccacaattggtgtactgatactcgacacagcattagtgtcgatttttcatataaaggtaattttg

SEQ ID NO: 27 - DNA, 1536 bp, Klebsiella variicola
  gene 1..1536: note = 16S
  source 1..1536: mol_type = genomic DNA
ttgaagagtttgatcatggctcagattgaacgctggcggcaggcctaacacatgcaagtcgagcggtagcacagagagcttgctctcgggtgacgagcggcggacgggtgagtaatgtctgggaaactgcctgatggagggggataactactggaaacggtagctaataccgcataacgtcgcaagaccaaagtgggggaccttcgggcctcatgccatcagatgtgcccagatgggattagctngtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttcgggttgtaaagcactttcagcggggaggaaggcgntnaggttaataaccttgtcgattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgatttggaggttgtgcccttgaggcgtggcttccggagctaacgcgttaaatcgaccgcctggggagtacggccgcaaggttaaaactcaaatgaattgacgggggcccgcacaagcggtggagcatgtggtttaattcgatgcaacgcgaagaaccttacctggtcttgacatccacagaactttccagagatggattggtgccttcgggaactgtgagacaggtgctgcatggctgtcgtcagctcgtgttgtgaaatgttgggttaagtcccgcaacgagcgcaacccttatcctttgttgccagcggtnnggccgggaactcaaaggagactgccagtgataaactggaggaaggtggggatgacgtcaagtcatcatggcccttacgaccagggctacacacgtgctacaatggcatatacaaagagaagcgacctcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 28 - DNA, 882 bp, Klebsiella variicola
  gene 1..882: note = nifH
  source 1..882: mol_type = genomic DNA
atgaccatgcgtcaatgcgctatctacggtaaaggcggtatcggtaaatccaccaccacccagaatctcgtcgcggccctcgccgagatgggtaagaaagtgatgatcgtcggctgcgatccgaaagcggactccacccgtctgatccttcacgctaaagcccagaacaccatcatggagatggcggcggaagtgggctcggtcgaggatctggagctcgaagacgttctgcaaatcggctatggcgatgtccgttgcgccgaatccggcggcccggagccaggcgtcggctgcgccggacgcggggtgatcaccgccatcaacttcctcgaggaagaaggcgcctatgaggaagatttggatttcgtcttctatgacgtcctcggcgacgtagtctgcggcggcttcgccatgccgatccgcgaaaacaaagcccaggagatctacatcgtctgctccggcgagatgatggcgatgtatgccgccaacaatatctccaaggggatcgtgaagtacgcgaaatctggcaaggtgcgcctcggcggcctgatctgtaactcgcgcaaaaccgaccgggaagacgaactgatcatcgccctggcggagaagcttggcacgcagatgatccacttcgttccccgcgacaacattgtgcagcgcgcggagatccgccggatgacggtgatcgagtacgacccgacctgtcagcaggcgaatgaatatcgtcaactggcgcagaagatcgtcaataacaccaaaaaagtggtgccaacgccgtgcaccatggacgagctggaatcgctgctgatggagttcggcatcatggaagaagaagacaccagcatcattggtaaaaccgccgctgaagaaaacgcggcctga

SEQ ID NO: 29 - DNA, 1449 bp, Klebsiella variicola
  gene 1..1449: note = nifD1
  source 1..1449: mol_type = genomic DNA
atgaccaacgcaacaggcgaacgtaaccttgcgctcatccaggaagtcctggaggtgtttcccgaaaccgcgcgcaaagagcgcagaaagcacatgatgatcagcgatccgcagatggagagcgtcggcaagtgcattatctcgaaccgtaaatcgcagcccggggtgatgaccgtgcgtggctgcgcctatgcgggctcgaaaggggtggtgtttgggccaatcaaagacatggcccatatctcgcacggccccatcggctgcggccagtactcgcgcgccggacggcgcaactactataccggcgtcagcggtgtcgacagcttcggcaccctgaacttcacctctgattttcaggagcgcgatattgttttcggcggcgataaaaagctgaccaaactgatcgaagagatggagctgctgttcccgctgaccaaagggatcaccatccagtcggagtgcccggtgggcctgatcggcgatgacatcagcgccgtggccaacgccagcagcaaggcgctggataaaccggtgatcccggtgcgctgcgaaggctttcgcggcgtatcgcaatcgctgggccaccatatcgccaacgacgtggtgcgcgactgggtgctgaacaatcgcgaagggcagccgtttgccagcaccccgtatgatgttgccatcattggcgattacaacatcggcggcgacgcctgggcctcgcgcattctgctggaagagatggggctgcgcgtagtggcgcagtggtccggcgacggcaccctggtggagatggagaacaccccattcgttaagcttaacctcgtccactgctaccgttcgatgaactatatcgcccgccatatggaggagaaacatcagatcccgtggatggaatataacttcttcggcccgaccaaaatcgccgaatcgctgcgcaagatcgccgatcaatttgatgacaccattcgcgccaatgcggaagcggtgatcgccaaatatgaggggcagatggcggccatcatcgccaaatatcgcccgcggctggaggggcgcaaagtgctgctgtacatgggggggctgcggccgcgccacgtcatcggcgcctatgaggatctcgggatggagatcatcgccgccggctacgagtttgcccataacgatgattacgaccgcaccctgccggacctgaaagagggcaccctgctgtttgacgatgccagcagctatgagctggaggccttcgtcaaagcgctgaaacctgacctcatcggctccgggatcaaagagaaatatatcttccagaaaatgggggtgccgttccgccagatgcactcctgggactattccggcccctatcacggctatgacggcttcgccatctttgcccgcgatatggatatgaccctgaacaatccggcgtggaacgaactgactgccccgtggctgaagtctgcgtga

SEQ ID NO: 30 - DNA, 1374 bp, Klebsiella variicola
  gene 1..1374: note = nifD2
  source 1..1374: mol_type = genomic DNA
atgaagggaaaggaaattctggcgctgctggacgaacccgcctgcgagcacaaccagaagcaaaaatccggctgcagcgctcctaagcccggcgcaaccgccggcggctgcgccttcgacggcgcgcagataacgctcctgcccatcgccgacgtcgcgcacctggtgcacggccccatcggctgcgcgggcagctcgtgggataaccgcggcagcgtcagcgccggcccggccctcaaccggctcggctttaccaccgatcttaacgaacaggatgtgattatgggccgcggcgaacgccgcctgttccacgccgtccgtcacatcgtcgaccgctatcatccggcggcggtctttatctacaacacctgcgtaccggcgatggagggggatgacctggaggccgtctgccaggccgcacagaccgccaccggcgtcccggtcatcgccattgacgccgccggtttctacggcagtaaaaatcttggcaaccgaatggcgggcgacgtgatgctcaggcaggtgattggccagcgcgaaccggccccgtggccagacaacacgccctttgccccggcccagcgccacgatatcggcctgattggcgaattcaatatcgccggcgagttctggcaggtccagccgctgctcgacgagctggggatccgcgtcctcggcagcctctccggcgacggccgctttgccgagatccagaccctgcaccgggcgcaggccaatatgctggtgtgctcgcgcgcgctgatcaacgtcgcccgggggctggagctgcgctacggcacgccgtggtttgaaggcagcttctacgggatccgcgccacctccgacgccttgcgccagctggcggcgctgctgggggatgacgacctgtgccgccgcaccgaggcgctgatcgcccgcgaagagcaggcggcggagcaggcgctggcgccgtggcgcgagcagctccgtgggcgcaaagtgttgctctacaccggcggcgtgaaatcctggtcggtggtatcagccctgcaggatctcggcatgaccgtggtggccaccggcacgcggaaatccaccgaggaggacaaacagcggatccgtgagctgatgggcgacgaggcggtgatgcttgaggagggcaatgcccgcaccctgctcgacgtggtgtaccgctatcaggccgacctgatgatcgccggcggacgcaatatgtacaccgcctggaaagcccggctgccgtttctcgatatcaatcaggagcgcgagcacgcctacgccggctatcagggcatcatcaccctcgcccgccagctctgtctgaccctcgccagtcccgtctggccgcaaacgcatacccgcgccccgtggcgctag

SEQ ID NO: 31 - DNA, 1386 bp, Klebsiella variicola
  gene 1..1386: note = nifK1
  source 1..1386: mol_type = genomic DNA
atggcagacattatccgcagtgaaaaaccgctggcggtgagcccgattaaaaccgggcaaccgctcggggcgatcctcgccagcctcgggctggcccaggccatcccgctggtccacggcgcccagggctgcagcgccttcgccaaagttttctttattcagcatttccatgacccggtgccgctgcagtcgacggccatggatccgaccgccacgatcatgggggccgacggcaatatcttcaccgcgctcgacaccctctgccagcgccacagcccgcaggccatcgtgctgctcagcaccggtctggcggaagcgcagggcagcgatatcgcccgggtggtgcgccagtttcgtgaggcgcatccgcgccataacggcgtggcgatcctcaccgtcaataccccggatttttttggctcgatggaaaacggctacagcgcggtgatcgagagcgtgatcgagcagtgggtcgcgccgacgccgcgtccggggcagcggccccggcgggtcaacctgctggtcagccacctctgttcgccaggggatatcgaatggctgggccgctgcgtggaggcctttggcctgcagccggtgatcctgccggacctctcgcagtcaatggatggccacctcggtgaaggggattttacgcccctgacccagggcggcgcctcgctgcgccagattgcccagatgggccagagtctgggcagcttcgccattggcgtgtcgctccagcgggcggcatcgctcctgacccaacgcagccgcggcgacgtgatcgccctgccgcatctgatgaccctcgaccattgcgatacctttatccatcagctggcgaagatgtccggacgccgcgtaccggcctggattgagcgccagcgcggccagctgcaggatgcgatgatcgactgccatatgtggcttcagggccagcgcatggcgatggcggcggagggcgacctgctggcggcgtggtgtgatttcgcccgcagccaggggatgcagcccggcccgctggtcgcccccaccagccaccccagcctgcgccagctgccggtcgatcaggtcgtgccgggggatcttgaggatctgcagcagctgctgagccaccaacccgccgatctgctggtggctaactctcacgcccgcgatctggcggagcagtttgccctgccgctgatccgcgtcggttttcccctcttcgaccggctcggtgagtttcgtcgcgtccgccaggggtacgccggtatgcgagatacgctgtttgagctggccaatctgctgcgcgaccgccatcaccacaccgccctctaccgctcgccgcttcgccagggcgccgaccccctgccggcttcaggagacgcttatgccgcccattaa

SEQ ID NO: 32 - DNA, 183 bp, Klebsiella variicola
  gene 1..183: note = nifK2
  source 1..183: mol_type = genomic DNA
gtgccgctgatccgtctgggctttccgctgttcgaccgccatcatctgcaccgccagaccacctggggctatgaaggcgcaatgaacatcgtcacgacgctggtgaacgccgtgctggaaaaactggaccacgacaccagccagttgggcaaaaccgattacagcttcgacctcgttcgttaa

SEQ ID NO: 33 - DNA, 1485 bp, Klebsiella variicola
  gene 1..1485: note = nifL
  source 1..1485: mol_type = genomic DNA
atgaccctgaatatgatgctcgataacgccgcaccggaggccatcgccggcgcgctgactcaacaacatccggggctgttttttaccatggtggaacaggcctcggtggccatatccctcaccgatgccagcgccaggatcatttacgccaacccagcgttttgccgccagaccggctattcgctggcgcaattgttaaaccagaacccgcgcctgctggccagcagccagacgccgcgcgcgatctatcaggagatgtggcataccctgctccagcgtcagccctggcgcggtcagctgattaatcagcgtcgggacggcggcctgtgcctggtggagattgacatcaccccggtgcttagcccgcaaggggaactggagcattatctggcgatgcagcgggatatcagcgtcagctacaccctcgaacaacggctgcgcaaccatatgaccctgatggaggcggtgctgaataatatccccgccgccgtggtggtggtggacgagcaggatcgggtggtgatggacaacctcgcctacaaaaccttctgcgctgactgcggcggccgggagctgctcaccgagctgcaggtctcccctggccggatgacgcccggcgtggaggcgatcctgccggtagcgctgcgcggggccgcgcgctggctgtcggtaacctgctggccgttgcccggcgtcagtgaagaggccagccgctactttatcgacagcgcgctggcgcggaccctggtggtgatcgccgactgtacccagcagcgtcagcagcaggagcaaggacgccttgaccggctgaagcagcaaatgaccgccggcaagctgctggcggcgatccgcgagtcgctggacgccgcgctgatccagctgaactgcccgattaatatgctggcggcagcccgtcggctgaacggcgagggaagcgggaatgtggcgctggaggccgcctggcgtgaaggggaagaggcgatggcgcggctccagcgctgtcgcccatcgctggaactcgaaaaccccgccgtctggccgctgcagccctttttcgacgatctgtgcgccctctaccgtacccgcttcgatcccgacgggctgcaggtcgacatggcctcaccgcatctgatcggctttggccagcgcaccccgctgctggcgtgcttaagcctgtggctcgaccgcaccctggccctcgccgccgaattgccctccgtgccgctggcgatgcagctctatgccgaggagaacgacggctggctgtcgctgtacctgactgataacgtaccgctgttgcaggtgcgctacgcccactcccccgacgcgctgaactcgccgggtaaaggcatggagctgcggctgatccagaccctggtggcgcaccatcgcggggccattgagctggcttcccgaccgcagggcggcacctgcctgaccctgcgtttcccgctgtttaacaccctgaccggaggtgaagcatga

SEQ ID NO: 34 - DNA, 1575 bp, Klebsiella variicola
  gene 1..1575: note = nifA
  source 1..1575: mol_type = genomic DNA
atgatccctgaatccgacccggacaccaccgtcagacgcttcgacctctctcagcagttcaccgccatgcagcggataagcgtggtgctgagccgggccaccgaggccagcaaaacgctgcaggaggtactcactgtattgcacaacgatgcctttatgcagcacgggatgatctgcctgtacgacagcgagcaggagatcctcagtatcgaagcgctgcagcaaaccggccagcagcccctccccggcagcacgcagatccgctatcgccccggcgagggactggtggggaccgtgctggcccaggggcagtcgctggtgctgccccgggtcgccgacgatcagcgttttctcgaccgcctgagcctctacgattacgatctgccgtttatcgccgtaccgttgatggggcccaacgcccggccaataggggtgctggcggcccagccgatggcgcgccaggaagagcggctgccggcctgcacccgttttctcgaaaccgtcgccaacctcgtcgcccagaccatccggctgatgatccttccggcctcacccgccctgtcgagccgccagccgccgaaggtggaacggccgccggcctgctcgtcgtcgcgcggcgtgggccttgacaatatggtcggcaagagcccggcgatgcgccagatcgtggaggtgatccgtcaggtttcgcgctgggacaccaccgtgctggtgcgcggtgaaagcggcaccgggaaagagctgatcgccaacgccatccatcaccattcgccacgggctggcgccgccttcgtcaaatttaactgcgcggcgctgccggacaccctgctggaaagcgaactgttcggccatgagaaaggcgcctttaccggggcggtgcgtcagcgtaaaggacgttttgagctggcggatggcggcaccctgttcctcgatgagattggtgaaagcagcgcctcgttccaggccaagctgctgcgtatcctccaggagggggagatggagcgggtcggcggcgatgagaccctgcgggtgaatgtccgcatcatcgccgccaccaaccgtcacctggaggaggaggtccggctgggccatttccgcgaggatctctattatcgtctgaacgtgatgcccatcgccctgcccccgctgcgcgagcgtcaggaggacatcgccgagctggcgcacttcctggtgcgcaaaatcggccagcatcaggggcgcacgctgcggatcagcgagggcgcgatccgcctgctgatggagtacagctggccgggtaacgttcgcgaactggagaactgcctcgaacgatcggcggtgatgtcggagagtggcctgatcgatcgcgacgtgatcctcttcactcaccaggatcgtcccgccaaagccctgcctgccagcgggccagcggaagacagctggctggacaacagcctggacgaacgtcagcgactgatcgccgcgctggaaaaagccggctgggtgcaggccaaggcggcacggctgctggggatgacgccgcgccaggtcgcttaccggatccagatcatggatatcaccctgccgcgtctgtag

SEQ ID NO: 35 - DNA, 2838 bp, Klebsiella variicola
  gene 1..2838: note = glnE
  source 1..2838: mol_type = genomic DNA
atgatgccgctttctccgcaattacagcagcactggcagacggtcgctgaccgtctgccagcggattttcccattgcagaactgagcccacaggccaggtcggtcatggcgttcagcgattttgtcgaacagagtgtgatcgcccagccgggctggctgaatgagcttgcggactcctcgccggaggcggaagagtggcggcattacgaggcctggctgcaggatcgcctgcaggccgtcactgacgaagcggggttgatgcgagagctgcgtctcttccgccgccagatgatggtccgcatcgcctgggcgcaggcgctgtcgctggtgagcgaagaagagaccctgcagcagctgagcgccctggcggagaccctgattgtcgccgcccgcgactggctctacgccgcctgctgtaaggagtggggaacgccatgcaatgccgagggccagccgcagccgctgctgatcctcgggatgggaaagctgggcggcggcgagctgaacttctcttccgatatcgatctgatctttgcctggcctgagcatggcgccacccgcggcggccgccgcgagctggataacgcccagttctttacccgtctggggcagcggctgatcaaggcccttgaccagccgacgcaggacggctttgtctatcgggttgacatgcgcctgcggccgtttggcgacagtgggccgctggtactcagctttgcggcactggaagattattaccaggagcagggtcgggactgggaacgctatgcgatggtgaaagcgcggatcatgggcgataacgacggcgtgtacgccagcgagttgcgcgcgatgctccgtcctttcgtcttccgccgttatatcgacttcagcgtgatccagtcgctgcgtaacatgaaaggcatgatcgcccgcgaagtgcggcgtcgcgggctgaaagacaacatcaagctcggcgccggcgggatccgtgaaattgagtttatcgttcaggtctttcagctgatccgcggtggtcgcgaacctgcactgcagcagcgcgccctgctgccgacgctggcggcgattgatgagctacatctgctgccggaaggcgacgcggcgctgctgcgcgaggcctatctgttcctgcgccggctggaaaacctgctgcaaagcatcaacgatgaacagacccagaccctgccgcaggatgaacttaaccgcgccaggctggcgtgggggatgcataccgaagactgggagacgctgagcgcgcagctggcgagccagatggccaacgtgcggcgagtgtttaatgaactgatcggcgatgatgaggatcagtccccggatgagcaactggccgagtactggcgcgagctgtggcaggatgcgctggaagaagatgacgccagcccggcgctggcgcatttaaacgataccgaccgccgtagcgtgctggcgctgattgccgattttcgtaaagagctggatcggcgcaccatcggcccgcgcggccgccaggtgctggatcagctgatgccgcatctgctgagcgaaatctgctcgcgtgccgatgcgccgctgcctctggcgcggatcacgccgctgttgaccgggatcgtcacccgtaccacctatcttgagctgctgagcgaattccccggcgcgctgaagcacctgatcacgctctgcgcggcgtcgccgatggtcgccagccagctggcgcgccacccgctgctgctggatgagctgctggatcccaacaccctctatcagccgacggcgaccgatgcctatcgcgacgagctgcgccagtacctgctgcgcgtgccggaagaggatgaagagcagcagctggaggcgttgcgccagtttaagcaggcgcagcagctgcatatcgcggcggcggatatcgctggtaccctgccggtgatgaaggtcagcgatcacttaacctggcttgccgaagcgatcctcgacgcggtggtgcagcaggcatgggggcagatggtcgctcgctacggtcagccgacccacctgcacgatcgccagggtcgcggcttcgccgttgtcggctacggtaagctcggcggctgggagctgggctacagctccgatctcgatctggtgttcctccatgactgcccggcggaggtgatgaccgacggcgagcgggagattgacggccgtcagttctacctgcggctggcccagcggatcatgcacctgttcagcacccgcacctcgtccggtattctctacgaagtggacgcccggctgcgtccttctggcgcggcggggatgctggtcaccaccgccgacgcgtttgctgactatcagcagaacgaagcctggacgtgggaacatcaggcgctggtgcgcgcccgcgtggtctatggcgacccggcgctgcaggcgcgctttgacgccattcgtcgcgatatcctgaccaccccgcgggaggggacgaccctgcagaccgaggttcgcgagatgcgcgagaagatgcgcgcccaccttggcaacaaacatcccgatcgttttgatatcaaagccgatgccggcgggatcaccgatattgaatttattactcagtatctggtcctacgctatgccagtgacaagccgaagctgacccgctggtctgacaacgtgcgtattcttgagctgctggcgcagaacgacatcatggacgaggaggaggcgcgcgccttaacgcatgcatacaccaccttgcgtgatgcgctccatcacctggccctgcaggagcagccgggacacgtggcgccagaggccttcagccgggagcgtcagcaggtcagcgccagctggcagaagtggctgatggcttaa

SEQ ID NO: 36 - DNA, 1287 bp, Klebsiella variicola
  gene 1..1287: note = amtB
  source 1..1287: mol_type = genomic DNA
atgaaaatggcaacaatgaaatcgggtctgggggcattagcccttcttccgggactggcaatggccgcgcccgcagtggcggacaaagccgataacgcgtttatgatgatttgcaccgcgctggttctgtttatgaccatcccggggatcgcgctgttttacggcggcctgatccgcggcaaaaacgtcctttccatgctgactcaggtgattgtgacctttggcctggtctgcgtactgtgggtgatttatggctataccctggccttcggcaccggcggcagcttcttcggtagctttgactgggtgatgctgaaaaatattgaactgaaagcgctgatgggcaccttctatcagtacatccacgtggccttccagggctcgttcgcctgtatcaccgtcgggctgatcgtgggggcgctggctgagcgtattcgtttctccgccgtgctgattttcgtggtggtgtggatgacgctctcttatgttccgattgcgcacatggtctggggcggcggtctgctggcgacccacggcgcgctggacttcgcgggcggcaccgttgtacacatcaacgccgcggttgccgggctggtgggtgcgtatatgatgggcaaacgtgtgggcttcggcaaagaagcgttcaaaccgcacaatctgccgatggtgttcaccggaaccgccatcctctacgtgggctggttcggcttcaacgccggctccgccagcgcagcgaacgaaattgccgcactggctttcgtcaacaccgtcgtcgccacagcggcagccatcctggcctggacctttggcgaatgggctctgcgcggcaaaccttcactgctgggcgcctgctccggggcgattgccggtctggttggcgtcacaccagcctgtgggtatatcggtgtcggtggggcgttgattgtgggtatcgcatctggtctggcgggcatctggggcgtaacggcgctgaaacgctggctgcgggttgatgacccttgcgacgtcttcggcgtccacggcgtctgcggcatcgtcggctgtatcctgaccggtatcttcgcggccacctctctgggcggcgtgggttatgcagaaggcgtcaccatgggccatcagctgctggtgcaactcgagagtatcgcgattaccatcgtctggtcgggcgttgtcgctttcattggctacaaagtggcggacatgaccgtggggctgcgcgtaccagaagagcaggagcgcgaaggactggacgtcaacagccatggcgaaaacgcctacaacgcctga

SEQ ID NO: 37 - DNA, 1435 bp, Achromobacter spiritinus
  gene 1..1435: note = 16S
  source 1..1435: mol_type = genomic DNA
ctgaagagtttgatcctggctcagattgaacgctagcgggatgccttacacatgcaagtcgaacggcagcacggacttcggtctggtggcgagtggcgaacgggtgagtaatgtatcggaacgtgcctagtagcgggggataactacgcgaaagcgtagctaataccgcatacgccctacgggggaaagcaggggatcgcaagaccttgcactattagagcggccgatatcggattagctagttggtggggtaanggctcaccaaggcgacgatccgtagctggtttgagaggacgaccagccacactgggactgagacacggcccagactcctacgggaggcagcagtggggaattttggacaatgggggaaaccctgatccagccatcccgcgtgtgcgatgaaggccttcgggttgtaaagcacttttggcaggaaagaaacgtcatgggntaataccccgtgaaactgacggtacctgcagaataagcaccggctaactacgtgccagcagccgcggtaatacgtagggtgcaagcgttaatcggaattactgggcgtaaagcgtgcgcaggcggttcggaaagaaagatgtgaaatcccagagcttaactttggaactgcatttttaactaccgggctagagtgtgtcagagggaggtggaattccgcgtgtagcagtgaaatgcgtagatatgcggaggaacaccgatggcgaaggcagcctcctgggataacactgacgctcatgcacgaaagcgtggggagcaaacaggattagataccctggtagtccacgccctaaacgatgtcaactagctgttggggccttcgggccttagtagcgcagctaacgcgtgaagttgaccgcctggggagtacggtcgcaagattaaaactcaaaggaattgacggggacccgcacaagcggtggatgatgtggattaattcgatgcaacgcgaaaaaccttacctacccttgacatgtctggaattctgaagagattcggaagtgctcgcaagagaaccggaacacaggtgctgcatggctgtcgtcagctcgtgtcgtgagatgttgggttaagtcccgcaacgagcgcaacccttgtcattagttgctacgaaagggcactctaatgagactgccggtgacaaaccggaggaaggtggggatgacgtcaagtcctcatggcccttatgggtagggcttcacacgtcatacaatggtcgggacagagggtcgccaacccgcgagggggagccaatcccagaaacccgatcgtagtccggatcgcagtctgcaactcgactgcgtgaagtcggaatcgctagtaatcgcggatcagcatgtcgcggtgaatacgttcccgggtcttgtacacaccgcccgtcacaccatgggagtgggttttaccagaagtagttagcctaacc

SEQ ID NO: 38 - DNA, 1528 bp, Achromobacter marplatensis
  gene 1..1528: note = 16S
  source 1..1528: mol_type = genomic DNA
ctgaagagtttgatcctggctcagattgaacgctagcgggatgccttacacatgcaagtcgaacggcagcacggacttcggtctggtggcgagtggcgaacgggtgagtaatgtatcggaacgtgcctagtagcgggggataactacgcgaaagcgtagctaataccgcatacgccctacgggggaaagcaggggatcgcaagaccttgcactattagagcggccgatatcggattagctagttggtggggtaanggctcaccaaggcgacgatccgtagctggtttgagaggacgaccagccacactgggactgagacacggcccagactcctacgggaggcagcagtggggaattttggacaatgggggaaaccctgatccagccatcccgcgtgtgcgatgaaggccttcgggttgtaaagcacttttggcaggaaagaaacgtcatgggttaataccccgtgaaactgacggtacctgcagaataagcaccggctaactacgtgccagcagccgcggtaatacgtagggtgcaagcgttaatcggaattactgggcgtaaagcgtgcgcaggcggttcggaaagaaagatgtgaaatcccagagcttaactttggaactgcatttttaactaccgggctagagtgtgtcagagggaggtggaattccgcgtgtagcagtgaaatgcgtagatatgcggaggaacaccgatggcgaaggcagcctcctgggataacactgacgctcatgcacgaaagcgtggggagcaaacaggattagataccctggtagtccacgccctaaacgatgtcaactagctgttggggccttcgggccttagtagcgcagctaacgcgtgaagttgaccgcctggggagtacggtcgcaagattaaaactcaaaggaattgacggggacccgcacaagcggtggatgatgtggattaattcgatgcaacgcgaaaaaccttacctacccttgacatgtctggaattcngaagagattnggaagtgctcgcaagagaaccggaacacaggtgctgcatggctgtcgtcagctcgtgtcgtgagatgttgggttaagtcccgcaacgagcgcaacccttgtcattagttgctacgaaagggcactctaatgagactgccggtgacaaaccggaggaaggtggggatgacgtcaagtcctcatggcccttatgggtagggcttcacacgtcatacaatggtcgggacagagggtcgccaacccgcgagggggagccaatcccagaaacccgatcgtagtccggatcgcagtctgcaactcgactgcgtgaagtcggaatcgctagtaatcgcggatcagcatgtcgcggtgaatacgttcccgggtcttgtacacaccgcccgtcacaccatgggagtgggttttaccagaagtagttagcctaaccgnaaggggggcgattaccacggtaggattcatgactggggtgaagtcgtaacaaggtagccgtatcggaaggtgcggctggatcacctccttt

SEQ ID NO: 39 - DNA, 1522 bp, Microbacterium murale
  gene 1..1522: note = 16S
  source 1..1522: mol_type = genomic DNA
tacggagagtttgatcctggctcaggatgaacgctggcggcgtgcttaacacatgcaagtcgaacggtgaacacggagcttgctctgtgggatcagtggcgaacgggtgagtaacacgtgagcaacctgcccctgactctgggataagcgctggaaacggcgtctaatactggatatgtgacgtggccgcatggtctgcgtctggaaagaatttcggttggggatgggctcgcggcctatcagcttgttggtgaggtaatggctcaccaaggcgtcgacgggtagccggcctgagagggtgaccggccacactgggactgagacacggcccagactcctacgggaggcagcagtggggaatattgcacaatgggcgcaagcctgatgcagcaacgccgcgtgagggatgacggccttcgggttgtaaacctcttttagcagggaagaagcgaaagtgacggtacctgcagaaaaagcgccggctaactacgtgccagcagccgcggtaatacgtagggcgcaagcgttatccggaattattgggcgtaaagagctcgtaggcggtttgtcgcgtctgctgtgaaatccggaggctcaacctccggcctgcagtgggtacgggcagactagagtgcggtaggggagattggaattcctggtgtagcggtggaatgcgcagatatcaggaggaacaccgatggcgaaggcagatctctgggccgtaactgacgctgaggagcgaaagggtggggagcaaacaggcttagataccctggtagtccaccccgtaaacgttgggaactagttgtggggtccattccacggattccgtgacgcagctaacgcattaagttccccgcctggggagtacggccgcaaggctaaaactcaaaggaattgacggggacccgcacaagcggcggagcatgcggattaattcgatgcaacgcgaagaaccttaccaaggcttgacatatacgagaacgggccagaaatggtcaactctttggacactcgtaaacaggtggtgcatggttgtcgtcagctcgtgtcgtgagatgttgggttaagtcccgcaacgagcgcaaccctcgttctatgttgccagcacgtaatggtgggaactcatgggatactgccggggtcaactcggaggaaggtggggatgacgtcaaatcatcatgccccttatgtcttgggcttcacgcatgctacaatggccggtacaaagggctgcaataccgcgaggtggagcgaatcccaaaaagccggtcccagttcggattgaggtctgcaactcgacctcatgaagtcggagtcgctagtaatcgcagatcagcaacgctgcggtgaatacgttcccgggtcttgtacacaccgcccgtcaagtcatgaaagtcggtaacacctgaagccggtggcctaacccttgtggagggagccgtcgaaggtgggatcggtaattaggactaagtcgtaacaaggtagccgtaccggaaggtgcggctggatcacctccttt

SEQ ID NO: 40 - DNA, 1537 bp, Kluyvera intermedia
  gene 1..1537: note = 16S
  source 1..1537: mol_type = genomic DNA
attgaagagtttgatcatggctcagattgaacgctggcggcaggcctaacacatgcaagtcgaacggtagcacagagagcttgctctcgggtgacgagtggcggacgggtgagtaatgtctgggaaactgcccgatggagggggataactactggaaacggtagctaataccgcataatgtcgcaagaccaaagagggggaccttcgggcctcttgccatcggatgtgcccagatgggattagcttgttggtgaggtaatggctcaccaaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttcgggttgtaaagcactttcagcggggaggaaggcgatncggttaataaccgtgttgattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggcttgagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaattnggcagagatgccttagtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcatatacaaagagaagcgacctcgcgagagcaagcggacctcataaagtatgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 41 - DNA, 882 bp, Kluyvera intermedia
  gene 1..882: note = nifH
  source 1..882: mol_type = genomic DNA
atgaccatgcgtcaatgcgccatttatggcaaaggtgggatcggcaaatccaccaccacgcaaaacctcgtcgccgctctcgcggaaatgggtaaaaaagtgatgatcgtcggctgcgacccgaaagcggactccacccgtctgatcctgcatgcgaaagcacagaacaccattatggagatggccgccgaagtgggttcagtggaagaccttgaactggaagatgtgctgcaaatcggttacggcggcgtgcgttgtgcagaatccggcggcccggagccaggcgtgggttgtgcaggccgcggcgttattaccgccattaacttccttgaagaagaaggcgcctatgtcagcgacctcgactttgtcttctatgacgtcctcggtgacgtggtctgcggcgggttcgccatgccgattcgtgaaaacaaagcgcaagagatctatatcgtctgctccggggaaatgatggcgatgtatgccgctaacaacatctccaaaggcatcgtgaaatacgctaaatccggcaaggtgcgcctgggcgggctgatttgtaactcccgtcagaccgaccgcgaagatgaactgatcatcgcgctggcagaaaaactgggcacccagatgattcactttgtgccacgcgacaacatcgtccagcgcgcggaaattcgccgtatgacggttatcgaatatgacccgaaatgcaaccaggccgacgaataccgcgcgctggcgaacaagatcgtcaacaacaccctgatggtcgtcccgaccccttgcaccatggatgaactggaagagctgctgatggaattcggcattatggatgtggaagacgccagcatcatcggtaaaaccgccgccgaagaaaacgcggcctga

SEQ ID NO: 42 - DNA, 660 bp, Kluyvera intermedia
  misc_feature 1..660: note = Dinitrogenase iron-molybdenum cofactor coding sequence
  source 1..660: mol_type = genomic DNA
atgaacgataacgatgtccttttctggcgcatgctggcgctatttcagtgtctgccggaactgcaacccgcgcagatcctggcctggctgacaggagaacgcgacgacgccttaaccccggcgtacctcgataagcttaacgtccgcgaactggaagcgaccttcccgtctgaaacggcgatgatgtcgcccgcacgctggagccgcgttaacgcgtgccttcacggtacgctgcccgcacacctgcaggtaaaaagcaccactcgtcaggggcaattacgggtagccttttgttcacaggatggattgctgatcaatggtcattttggtcaggggcggctgttttttatctacgcctttgatgaacagggcggatggctacacgcgttacgccgtcttccctcggccccgcaaacccaggagccgaatgaagttcgcgcgcagctcctgagtgattgccacctgctgttttgtgaagccattggcggccctgcggcggcccggctgattcgtcacaatatccacccgatgaaagtgtcgccagggatgtccattgccgcccagtgtgatgccattaccgcactgctgagcggacgtctgccaccgtggctggcaaaacgtcttgagaaagccaacccgctggaagagcgggtgttttaa

SEQ ID NO: 43 - DNA, 1374 bp, Kluyvera intermedia
  gene 1..1374: note = nifD1
  source 1..1374: mol_type = genomic DNA
atgaagggaaatgacattctcgcgctgctggatgaacccgcctgcgaacacaatcacaaacagaaatccggctgtagcgcccctaaacccggtgccacggcgggcggttgcgcgttcgacggcgcgcaaatcaccctgttgccgctgtcggatgtggcgcacctggtccacggaccgattggctgcacgggaagctcctgggataaccggggcagtatgagctccggccccagtctcaaccggctcggctttaccaccgacctgaacgagcaggatgtcattatggggcgcggcgaacggcggcttttccacgcggtgcgtcatatcgtcaaccgttatcaccctgccgccgtgtttatctataacacctgcgttccggcgatggagggtgatgatattgacgccgtctgtcaggcggcggaaaccgccaccggcgtgccagtgattgccgttgatgccgccgggttctatggcagcaaaaaccttggcaaccgtctcgcgggtgaagtgatggttaacaaggtcattggacggcgcccgcccgccccctggccggacgatacccccttcgcgccggaacaccgccacgatatcggcctgattggcgaatttaatatcgccggggagttctggcacgttcagccgctgctcgatgagctgggtattcgcgtgctgggcagcctttccggggatggccgttttagtgaaatccagaccctgcaccacgcgcaggtcaatatgctggtctgctcaagagcgctgatcaatgttgcccgcaccctggaacagcgctatggcaccccctggtttgagggcagtttttacggcgtgcgcgctacctccgatgccctgcgtcaactggcatccctgcttggcgacagcgatctgattgcccgcaccgaagccgttattgcccgcgaagaagccacggcaaatcaggcgctcgccccgtggcgcgaacggctacagggtcgcaaagtgctgctctataccggtggggtgaaatcctggtcggtggtctccgcattgcaggatttagggatgaccgtcgtggcgactggcacccgcaaatctaccgaagaagataagcagcgtattcgcgaattaatgggcgatgacgcgctaatgctggaagaaggcaacgcccgcaccctgctggatgtggtgtaccgctatcaggcggatttgatgatcgctggggggcgtaacatgtataccgcgtacaaagcgcggctgccgtttctggatatcaaccaggagcgtgaacacgcctttgcgggttatcgcggcatcgtcaccctcgcccaacagctttgccagactattgaaagccccgtctggccgcaaacacacgcccgcgcgccgtggcaataa

SEQ ID NO: 44 - DNA, 1449 bp, Kluyvera intermedia
  gene 1..1449: note = nifD2
  source 1..1449: mol_type = genomic DNA
atgagcaatgcaacaggcgaacgtaatctggaaattatccaggaagtgctggagatctttcccgaaaaaacgcgcaaagaacgcagaaagcacatgatggtgaccgacccggagatggaaagcgtcgggaaatgcatcatctctaaccgcaaatcgcagccgggtgtgatgactgtccgcggctgctcctacgccgggtcgaaaggcgtggtttttgggccgattaaagatatggcccacatctcccacggcccgatcggctgtgggcagtactcccgtgccgggcggcgcaactactacaccggggtcagcggcgttgattccttcgggacgctgaactttacctctgattttcaggagcgcgatatcgtcttcggcggcgataaaaagctcaccaaactgattgaggagatggaggaactgttcccgctgaccaaaggcatctccattcagtcggagtgcccggtaggtttaatcggtgacgatatcgaagcggtggcgaatgccagtaaaaaagcgctcaacaagccggtgatcccggtgcgttgcgaaggctttcgcggcgtgtcgcagtcgctcggtcaccatatcgccaacgacgttatccgcgactgggtgctggataaccgcgaagggaagcccttcgaatctaccccctatgacgtggccatcatcggcgattacaacatcgggggggatgcctgggcgtcgcgcattctgcttgaagagatggggttacgcgtggtggcgcagtggtccggtgacggcacgctggtagagatggaaaacaccccgttcgtcaagctgaacctggtgcactgctaccgctctatgaactacatctctcgccatatggaagagaaacacggtatcccgtggatggagtacaacttcttcggcccgaccaaaatcgccgaatcgctgcgtaagatcgccgatcaatttgacgacaccatccgcgccaatgcggaagcggtgatcgccaaatatcaggcgcaaaacgatgcgattatcgccaaataccgcccgcgtctcgaaggccgcaaggtgctgctctatatgggtggcctgcgtcctcgccacgtgattggcgcgtatgaggatttgggcatggagattgtcgccgccgggtatgaatttgcccataacgacgattacgaccgcaccctgccggacctcaaagagggcacgctgttgttcgacgatgccagcagttatgaactggaagccttcgtgaaggcgattaagccggacctcattggctcaggcatcaaggaaaaatacattttccagaaaatgggggtaccgtttcgccagatgcactcctgggattactccggcccgtatcacggctatgacggctttgccatctttgcccgcgatatggacatgacgctcaacaatcccgcctggggcgagttgaccgcaccctggctgaaatcagcctga

SEQ ID NO: 45 - DNA, 1383 bp, Kluyvera intermedia
  gene 1..1383: note = nifK1
  source 1..1383: mol_type = genomic DNA
atggcagatatcatccgtaatcagaaaccgctggcggtaagcccggtaaaaagcggccagccgttaggcgccattctggcgagcctcggctttgagcacagtattccactggtgcacggtgcgcagggatgcagcgcgttcgccaaagtgttttttatccaacattttcatgaccctattccgctgcaatccacggcgatggaccccacctcaacggtcatgggggcggacggcaatatccttgccgcgctcaatacgctgtgccagcgcaacaccccgaaagctatcgtcctgttgagtaccggcctgtctgaggcgcagggcagcgatatcagccgcgtggtacgtcagtttcgtgaggattttccccgccacaaaaatatcgccctcctgacggtcaacaccccggatttttacggcacgctggagaacggctttagtgcggtggtggaaagcgtcatcgaacagtgggtgccggaaaagcctcagcatggcctgcgtaaccggcgggtcaacttgttgttaagtcacctgctgacgcccggtgatgttgagttgctgcgcagctacgtggaggcttttggcctgcaaccggtgatcgtgccggatctttcacagtcgctggatggtcacctggcaagcggtgatttttcgccggtcactcaggggggaacgcccctgcgcattatcgaacagatgggacagagcctgtgcacgtttgctattggcgtgtcgctgtcccgtgcggcatcgctgctggcacagcgtagccgtggcgaggtgatcgtgcttccccatctgatgaccatggaacattgcgaccgttttattcatcaactgaagatcatttccgggcgcgaggttcccgcctggattgagcgccagcgcggacaattgcaggatgcgatgatcgattgtcatatgtggttgcaggatacccggctcgcgctggccgccgagggcgatctgctggcgggctggtgtgatttcgcccgtagccagggcatgctccccggccccgttgtggcgccggtcagccagccgggcctgcaacagcttcccgtggagaaagtggtcattggcgatctggaagatatgcaggatttactctgcgctatgcctgctgacctgctggtcgccaactcccatgccgcagacctggccgaacaattctccatcccgctgatccgcgccgggttccctatcttcgacaggcttggcgaatttcgtcgcgtgcgtcagggataccccggcattcgcgacacgctgtttgagctggcgaacctgatgcgcgaacgtcatcaccacctgcccgtctaccgctcccccctgcgccagcaatttgcccaggacgctgacggaggccgctatgcaacatgttaa

SEQ ID NO: 46 - DNA, 1563 bp, Kluyvera intermedia
  gene 1..1563: note = nifK2
  source 1..1563: mol_type = genomic DNA
atgagccaaactgctgagaaaattgtcacctgtcatccgctgtttgaacaggacgaataccagacgctgtttcgcaataagcgcggtctggaagaggcgcacgacccgcagcgcgtgcaagaggtttttgaatggaccaccacggcggagtatgaagcgctgaactttaagcgtgaagcgttaaccgtcgatccggcaaaggcctgccagcctttaggatcggtactctgctcgctgggttttgccaatacgctgccttatgtgcacggttcccagggctgtgtggcctatttccgcacctattttaaccgtcatttcaaagagccgatcgcttgcgtttccgactctatgacagaggatgcggcggtcttcggcggcaacaacaaccttaacaccgggttgcaaaatgccagcgccctgtacaaaccggaaattgtcgctgtctccactacctgtatggcggaggtcatcggcgatgacctgcaggcctttatcgccaacgccaaaaaggacgggtttattgatgccgccattccggtgccctacgcccatacgccaagttttatcggtagccacatcaccggctgggacaacatgtttgaaggtttcgcccgggcatttaccgccgatcacgtggcgcaaccgggcaaactggcgaagctaaacctggtgaccggttttgaaacctatcttggcaattaccgcgtgctcaaacgcatgatggcccagatggaggtgccctgtagcctgctgtctgacccgtctgaggtgttagatacgccagccgacggccactatcgcatgtatgcgggcggcacaacgcaacaagagatgcgcgacgcccccgatgctatcgacaccctgctgctgcaaccctggcatctggtgaagagtaaaaaagtggtgcaggagtcctggggccagcccgccacagaagtgtccatcccaatgggactgaccgggaccgacgaactgctgatggcagtcagtcagttaaccggcaaaccggtggccgatgaactgacgctggagcgtgggcgcctggtggatatgattctcgattcacacacctggctgcacggtaagaaattcggtctctacggcgatccggattttgtgatggggctgacgcgtttcctgctggaactgggctgcgagccgacggttatcctctgtcataacggtagcaagcgctggcagaaagcgatgaagaaaatgcttgaggcatcgccctacggtcaggagagcgaagtgttcatcaactgcgatctgtggcatttccgctcgctgatgtttacccgcaaaccggactttatgatcggcaactcgtacgccaaattcatccagcgtgacacgctggcgaaaggcgaacagtttgaagttccgctgatccgtcttggcttcccgttgttcgaccgccaccacctgcatcgccagaccacatggggttatgaaggggcgatgaatatcgtcaccaccctggtcaacgccgtgctggaaaaagtcgaccgcgataccatcaaactgggcaaaacggactacagcttcgaccttgtccgctaa

SEQ ID NO: 47 - DNA, 1488 bp, Kluyvera intermedia
  gene 1..1488: note = nifL
  source 1..1488: mol_type = genomic DNA
atgacctttaatatgatgctggagaccagcgcaccgcagcacattgcgggcaacctctcacttcaacatcccggactgttttccacgatggttgaacaggctccgatcgcgatttcgctgaccgacccggacgcgaggattctgtacgctaatccggccttttgtcgccagaccggttatagcctggaagagctgctcaaccagaaccatcgcatactggcaagccaacagacgccgcgcagcatttatcaggaactgtggcaaacgctgctgcaacagatgccctggcgcggtcagctcatcaatcgccgtcgggatggcagcctttatctggctgaggtcgatatcaccccggtcgtcaacaaacagggcgaactggaacactacctcgccatgcaacgtgatatcagcgccagctatgcgctcgaacagcgattgcgcaatcacaccaccatgagcgaggcggtgctgaacaacattcctgccgccgtggtggtggtcaacgagcaggaccaggtagtcatggacaacctcgcctacaaaaccttctgtgccgactgcggtggcaaggagctgctcaccgaactggatttctcccggcgcaaaagcgatctctatgccgggcaaatactgcctgtggtgctgcgcggcgccgtgcgctggctctctgtcacctgctggaccttgccgggggtgagcgaagaagccagccgctactttattgataccgcgctgccccgcaccctggtggtgatcaccgactgcacccagcaacaacaacaggccgaacagggccgtctcgatcgtctcaaacaggagatgaccaccgggaagctgctggccgcgatccgtgaatcgttggatgccgcgctggttcagctaaactgccccatcaatatgctggcggcggcgcgacgtctcaacggtgaagataaccataacgtggcgctggatgccgcgtggcgcgagggggaagaggcgctggcccgcctgcaacgctgccgcccttctctcgatctggaagagagcgcgctgtggcctctgcaaccgctgtttgacgacctgcgcgccctttaccatacccgctataacaatggcgaaaatctgcacgttgaaatggcctctccgcatctggcggggtttggtcagcgcacgcagatccttgcctgtctcagtttgtggctcgaccgtacgctggccctcgccgccgcgctaccggacagaacgctgcatacccagctttacgcccgtgaagaagatggctggctgtccatttggctgacagataatgtgccgctcatccatgtgcgatacgcccactcccccgatgccctgaacgcccccggcaaagggatggagctgcgattgattcaaaccctggttgcccatcatcgcggcgcaatagaactaactacccgccctgaaggcggtacctgcctgaccctgcgattcccgttatttcattcactgaccggaggcccacgatga

SEQ ID NO: 48 - DNA, 1572 bp, Kluyvera intermedia
  gene 1..1572: note = nifA
  source 1..1572: mol_type = genomic DNA
atgacccagcgacccgagtcgggcaccaccgtctggcgttttgatctctcacagcaatttaccgccatgcagcgcatcagcgtggtgttgagtcgcgcaaccgagataagccagacgctgcaggaggtgctgtgtgttctgcataatgacgcatttatgcaacacggcatgctgtgtctgtatgacaaccagcaggaaattctgagtattgaagccttgcaggaggcagaccaacatctgatccccggcagctcgcaaattcgctatcgccctggcgaagggctggtaggagccgtactgtcccagggacaatctcttgtgctgccgcgtgtcgccgacgatcaacgctttctcgacaggcttggcatctatgattacaacctgccgtttatcgccgtccccttaatggggccaggcgcgcagacgattggcgtgctcgccgcgcagccgatggcgcgtctggaggagcggcttccttcctgtacgcgctttctggaaaccgtcgccaatctggtcgcacagacagtccggctgatgaccccgcctgccgccgccacaccgcgcgccgcgattgcccagaccgaacgccagcgcaactgtggcactcctcgccccttcggctttgagaatatggtgggcaaaagcccggccatgcagcagacaatggacattatccgccaggtttcgcgctgggataccacggtactggtgcgcggcgaaagcggcaccggtaaagaacttatcgccaatgctattcatcacaactcccctcgcgccgccgcgccctttgtgaaatttaactgcgcggcgctaccggatacgctactggagagcgaattgttcggccatgaaaaaggggcgttcaccggcgcggttcgccagcgtaaaggacgttttgaactggccgatggcggcacactgtttcttgatgaaattggcgaaagcagcgcctcgttccaggccaaactgctgcgtattttgcaggagggtgaaatggagcgcgttggcggcgacgaaaccctgcgcgtcaatgtgcgtatcatcgccgccaccaaccggaatctggaagaagaggtgcggatgggcaatttccgcgaggatctctattatcgcctcaacgtaatgcccatctccctgcccccgctgcgtgaacgtcaggaggacattgccgagctggcgcactttctggtgcgcaaaatcgcccataaccaggggcgtacgctgcgcatcagtgatggcgccatccgtctgctgatgggttacaactggcccggtaacgtgcgtgagctggaaaattgcctggaacgttcggcagtgatgtcagaaaacggcctgatcgaccgcgatgtggtgctctttaaccaccgtgagaacacgccaaaactcgctatcgccgccgcgccaaaagaggatagctggcttgatcaaacgctggatgaacgtcaacggctgattgccgcgctggaaaaagccgggtgggtgcaggccaaagcggcgcgtctgctgggtatgacgccccgtcaggtcgcctatcggatacaaattatggatatcagcatgcccaggatgtga

SEQ ID NO: 49 - DNA, 2853 bp, Kluyvera intermedia
  gene 1..2853: note = glnE
  source 1..2853: mol_type = genomic DNA
atgatgccgcactctccacagctacagcagcactggcaaactgtactggcccgcttgcctgagtcattcagtgaaacaccgcttagtgaacaagcgcagttagtgcttactttcagtgattttgtgcaggatagccttgccgcgcatcctgactggctggctgagctggaaagcgcaccgccacaggcggacgagtggaagcagtatgcgcaaacccttcgcgaatcgctggaaggtgtgggagatgaggcatcattaatgcgtgcgctgcgcctgttccgtcgccatatgatggtgcgcattgcctgggcgcagtcgctggcgctggtggcagaagatgagacgttgcagcagttgagcgtactggcggagaccctgatcgtcgctgcacgcgactggctttacgatgcctgctgtcgcgagtggggaacgccgtgcaatcagcagggggaaccgcagccgttgctgatcctgggcatgggcaagctgggtggcggggagcttaacttttcgtccgatatcgatctgatttttgcctggccggaaaacggttcaacgcgcggtgggcgacgcgaacttgataacgcccagttttttactcgcttgggacagcgcctgatcaaagtgctcgaccagccgacgcaggatggctttgtctatcgcgtggatatgcggctgcgcccgtttggcgacagcggtccgctggtgctgagttttgccgcgctggaagattattatcaggagcaggggcgcgactgggaacgttatgcgatggtgaaagcccgcattatgggcgataaggacgatgtttacgctggcgaattacgggccatgctgcggccgttcgtcttccgtcgctatatcgatttcagcgttattcagtctctgcgtaacatgaaagggatgattgcccgcgaagtgcgccgccgtggtctgaaagataacattaagctgggcgcgggcggcatccgtgagattgagtttatcgttcaggtgttccagttgatacgcggtgggcgcgagccgtcgttgcagtcccgttcactgttaccgacgctggacgctatcgataagctgggtttgctgccgcctggcgatgcaccggcgttacgccaggcctatttgtatctgcgccgtctggaaaacctgctgcaaagcattaacgacgaacaaacgcagacgctgccgacagatgaactcaatcgcgcgcgtctggcctgggggatgcgggtcgcagactgggaaaccctgaccgctgagcttgaaaagcagatgtctgccgtacgagggatattcaacaccctgattggcgatgacgaagccgaagagcagggggatgcgctctgcgggcaatggagtgagttgtggcaggatgcgtttcaggaagatgacagcacgcctgtgctggcgcacctttctgacgatgatcgccgccgcgtggtcgcgatgattgctgattttcgcaaagagctggataaacgcaccattggcccacgcggccgccaggtgctcgaccatctgatgccgcatctgttgagtgatgtctgctcccgtgaggatgcccctgtaccgttgtctcgcgtgacgccgctgttaacgggaattgtcacgcgtacgacgtatcttgagctgctcagcgagtttcctggtgcgcgtaagcatctgatttcactctgtgccgcctcgccgatggtggccagtaagctggcgcgctatccgttattgctggatgagttgctcgatccgaataccctttatcagcccacggcgatgaatgcctaccgggatgagctacgtcagtatctgctgcgtgtgccggatgacgatgaagagcagcaactggaggcgttacgccagtttaaacaggctcaattgttgcgtgtggcggcagcagatctggcaggcacactccccgtgatgaaagtgagcgatcacttaacatggcttgccgaagccatcattgaagccgtggtacaacaggcgtggagcctgatggtatcgcgttatgggcagccgaaacacttacgcgaccgtgaaggccgtgggtttgcagtggtcggttacggcaaactgggcggttgggagctgggctatagttccgatctggatttgattttccttcatgactgtccggtggacgtgatgactgacggcgagcgggaaatcgatggccgccaattttatctgcgccttgcccagcgcgtgatgcacctgttcagtacgcgcacctcatccgggatcctgtatgaggtagacgcgcgcttgcgcccgtccggtgcggcgggaatgctggtgacctcaaccgaatcctttgccgactaccagcgcaccgaagcctggacctgggaacatcaggcgctggttcgcgcccgcgttgtctatggcgatccacaattaaacgcgcaatttgatgccatccgccgcgatatcaccatgaccgtgcgtaatggtgcaacgttacaaaccgaggtgcgcgagatgcgcgaaaaaatgcgcgcccacttgagcaataagcacaaggatcgctttgatattaaagccgatgagggtggaattaccgatatcgaatttatcacccagtatctggtgctgcgttatgcccatgccaaaccgaaactgacgcgctggtcggacaatgtccgcattctggaagggctggcgcaaaacggcattatggaagagcaggaagcgcaggcacttaccaccgcctatacaacgttgcgtgatgagctgcatcacctggcgctacaggagctgccaggacatgttccggaggcatgttttgtcgctgaacgcgcgatggtgcgagcctgctggaacaagtggttggtggagccgtgcgaggacgcgtaa

SEQ ID NO: 50 - DNA, 1290 bp, Kluyvera intermedia
  gene 1..1290: note = amtB
  source 1..1290: mol_type = genomic DNA
atgaagaaagcactattaaaagcgggtctggcctcgctggcattactgccgtgtctggctatggcagccgatccggttgtcgtcgataaagccgacaatgcctttatgatgatttgcaccgcgctggtgctgtttatgtcaattccgggcatcgccctgttctatggtggtttaatccgcggtaaaaacgtcctttctatgctgacacaggttgcggttacgttcgcactggtgtgcgtgctgtgggtggtttacggctactctctggcctttggcactggcggcagcttcttcggtagcttcgactgggtgatgctgaaaaatattgagctgaaagcgctgatgggcaccatctatcagtacattcacgttgcgttccagggctcgtttgcctgtattaccgtcggcctgattgtcggtgcgctggcagaacgtatccgtttctccgcagtactgattttcgtcgtggtatggctgacgctgtcctacgtgccgatcgcacacatggtctggggcggcggtctgctggcaacccatggcgccatggattttgcgggcggtacagtcgttcacatcaacgcagccgttgcaggcctggtgggtgcttacctgattggcaaacgtgtcggtttcggtaaagaagcgtttaaaccgcacaacctgccgatggtgtttaccggtacggcaatcctctactttggctggttcggattcaacgcgggttctgcaagcgcggcgaacgaaattgcgggtctggcttttgttaacaccgtcgtggcaacagcgggtgcaatcctctcctgggtcttcggtgagtgggcgctgcgcggcaaaccgtctctgttgggtgcctgttctggtgcgattgctggcctcgtgggtatcaccccggcgtgtggttacgttggtgtgggtggcgcgctgatcgtgggcatcgttgcaggcctggcgggtctgtggggcgttaccgcgctgaaacgctggctgcgtgttgacgacccgtgtgatgtcttcggtgttcacggcgtgtgcggtatcgtaggttgtatcatgacaggtatcttcgcagccacttcactgggcggcgtgggttatgccgaaggcgtgaccatgggccatcaggttctggtacaactggaaagtatcgccattactatcgtatggtctggtatcgtcgcctttatcggttacaaactggctgatatgacagtgggtctgcgtgttccggaagatcaggaacgcgaagggctggacgtcaacagccacggcgagaacgcctacaacgcctga

SEQ ID NO: 51 - DNA, 498 bp, Kluyvera intermedia
  gene 1..498: note = PinfC
  source 1..498: mol_type = genomic DNA
ctggggtcactggagcgctttatcggcatcctgaccgaagaatttgccggtttcttcccgacctggctggcccctgttcaggttgtggtgatgaatatcactgattctcaagctgaatatgtcaacgaattgacccgtaaattgcaaaatgcgggcattcgtgtaaaagcggacttgagaaacgagaagattggctttaaaatccgcgagcacactttacgtcgtgtcccttatatgttggtctgtggtgataaagaggtggaagcaggcaaagtggccgttcgcacccgccgcggtaaagacctgggcagcctggacgtaagtgaagtgattgagaagctgcaacaagagattcgcagccgcagtcttcaacaactggaggaataaggtattaaaggcggaaaacgagttcaaacggcacgtccgaatcgtatcaatggcgagattcgcgcccaggaagttcgcttaactggtctggaaggtgagcagctgggtatt

SEQ ID NO: 52 - DNA, 1537 bp, Kosakonia pseudosacchari
  gene 1..1537: note = 16S
  source 1..1537: mol_type = genomic DNA
attgaagagtttgatcatggctcagattgaacgctggcggcaggcctaacacatgcaagtcgaacggtagcacagagagcttgctctcgggtgacgagtggcggacgggtgagtaatgtctgggaaactgcctgatggagggggataactactggaaacggtagctaataccgcataacgtcgcaagaccaaagagggggaccttcgggcctcttgccatcagatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggcganacggttaataaccgtgttgattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcatccgaaactggcaggcttgagtctcgtagagggaggtagaattccaggtgtagcggtgaaatgcgtagagatctggaggaataccggtggcgaaggcggcctcctggacgaagactgacgctcaggtgcgaaagcgtggggagcaaacaggattagataccctggtagtccacgccgtaaacgatgtctatttggaggttgtgcccttgaggcgtggcttccggagctaacgcgttaaatagaccgcctggggagtacggccgcaaggttaaaactcaaatgaattgacgggggcccgcacaagcggtggagcatgtggtttaattcgatgcaacgcgaagaaccttacctggtcttgacatccacagaacttgccagagatggcttggtgccttcgggaactgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 53 - DNA, 882 bp, Kosakonia pseudosacchari
  gene 1..882: note = nifH
  source 1..882: mol_type = genomic DNA
atgaccatgcgtcaatgcgccatttacggcaaaggtgggatcggtaaatcgaccaccacacagaacctggtcgccgcgctggcggagatgggtaagaaagtcatgatcgtcggctgcgatccgaaagccgactccacgcgtttgatcctgcatgcgaaagcgcagaacaccattatggagatggccgccgaagtcggctccgtcgaagacctggaattagaagacgtgctgcaaatcggttacggcggcgtgcgctgcgcggaatccggtggcccggagccaggtgtgggttgtgccggtcgtggcgtgatcaccgcgattaacttcctcgaagaagaaggcgcttacgtgccggatctggattttgttttctacgacgtgctgggcgacgtggtatgcggtggtttcgccatgccgattcgtgaaaacaaagcgcaggagatctacatcgtttgctctggcgaaatgatggcgatgtacgccgccaataacatctccaaaggcatcgtgaaatatgccaaatccggtaaagtgcgcctcggcgggctgatttgtaactcgcgccagaccgaccgcgaagatgaactcatcattgcgctggcggaaaaactcggcacgcaaatgatccactttgttccccgcgacaacattgtgcagcgtgcggaaatccgccgtatgacggttatcgaatatgacccgacctgcaatcaggccaacgaatatcgcagccttgccagcaaaatcgtcaacaacaccaaaatggtggtaccaaccccctgcaccatggatgaactggaagaactgctgatggagttcggcattatggatgtggaagacgccagcatcattggtaaaaccgccgccgaagaaaacgccgtctga

SEQ ID NO: 54 - DNA, 1449 bp, Kosakonia pseudosacchari
  gene 1..1449: note = nifD1
  source 1..1449: mol_type = genomic DNA
atgagcaatgcaacaggcgaacgtaacctggaaatcatcgagcaggtgctggaggttttcccggaaaagacgcgcaaagagcgcagaaaacacatgatggtgacggacccggagcaggagagcgtcggcaagtgcatcatctctaaccgcaaatcgcagccgggcgtgatgaccgtgcgtggctgctcgtatgccggatcaaaaggggtggtatttgggccaatcaaagatatggcgcatatctcccacggcccgatcggctgcgggcagtactcccgcgccgggcggcgtaactactataccggcgtcagcggcgtggacagtttcggcacgctcaacttcacctccgatttccaggagcgcgacatcgtgtttggcggcgacaaaaagctcgccaaactgattgaagagctggaagaactgtttccgctgaccaaaggcatttcgattcagtcggaatgcccggtcggcctgattggcgatgatattgaagccgtggcgaacgccagccgcaaagcgatcaacaaaccggttattccggtgcgttgcgaaggctttcgcggcgtgtcgcaatccctcggtcaccatattgccaacgatgtgatccgcgactgggtactggataaccgcgaaggcaaaccgtttgaatccaccccttacgatgtggcgatcatcggcgattacaacatcggtggcgacgcctgggcctcgcgcattttgctcgaagagatggggttgcgggtggtcgcgcagtggtccggcgacggtacgctggtggagatggaaaacacgccgttcgtcaaactgaacctggtgcactgctaccgctcgatgaactacatctcgcgccatatggaggagaagcacggtattccgtggatggaatacaacttctttggcccgacgaaaatcgcggaatcgctgcgcaaaatcgccgacctgttcgacgacaccattcgcgccaacgccgaagcggtgatcgcccgataccaggcgcagaacgacgccattatcgccaaatatcgcccacgtctggagggtcgcaaagtgttgctctatatgggcgggctgcgtccgcgccatgtgattggcgcctatgaagatctgggaatggagatcatcgccgccggttatgagtttggtcataacgacgattacgaccgcaccctgccggatctgaaagagggcacgctgctgtttgatgacgccagcagctatgagctggaggcgtttgtcaacgcgctgaaaccggatctcatcggttccggcatcaaagagaagtacatctttcagaaaatgggcgtgccgtttcgccagatgcactcctgggattactccggcccgtaccacggctatgacggcttcgccatcttcgcccgcgatatggatatgacgctcaacaaccccgcctggggtcagttgaccgcgccgtggcttaaatccgcctga

SEQ ID NO: 55 - DNA, 1374 bp, Kosakonia pseudosacchari
  gene 1..1374: note = nifD2
  source 1..1374: mol_type = genomic DNA
atgaaggggaacgacatcctggctctgctcgatgaaccagcctgcgagcataaccataaacagaaaaccggctgtagcgcgccaaaacccggcgccaccgccggaggctgcgccttcgacggcgcacagatcaccctgctgccactttccgatgtggcgcatctggtacatggcccgattggctgcgccggcagctcatgggataaccgtggcagcctgagttctggcccgctgattaaccgactcggattcaccactgatttgaacgaacaggatgtcatcatggggcgcggcgagcggcggttgtttcacgcggtgcgccatattgtcgagcgctatcacccggcggcggtatttatttacaacacctgcgttccggctatggaaggcgatgacattgacgcggtctgccaggccgccgcgaccgccaccggtgtgcccgtgattgccgtagatgtggccggtttttacggtagcaaaaacctgggtaaccgcctcgcgggcgaggtgatggtgaaaaaagttatcggcgggcgcgaacccgcgccgtggccggacaatacaccttttgccccggcgcaccgccatgacataggcctgattggcgaatttaacatcgccggcgagttctggcatatccagccgctgcttgatgagctgggtattcgcgtccttggctccctttccggcgacgggcgctttgccgagatccagacgttgcaccgcgcgcaggtcaatatgctggtgtgctccagggcgctgattaatgtcgccagatcgcttgaacaacgttatggcacaccctggtttgaaggcagtttttatggcgttcgcgccacctccgatgccctgcgccagctggcaacactcaccggcgatagcgatttaatggcgcgaaccgaacggctgatcgcacgtgaagagcaagccacagaacaggcgctagcaccgctgcgtgaacggttacacggccggaaagtgctgctctataccggtggcgtgaaatcctggtcggtggtttcggcgctgcaggatctcggcatgacggtcgttgctaccggaacgcgcaaatccaccgaagaggataaacaacgcatccgtgaactgatgggcgatgacgccatcatgctggatgaaggcaatgcccgcgccttgctggatgtggtctatcgctacaaagccgacatgatgatcgcgggcgggcgcaacatgtacaccgcctataaagcgcgtctgccctttctggatatcaaccaggagcgtgaacacgcgtttgccggttatcgcggcatcatcacgcttgccgaacaactttgtcagacgctggaaagcccggtctggccgcaaacacatgcccgcgccccgtggcaataa

SEQ ID NO: 56 - DNA, 1563 bp, Kosakonia pseudosacchari
  gene 1..1563: note = nifK1
  source 1..1563: mol_type = genomic DNA
atgagccagactgctgagaaaatacagaattgccatcccctgtttgaacaggacgcctaccagacactatttgccggtaaacgggcactcgaagaggctcactcgccggagcgggtgcaggaagtgtttcaatggaccaccaccccggaatacgaagcgctgaacttcaaacgcgaagcgctgactatcgacccggcaaaagcctgccagccgctgggggcggtgctctgttcgctggggtttgccaacaccctgccgtatgtgcacggttcacagggttgtgtggcctatttccgtacgtactttaaccgccacttcaaagaaccggtggcctgcgtgtcggattcgatgacggaagacgcggccgtgttcggcgggaataacaacctcaacaccgggttacaaaacgccagcgcactgtataaaccggagattatcgccgtctctaccacctgtatggcggaagtgatcggtgatgatttacaggcgtttatcgccaacgccaaaaaagatggttttctcgatgccgccatccccgtgccctacgcccacacccccagttttatcggtagccatatcaccggctgggacaacatgtttgaaggttttgcccgtacctttaccgcaaaccatcagccacagcccggtaaactttcacgcctgaacctggtgaccgggtttgaaacctatctcggcaatttccgcgtgctgaaacgcatgatggaacaaatggaggtgcaggcgagtgtgctctccgatccgtcggaggtgctggacacccccgccaatggccattaccagatgtacgcgggcggtacgacgcagcaagagatgcgcgaggcaccggatgccatcgacaccctgctgctgcaaccgtggcagctggtgaaaagcaaaaaagtggtgcaggagatgtggaatcagcccgccaccgaggttgccattcccgtcgggctggcaggcacagacgaactgttgatggcgattagccagttaaccggcaaagccattcccgattcgctggcgctggagcgcgggcggctggtcgatatgatgctcgactcccacacctggttacacggtaaaaaattcggtctgtttggcgatccggattttgtcatgggattgacccgcttcctgctggaactgggctgtgaacctgccgtcatcctctgccataacggtaacaaacgctggcaaaaagcgatgaagaaaatgctcgatgcttcaccgtacggccaggagagcgaagtgtttatcaactgcgacttgtggcatttccgctcgctgatgttcacccgccagccggattttatgattggcaactcgtacgccaagtttattcagcgcgacaccttagccaagggcgaacagtttgaagtcccgctgatccgcctcggttttccgctgttcgaccgtcaccatctgcaccgccagaccacctggggctacgagggcgcgatgagcattctcacgacgctggtgaatgcggtactggagaaagtggacaaagagaccatcaagctcggcaaaaccgactacagcttcgatcttatccgttaa

SEQ ID NO: 57 - DNA, 1386 bp, Kosakonia pseudosacchari
  gene 1..1386: note = nifK2
  source 1..1386: mol_type = genomic DNA
atggctgatattgttcgtagtaaaaaaccgctggcggtgagcccgataaaaagcggccagccgctgggggcgatcctggcaagcctgggtttcgaacagtgcataccgctggtacacggcgctcaggggtgcagcgcgttcgcgaaagtgttctttattcaacattttcacgacccgatcccgctgcaatcgacggcgatggacccgacttccaccattatgggcgccgatgaaaacatttttaccgcgctcaatgttctctgccagcgcaacgccgcgaaagccatcgtgctgctcagcaccgggctgtcagaagcccagggcagcgatatttcacgagtggtgcgccagtttcgtgatgactttccgcggcataaaaacgtggcgctgctcaccgtcaacaccccggatttctacggctcgctggaaaacggctacagcgccgtgctggaaagcatgattgaacagtgggtgcccgcgcagcccgccgccagcctgcgcaaccgtcgcgtcaacctgctggtcagccatttactgacgccgggcgatatcgaactgttacgcagttatgtggaagcattcggtctgcaaccggtgattgtgccggatctatcgcagtcgctggacggacatctggccaacggtgatttttcgcccgtcacccaggggggaacaccgctgcgcatgattgaacagatggggcaaaacctggccacttttgtgattggccactcgctggggcgggcggcggcgttactggcgcagcgcagccgtggcgaggtgatcgccctgccgcatctgatgacgcttgatgcgtgcgacacctttatccatcgcctgaaaaccctctccgggcgcgacgtgcccgcgtggattgagcgccagcgcgggcaagtgcaggatgcgatgatcgattgccatatgtggttgcagggcgcggctatcgccatggccgcagaaggcgatcacctggcggcatggtgcgatttcgcccgcagccagggcatgatccccggcccggttgtcgcgccggtcagccagccggggttgcaaaatctgccggttgaaatggtggtcatcggcgatctggaagatatgcaggatcggctttgcgcgacgcccgccgcgttactggtggccaattctcatgccgccgatctcgccacgcagtttgatatgtcgcttatccgcgccgggtttccggtgtatgaccggctgggggaatttcgtcggctgcgccaggggtatagcggcattcgtgacacgctgtttgagctggcgaatgtgatgcgcgaacgccattgcccgcttgcaacctaccgctcgccgctgcgtcagcgcttcggcgacaacgttacgccaggagatcggtatgccgcatgttaa

SEQ ID NO: 58 - DNA, 1488 bp, Kosakonia pseudosacchari
  gene 1..1488: note = nifL
  source 1..1488: mol_type = genomic DNA
atgaccctgaatatgatgatggatgccagcgcgcccgaggccatcgccggtgcgctttcgcaacaacatcctgggctgttttttaccatcgttgaagaagcccccgtcgctatttcactaaccgatgccgaggcacgtattgtctatgccaacccggcattctgccgccagaccggctatgagcttgaggagttgttgcagcaaaatccccgcctgcttgccagtcagcagaccccacgggaaatctaccaggatatgtggcacaccctgttacaacgtcgaccatggcgcgggcaattgatcaaccgccaccgtgacggcagcctttttctggttgagatcgatatcaccccggtgattaacccgtttggcgaactggaacactacctggccatgcagcgcgatatcagcgccggttatgcgctggagcagcggttgcgtaatcacatggcgctgaccgaagcggtgctgaataacattccggcggcggtggtcgtggtcgatgaacgcgatcgtgtggttatggataacctcgcctataaaactttctgtgctgattgcggcggaaaagagctactgagcgaactccatttttcagcccgtaaagcggagctggcaaacggccaggtcttaccggtggtgctgcgcggcgcggtgcgctggttgtcggtcacctgctgggcgctgccaggcgtcagcgaagaagccagtcgctactttattgataataccttgacgcgcacgctggtggtcatcaccgacgacacccagcagcgccagcagcaagagcaaggacggcttgaccgccttaaacagcagatgaccagcggcaaactgctggcggcgatccgcgaagcgcttgacgccgcgctgatccagcttaactgccccatcaatatgctggcggcggcgcggcgtttaaacggcagtgataacagcaacgtagcgctggacgccgcgtggcgcgaaggtgaagaagcgatggcgcggctgaaacggtgccgcccgtcgctggagctggaaagtgccgccgtctggccgctgcaacccttttttgacgacttgcgcgcgctttatcacacccgctacgagcagggtaaaaatttgcaggtcacgctggattcgacgcatctggtgggatttggtcagcgaacccaactgctggcctgcctgagtctgtggctcgatcgcacgctggatattgccgtcgggctgcgtgatttcaccgcccaaacgcagatttacgcccgggaagaagcgggctggctctcgttgtatatcactgacaatgtgccgttgattccgctgcgccatacccattcgccggatgcgcttaacgcaccgggaaaaggtatggagttgcggctgatccagacgctggtagcgcatcacaacggcgcgatagaactcacttcacgccccgaagggggaagctgcctgaccctacgattcccgctatttcattcactgaccggaggttcaaaatga

SEQ ID NO: 59 - DNA, 1575 bp, Kosakonia pseudosacchari
  gene 1..1575: note = nifA
  source 1..1575: mol_type = genomic DNA
atgacccagcgaaccgagtcgggtaataccgtctggcgcttcgatttatcccagcagttcaccgcgatgcagcggataagcgtggttctcagccgggcgaccgaggttgaacagacactccagcaggtgctgtgcgtattgcacaatgacgcctttttgcagcacggcatgatctgtctgtacgacagccagcaggcgattttgactattgaagcgttgcaggaagccgatcagcagttgatccccggcagctcgcaaattcgctaccgtccgggtgaagggctggtcgggacggtgctttcgcaggggcaatcgttagtgctggcgcgtgtggctgacgatcagcgctttcttgaccgcctgggactgtatgattacaacctgccgtttatcgccgtgccgctgatagggccggatgcgcagacttttggcgtgctgacggcgcaaccgatggcgcgttacgaagagcggttacccgcctgcacccgctttctggaaacggtcgcgaatctggtggcgcagaccgtgcgtttgatgacgccgccggctgcacgcccttccccacgcgctgccatcacgccaaccgccagcccgaaatcgtgcagtacttcacgcgcgttcggcttcgaaaatatggtcggcaacagcccggcaatgcgccagaccatggagattatccgtcaggtttcgcgctgggataccaccgttctggtgcgcggcgagagcggcaccggcaaggaactgattgccaacgccatccatcacaattcgccgcgcgccagtgcgccatttgtgaaattcaactgtgcggcgctgccggacacattgcttgaaagcgaattatttggtcatgaaaaaggcgcctttaccggcgcggtacgccagcgtaaaggccgttttgagctggccgatggcggcacgctgtttcttgacgaaattggggaaagcagcgcctcgtttcaggctaagctgctgcgtattttgcaggagggcgaaatggaacgcgtcggtggtgacgagacattgcaagtgaatgtgcgcatcattgccgcgacgaaccgcaaccttgaagatgaagtacgcctgggacattttcgcgaagatctctattaccgcctgaatgtgatgcccatcgccctgccgccgctgcgcgaacgccaggacgacatcgccgaactggcacattttctggtgcgtaaaatcgcccacaaccagaaccgcacgctgcgcattagcgagggcgctatccgcctgctgatgagctacagctggcccggcaatgtgcgcgaactggaaaactgccttgagcgctctgcggtgatgtcggaaaacggtctgatcgatcgggacgtgattttatttaatcatcgcgaccagccagccaaaccgccggttatcagcgtcacgcccgacgataactggctcgataacacccttgacgagcgccagcggctgattgccgcgctggaaaaagcgggatgggtacaagccaaagccgcccgcttgctggggatgacgccgcgccaggtcgcttatcgtattcagaccatggatatcaccctgccaaggctataa

SEQ ID NO: 60 - DNA, 2850 bp, Kosakonia pseudosacchari
  gene 1..2850: note = glnE
  source 1..2850: mol_type = genomic DNA
atgccgcaccacgcaggattgtcgcagcactggcaaacggttttttctcgtctgccggaagcgctcaccgcgcaaccattgagcgcgcaggcgcagtcagtgctcacttttagtgattttgttcaggacagcatcatcgtgcatcctgagtggctggcagagcttgaaagcgcaccgccgccagcgaacgagtggcaacactacgcgcaatggctgcaagcggcgctggagggcgtcaccgatgaaacctcgctgatgcgcacgctgcggctgtttcgccgtcgcattatggtgcgcatcgcctggagtcaggcgctacagttggtggcggaagaggatatcctgcaacagctcagcgtgctggcggaaactctgatcgtcgccgcgcgcgactggctctatgacgcctgctgccgtgagtggggaacgccgtgcaatccgcaaggcgtcgcgcagccgatgctggtgctcggcatgggcaaacttggcggcggcgaactcaatttctcatccgatatcgatttgatttttgcctggccggaaaatggcaccacgcgcggcggacgccgtgaactggataacgcgcagttttttacccgccttggtcaacggctaattaaagtcctcgaccagcccacgcaggatggctttgtctaccgcgtcgatatgcgcttgcgtccctttggcgacagcggcccgctggtgctgagttttgccgcgctggaagattactaccaggagcaggggcgcgactgggaacgatacgcgatggtgaaagcgcgcattatgggggacaacgacggcgaccatgcgcgagagttgcgcgccatgctgcgcccgttcgttttccgccgctatatcgacttcagcgtgatccagtctctgcgcaacatgaaaggcatgattgcccgcgaagtgcggcgtcgcggcctgaaggacaacataaaactcggcgcgggcggtattcgcgaaatagagtttatcgtgcaggttttccagttgattcgcggcggtcgcgagcctgcgctgcaatcgcgttcgctgttgccgacgcttgctgccattgatcaactacatctgctgccggatggtgatgcaccccggctgcgcgaggcgtatttgtggctgcgacggctggaaaacttgctgcaaagcattaatgacgaacagacacagacgctgccggccgatgatttgaatcgcgcgcgcctcgcctggggaatgggcaaagagagctgggaagcgctctgcgaaacgctggaagcgcatatgtcggcggtgcggcagattttcaacgatctgattggcgatgatgaaacggattcgccggaagatgcgctttctgagggctggcgcgaattgtggcaggatgcgttgcaggaagaggactctacgcccgtgctggcgcatctttccgaggacgatcgccgccgcgtggtggcgctgattgctgattttcgcaaagagctggataaacgcaccattggcccgcgcgggcgacaggtactcgatcacttaatgccgcatctgctcagcgatgtatgctcgcgtgacgatgcgccagtgccgctgtcgcgtctgacgccgctgctcaccggtattattacgcgcaccacttaccttgagctgctgagtgaattccccggtgcgctgaaacacctcatttccctgtgcgccgcgtcgccgatggtggccagccaactggcgcgctacccgatcctgctcgatgaactgctcgacccgaacacgctctatcaaccgacggcgatgaacgcctatcgcgatgaactgcgacaatacctgttgcgcgtgccggaagaggatgaagagcagcaactggaggcgctacggcagtttaagcaggcgcagttgttgcgcgtagcggcggcggatatcgccggtacgttacccgtcatgaaagtgagcgatcacttaacctggctggcggaagcgattatcgatgcggtggtgcagcaagcctggaaccagatggtggcgcgttacggccagccgacgcatctgcacgatcgcgaagggcgcggtttcgccgtggtcggttacggcaaacttggcggctgggaattaggttacagctccgatctggatctggtgttcctgcacgactgccccatggatgtgatgaccgatggcgagcgtgaaatcgatggccgccagttctatttgcgcctcgcgcagcgcgtgatgcacctgttcagcacgcgcacgtcgtccggcattctttatgaagtcgatgcgcgtttgcgcccgtccggcgcggccggaatgctggtgaccactgcggaagcgttcgccgattatcaaaaaaatgaagcctggacatgggagcatcaggcgctggcgcgtgcgcgcgtggtgtacggcgatccgcaactgaccgccgaatttgacgccattcgccgcgatatcctgatgacctcccgcgatgccgctaccctgcaaaccgaagtgcgggaaatgcgtgagaaaatgcgcgcccatcttggtaacaagcacaaagaccgtttcgatctgaaagccgatgaaggcggtatcaccgatattgagtttatcgctcagtatctggtgctgcgctttgcccatgagaagccgaaactgacgcgctggtcggataatgtgcgcatcctcgaagggctggcgcaaaacggcatcatggatgagcaggaagcgcaggcattgacgctggcgtacaccacgttgcgtgatgagctgcaccacctggcgctgcaagagctgccaggacatgtggcgctctcctgttttgtcgccgagcgtgcgcttatcaaaaccagctgggacaagtggctggtggaaccgtgcgccccggcgtaa

SEQ ID NO: 61 - DNA, 1287 bp, Kosakonia pseudosacchari
  gene 1..1287: note = amtB
  source 1..1287: mol_type = genomic DNA
atgaaaaacacaacattaaaaacggctcttgcttcgctggcgttgctgccaggcctggcgatggcggctcccgctgtggcggataaagccgacaacggctttatgatgatttgcaccgcgctggtgctgtttatgaccattccgggcattgcgctgttctacggcggtttgatccgcggtaaaaacgtgctgtcgatgctgacgcaggttgccgtcaccttcgctctggtgtgcatcctgtgggtggtttacggctactctctggcatttggcgagggcaacagcttcttcggcagtttcaactgggcgatgttgaaaaacatcgaattgaaagccgtgatgggcagcatttatcagtacatccacgtggcgttccagggctcctttgcttgtatcaccgttggcctgattgtcggtgcgctggctgagcgtattcgcttctctgcggtgctgatttttgtggtggtatggctgacgctttcttatgtgccgattgcgcacatggtctggggtggcggtctgctggcaacccacggcgcgctggatttcgcgggcggtacggttgttcacatcaacgccgcgatcgcaggtctggtgggggcttacctgattggcaaacgcgtgggctttggcaaagaagcgttcaaaccgcataacctgccgatggtcttcaccggcaccgcgatcctctatgttggctggtttggcttcaacgccggctctgcaagctcggcgaacgaaatcgctgcgctggctttcgtgaacacggttgttgccactgcggccgctattctggcgtgggtatttggcgagtgggcaatgcgcggtaagccgtctctgctcggtgcctgttctggtgccatcgcgggtctggttggtatcaccccggcgtgcggttatgtgggtgtcggcggcgcgctgattgtgggtctgattgccggtctggcagggctgtggggcgttactgcactgaaacgtatgttgcgtgttgatgacccatgcgatgtcttcggtgtgcacggcgtgtgcggcatcgtgggttgtatcctgaccggtatcttcgcgtctacgtcgctgggcggtgtcggtttcgctgaaggggtgaccatgggccatcaggtactggtacagctggaaagcgttgccatcactatcgtgtggtctggcgtggtggcctttatcggttacaaactggcggatatgacggtaggcctgcgcgtaccggaagagcaagagcgtgaagggctggatgtgaacagccacggcgaaaatgcgtataacgcctga

SEQ ID NO: 62 - DNA, 500 bp, Kosakonia pseudosacchari
  gene 1..500: note = PinfC
  source 1..500: mol_type = genomic DNA
ttcttggttctctggagcgctttatcggcatcctgactgaagaatttgcaggcttcttcccaacctggcttgcacccgtgcaggtagttgtgatgaacatcactgattcgcaggctgaatacgttaacgaattgacccgtaaactgcaaaatgcgggcattcgtgtaaaagcagacttgagaaacgagaagattggctttaaaatccgcgagcacactttacgtcgtgtcccttatatgctggtttgtggtgacaaagaggtcgaagccggcaaagttgctgtgcgtacccgtcgcggtaaagacctgggtagcctggacgtaaatgatgttatcgagaagctgcaacaagagattcgcagccgcagtcttcaacaactggaggaataaggtattaaaggcggaaaacgagttcaaacggcgcgtcccaatcgtattaatggcgagattcgcgccacggaagttcgcttaacaggtctggaaggcgagcagcttggtatt

SEQ ID NO: 63 - DNA, 348 bp, Kosakonia pseudosacchari
  gene 1..348: note = Prm1
  source 1..348: mol_type = genomic DNA
cgttctgtaataataaccggacaattcggactgattaaaaaagcgccctcgcggcgctttttttatattctcgactccatttaaaataaaaaatccaatcggatttcactatttaaactggccattatctaagatgaatccgatggaagctcgctgttttaacacgcgttttttaaccttttattgaaagtcggtgcttctttgagcgaacgatcaaatttaagtggattcccatcaaaaaaatattctcaacctaaaaaagtttgtgtaatacttgtaacgctacatggagattaactcaatctagagggtattaataatgaatcgtactaaactggtactgggcgc

SEQ ID NO: 64 - DNA, 339 bp, Kosakonia pseudosacchari
  gene 1..339: note = Prm7
  source 1..339: mol_type = genomic DNA
cgcgtcaggttgaacgtaaaaaagtcggtctgcgcaaagcacgtcgtcgtccgcagttctccaaacgttaattggtttctgcttcggcagaacgattggcgaaaaaacccggtgcgaaccgggtttttttatggataaagatcgtgttatccacagcaatccattgattatctcttctttttcagcatttccagaatcccctcaccacaaagcccgcaaaatctggtaaactatcatccaattttctgcccaaatggctgggattgttcattttttgtttgccttacaacgagagtgacagtacgcgcgggtagttaactcaacatctgaccggtcgat

SEQ ID NO: 65 - DNA, 1538 bp, unidentified
  misc_feature 1..1538: note = Enterobacter sp.
  gene 1..1538: note = 16S
  source 1..1538: mol_type = genomic DNA
ttgaagagtttgatcatggctcagattgaacgctggcggcaggcctaacacatgcaagtcgngcggaagcacaggagagcttgctctctgggtgacgagcggcggacgggtgagtaatgtctgggaaactgcctgatggagggggataactactggaaacggtagctaataccgcataacgtcgcaagaccaaagagggggaccttcgggcctcttgccatcagatgtgcccagatgggattagctagtaggtggggtaacggcncacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggtgttgnggttaataaccncagcaattgacgttacccgcagaagaagcaccggctaactccgtgccagcagccgcggtaatacggaggntgcaagcgttanncggaatnantgggcgtaaagcgtncgcaggcggtntgtnaagtcggatgtgaaatccccgggctcaacctgggaactgcattcgaaactggcaggctagagtnnngtagaggngggtagaattccnggtgtagcggtgaaatgcgtagagatcnggangaanaccngtggcgaaggcggcccnctggacaaagactgacgctnaggngcgaaagcgtggggagcaaacaggattagataccctngtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaacttnncagagatgnnttggtgccttcgggaactctgagacaggtgctgcatggctgtcgtcagctcgtgttgtgaaatgttgggttaagtcccgcaacgagcgcaacccttatcctttgttgccagcggtncggccgggaactcaaaggagactgccagtgataaactggaggaaggtggggatgacgtcaagtcatcatggcccttacgagtagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 66 - DNA, 882 bp, unidentified
  misc_feature 1..882: note = Enterobacter sp.
  gene 1..882: note = nifH
  source 1..882: mol_type = genomic DNA
atgaccatgcgtcaatgtgccatttacggcaaaggtggtatcggtaaatccactaccacgcaaaacctggtcgccgcgctggcggagatgggcaagaaagtaatgatcgtcggctgcgacccgaaagcagactccactcgtctgatcctgcatgcgaaagcgcagaacaccattatggagatggcggctgaagtcggctccgtggaagaccttgaactggaagatgtgctgcaaatcggttacggcgacgtacgctgcgcagaatccggcggcccggaaccaggcgttggctgtgctggtcgcggggtaattaccgccatcaacttcctggaagaagaaggcgcctatgttcccgacctcgatttcgtcttttacgacgtgttgggcgacgtggtgtgcggggggttcgccatgccgattcgcgaaaacaaagcgcaggagatctacatcgtctgctccggcgaaatgatggcgatgtacgccgccaacaacatctctaaaggcatcgtgaaatacgccaaatccggcaaagtgcgccttggcgggctgatctgtaactcccgtcagaccgaccgcgaagatgagctgatcatagcgctggcggaaaaactcggcacccagatgatccacttcgtgccgcgcgacaacatcgtgcaacgcgctgaaatccgccgtatgacggtgattgagtacgatccgaaatgcaaccaggccaatgaataccgcacgctggcgaacaagatcgtcaacaacaccaaaatggtcgtgccaacgcccatcaccatggacgaactggaagagctgttgatggaattcggcattatggatgtggaagacaccagcattatcggtaaaaccgccgcagaagaaaacgcggtttga

SEQ ID NO: 67 - DNA, 1449 bp, unidentified
  misc_feature 1..1449: note = Enterobacter sp.
  gene 1..1449: note = nifD1
  source 1..1449: mol_type = genomic DNA
atgagcaatgcaacaggcgaacgtaatctggagatcatccaggaagtgctggagatctttccggaaaaaacgcgcaaagaacgcagaaagcacatgatggtgagcgacccggagatggaaagcgtcgggaaatgcatcatctccaaccgtaagtcgcagcccggcgtaatgaccgtgcgcggttgctcttacgccggttctaaaggggtggtattcgggccgatcaaagatatggcccatatttcccacggcccggtcggctgcggtcagtactcccgcgccgggcggcgtaactactacaccggcgtcagcggtgtggatagcttcggtacgctcaactttacctccgattttcaggagcgcgatatcgtgtttggcggcgataaaaagctgaccaaactgattgaagagatggagacgctgttcccgctgaccaaagggatctccattcagtccgaatgcccggtcggcctgattggcgacgacattgaagccgttgccaacgccagccgcaaagccatcaataaaccggtcattccggtgcgctgcgaaggttttcgcggcgtttcccagtcactcggtcaccacattgccaacgacgtgatccgcgactgggtactggataaccgcgaaggcaagccgtttgaggccggtccttatgacgtggcgatcatcggcgattacaacatcggcggcgatgcctgggcgtcgcgcattttgctcgaagagatgggcctgcgcgtggtggcgcagtggtccggcgacggcacgctggttgagatggagaacacgccgttcgtcaaactcaaccttgtgcactgctaccgctcaatgaactatatctcccgccatatggaggagaaacacggtattccgtggatggagtacaacttcttcggtccgaccaaagtcgccgaatcgttgcgcaaaatcgccgatatgtttgatgacaccattcgcgccaacgccgaagcggtgatcgccaaatatcaggcgcagaacgacgccatcatcgccaaataccgtccgcgtctggaaggccgcaaagtgctgctgtatatgggcggtttacgtcctcgccatgtgattggcgcttatgaagatctggggatggaaattatcgctgcgggttatgaattcgcccacaacgatgactacgaccgcaccctgccggatctgaaagaaggcaccttgctgttcgacgatgccagcagttatgaactggaagcctttgtcaaagcgctgaagccggatctgatcggctccggcattaaagagaagtacatcttccagaaaatgggcgtgccgtttcgccagatgcactcctgggattactccggcccctatcacggttatgacggctttgccatcttcgcccgcgatatggatatgacgatcaacaaccccgcgtggggccagttgaccgcgccgtggctgaaatccgcctga

SEQ ID NO: 68 - DNA, 1092 bp, unidentified
  misc_feature 1..1092: note = Enterobacter sp.
  gene 1..1092: note = nifD2
  source 1..1092: mol_type = genomic DNA
atgggacgcggcgagcgccgcctgttccatgccgtgcgccacatcgtcaaccgctaccacccggccgccgtctttatctataacacctgcgttcccgcgatggagggcgacgatatcgaagccgtctgccaggcggcagaaaccgccatcggcgtaccggtgattgccgttgatgtcgccgggttttacggcagcaaaaatctcggcaaccggttggccggtgaagtgatggtgaaaaaggtgattggcgggcgtgaacccgcgccgtggccggaagataccccttttgccccggcgcaccgccacgatatcgggctgattggcgaattcaatattgccggagagttctggcatattcagccgctgctcgatgagctgggtattcgcgtgctcggcagcctctccggcgacgggcgcttcagtgaaatccagacgctgcaccgggcgcaggtcaatatgctggtctgctccagggcgctgatcaacgtcgcccgctcgctggagcagcgctacggcacgccgtggtttgaaggcagtttttatggtgttcgcgccacctctgacgccctgcgccaactggcggcgctgaccggagaccgcgatctgatgcagcgcaccgaacagctcattgcccgcgaagagcagcaaacagagcaggcgctggccccgctgcgcgagcgcctgcgcgggcgcaaagcgctgctctatnccggcggcgtgaaatcctggtcggtggtttcggcgcttcaggatctgggcatggaagtggtggcgaccggcacgcgcaaatccaccgaagaggataaacagcgcatccgcgaactgatgggcgccgacgcgctgatgcttgatgaaggtaacgcccgctcgctgctggacgtggtttaccgctacaaggcggacatgatgatcgccgggggacgcaatatgtacaccgcctacaaagcgcggctgccgttcctcgatatcaatcaggagcgcgagcacgcctttgccggctaccgcggcattgtcaccctggccgaacagctctgcctgaccatggaaagcccggtctggccgcaaacccattcccgcgcaccgtggcaataa

SEQ ID NO: 69 - DNA, 846 bp, unidentified
  misc_feature 1..846: note = Enterobacter sp.
  gene 1..846: note = nifK
  source 1..846: mol_type = genomic DNA
atgatggagcaaatggacgtgccgtgcagcctgctttccgatccctccgaagtgctggataccccggctgacgggcattaccacatgtatgcgggcggtacgacccagcaggagatgcgcgaagcgcctgacgctatcgacaccctgctgctgcaaccctggcaactggtgaaaaccaaaaaagtggtgcaggaaagctggaaccagcccgctaccgaggtgcaaatcccaatggggctggccggaaccgacgagctgctgatgacggtaagccagttaaccggcaaagccattccggatagcttagcgctggaacgcggtcggctggtggatatgatgctcgactcccacacctggctgcacggcaagaaattcggcctgttcggtgacccggattttgtcatggggctgacccgcttcctgctggaactgggctgcgaaccgacggtgattctgtgccataacggcagcaagcgctggcagaaagcgatgaagaaaatgcttgaagcctcgccgtacgggaaagagagcgaagtctttatcaactgcgatttgtggcatttccgctcgctgatgtttacccgtcagccggactttatgatcggcaactcctacgccaagtttatccagcgcgatacgctggcgaagggtgagcagtttgaagtgccgctgatccgcctggggttcccgctgttcgatcgccaccatctgcaccgccagaccacctggggttacgaaggggccatgagtatcctcaccacgctggttaatgcggtgctggagaaagtcgacagagagaccatcaagctcggcaaaaccgactacagcttcgatcttatccgttaa

SEQ ID NO: 70 - DNA, 1095 bp, unidentified
  misc_feature 1..1095: note = Enterobacter sp.
  gene 1..1095: note = nifL
  source 1..1095: mol_type = genomic DNA
atgcagcgcgacatcagcaccagctacgcgctggaacaacggctgcgcaatcatatgacgctgaccgaagccgtcttgaataacattccggcggcggttgtagtggtggatgaacgcgatcgggtggtgatggataacctcgcctacaaaaccttttgcgccgattgcggcggtaaagaactactcaccgaaatcaacttttccgcccataaggcggagctggcgcagggcctggtactgccggtagtgctgcgcggcaccgtgcgctggttgtccgttacctgttgggcgctgccgggcgtcagcgaagaagcaggccgctactttattgatagcgccgtgccgcgcacgctggtggtgatcaccgataatactcagcagcagcaacaacaggagcaggggcgtcttgatcgtctgaagcagcagataaccagcggtaaattgctggcggcgatccgcgaatcgctggacgccgcgctggtacaactcaattgcccaattaatatgctggccgccgcacgccgcttaaatggcgacgagcatagcaatctggcgctggatgccgcatggcgtgaaggcgaagaagcgatggcgcggttgcagcgctgccgcccgtcgctggaactggaaagcccggcagtctggccgctccagccgttccttgacgatctgcgtgccctgtatcacacccgatataaccagggcgaaaacctgcaaattgagctggaatcccccgacctggtgggctttggccagcgaacacaactgcttgcctgcctgagcctgtggctcgacagaaccctggatattgccgcggagctacgtgatttcacggtacagactcaactttacgcccgcgaagagagcggctggctgtcgttctatttaaacgacaatgtgccgctgattcaggtgcgctacacccattcacccgatgcactnaatgcgcccggtaaaggcatggagctgcggctgatccagacgctggtcgcccaccatcgaggcgcaatagaactgacctcacgccctcagggaggcacctgtctgatcctgcgtttcccattattttactcgctgacaggaggctcactatga

SEQ ID NO: 71 - DNA, 219 bp, unidentified
  misc_feature 1..219: note = Enterobacter sp.
  gene 1..219: note = nifA partial gene
  source 1..219: mol_type = genomic DNA
atgactcagcgaaccgagtcgggtacaaccgtctggcgctttgacctctcccaacagtttacagccatgcagcgtatcagtgtggtgttaagccgcgcgacggagatcgggcagacgctacaggaagtgctgtgcgtgctgcacaacgatgcctttatgcagcacgggatgatctgtccgtacgcgcgggtgcgcgtcttcgcgagcgtatggctttga

SEQ ID NO: 72 - DNA, 1635 bp, unidentified
  misc_feature 1..1635: note = Enterobacter sp.
  gene 1..1635: note = glnE
  source 1..1635: mol_type = genomic DNA
atgcgcgtggaagactggtcaacgctgaccgaacggctcgatgcccatatggcaggcgtgcgccgaatctttaacgaactgatcggtgatgacgaaagtgagtcgcaggacgatgcgctctccgagcactggcgcgagctgtggcaggacgcgcttcaggaagatgacaccacgccggtgctgacgcacttaaccgacgacgcgcgccatcgcgtggtggcgctgatcgctganttccgtcttgagctgaacaaacgcgccatcggcccgcgtngtcgccaggtgctggatcacctgatgccgcacctgctgagcgaagtctgctcgcgtgccgatgcgccggtgccgctgtcgcggatgatgcccctgctgagcgggattatcacccgtactacctaccttgaactcctgagcgagttccctggcgcgcttaagcacctgatttcactctgcgccgcgtcgccgatggtggccaacaagctggcgcgttacccgctgctgctggatgagctgctcgatccgaataccctttatcaaccgacggcgaccgacgcctaccgggacgaactgcgtcagtatctgctgcgcgtgccggaagaagacgaagagcaacagctggaggcgctgcgtcagtttaagcaggcccagatgctgcgcgtggcggccgcagatattgccggaacgctgccggtgatgaaagtgagcgatcacttaacctggcttgcggaagcgattatcgacgcggtggtgcatcaggcctgggtgcagatggtggcgcgctatggccagccgaaacatctggctgaccgtgatggtcgcggcttcgcggtggtgggttacggtaagctcggcggttgggagctgggctatagctccgatctggatttaatcttcctccacgactgcccggttgatgtgatgaccgacggcgagcgcgagattgacgggcgtcagttctacctgcgcctggcgcagcgcatcatgcacctgttcagcacccgcacctcgtcgggcattttgtatgaagtggatgcccgtctgcgcccgtccggcgcggcgggcatgctggtcacctcgacggagtccttcgctgattaccagaagaatgaagcctggacgtgggagcatcaggcgctggtgcgcgcccgtgtggtgtatggcgatccgctgctgaaaacgcagtttgacgtgattcgtaaggaagtcatgaccaccgtgcgcgatggcagcacgctgcaaacggaagtgcgcgaaatgcgcgagaaaatgcgcgcgcacttaggcaataaacatcgcgatcgctttgatattaaagccgatgagggcggtattaccgatattgagtttattacccagtatctggtgttgctgcacgcgcatgacaagccgaagctgacgcgctggtcggataacgtgcgcattctggaactgctggcgcaaaacgacattatggacgagcaggaggcgcaggccttaacccgtgcctatacaacgcttcgcgatgagctccatcatctggcgttgcaggagcagcngggacacgtggcgctggactgtttcaccgctgaacgcgctcaggtaacggccagctggcagaagtggctggtggaaccgtgcgtaacaaatcaagtgtga

SEQ ID NO: 73 - DNA, 1316 bp, unidentified
  misc_feature 1..1316: note = Enterobacter sp.
  gene 1..1316: note = 16S
  source 1..1316: mol_type = genomic DNA
agatgtgcccagatgggattagctagtaggtggggtaacggcncacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggtgttgtggttaataaccncagcaattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaacttnncagagatgnnttggtgccttcgggaactctgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgagtagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 74 - DNA, 882 bp, unidentified; identical to SEQ ID NO: 66
  misc_feature 1..882: note = Enterobacter sp.
  gene 1..882: note = nifH
  source 1..882: mol_type = genomic DNA

SEQ ID NO: 75 - DNA, 1449 bp, unidentified; identical to SEQ ID NO: 67
  misc_feature 1..1449: note = Enterobacter sp.
  gene 1..1449: note = nifD1
  source 1..1449: mol_type = genomic DNA

SEQ ID NO: 76 - DNA, 1374 bp, unidentified
  misc_feature 1..1374: note = Enterobacter sp.
  gene 1..1374: note = nifD2
  source 1..1374: mol_type = genomic DNA
atgaaggggaacgagatcctggctttgctcgatgaacctgcctgcgagcacaaccataaacagaaatccggctgcagcgcgccgaaacccggcgcgacagcgggcggctgcgcctttgacggtgcgcagatcaccctgctgccactctccgatgttgcccacctggtacacggccccattggttgtaccggtagctcatgggataaccgtggcagcttcagttccggcccgacgatcaaccggctgggttttaccaccgatctgagcgaacaggatgtgatcatgggacgcggcgagcgccgcctgttccatgccgtgcgccacatcgtcaaccgctaccacccggccgccgtctttatctataacacctgcgttcccgcgatggagggcgacgatatcgaagccgtctgccaggcggcagaaaccgccatcggcgtaccggtgattgccgttgatgtcgccgggttttacggcagcaaaaatctcggcaaccggttggccggtgaagtgatggtgaaaaaggtgattggcgggcgtgaacccgcgccgtggccggaagataccccttttgccccggcgcaccgccacgatatcgggctgattggcgaattcaatattgccggagagttctggcatattcagccgctgctcgatgagctgggtattcgcgtgctcggcagcctctccggcgacgggcgcttcagtgaaatccagacgctgcaccgggcgcaggtcaatatgctggtctgctccagggcgctgatcaacgtcgcccgctcgctggagcagcgctacggcacgccgtggtttgaaggcagtttttatggtgttcgcgccacctctgacgccctgcgccaactggcggcgctgaccggagaccgcgatctgatgcagcgcaccgaacagctcattgcccgcgaagagcagcaaacagagcaggcgctggccccgctgcgcgagcgcctgcgcgggcgcaaagcgctgctctataccggcggcgtgaaatcctggtcggtggtttcggcgcttcaggatctgggcatggaagtggtggcgaccggcacgcgcaaatccaccgaagaggataaacagcgcatccgcgaactgatgggcgccgacgcgctgatgcttgatgaaggtaacgcccgctcgctgctggacgtggtttaccgctacaaggcggacatgatgatcgccgggggacgcaatatgtacaccgcctacaaagcgcggctgccgttcctcgatatcaatcaggagcgcgagcacgcctttgccggctaccgcggcattgtcaccctggccgaacagctctgcctgaccatggaaagcccggtctggccgcaaacccattcccgcgcaccgtggcaataa

SEQ ID NO: 77 - DNA, 1563 bp, unidentified
  misc_feature 1..1563: note = Enterobacter sp.
  gene 1..1563: note = nifK1
  source 1..1563: mol_type = genomic DNA
atgagccaaagtgctgagaaaattcaaaactgtcatccgctgtttgaacaggatgcgtaccagatgctgtttaaagataaacggcaactggaagaggcccacgatccggcgcgcgtgcaggaggtctttcaatggaccaccaccgccgagtatgaagcgcttaactttcaacgcgaagcgctgactatcgatccggccaaagcctgccagccgctgggtgcggtactgtgctcgctgggctttgccaataccctgccctatgttcacggctcccaggggtgcgtggcctatttccgcacctattttaaccgtcactttaaagagccgattgcctgtgtttctgactcgatgacggaagatgcggcagtattcggcggcaacaacaacctgaacaccgggttgcagaacgccagcgccctctacaagccggaaatcattgccgtctccaccacctgtatggcggaggtcatcggcgacgacctgcaggcgtttattgctaacgccaaaaaagacggctttatcgacgcggcgatcccggtgccttacgcgcacacgccaagctttatcggcagccatatcaccggctgggacaatatgtttgagggcttcgcccgtacctttaccgccgattacagcggacaaccgggcaaattaccgcgtatcaatctggtcagcggatttgaaacctatctcggtaatttccgcgtgctgaaacgcatgatggagcaaatggacgtgccgtgcagcctgctttccgatccctccgaagtgctggataccccggctgacgggcattaccacatgtatgcgggcggtacgacccagcaggagatgcgcgaagcgcctgacgctatcgacaccctgctgctgcaaccctggcaactggtgaaaaccaaaaaagtggtgcaggaaagctggaaccagcccgctaccgaggtgcaaatcccaatggggctggccggaaccgacgagctgctgatgacggtaagccagttaaccggcaaagccattccggatagcttagcgctggaacgcggtcggctggtggatatgatgctcgactcccacacctggctgcacggcaagaaattcggcctgttcggtgacccggattttgtcatggggctgacccgcttcctgctggaactgggctgcgaaccgacggtgattctgtgccataacggcagcaagcgctggcagaaagcgatgaagaaaatgcttgaagcctcgccgtacgggaaagagagcgaagtctttatcaactgcgatttgtggcatttccgctcgctgatgtttacccgtcagccggactttatgatcggcaactcctacgccaagtttatccagcgcgatacgctggcgaagggtgagcagtttgaagtgccgctgatccgcctggggttcccgctgttcgatcgccaccatctgcaccgccagaccacctggggttacgaaggggccatgagtatcctcaccacgctggttaatgcggtgctggagaaagtcgacagagagaccatcaagctcggcaaaaccgactacagcttcgatcttatccgttaa

SEQ ID NO: 78 - DNA, 1386 bp, unidentified
  misc_feature 1..1386: note = Enterobacter sp.
  gene 1..1386: note = nifK2
  source 1..1386: mol_type = genomic DNA
atggcagaaattatccgtagtaaaaagccgctggccgtcagcccggtaaaaagtggccagccgctgggcgcgattctggcgagcatgggctttgaacagagcattccgctggttcatggcgctcaggggtgcagcgccttcgcgaaggtcttttttatccagcattttcacgatccgatcccgctgcaatcgacggcaatggacccgacatcgaccattatgggtgccgatgagaacatctttaccgcgctgaatgtgctgtgttcacgcaacaacccgaaagcgattgttctgctgagcactggcctttccgaggcgcagggcagcgatatttcgcgcgtggtgcgccagttccgcgatgaatatccgcgccataaaggggtggcgctgctgaccgtcaacacgccggatttttacggcagcctggaaaacggctacagcgcggtgctggagagcatggttgaacagtgggtgccggaaaaaccgcagccgggcgtgcgcaatcgccgcgtgaacctgctgctcagccatttgcttacgccgggcgacattgagctgctgcgaagttatgtcgaggcatttggcctgcagccggtgatggtgccggatctttcccagtcgctggatggccatctcgccagcggggatttctcgccaattacccagggcggcagcagcctgcggctgattgaacagatgggacagagtcttggcacgttcgccattggcgtatccctctcccgcgccgcgcaattgctggcgcagcgcagccatgcggaagtggtcaccctgccgcatctgatgaccatgagccagtgcgatacgtttattcatcaactgaagcgcctctccgggcgcgatgttccggcgtggatcgaacgccagcgcgggcaactgcaggatgcgatgatcgattgtcatatgtggttgcagggcgcgcctgtcgcgctggccgccgagggcgatctgctcgccgcctggtgcgatttcgcctgcgatatgggcatggtgcccggcccggtggtggcgccggtgagccagaaagggttgcaggatctgccggtcgaaaaagtcattatcggcgatctggaggatatgcaggatctgttgtgtgaaacgcctgcatcgctgctcgtctctaattctcacgccgctgatttggccgggcagttcgacattccgctggtgcgcgccggtttccccctgttcgaccgtctgggcgagtttcgccgcgtgcgccagggttacgccgggatgcgcgacaccttgtttgagctggcgaatgcgctgcgcgatcgccatcatcatcttgccgcttatcactcgccgctgcgccagcgtttttacgaacccgcatcttcgggaggtgactatgcaacatgttaa

SEQ ID NO: 79 - DNA, 1488 bp, unidentified
  misc_feature 1..1488: note = Enterobacter sp.
  gene 1..1488: note = nifL
  source 1..1488: mol_type = genomic DNA
atgaccctgaatatgatgatggacgccaccgcgcccgccgagatcgccggagcgctctcacaacagcatcccgggttgtttttcaccatggttgaacaggcgcccgtcgcgatttcactgaccgatgccgatgcccacattctctacgccaaccccgcgttttgtcgccagtcggggtatgaactggaagagttgttgcagcaaaacccgcgcctgcttgccagtaagcagacgccgcgtgaaatctaccaggaaatgtggcacaccctgctgcaacaccgtccgtggcgcggacaactgatcaaccgtcgccgcgacggcagcctgtttctggtggaaatcgacatcaccccactgtttgatgcgttcggcaaactcgaacattacctggccatgcagcgcgacatcagcaccagctacgcgctggaacaacggctgcgcaatcatatgacgctgaccgaagccgtcttgaataacattccggcggcggttgtagtggtggatgaacgcgatcgggtggtgatggataacctcgcctacaaaaccttttgcgccgattgcggcggtaaagaactactcaccgaaatcaacttttccgcccataaggcggagctggcgcagggcctggtactgccggtagtgctgcgcggcaccgtgcgctggttgtccgttacctgttgggcgctgccgggcgtcagcgaagaagcaggccgctactttattgatagcgccgtgccgcgcacgctggtggtgatcaccgataatactcagcagcagcaacaacaggagcaggggcgtcttgatcgtctgaagcagcagataaccagcggtaaattgctggcggcgatccgcgaatcgctggacgccgcgctggtacaactcaattgcccaattaatatgctggccgccgcacgccgcttaaatggcgacgagcatagcaatctggcgctggatgccgcatggcgtgaaggcgaagaagcgatggcgcggttgcagcgctgccgcccgtcgctggaactggaaagcccggcagtctggccgctccagccgttccttgacgatctgcgtgccctgtatcacacccgatataaccagggcgaaaacctgcaaattgagctggaatcccccgacctggtgggctttggccagcgaacacaactgcttgcctgcctgagcctgtggctcgacagaaccctggatattgccgcggagctacgtgatttcacggtacagactcaactttacgcccgcgaagagagcggctggctgtcgttctatttaaacgacaatgtgccgctgattcaggtgcgctacacccattcacccgatgcactcaatgcgcccggtaaaggcatggagctgcggctgatccagacgctggtcgcccaccatcgaggcgcaatagaactgacctcacgccctcagggaggcacctgtctgatcctgcgtttcccattattttactcgctgacaggaggctcactatga

SEQ ID NO: 80 - DNA, 1575 bp, unidentified
  misc_feature 1..1575: note = Enterobacter sp.
  gene 1..1575: note = nifA
  source 1..1575: mol_type = genomic DNA
atgactcagcgaaccgagtcgggtacaaccgtctggcgctttgacctctcccaacagtttacagccatgcagcgtatcagtgtggtgttaagccgcgcgacggagatcgggcagacgctacaggaagtgctgtgcgtgctgcacaacgatgcctttatgcagcacgggatgatctgtctgtacgacagtaagcaagcgatcctttccattgaagccttgcatgaggccgatcagcagttaattcccggcagttcacagattcgctaccgtccgggcgaagggctggtaggcacggtgctttcacagggacagtcgctggtactgccctgtgtctccgacgatcggcgttttctcgatcgcctgggattgtatgattacagcttgccgtttatcgccgtgccgctgatggggccaaactcgcagcctatcggcgtgctggccgcccagcctatggcgcgttacgaggagcggctgcccgcctgcacgcgttttcttgaaaccgtcgccaatctggtggcgcaaaccgttcgcctgatgacaccgcccagcgtcgcgtctccaccccgtgctgctgccgcgcagattgccagccagcgcgggtgcgcgtcttcgcgagcgtatggctttgaaaacatggtcggtaaaagcgcggctatgcgtcagacgctggaaattattcgccaggtatcacgctgggacaccaccgtgctggtgcgtggcgaaagcggaaccggtaaagagttgatagccaacgctatccaccacaattcaccgcgcgccgccgcgccgtttgtcaaattcaactgcgcggcgctgcccgatacgctgctggagagtgaactcttcggtcatgaaaaaggcgcgtttaccggcgcggtgcgccagcgcaaaggccgtttcgaactggcggatggcggtacgctgtttcttgatgagatcggcgaaagtagcgcctcgtttcaggcgaaattgctgcgtatcttgcaggaaggcgaaatggaacgcgtcggcggcgacgaaacgctgcgggtgaatgtacggatcattgccgccaccaaccgcaatctggaagaggaagtgcggctgggtaattttcgcgaagatctctactatcgccttaatgtgatgccgatctccctgcccccgctccgcgagcgtcaggaggacatcgtcgagctggcgcattttctggtgcgcaaaatcgcgcaaaaccagaaccgcacgctgcgcatcagcgatggcgcgatccgtttgttgatgagctatagctggcctggaaacgtgcgtgagctggaaaactgccttgagcgatcggcggtgatgtcggaaaacgggctgatcgatcgcgacgtgattttgtttcaccacagggaaaatctgccaaaaacgccacagaccagtgcgccgcgcgaagagagctggctcgatcagaacctcgatgagcgacaaagattgatcgccgcgctggagaaagccggttgggtacaggcaaaagccgcgcgcctgctgggaatgaccccgcgccaggtggcctatcgtattcagacgatggacattgccatgccgagattgtag

SEQ ID NO: 81 - DNA, 2856 bp, unidentified
  misc_feature 1..2856: note = Enterobacter sp.
  gene 1..2856: note = glnE
  source 1..2856: mol_type = genomic DNA
atgccgctttcttcgcagttacagcagcagtggcagaccgtttgcgaacgtctgcctgagtcattaccggcgtcatcgttaagcgagcaggcaaagagcgtgctcgtcttcagtgattttgtgcaggaaagtatcaccgccaacccgaactggctggcggaacttgagaacgcaccaccgcaggcagaagagtggcggcactatgctggctggctgcaaactgtactcgaagacgttacggatgaggccacgctgatgcgcgtcctgcgccagttccgtcgtcgggtgatggtgcgcattgcctgggctcaggcgctggaactggtgagcgaagagagtacgctgcagcagttaagcgagctggcgcaaacgttgattgtcgccgcgcgagactggctctatgccgcctgctgtaaagagtggggcacgccgtgcagcgaggaaggggttcctcagccgctgttgattctggggatgggaaagctgggcggctgcgagctgaacttctcctctgatatcgacctgatttttgcctggccggagaacggctccacgcgcggaggccgccgcgagctggacaacgcgcagttctttacccgtctcggccagcgcctgattaaagcgctggatcagcccacgcaggacggttttgtttaccgcgtggacatgcgcctgcgtccgtttggcgacagcgggccgctggtgctgagctttgcggcgctggaagattattaccaggagcaaggtcgcgactgggagcgttacgcgatggtcaaagcgcggatcatgggcgacagcgacgacgcttatgccaacgagctgcgcgccatgctgcgtccgttcgtgttccgtcgctatatcgacttcagcgtcatccagtccctgcgaaatatgaaagggatgattgcccgcgaggtgcgccgccgtgggctgaaagacaatatcaagctcggtgcgggcggcatccgcgaaatcgaatttatcgtccaggtcttccagcttattcgcggcggacgcgagccgtcgctgcagtcccgttccttattaccgacgctgagcgccattgcgcagctgcatctcctgccggacggcgacgcgcaaaccctgcgcgaggcctatcttttcctgcgtcgtctggaaaacctgctgcaaagcattaatgacgaacagacccaaaccctgccgggcgacgaccttaaccgggcgcgtctggcctggggaatgcgcgtggaagactggtcaacgctgaccgaacggctcgatgcccatatggcaggcgtgcgccgaatctttaacgaactgatcggtgatgacgaaagtgagtcgcaggacgatgcgctctccgagcactggcgcgagctgtggcaggacgcgcttcaggaagatgacaccacgccggtgctgacgcacttaaccgacgacgcgcgccatcgcgtggtggcgctgatcgctgatttccgtcttgagctgaacaaacgcgccatcggcccgcgtggtcgccaggtgctggatcacctgatgccgcacctgctgagcgaagtctgctcgcgtgccgatgcgccggtgccgctgtcgcggatgatgcccctgctgagcgggattatcacccgtactacctaccttgaactcctgagcgagttccctggcgcgcttaagcacctgatttcactctgcgccgcgtcgccgatggtggccaacaagctggcgcgttacccgctgctgctggatgagctgctcgatccgaataccctttatcaaccgacggcgaccgacgcctaccgggacgaactgcgtcagtatctgctgcgcgtgccggaagaagacgaagagcaacagctggaggcgctgcgtcagtttaagcaggcccagatgctgcgcgtggcggccgcagatattgccggaacgctgccggtgatgaaagtgagcgatcacttaacctggcttgcggaagcgattatcgacgcggtggtgcatcaggcctgggtgcagatggtggcgcgctatggccagccgaaacatctggctgaccgtgatggtcgcggcttcgcggtggtgggttacggtaagctcggcggttgggagctgggctatagctccgatctggatttaatcttcctccacgactgcccggttgatgtgatgaccgacggcgagcgcgagattgacgggcgtcagttctacctgcgcctggcgcagcgcatcatgcacctgttcagcacccgcacctcgtcgggcattttgtatgaagtggatgcccgtctgcgcccgtccggcgcggcgggcatgctggtcacctcgacggagtccttcgctgattaccagaagaatgaagcctggacgtgggagcatcaggcgctggtgcgcgcccgtgtggtgtatggcgatccgctgctgaaaacgcagtttgacgtgattcgtaaggaagtcatgaccaccgtgcgcgatggcagcacgctgcaaacggaagtgcgcgaaatgcgcgagaaaatgcgcgcgcacttaggcaataaacatcgcgatcgctttgatattaaagccgatgagggcggtattaccgatattgagtttattacccagtatctggtgttgctgcacgcgcatgacaagccgaagctgacgcgctggtcggataacgtgcgcattctggaactgctggcgcaaaacgacattatggacgagcaggaggcgcaggccttaacccgtgcctatacaacgcttcgcgatgagctccatcatctggcgttgcaggagcagccgggacacgtggcgctggactgtttcaccgctgaacgcgctcaggtaacggccagctggcagaagtggctggtggaaccgtgcgtaacaaatcaagtgtga

SEQ ID NO: 82 - DNA, 1290 bp, unidentified
  misc_feature 1..1290: note = Enterobacter sp.
  gene 1..1290: note = amtB
  source 1..1290: mol_type = genomic DNA
atgaagatagcaacacttaaaacgggtctgggttcgctggcactgctgccgggcctggcgctggctgctgcacctgcggtggcagacaaagccgataacgcctttatgatgatcagcaccgcgctggtgctgttcatgtccattccgggcattgcgctgttctatggcggcctgatccgtggcaaaaacgttctctccatgctgacgcaggttgccgtaacgttcgcgctggtctgcgtactgtgggtggtttacggttactcgctggctttcggcacgggcaacgcgttctttggtaacttcgactgggtgatgctgaaaaatattgaactgaccgcgctgatgggcagtttctaccagtatattcacgttgctttccagggctcgttcgcctgcattaccgtcgggctgattgtaggcgcgcttgccgagcgtattcgtttctctgcggtcctgatcttcgtggtggtctggctgacgctctcctatgtgccgattgcgcacatggtctggggtggcggtctgctggcgacgcatggcgcgctggacttcgcgggcggtaccgttgtgcacattaacgccgcggtagcgggtctggttggcgcatacctgattggcaaacgcgtgggcttcggtaaagaagcgttcaaaccgcacaacctgccgatggtcttcaccggtaccgcgatcctctactttggctggtttggtttcaacgccggctcagcaagtgccgcgaacgaaatcgccgcgctggccttcgtgaataccgttgtggccacggcaggtgcaatcctctcctgggtctttggcgagtgggctgtgcgcggtaaaccttctctgctgggtgcctgttcgggggcgattgctggtctggtcggtatcaccccagcatgtggttatgtcggtgtgggtggcgcgctgctggtcggcctggtgtcaggtctggcgggtctgtggggcgtgacggcgctgaaacgtattctgcgcgttgatgacccttgcgatgtgtttggcgtgcacggcgtgtgcggcatcgtcggctgtatcatgaccggtatctttgcagcgaaatcgctgggtggcgtgggctacgcagaaggcgtcaccatggcccatcaggtgctggtgcagctggaaagtattgctgtcaccgtggtgtggtctgccgttgtcgctttcattggctacaaactggcggacatgacggttggtctgcgcgtgccggaagagcaggaacgcgaaggtctggacgtcaacagccacggcgagaatgcgtataacgcatga

SEQ ID NO: 83 - DNA, 1299 bp, Azospirillum lipoferum
  gene 1..1299: note = 16S
  source 1..1299: mol_type = genomic DNA
gccgagagaggggcccgcgtcggattaggtagttggtgaggtaatggctcaccaagccttcgatccgtagctggtctgagaggatgatcagccacactgggactgagacacggcccagactcctacgggaggcagcagtggggaatattggacaatgggcgcaagcctgatccagcaatgccgcgtgagtgatgaaggccttagggttgtaaagctctttcgcacgcgacgatgatgacggtagcgtgagaagaagccccggctaacttcgtgccagcagccgcggtaatacgnagggngcnagcgttnntcggaattactgggcgtaaagngcgcgtaggcggcntgttnagtcagaagtgaaagccccgggctcaacctgggaatagcttttgatactggcaggcttgagttccggagaggatggtggaattcccngtgtagnggtgaaatncgtagatattgggangaacaccngtggcgaaggcggcnatctggacgganactgacgctgaggcgcgaaagcgtggggagcaaacaggattagataccctngtagtccacgccgtaaacgatgaatgctagacgtcggggtgcatgcacttcggtgtcgccgctaacgcattaagcattccgcctggggagtacggccgcaaggttaaaactcaaaggaattgacgggggcccgcacaagcggtggagcatgtggtttaattcgaagcaacgcgcagaaccttaccaacccttgacatgtccactttgggctcgagagatngggtccttcagttcggctgggtggaacacaggtgctgcatggctgtcgtcagctcgtgtcgtgagatgttgggttaagtcccgcaacgagcgcaacccctaccgtcagttgccatcattcagttgggcactctggtggaaccgccggtgacaagccggaggaaggcggggatgacgtcaagtcctcatggcccttatgggttgggctacacacgtgctacaatggcggtgacagtgggaagcgaagtcgcgagatggagcaaatccccaaaagccgtctcagttcggatcgcactctgcaactcgagtgcgtgaagttggaatcgctagtaatcgcggatcagcacgccgcggtgaatacgttcccgggccttgtacacaccgcccgtcacaccatgggagttggttttacccgaaggtggtgcgctaaccgcaaggaggcagccaaccacggtaaggtcagcgactggggtgaagtcgtaacaaggtagccgtaggggaacctgcggctggatcacctccttt

SEQ ID NO: 84 - DNA, 897 bp, Azospirillum lipoferum
  gene 1..897: note = nifH
  source 1..897: mol_type = genomic DNA
atggccaaagcgcctctgcgtcagatcgccttttacggcaagggcggtatcggcaagtccaccacctctcagaacacgctggccgcgctggtcgagctggatcagaggatcctgatcgtcggctgcgacccgaaggccgactcgacccgcctgatcctgcacgcaaaggcccaggacaccgtcctgcatctggccgccgaggccggctcggtcgaggatctggagctcgaggacgttctcaagatcggctacaagaacatcaagtgcgtcgagtccggcggtccggagccgggggtcggctgcgccggccgcggcgtcatcacctcgatcaacttcctggaagagaacggcgcctacgacgacgtggactatgtgtcctacgacgtgctgggcgacgtggtctgcggcggcttcgccatgccgatccgcgagaacaaggcccaggaaatctacatcgtcatgtccggcgagatgatggcgctgtacgccgccaacaacatcgccaagggcatcctgaagtacgcgcacagcggcggcgtccgtctcggcggcctgatctgcaacgagcgccagaccgacaaggaatgggatctggccgacgcgctggccaagcgcctgggctccaagctgatccacttcgtgccgcgcgacaacatcgtccagcacgccgagctgcgccgcatgacggtcatcgagtacgccccggacagcaagcaggccggcgaataccgcgcgctcgccaacaagatccatgcgaactccggccagggttgcatcccgaccccgatcaccatggaagagctggaagagatgctgatggacttcggcatcatgaagaccgaggagcagcagctcgccgagctcgccgccaaggaagcggcgaaggccggcgcctga

SEQ ID NO: 85 - DNA, 1440 bp, Azospirillum lipoferum
  gene 1..1440: note = nifD1
  source 1..1440: mol_type = genomic DNA
atgagcctgtccgagaacaccacggtcgacgtcaagaacctcgtcaacgaagtcctcgaagcctatcccgaaaaatcccgcaagcgccgcgccaagcacctgaacgtgctggaggccgaggccaaggactgcggcgtcaagtcgaacgtcaagtccatccccggcgtcatgaccatccgcggctgcgcctatgccggctccaagggcgtggtgtggggtccgatcaaggacatgatccacatctcccacggtccggtcggctgcggctactactcctggtccggccgccgcaactactacatcggcgacaccggtgtggacagctggggcacgatgcacttcacctccgacttccaggagaaggacatcgtcttcggcggcgacaagaagctgcacaaggtcatcgaggaaatcaacgagctgttcccgctggtgaacggcatctcgatccagtcggaatgcccgatcggcctgatcggcgacgacatcgaggctgtcgcccgcgccaagtcggcggaaatcggcaagccggtcatccccgtgcgctgcgaaggcttccgcggcgtgtcccagtcgctgggccaccacatcgccaacgacgccatccgagactgggtgttcgagaagacggaacccaaggccggcttcgtctccaccccctatgacgtcaccatcatcggcgactacaacatcggcggcgacgcctggtcgtcccgcatcctgctggaggagatcggcctgcgcgtgatcgcccagtggtcgggcgacggcacgctcgccgaactggagaacacgccgaaggccaaggtcaacctgatccactgctaccgctcgatgaactacatcgcgcgccacatggaagagaagttcaacattccttggatggaatacaacttcttcggcccgagccagatcgccgaatccctgcgcaagatcgccgctctcttcgacgacaagatcaaggagaacgccgagaaggtcatcgcccgctaccagccgatggtcgatgcggtcatcgccaagtacaagccgcggctcgaaggcaagaaggtcatgatctacgtcggcggcctgcgtccccgccacgtcgtcgatgcctaccatgacctcggcatggagatcaccggcaccggctacgagttcgcccacaacgacgactatcagcgcacgcagcactacgtgaaggaaggcacgctgatctacgacgacgtcaccgcgttcgaactggagaagttcgtcgaggcgatgcgtcccgacctcgtcgcgtcgggcatcaaggaaaagtacgtgttccagaagatgggcctgccgttccgccagatgcacagctgggactactccggcccgtaccacggctatgacggcttcgcgatcttcgcccgcgacatggacctggccatcaacaaccccgtctggggcgtgatgaaggccccgttctga

SEQ ID NO: 86 - DNA, 1419 bp, Azospirillum lipoferum
  gene 1..1419: note = nifD2
  source 1..1419: mol_type = genomic DNA
atgctccaggacaagatccaggatgtcttcaacgaaccgggctgcgcgaccaaccaagccaaatcggccaaggagaagaagaagggctgcaccaagtcgctgaaaccgggggcggcagccggcggctgcgcctatgacggggcgatgatcgtgctccagccgatcgccgacgccgcccatctggtccatggccccatcgcctgcctcggaaacagttgggacaaccgcggctccaaatcctccggctcgcagctctaccgcaccggcttcaccaccgatctgtcggaactggacgtcatcggcggcggcgagaagaagctctaccgcgccatcaaggagatcgttcagcaatacgacccgccggccgtcttcgtctatcagacctgcgtgcccgccatgaccggcgacgacatcgccgcggtctgcaagttcgccacgcagaagctgggcaagccggtgatcccggtggactcgccgggcttcgtcgggtcgaagaatctcggcaacaagctggccggcgaagccctgctggagcatgtcatcggcacggtcgaaccggactacaccaccccgaccgacgtctgcatcatcggcgaatacaaccttgccggcgagctgtggctggtcaagccgctgctggacgagatcggcatccgcctcctgtcctgcatttccggcgacggccgctaccgggaggtggcgcaggcccaccgcgcccgcgtcaccatgatggtgtgcagccaggcgctggtgaatgtcgggcgcaagatggaggagcgctacggcatcccctatttcgaggggtccttctacggcgtgtccgacatgtcggacaccctgcgcaccatgacccgcatgctggtggagcgcggcgccgacaagggcctgatcgaccgggcggagggcgtgatcgcgcgggaggaaagccgggtctggcgccggctggaaccctacaagccgcgcttcgacggcaagcgcgtccttctcttcaccggcggcgtcaagagctggtcgatggtcagcgcgctggagggtgcggggctgaccatcctcggcacctccaccaagaaatcgaccagggaggacaaggagcgcatcaagaagatgaagggcgaagagttccaccagtgggacgatttgaagccgcgcgacatctacaggatgctggccgacgatcaggccgacatcatgatgtccggcggccgctcgcagttcatctcgctgaaggccaaggttccctggctcgacatcaaccaggagcgccaccacgcctatgccggctatgacggcatcgtcaatctctgcgaggagatcgacaaaacgctgtcgaatccgatctggcgtcaggtgcgtcagccggcaccgtgggagtccggcgcgtcctccacccttctggcttcctcgatggcggcggagtga

SEQ ID NO: 87 - DNA, 1383 bp, Azospirillum lipoferum
  gene 1..1383: note = nifK
  source 1..1383: mol_type = genomic DNA
atgtcccacatccagcgcttcccctccgccgccaaggccgcctccaccaacccgctgaagatgagccagccgctgggtgcggctctggcctatctcggcgtcgaccgctgcctgccgctgttccatggctcgcagggctgcaccgccttcgggctggtcctgctggtgcgccatttccgcgaggcgatcccgctccagaccacggcgatggatcaggtcgccaccatcctcggcggctacgacaatctggagcaggcgatccgcaccatcgtcgagcgcaaccagcccgccatgatcggcgtcgccaccaccggcgtcaccgagaccaagggcgaggatatggccggacagtacacgctgttccgccagcgcaaccccgacttggccgacacggccctggtcttcgccaacacccccgacttcgccggcggcttcgaggacggcttcgccgccgcggtcaccgcgatggtcgagcggttggtcgaaccgtcgccggtgcgcatcccgacccaggtcaacgtgctggccggctgccatctgtcccccggcgacgtggaggaactgcgcgacatcatcgaaggcttcggcctgtcgccgatcttcctgcccgacctgtcgctgtcgatggcgggccgccagccggccgacttcaccgccacctcgctgggcggcgtgaccgtcgatcagatccgcgccatgggcgcttcggccctcaccatcgtggtcggtgagcatatgcgggtggccggtaacgcgctggagctgaagaccgacgtgcccagccatttcttcaaccgcctgaccgggctggaggcgacggacaagctggtccggctgctgatggagttgtcgggcaagccggcgcccgcccggctgcggcgccagcgcgaaagcctggtcgatgccatgctcgacgggcatttcttctacagccgcaagcgcatcgccgtcgcgctggagcccgacctgctctatgccgtcaccggcttcctcgccgacatgggggccgaggtgatcgccgcggtgtccccgacgcagagcccggtgctggagcggttgaaggccgccaccatcatggtcggcgatcattccgacgtggagacgctggcccgcgacgccgacctgatcgtctccaactcgcacgggcggcagggagccgcgcggatcggcgtggctctgcaccgcatgggcctgccgctgttcgaccggctgggggccggcctgcgcgtccaggtcggctaccgcggcacgcgggaactgctgtgcgacatcggcaacctgttcctcgcccgcgagatggaccacgagcacgggcacgagagccacgaccacggggaatcccacggctgcggaggcggatcatgcggatgcaacgccgtctga

SEQ ID NO: 88 - DNA, 1560 bp, Azospirillum lipoferum
  gene 1..1560: note = nifK1
  source 1..1560: mol_type = genomic DNA
atgaccgacaagctttcgcagagcgccgacaaggtcctcgaccactacaccctcttccggcagcccgaatacgcggcgatgttcgagaagaagaagaccgagttcgagtacggccattcggacgaggaagtcgcccgcgtgtccgaatggaccaagtccgaggactacaaggcgaagaacttcgcccgtgaagcggtcgtcatcaacccgaccaaggcctgccagccgatcggcgcaatgttcgccgcccagggcttcgaaggcaccctgcccttcgtccacggctcccagggctgcgtcgcctattaccgcacccacctgacccgtcacttcaaggagccgaacagcgcggtctcctcgtcgatgacggaggacgcggcggtgttcggcggcctgaacaacatgatcgacggcctggcgaacgcctatgcgctctacaagccgaagatgatcgcggtgatgaccacctgcatggccgaagtcatcggcgacgatttgcagggcttcatcgccaatgcgaagaccaaggacagcgtcccggccgacttcccggtcccctacgcccacaccccggccttcgtcggcagccacatcgtcggctacgacaacatgatcaaggggatcctgaccaacttctggggtacgtcggagaatttcgacacacccaagaccgagcagatcaacctgatcccgggcttcgacggcttcgccgtcggcaacaaccgcgaactgaagcgcatcgccggcgaattcggcgtgaagctgcaaatcctgtccgacgtgtccgacaatttcgacacgccgatggatggcgagtaccgcatgtatgacggcggcaccaccatcgaggagaccaaggaggccctgcacgccaaggccaccatctccatgcaggagtacaacacgacccagaccctgcaattctgcaaggagaagggtcaggaagtcgccaagttcaactacccgatgggcgtcaccggcaccgacgagctgctgctgaagctcgccgaactgtcgggcaagccggtcccggccagcctgaagctggagcgcggccgtctggtcgacgccatcgccgacagccacacccacatgcacggcaagcgcttcgccgtctatggcgacccggacttctgcctgggcatgtccaagttcctgctggagctgggtgcggagccggtgcacatcctgtcgacgtcgggctccaagaagtgggagaagcaggtccagaaggtgctggacggctcgcccttcggcgcctcgggcaaggcccatggcggcaaggatctgtggcacctgcgttcgctgatcttcaccgacaaggtggactacatcatcggcaacagctacggcaagtatctggagcgcgacaccaaggttccgctgatccgcctgacctacccgatcttcgaccgccaccaccaccaccgctacccgacctggggctaccagggcgcgctgaacgtgctggtacggatcctggaccggatcttcgaggacatcgacgccaacaccaacatcgtcggccagaccgactactcgttcgacctgatccgctga

SEQ ID NO: 89 - DNA, 1530 bp, Azospirillum lipoferum
  gene 1..1530: note = nifA
  source 1..1530: mol_type = genomic DNA
atgttgacctctgatattgttggcaaattgcgctgcatcgcagcagaccccaaagcgggcatcgcaaggggcctcgacaccgggacgacgaagatcggtcccgtttgggagggtgacgtgggcgacaccgtggatttcgaagcgctgcgccagcgggcggtccactccctgttcgaacatctggaatccatgtgcgtcggcgccgtcgccgtcgaccacaccggccgcatcgcctggatggacgagaagtacaaggctctgctgggcgttcccgacgacccgcgcggccggcaggtggaggacgtcatccccaacagccagctgcgccgggtgatcgacagcggccagccgcagccgctggacatcatggagttcgacgaccggtccttcgtggtgacgcggatgccgctgttcggcaccgacggttcgatcatcggcgccatcggcttcgtgctgttcgaccgcgccgaatatctccgcccgctggtccgcaaatacgagaagatgcaggaggagctggcccgcacccagcaggagctggcgcatgagcgccgcgccaaatactccttctcgcagttcctgggcgccagcgaatcgatccgcgagatcaagcggctggggcgccgcgccgcccagatggattcgaccgtcctgctgctgggcgaaaccgggaccggcaaggagctgctggcccaggccatccattccgccagcccgcgggcgtccaagcccttcgtcggcgtcaatgtcgccgccattccggaaaccctgctggaggcggagttcttcggcgtcgcccccggcgccttcaccggcgccgaccgccgccaccgcgacggcaagttccagctcgccaacggcggcaccctgttcctcgacgagatcggcgacatgccgctgccggtgcaggccaagcttctgcgcgtgctgcaggagcgggagatcgagccgctcggctccaacaaggtggtgcgggtcgatgtccgcatcatcgccgccaccagccgtgacctgcacgccctggtgcgtgagaagcagttccgcgccgacctctattaccggctgaatgtggtgccgatcaccctgccgccgctgcgcgaccggccggaggacatcgagagcatcgccgaccgcatcctggaacagctggcgatccagcagggcacgccgccgcgcgagctgctggaatcggcggtgcaggtgctgcgcgactatgactggcccggcaatgtgcgcgagctttacaacacgctggaacgggtggtggcgctgaccgatgcgccgatcctgaccgcgccgcacatccgcagcgtgctgcccggccagcatccggccggcgcgtcggccctgccgctggcggccggcgcgcggccgttgcaggaggtgctgcacgccgccgagcgccacgccatcgccgcggcgcttgaggaggcgaacggcgtcaaggcgcgggcggcgaagctgctgggcatttcgcgcgcgtcgctgtacgaacgcatggtgacgctggggttgggggcgacgcagtag

SEQ ID NO: 90 - DNA, 3030 bp, Azospirillum lipoferum
  gene 1..3030: note = glnE
  source 1..3030: mol_type = genomic DNA
atgccgagtcccatcgcgttctcaagccccttgccgaagcctttcgacagcgcgcaggcggcgctggggatggagcgctggcgccagcaggccgccgcggcggagccggagacccgcgcctgggcggaagccttcgccgattcggagaccggccgggcgctgatcggggcggtgtgcggcaacagcccgtatctcggccacagcctgacgcgggagttgcccttcgtcgcccgtacagtgcaggacggcttcgacgacaccttcgccgcgctgatcgccgctctccatgccgagcatggcgaggagaaatcgatggaccggctgatggccggcctgcgggtggcgaagcggcgggcggcgctgctgatcgcgctggccgacatcgccggcgcgtggccgctgttccgcgtcaccggcgccctgtcggagctggcggagacgggggtgcagctggccgcgaatttcctgctgcgccgcgccagggaggcggggacgctgacgctgccggatccgcagcgaccgtgggtcggttcgggcctgatcgttttgggcatgggtaagcttggcgggcgcgaactcaactattccagcgacatcgacctgatcgtcctgtatgacgacgctgttgtgcagacgccccagccggacaacctcgcgcgaaccttcatcaggctcgcacgcgatcttgtccgcattatggatgaacggaccaaggacggctacgtcttccgcaccgaccttcggcttaggcccgatcccggcgccacgccgctggcggtttccgtctccgcagccgaaatttattacggcagcgtcggtcagaactgggaacgcgcggcgatgatcaaggcccgtcccatcgccggcgatctggaggcgggcgcctcctttgtccgcttcctggagcccttcgtctggcgccgcaacctggatttcgccgccatccaggacatccattcgatcaaacgccagatcaacgcccacaagggccaccgcgaggtgacggtcaacggccacgacatcaaggtcggccgcggcggcatccgcgagatcgagttcttcgcccagacccagcagctgatcttcggcgggcgcgacccgcgcgtgcgaatcgctccgaccctgatggcgaacgaggcgctgcgcgacgtcggccgcgtgccgccgcagacggtggaagagcttgccggggcctatcatttcctgcgccgtgtcgaacatcgcatccagatgatcgacgaccagcagacccatcgtattcccgccgacgatgccggggtggcgcatttggccaccttcctcggctatgacgaccccgccgccttccgggcggaactgctggcgacgctggggcaggtggaggaccgctatgccgagctgttcgaggaggcgccgtcgctttccggccccggcaatctggtcttcaccggcaccgaccccgatccgggcacgatggagacgctgaagggcatgggcttcgccgatccggcccgcgtcatcagcgtggtgtcggcctggcatcgcggccgctaccgcgccacccggtcgggccgggcgcgggagctgctgacggagctgacgccggccctgctgagtgcgctgaccaagaccccggcccccgattcggcgctgatgaacttcgacgatttcctcggcaagctgccggccggcgtcggtctgttctcgctgttcgtcgccaatccctggctcctggagctggtggcggagatcatgggcatcgcgccgcagatggcgcagacgctgtcgcgcaacccgtcgctgctcgacgccgtgctgtcgccggacttcttcgacccgctgccgggcaaggaggacgggctggccgacgaccacgcccgcgtgatggcgccggcccgcgatttcgaggatgcgctgaccctgtcgcggcgctggaccaacgaccagcgcttccgcgccggggtgcatatcctgcgcggcatcaccgatggcgaccgctgcggcgccttcctggccgatctggccgacatcgtcgtccccgaccttgcccgccgggtggaggaggagttcgcccagcgccacggccatattcccggcggcgcctgggtggtggtggcgatgggcaagctcggcagccggcagctgaccatcacgtccgacatcgacctgatcgtcatctacgatgtggcgccgggccaagggggcgggggcggtccccgcttgtcggatggtgccaagccgctgtcgcccaacgagtattacatcaagctgactcagcgtctgaccaacgccattaccgcgccgatgggcgacggccggctctacgaggtcgacatgcggctgcgcccgtcgggcaacgccgggccgctcgccacctcgctggacgctttcctgaaatatcaggcgaccgatgcctggacctgggagcatatggccctgacccgcgcccgggtgatcggcggtgatgcggagctggccgggcgggtgtcggcagcgatccgctcggtgctgacggcgccgcgcgatgccgaccggctgctgtgggacgtggccgacatgcggcggcggatcgagaaggagttcgggacgaccaatgtctggaacgtcaaatacgcccgcggcggcctgatcgacatcgagttcatcgcccagtacctgcaactgcgccatggtcacgagcggccggacatcctgcacatcggcaccgccaaggcgctgggctgcgccgcccggacgggcgcgctggcgccggaggtggcggaggatctggagacgacgctgcggctgtggcggcgggtgcagggctttctgcggttgaccaccgccggggtgctcgatcccaatcaggtgtcgcccagcctgctggccgggctggtccgcgccgcctttcctgctgactttcagggcgagcgtgagcctggcactgttgacttccccgaactggaccacaaaatccgtgccgtcgccgcccgcgcccatggtcatttcaagaccctggtcgaggaaccggcgggccgtctggccccacccgccaccacgcctccagcctga

SEQ ID NO: 91 - DNA, 1440 bp, Azospirillum lipoferum
  gene 1..1440: note = amtB1
  source 1..1440: mol_type = genomic DNA
atgaaccgtctgttccttatggccgcaccgatgatggcggttgctctgggcgcggtcggcatgccggccgcagcccttgcccaggatccggcggctgccgccgctgccgcggctgcggctgcggctgccgccgctgctgccgcaccggcggctccggcgctgaatggcggcgacaccgcctggatgctcatctccaccgcgctggtgctgatgatgaccatccccggcctggcgctgttctacggcggcatggtccgcaagatgaacgtgctgtcgacggtgatgcagagcttcgccatcacctgcctgatcagcgtcctgtggtacgtcatcggctacagcctggccttcaccggcaccggtgcctatgtcggcggtctcgaccggctgttcctcaacgggctcgacttcacgaaggccttcgtgctgggcgaggcgaccgggtcgggcgtcccgacgaccatccccgagccggtcttcatgatgttccagatgacctttgcgatcatcaccccggccctgatcaccggcgccttcgccgaccgcatgaagttctcctccctgctggtcttcaccgcgctgtggtcgatcgtggtctatgcgccgatcgcccactgggtctggtacccgtcgggcttcctgttcggcctgggcgtgctggacttcgccggcggcacggtcgtgcacatcaacgccggcgtcgccggcctggtcgccgcgctggtgatcggcaagcgcaagggctacccgaaggaagccttcatgccgcacaacctggtgctgtcgctgatcggcgcctcgctgctgtgggtcggctggttcggcttcaacgccggttcggccctgaccgccggtccgcgtgccggcatggcgctggccgccacgcacatcgccaccgccggtgccgccatgggctggctgttcgcggagtggatcgtcaagggcaagccgtcgatcctcggcatcatctccggcgccgtcgccggcctggtcgcggtgaccccggccgccggcttcgtcgacccgacgggcgccatcgtcatcggcatcgtcgccggcgtggtctgcttctggtcggccaccagcctcaagcacatgctgggctatgacgacagcctggacgccttcggcgtgcacggcgtcggcggcctgatcggcgccatcctgaccggcgtcttcgccaagatgtcggtgtccaacagcgaaggcggcttcgcctccgtcctgcaggccgacccgaaggccacgctgggcctgctggaaggcaacgccgccgccgtctggatccaggtccagggcgtcctctacaccatggtctggtgcgccatcgccaccttcgtcctgctgaagatcgtcgatgtggtcatgggcctgcgcgtcgaagaggatgtggagcgcgacggtctcgacctcgccctgcatggcgagagcatccactaa

SEQ ID NO: 92 - DNA, 1227 bp, Azospirillum lipoferum
  gene 1..1227: note = amtB2
  source 1..1227: mol_type = genomic DNA
atggatgcggcaaagacgggtggcgacgtccttttcgtgctgatgggcgcggtgatggtgctggcgatgcattgcggcttcgccctgctggaggtcgggacggtccggcgcaagaatcaggtcaacgcgctggtgaagatcctgtcggacttcgccatgtcgaccatcgcctattttttcgtcggttatgccgtggcctacggcatcgacttcttcgccgacgcccacacgctggtcggcaagggaagcggcgggttcgcggcctatggctacgatctggtgaagttcttcttcctggcgaccttcgccgccgcggtgccggccatcgtctcgggcggcatcgccgagcgtgctaggttctggccgcaggccgccgccacgctggcgctgatcgcgctgttctatccattgctggaaggcacggtctggggcacccgcttcggcctgcaaagctggatggccgcgaccttcggccagcctttccacgacttcgccggatctgtggtggtgcatgccttcggcggctgggtggcgctgggtgccgtgctgaacctcggcaaccgccgcggccgctaccgtccgaacggctcgctgatcgccattccgccgtcgaacatccccttcctggcgctgggcgcctgggtgctgtgcgtggggtggttcggcttcaacgtgatgagcgcccaggtgctggatggcgtgacgggtctggtggcgctgaactcgctgatggcgatggtcggcggcatcgtcacctcgctggtgatcagccgcaccgatcccggcttcgtccacaacggcgcgctggccggtctggtggcggtctgcgccgggtccgacgtgatgcacccgctgggcgcgctggtcaccggcggcatcgccgggctgctgttcgtctgggccttcaacaaatgccagatcgactggaagatcgacgacgtgctgggcgtctggccgctgcacggtctgtgcggcctgaccggcggcctgctggccggcgtcttcgggcaggaggcactgggcggccttggcggcgtgtcgatcctcagccagatcgtcggcacggcaagcggcgccagcttcggattcgtctcgggtctggcggtctacggcctgctgcgcgtcaccgtcggcatccgcctcgatcccgagcaggagtacaagggcgccgacttgtcgttgcaccatatcaccgcgtacccggaagaggacgcgccgaccctgtaa

SEQ ID NO: 93 - DNA, 613 bp, synthetic construct
  misc_feature 1..613: note = Synthetic polynucleotide
  misc_feature 1..613: note = delta-nifL::PinfC
  source 1..613: mol_type = other DNA
atgaccctgaatatgatgatggattcttggttctctggagcgctttatcggcatcctgactgaagaatttgcaggcttcttcccaacctggcttgcacccgtgcaggtagttgtgatgaacatcactgattcgcaggctgaatacgttaacgaattgacccgtaaactgcaaaatgcgggcattcgtgtaaaagcagacttgagaaacgagaagattggctttaaaatccgcgagcacactttacgtcgtgtcccttatatgctggtttgtggtgacaaagaggtcgaagccggcaaagttgctgtgcgtacccgtcgcggtaaagacctgggtagcctggacgtaaatgatgttatcgagaagctgcaacaagagattcgcagccgcagtcttcaacaactggaggaataaggtattaaaggcggaaaacgagttcaaacggcgcgtcccaatcgtattaatggcgagattcgcgccacggaagttcgcttaacaggtctggaaggcgagcagcttggtattgcgatagaactcacttcacgccccgaagggggaagctgcctgaccctacgattcccgctatttcattcactgaccggaggttcaaaatga

SEQ ID NO: 94 - DNA, 1613 bp, synthetic construct
  misc_feature 1..1613: note = Synthetic polynucleotide
  misc_feature 1..1613: note = delta-nifL::PinfC with 500bp flank
  source 1..1613: mol_type = other DNA
accggataagagagaaaagtgtcgacgtcggtccggttgatattgaccggcgcatccgccagctcgcccagtttttggtggatctgtttggcgattttgcgggtcttgccggtgtcggtgccgaaaaaaataccaatatttgccataacacacgctcctgttgaaaaagagatcccgcggggaaaatgcggtgaacatgtcagctattgcgaagagtgtgccagttttgctcacgggcaaaagctgcaccagaatgggtattaatgcaccagcctggcgctttttttcgcggcacgtcccctcgctaatgcccgtctggcgcggctttgacgctgataaggcgctgaataccgatctggatcaaggttttgtcgggttatcgtccaaaaggtgcactctttgcatggttataagtgcctgacatggtgtccgggcgaacgtcgccaggtggcacaaattgtcagaactacgacacgactaaccgaccgcaggagtgtgcgatgaccctgaatatgatgatggattcttggttctctggagcgctttatcggcatcctgactgaagaatttgcaggcttcttcccaacctggcttgcacccgtgcaggtagttgtgatgaacatcactgattcgcaggctgaatacgttaacgaattgacccgtaaactgcaaaatgcgggcattcgtgtaaaagcagacttgagaaacgagaagattggctttaaaatccgcgagcacactttacgtcgtgtcccttatatgctggtttgtggtgacaaagaggtcgaagccggcaaagttgctgtgcgtacccgtcgcggtaaagacctgggtagcctggacgtaaatgatgttatcgagaagctgcaacaagagattcgcagccgcagtcttcaacaactggaggaataaggtattaaaggcggaaaacgagttcaaacggcgcgtcccaatcgtattaatggcgagattcgcgccacggaagttcgcttaacaggtctggaaggcgagcagcttggtattgcgatagaactcacttcacgccccgaagggggaagctgcctgaccctacgattcccgctatttcattcactgaccggaggttcaaaatgacccagcgaaccgagtcgggtaataccgtctggcgcttcgatttatcccagcagttcaccgcgatgcagcggataagcgtggttctcagccgggcgaccgaggttgaacagacactccagcaggtgctgtgcgtattgcacaatgacgcctttttgcagcacggcatgatctgtctgtacgacagccagcaggcgattttgactattgaagcgttgcaggaagccgatcagcagttgatccccggcagctcgcaaattcgctaccgtccgggtgaagggctggtcgggacggtgctttcgcaggggcaatcgttagtgctggcgcgtgtggctgacgatcagcgctttcttgaccgcctgggactgtatgattacaacctgccgtttatcgccgtgccgctgatagggccggatgcgcagacttttggcgtgctgacggcgcaaccgatggcgcgttacgaagagcggttacccgcctgcacccgctttctggaaacggtc

SEQ ID NO: 95 - DNA, 1185 bp, synthetic construct
  misc_feature 1..1185: note = Synthetic polynucleotide
  misc_feature 1..1185: note = glnE-delta-AR-2
  source 1..1185: mol_type = other DNA
tccctgtgcgccgcgtcgccgatggtggccagccaactggcgcgctacccgatcctgctcgatgaactgctcgacccgaacacgctctatcaaccgacggcgatgaacgcctatcgcgatgaactgcgacaatacctgttgcgcgtgccggaagaggatgaagagcagcaactggaggcgctacggcagtttaagcaggcgcagttgttgcgcgtagcggcggcggatatcgccggtacgttacccgtcatgaaagtgagcgatcacttaacctggctggcggaagcgattatcgatgcggtggtgcagcaagcctggaaccagatggtggcgcgttacggccagccgacgcatctgcacgatcgcgaagggcgcggtttcgccgtggtcggttacggcaaacttggcggctgggaattaggttacagctccgatctggatctggtgttcctgcacgactgccccatggatgtgatgaccgatggcgagcgtgaaatcgatggccgccagttctatttgcgcctcgcgcagcgcgtgatgcacctgttcagcacgcgcacgtcgtccggcattctttatgaagtcgatgcgcgtttgcgcccgtccggcgcggccggaatgctggtgaccactgcggaagcgttcgccgattatcaaaaaaatgaagcctggacatgggagcatcaggcgctggcgcgtgcgcgcgtggtgtacggcgatccgcaactgaccgccgaatttgacgccattcgccgcgatatcctgatgacctcccgcgatgccgctaccctgcaaaccgaagtgcgggaaatgcgtgagaaaatgcgcgcccatcttggtaacaagcacaaagaccgtttcgatctgaaagccgatgaaggcggtatcaccgatattgagtttatcgctcagtatctggtgctgcgctttgcccatgagaagccgaaactgacgcgctggtcggataatgtgcgcatcctcgaagggctggcgcaaaacggcatcatggatgagcaggaagcgcaggcattgacgctggcgtacaccacgttgcgtgatgagctgcaccacctggcgctgcaagagctgccaggacatgtggcgctctcctgttttgtcgccgagcgtgcgcttatcaaaaccagctgggacaagtggctggtggaaccgtgcgccccggcgtaa

SEQ ID NO: 96 - DNA, 2085 bp, synthetic construct
  misc_feature 1..2085: note = Synthetic polynucleotide
  misc_feature 1..2085: note = glnE-delta-AR-2 with 500bp flank
  source 1..2085: mol_type = other DNA
taaagcgagcgctcacttacgtgatctgttgacgcagtccgaagcgaccattacttcagccgtttcagcagatacggcggtgtggagtgcgcaatcagccctggcgaaactggtgctcaccgagtggttagtgacgcagggctggcgaaccttccttgatgaaaaagcgcaggctaagtttgccgactcctttaaacgctttgctgacgttcatctgtcacgcagcgccgccgagctgaaaaaagcctttgcccagccgctgggcgacagctatcgcgaccagttaccgcggctggcgcgtgatatcgacagcgcgttattgctggccggacattacgatcgcgcgcgcgccgtggagtggctggaaaactggcaggggcttcagcacgctattgaaacgcgccagagagttgaaatcgaacatttccgtaataccgccattacccaggagccgttctggttgcacagcggaaaacgttaacgaaaggatatttcgcatgtccctgtgcgccgcgtcgccgatggtggccagccaactggcgcgctacccgatcctgctcgatgaactgctcgacccgaacacgctctatcaaccgacggcgatgaacgcctatcgcgatgaactgcgacaatacctgttgcgcgtgccggaagaggatgaagagcagcaactggaggcgctacggcagtttaagcaggcgcagttgttgcgcgtagcggcggcggatatcgccggtacgttacccgtcatgaaagtgagcgatcacttaacctggctggcggaagcgattatcgatgcggtggtgcagcaagcctggaaccagatggtggcgcgttacggccagccgacgcatctgcacgatcgcgaagggcgcggtttcgccgtggtcggttacggcaaacttggcggctgggaattaggttacagctccgatctggatctggtgttcctgcacgactgccccatggatgtgatgaccgatggcgagcgtgaaatcgatggccgccagttctatttgcgcctcgcgcagcgcgtgatgcacctgttcagcacgcgcacgtcgtccggcattctttatgaagtcgatgcgcgtttgcgcccgtccggcgcggccggaatgctggtgaccactgcggaagcgttcgccgattatcaaaaaaatgaagcctggacatgggagcatcaggcgctggcgcgtgcgcgcgtggtgtacggcgatccgcaactgaccgccgaatttgacgccattcgccgcgatatcctgatgacctcccgcgatgccgctaccctgcaaaccgaagtgcgggaaatgcgtgagaaaatgcgcgcccatcttggtaacaagcacaaagaccgtttcgatctgaaagccgatgaaggcggtatcaccgatattgagtttatcgctcagtatctggtgctgcgctttgcccatgagaagccgaaactgacgcgctggtcggataatgtgcgcatcctcgaagggctggcgcaaaacggcatcatggatgagcaggaagcgcaggcattgacgctggcgtacaccacgttgcgtgatgagctgcaccacctggcgctgcaagagctgccaggacatgtggcgctctcctgttttgtcgccgagcgtgcgcttatcaaaaccagctgggacaagtggctggtggaaccgtgcgccccggcgtaagtgtggtatcatcgcgcgcaaattttgtatctctcaggagacaggaatgaaagttacgctgccagagttcaatcaagccggtgtcatggtggtgggtgatgtgatgctggatcgctactggtacggcccaaccagccgcatttctccggaagcgccagttccggttgttaaagtcgatactattgaagagcgaccgggcggtgcggcaaacgtggcgatgaacattgcctcgctgggcgcaacggcgcgtctggttggcctgactggcattgatgatgcggcgcgcgcgctgagcaaagcgctggcggatgttaatgttaaatgtgacttcgtttctgttccgactcaccccaccatcactaagctgcgcgtgctgtcgcgtaaccagcaactgattcgc

SEQ ID NO: 97 - DNA, 461 bp, synthetic construct
  misc_feature 1..461: note = Synthetic polynucleotide
  misc_feature 1..461: note = delta-nifL::Prm1
  source 1..461: mol_type = other DNA
atgaccctgaatatgatgatggatgccagccgttctgtaataataaccggacaattcggactgattaaaaaagcgccctcgcggcgctttttttatattctcgactccatttaaaataaaaaatccaatcggatttcactatttaaactggccattatctaagatgaatccgatggaagctcgctgttttaacacgcgttttttaaccttttattgaaagtcggtgcttctttgagcgaacgatcaaatttaagtggattcccatcaaaaaaatattctcaacctaaaaaagtttgtgtaatacttgtaacgctacatggagattaactcaatctagagggtattaataatgaatcgtactaaactggtactgggcgcaactcacttcacgccccgaagggggaagctgcctgaccctacgattcccgctatttcattcactgaccggaggttcaaaatga

SEQ ID NO: 98 - DNA, 1461 bp, synthetic construct
  misc_feature 1..1461: note = Synthetic polynucleotide
  misc_feature 1..1461: note = delta-nifL::Prm1 with 500bp flank
  source 1..1461: mol_type = other DNA
accggataagagagaaaagtgtcgacgtcggtccggttgatattgaccggcgcatccgccagctcgcccagtttttggtggatctgtttggcgattttgcgggtcttgccggtgtcggtgccgaaaaaaataccaatatttgccataacacacgctcctgttgaaaaagagatcccgcggggaaaatgcggtgaacatgtcagctattgcgaagagtgtgccagttttgctcacgggcaaaagctgcaccagaatgggtattaatgcaccagcctggcgctttttttcgcggcacgtcccctcgctaatgcccgtctggcgcggctttgacgctgataaggcgctgaataccgatctggatcaaggttttgtcgggttatcgtccaaaaggtgcactctttgcatggttataagtgcctgacatggtgtccgggcgaacgtcgccaggtggcacaaattgtcagaactacgacacgactaaccgaccgcaggagtgtgcgatgaccctgaatatgatgatggatgccagccgttctgtaataataaccggacaattcggactgattaaaaaagcgccctcgcggcgctttttttatattctcgactccatttaaaataaaaaatccaatcggatttcactatttaaactggccattatctaagatgaatccgatggaagctcgctgttttaacacgcgttttttaaccttttattgaaagtcggtgcttctttgagcgaacgatcaaatttaagtggattcccatcaaaaaaatattctcaacctaaaaaagtttgtgtaatacttgtaacgctacatggagattaactcaatctagagggtattaataatgaatcgtactaaactggtactgggcgcaactcacttcacgccccgaagggggaagctgcctgaccctacgattcccgctatttcattcactgaccggaggttcaaaatgacccagcgaaccgagtcgggtaataccgtctggcgcttcgatttatcccagcagttcaccgcgatgcagcggataagcgtggttctcagccgggcgaccgaggttgaacagacactccagcaggtgctgtgcgtattgcacaatgacgcctttttgcagcacggcatgatctgtctgtacgacagccagcaggcgattttgactattgaagcgttgcaggaagccgatcagcagttgatccccggcagctcgcaaattcgctaccgtccgggtgaagggctggtcgggacggtgctttcgcaggggcaatcgttagtgctggcgcgtgtggctgacgatcagcgctttcttgaccgcctgggactgtatgattacaacctgccgtttatcgccgtgccgctgatagggccggatgcgcagacttttggcgtgctgacggcgcaaccgatggcgcgttacgaagagcggttacccgcctgcacccgctttctggaaacggtc

SEQ ID NO: 99 - DNA, 1185 bp, synthetic construct; identical to SEQ ID NO: 95
  misc_feature 1..1185: note = Synthetic polynucleotide
  misc_feature 1..1185: note = glnE-delta-AR-2
  source 1..1185: mol_type = other DNA

SEQ ID NO: 100 - DNA, 2085 bp, synthetic construct; identical to SEQ ID NO: 96
  misc_feature 1..2085: note = Synthetic polynucleotide
  misc_feature 1..2085: note = glnE-delta-AR-2 with 500bp flank
  source 1..2085: mol_type = other DNA

SEQ ID NO: 101 - DNA, 461 bp, synthetic construct; identical to SEQ ID NO: 97
  misc_feature 1..461: note = Synthetic polynucleotide
  misc_feature 1..461: note = delta-nifL::Prm1
  source 1..461: mol_type = other DNA

SEQ ID NO: 102 - DNA, 1461 bp, synthetic construct; identical to SEQ ID NO: 98
  misc_feature 1..1461: note = Synthetic polynucleotide
  misc_feature 1..1461: note = delta-nifL::Prm1 with 500bp flank
  source 1..1461: mol_type = other DNA

SEQ ID NO: 103 - DNA, 1185 bp, synthetic construct; identical to SEQ ID NO: 95
  misc_feature 1..1185: note = Synthetic polynucleotide
  misc_feature 1..1185: note = glnE-delta-AR-2
  source 1..1185: mol_type = other DNA

SEQ ID NO: 104 - DNA, 2085 bp, synthetic construct; identical to SEQ ID NO: 96
  misc_feature 1..2085: note = Synthetic polynucleotide
  misc_feature 1..2085: note = glnE-delta-AR-2 with 500bp flank
  source 1..2085: mol_type = other DNA

SEQ ID NO: 105 - DNA, 452 bp, synthetic construct
  misc_feature 1..452: note = Synthetic polynucleotide
  misc_feature 1..452: note = delta-nifL::Prm7
  source 1..452: mol_type = other DNA
atgaccctgaatatgatgatggatgccagccgcgtcaggttgaacgtaaaaaagtcggtctgcgcaaagcacgtcgtcgtccgcagttctccaaacgttaattggtttctgcttcggcagaacgattggcgaaaaaacccggtgcgaaccgggtttttttatggataaagatcgtgttatccacagcaatccattgattatctcttctttttcagcatttccagaatcccctcaccacaaagcccgcaaaatctggtaaactatcatccaattttctgcccaaatggctgggattgttcattttttgtttgccttacaacgagagtgacagtacgcgcgggtagttaactcaacatctgaccggtcgataactcacttcacgccccgaagggggaagctgcctgaccctacgattcccgctatttcattcactgaccggaggttcaaaatga

SEQ ID NO: 106 - DNA, 1451 bp, synthetic construct
  misc_feature 1..1451: note = Synthetic polynucleotide
  misc_feature 1..1451: note = delta-nifL::Prm7 with 500bp flank
  source 1..1451: mol_type = other DNA
accggataagagagaaaagtgtcgacgtcggtccggttgatattgaccggcgcatccgccagctcgcccagtttttggtggatctgtttggcgattttgcgggtcttgccggtgtcggtgccgaaaaaaataccaatatttgccataacacacgctcctgttgaaaaagagatcccgcggggaaaatgcggtgaacatgtcagctattgcgaagagtgtgccagttttgctcacgggcaaaagctgcaccagaatgggtattaatgcaccagcctggcgctttttttcgcggcacgtcccctcgctaatgcccgtctggcgcggctttgacgctgataaggcgctgaataccgatctggatcaaggttttgtcgggttatcgtccaaaaggtgcactctttgcatggttataagtgcctgacatggtgtccgggcgaacgtcgccaggtggcacaaattgtcagaactacgacacgactaaccgaccgcaggagtgtgcgatgaccctgaatatgatgatggatgccagccgcgtcaggttgaacgtaaaaaagtcggtctgcgcaaagcacgtcgtcgtccgcagttctccaaacgttaattggtttctgcttcggcagaacgattggcgaaaaaacccggtgcgaaccgggtttttttatggataaagatcgtgttatccacagcaatccattgattatctcttctttttcagcatttccagaatcccctcaccacaaagcccgcaaaatctggtaaactatcatccaattttctgcccaaatggctgggattgttcattttttgtttgccttacaacgagagtgacagtacgcgcgggtagttaactcaacatctgaccggtcgataactcacttcacgccccgaagggggaagctgcctgaccctacgattcccgctatttcattcactgaccggaggttcaaaatgacccagcgaaccgagtcgggtaataccgtctggcgcttcgatttatcccagcagttcaccgcgatgcagcggataagcgtggttctcagccgggcgaccgaggttgaacagacactccagcaggtgctgtgcgtattgcacaatgacgcctttttgcagcacggcatgatctgtctgtacgacagccagcaggcgattttgactattgaagcgttgcaggaagccgatcagcagttgatccccggcagctcgcaaattcgctaccgtccgggtgaagggctggtcgggacggtgctttcgcaggggcaatcgttagtgctggcgcgtgtggctgacgatcagcgctttcttgaccgcctgggactgtatgattacaacctgccgtttatcgccgtgccgctgatagggccggatgcgcagacttttggcgtgctgacggcgcaaccgatggcgcgttacgaagagcggttacccgcctgcacccgctttctggaaacggt

SEQ ID NO: 107 - DNA, 1191 bp, synthetic construct
  misc_feature 1..1191: note = Synthetic polynucleotide
  misc_feature 1..1191: note = glnE-delta-AR-2
  source 1..1191: mol_type = other DNA
atggcgctgaagcacctgatcacgctctgcgcggcgtcgccgatggtcgccagccagctggcgcgccacccgctgctgctggatgagctgctggatcccaacaccctctatcagccgacggcgaccgatgcctatcgcgacgagctgcgccagtacctgctgcgcgtgccggaagaggatgaagagcagcagctggaggcgttgcgccagtttaagcaggcgcagcagctgcatatcgcggcggcggatatcgctggtaccctgccggtgatgaaggtcagcgatcacttaacctggcttgccgaagcgatcctcgacgcggtggtgcagcaggcatgggggcagatggtcgctcgctacggccagccgacccacctgcacgatcgccagggtcgcggcttcgccgtcgtcggctacggtaagcttggcggctgggagctgggctacagctccgatctcgatctggtgttcctccatgactgcccggcggaggtgatgaccgacggcgagcgggagattgacggccgtcagttctacctgcggctggcccagcggatcatgcacctgttcagcacccgcacctcgtccggtattctctacgaagtggacgcccggctgcgtccttctggcgcggcggggatgctggtcaccaccgccgacgcgtttgctgactatcagcagaacgaagcctggacgtgggaacatcaggcgctggtgcgcgcccgcgtggtctatggcgacccggcgctgcaggcgcgctttgacgccattcgtcgcgatatcctgaccaccccgcgggaggggatgaccctgcagaccgaggttcgcgagatgcgcgagaagatgcgcgcccaccttggcaacaaacatcccgatcgttttgatatcaaagccgatgccggcgggatcaccgatattgaatttattactcagtatctggtcctacgctatgccagtgacaagccgaagctgacccgctggtctgacaacgtgcgtattcttgagctgctggcgcagaacgacatcatggacgaggaggaggcgcgcgccttaacgcatgcgtacaccaccttgcgtgatgcgctccatcacctggccctgcaggagcagccgggacacgtggcgccagaggccttcagccgggagcgtcagcaggtcagcgccagctggcagaagtggctgatggcttaa

SEQ ID NO: 108 - DNA, 2191 bp, synthetic construct
  misc_feature 1..2191: note = Synthetic polynucleotide
  misc_feature 1..2191: note = glnE-delta-AR-2 with 500bp flank
  source 1..2191: mol_type = other DNA
cgtaaggcgaccacccagctccgcgcgttgctgaacgacgctgaagccgttctgctggccgcggacaccgccgacgaggcgttattccgcaccgaggtcgtcggcgccaaactggccctgactgaatggctggtccagcgcggctggcgtccgttcctcaacgaggcaggagagaaaaaaatagccggatcgttcaaacggtttgccgatattaacctctcgcgggtggcggccgagctgcgcagcgccgtgcagcatctggcggttgaagatgccgccgaccagttgccgaagctgtcccgcgacatcgacagcgtccagctgctggcgggcgcctatggcgacgccgtcgcgccgtggctggagaactggcaggagcttcaccgtgcaatagcacatgacgatcgcagcgtctttgaatatttccgtcgccaggcgctggctgccgagccgttctggctgcatagtggaaaacgataatttcaggccagggagcccttatggcgctgaagcacctgatcacgctctgcgcggcgtcgccgatggtcgccagccagctggcgcgccacccgctgctgctggatgagctgctggatcccaacaccctctatcagccgacggcgaccgatgcctatcgcgacgagctgcgccagtacctgctgcgcgtgccggaagaggatgaagagcagcagctggaggcgttgcgccagtttaagcaggcgcagcagctgcatatcgcggcggcggatatcgctggtaccctgccggtgatgaaggtcagcgatcacttaacctggcttgccgaagcgatcctcgacgcggtggtgcagcaggcatgggggcagatggtcgctcgctacggccagccgacccacctgcacgatcgccagggtcgcggcttcgccgtcgtcggctacggtaagcttggcggctgggagctgggctacagctccgatctcgatctggtgttcctccatgactgcccggcggaggtgatgaccgacggcgagcgggagattgacggccgtcagttctacctgcggctggcccagcggatcatgcacctgttcagcacccgcacctcgtccggtattctctacgaagtggacgcccggctgcgtccttctggcgcggcggggatgctggtcaccaccgccgacgcgtttgctgactatcagcagaacgaagcctggacgtgggaacatcaggcgctggtgcgcgcccgcgtggtctatggcgacccggcgctgcaggcgcgctttgacgccattcgtcgcgatatcctgaccaccccgcgggaggggatgaccctgcagaccgaggttcgcgagatgcgcgagaagatgcgcgcccaccttggcaacaaacatcccgatcgttttgatatcaaagccgatgccggcgggatcaccgatattgaatttattactcagtatctggtcctacgctatgccagtgacaagccgaagctgacccgctggtctgacaacgtgcgtattcttgagctgctggcgcagaacgacatcatggacgaggaggaggcgcgcgccttaacgcatgcgtacaccaccttgcgtgatgcgctccatcacctggccctgcaggagcagccgggacacgtggcgccagaggccttcagccgggagcgtcagcaggtcagcgccagctggcagaagtggctgatggcttaactataaaatcgggtgtgctattatcgcgcgcaaagtttgcgtctcgcaggagagagtcatgaaagtaacgctgccggagtttgaacgtgcaggagtgttggtggtgggtgatgtgatgctggaccgctactggtacggccccaccagtcgtatttccccggaagccccggtgccggtggtgaaggtggaaaatatcgaagaacgtcctggcggcgcggcaaacgtagcgatgaacatcgcctccctgggggcaacgtcgcgcctggtgggattgaccgggattgatgacgctgcccgcgcgctgagccaggcgctggccaatgtgaatgtgaagtgcgacttcgtctccgtcccgactcacccgaccatcaccaagctgcgggtgctgtcgcgcaatcagcagctgatccgcctcgactttgaagagggcttctccggcgtggatccgcagccgatgcatgagcgcattcagcaggcgctgggagccattggcgcactgg

SEQ ID NO: 109 - DNA, 613 bp, synthetic construct
  misc_feature 1..613: note = Synthetic polynucleotide
  misc_feature 1..613: note = delta-nifL::PinfC
  source 1..613: mol_type = other DNA
atgaccctgaatatgatgctagaagcgtcaggtaccggtcatgattcaccgtgcgattctcggttccctggagcgcttcattggcatcctgaccgaagagttcgctggcttcttcccaacctggattgcaccagtgcaggtagtggtcatgaatattaccgattctcaggctgaatacgttaacgaattgacgcgtaaactacaaaatgcgggcattcgtgtaaaagcagacttgagaaatgagaagattggctttaaaatccgcgagcacactttacgtcgtgtcccgtatatgttggtctgtggcgacaaagaagtcgaagccggcaaagtggccgtgcgcacccgtcgcgggaaagacctcggcagcatggacgtaagtgaagtgattgagaagctgcaacaagagattcgcagccgcagtcttcaacaactggaggaataaggtattaaaggcggaaaacgagttcaaacggcacgtccgaatcgtatcaatggcgagattcgcgccctggaagttcgcgccattgagctggcttcccgaccgcagggcggcacctgcctgaccctgcgtttcccgctgtttaacaccctgaccggaggtgaagcatga

SEQ ID NO: 110 - DNA, 1613 bp, synthetic construct
  misc_feature 1..1613: note = Synthetic polynucleotide
  misc_feature 1..1613: note = delta-nifL::PinfC with 500bp flank
  source 1..1613: mol_type = other DNA
ggccgtcgcccagcgtcggcgtccccaacagcagggccgggtaggccagcaggtccgccagcgtggcgcggttaatattgaccggggcggcggcggcctcccccagctgcttgtggatcattttcgcgatcttgcgggttttaccggtatcggtaccaaagaaaatgccaatgttcgccatagtacgctcctgtcggaatggtgttgaaaaaaggaatgacgacagaggtattgcgaaggctgtgccaggttgccctgcaccgcgacggcccatccctgccccatcaggatcgcttcgcatcacgatgccgcgcgccaaaggcgcacccggcggggcgaaaggtaaaaatccgtgaattttccccctgtcggatcaatgtttcgcgtggtcgttccgataagggcgcacactttgcatggttatccgggttcggcttaccccgccgcgttttgcgcacggtgtcggacaatttgtcataactgcgacacaggagtttgcgatgaccctgaatatgatgctagaagcgtcaggtaccggtcatgattcaccgtgcgattctcggttccctggagcgcttcattggcatcctgaccgaagagttcgctggcttcttcccaacctggattgcaccagtgcaggtagtggtcatgaatattaccgattctcaggctgaatacgttaacgaattgacgcgtaaactacaaaatgcgggcattcgtgtaaaagcagacttgagaaatgagaagattggctttaaaatccgcgagcacactttacgtcgtgtcccgtatatgttggtctgtggcgacaaagaagtcgaagccggcaaagtggccgtgcgcacccgtcgcgggaaagacctcggcagcatggacgtaagtgaagtgattgagaagctgcaacaagagattcgcagccgcagtcttcaacaactggaggaataaggtattaaaggcggaaaacgagttcaaacggcacgtccgaatcgtatcaatggcgagattcgcgccctggaagttcgcgccattgagctggcttcccgaccgcagggcggcacctgcctgaccctgcgtttcccgctgtttaacaccctgaccggaggtgaagcatgatccctgaatccgacccggacaccaccgtcagacgcttcgacctctctcagcagttcaccgccatgcagcggataagcgtggtgctgagccgggccaccgaggccagcaaaacgctgcaggaggtgctcagcgtattacacaacgatgcctttatgcagcacgggatgatctgcctgtacgacagcgagcaggagatcctcagtatcgaagcgctgcagcaaaccggccagcagcccctccccggcagcacgcagatccgctatcgccccggcgagggactggtggggaccgtgctggcccaggggcagtcgctggtgctgccccgggtcgccgacgatcagcgttttctcgaccgcctgagcctctacgattacgatctgccgtttatcgccgtaccgttgatggggcccaacgcccggccaataggggtgctggcggcccagccgatggcgcgccaggaagagcggctgccggcctgcacccgttttctcgaaaccgtc

SEQ ID NO: 111 - DNA, 1155 bp, synthetic construct
  misc_feature 1..1155: note = Synthetic polynucleotide
  misc_feature 1..1155: note = glnE-delta-AR-2 36bp deletion
  source 1..1155: mol_type = other DNA
atggcgctgaagcacctgatcacgctctgcgcggcgtcgccgatggtcgccagccagctggcgcgccacccgctgctgctggatgagctgctggatcccaacaccctctatcagccgacggcgaccgatgcctatcgcgacgagctgcgccagtacctgctgcgcgtgccggaagaggatgaagagcagcagctgcatatcgcggcggcggatatcgctggtaccctgccggtgatgaaggtcagcgatcacttaacctggcttgccgaagcgatcctcgacgcggtggtgcagcaggcatgggggcagatggtcgctcgctacggccagccgacccacctgcacgatcgccagggtcgcggcttcgccgtcgtcggctacggtaagcttggcggctgggagctgggctacagctccgatctcgatctggtgttcctccatgactgcccggcggaggtgatgaccgacggcgagcgggagattgacggccgtcagttctacctgcggctggcccagcggatcatgcacctgttcagcacccgcacctcgtccggtattctctacgaagtggacgcccggctgcgtccttctggcgcggcggggatgctggtcaccaccgccgacgcgtttgctgactatcagcagaacgaagcctggacgtgggaacatcaggcgctggtgcgcgcccgcgtggtctatggcgacccggcgctgcaggcgcgctttgacgccattcgtcgcgatatcctgaccaccccgcgggaggggatgaccctgcagaccgaggttcgcgagatgcgcgagaagatgcgcgcccaccttggcaacaaacatcccgatcgttttgatatcaaagccgatgccggcgggatcaccgatattgaatttattactcagtatctggtcctacgctatgccagtgacaagccgaagctgacccgctggtctgacaacgtgcgtattcttgagctgctggcgcagaacgacatcatggacgaggaggaggcgcgcgccttaacgcatgcgtacaccaccttgcgtgatgcgctccatcacctggccctgcaggagcagccgggacacgtggcgccagaggccttcagccgggagcgtcagcaggtcagcgccagctggcagaagtggctgatggcttaa

SEQ ID NO: 112 - DNA, 2155 bp, synthetic construct
  misc_feature 1..2155: note = Synthetic polynucleotide
  misc_feature 1..2155: note = glnE-delta-AR-2 36bp deletion
  source 1..2155: mol_type = other DNA
cgtaaggcgaccacccagctccgcgcgttgctgaacgacgctgaagccgttctgctggccgcggacaccgccgacgaggcgttattccgcaccgaggtcgtcggcgccaaactggccctgactgaatggctggtccagcgcggctggcgtccgttcctcaacgaggcaggagagaaaaaaatagccggatcgttcaaacggtttgccgatattaacctctcgcgggtggcggccgagctgcgcagcgccgtgcagcatctggcggttgaagatgccgccgaccagttgccgaagctgtcccgcgacatcgacagcgtccagctgctggcgggcgcctatggcgacgccgtcgcgccgtggctggagaactggcaggagcttcaccgtgcaatagcacatgacgatcgcagcgtctttgaatatttccgtcgccaggcgctggctgccgagccgttctggctgcatagtggaaaacgataatttcaggccagggagcccttatggcgctgaagcacctgatcacgctctgcgcggcgtcgccgatggtcgccagccagctggcgcgccacccgctgctgctggatgagctgctggatcccaacaccctctatcagccgacggcgaccgatgcctatcgcgacgagctgcgccagtacctgctgcgcgtgccggaagaggatgaagagcagcagctgcatatcgcggcggcggatatcgctggtaccctgccggtgatgaaggtcagcgatcacttaacctggcttgccgaagcgatcctcgacgcggtggtgcagcaggcatgggggcagatggtcgctcgctacggccagccgacccacctgcacgatcgccagggtcgcggcttcgccgtcgtcggctacggtaagcttggcggctgggagctgggctacagctccgatctcgatctggtgttcctccatgactgcccggcggaggtgatgaccgacggcgagcgggagattgacggccgtcagttctacctgcggctggcccagcggatcatgcacctgttcagcacccgcacctcgtccggtattctctacgaagtggacgcccggctgcgtccttctggcgcggcggggatgctggtcaccaccgccgacgcgtttgctgactatcagcagaacgaagcctggacgtgggaacatcaggcgctggtgcgcgcccgcgtggtctatggcgacccggcgctgcaggcgcgctttgacgccattcgtcgcgatatcctgaccaccccgcgggaggggatgaccctgcagaccgaggttcgcgagatgcgcgagaagatgcgcgcccaccttggcaacaaacatcccgatcgttttgatatcaaagccgatgccggcgggatcaccgatattgaatttattactcagtatctggtcctacgctatgccagtgacaagccgaagctgacccgctggtctgacaacgtgcgtattcttgagctgctggcgcagaacgacatcatggacgaggaggaggcgcgcgccttaacgcatgcgtacaccaccttgcgtgatgcgctccatcacctggccctgcaggagcagccgggacacgtggcgccagaggccttcagccgggagcgtcagcaggtcagcgccagctggcagaagtggctgatggcttaactataaaatcgggtgtgctattatcgcgcgcaaagtttgcgtctcgcaggagagagtcatgaaagtaacgctgccggagtttgaacgtgcaggagtgttggtggtgggtgatgtgatgctggaccgctactggtacggccccaccagtcgtatttccccggaagccccggtgccggtggtgaaggtggaaaatatcgaagaacgtcctggcggcgcggcaaacgtagcgatgaacatcgcctccctgggggcaacgtcgcgcctggtgggattgaccgggattgatgacgctgcccgcgcgctgagccaggcgctggccaatgtgaatgtgaagtgcgacttcgtctccgtcccgactcacccgaccatcaccaagctgcgggtgctgtcgcgcaatcagcagctgatccgcctcgactttgaagagggcttctccggcgtggatccgcagccgatgcatgagcgcattcagcaggcgctgggagccattggcgcactgg

SEQ ID NO: 113 - DNA, 412 bp, synthetic construct
  misc_feature 1..412: note = Synthetic polynucleotide
  misc_feature 1..412: note = delta-nifL::Prm8.2
  source 1..412: mol_type = other DNA
atgaccctgaatatgatgctcgacgccgtcctcgcagtaccattgcaaccgactttacagcaagaagtgattctggcacgcatggaacaaattcttgccagtcgggctttatccgatgacgaacgcgcacagcttttatatgagcgcggagtgttgtatgatagtctcggtctgagggcattagcgcgaaatgatttttcacaagcgctggcaatccgacccgatatgcctgaagtattcaattacttaggcatttacttaacgcaggcaggcaattttgatgctgcctatgaagcgtttgattctgtacttgagcttgatcgccattgagctggcttcccgaccgcagggcggcacctgcctgaccctgcgtttcccgctgtttaacaccctgaccggaggtgaagcatga

SEQ ID NO: 114 - DNA, 1389 bp, synthetic construct
  misc_feature 1..1389: note = Synthetic polynucleotide
  misc_feature 1..1389: note = delta-nifL::Prm8.2 with 500bp flank
  source 1..1389: mol_type = other DNA
cccaacagcagggccgggtaggccagcaggtccgccagcgtggcgcggttaatattgaccggggcggcggcggcctcccccagctgcttgtggatcattttcgcgatcttgcgggttttaccggtatcggtaccaaagaaaatgccaatgttcgccatagtacgctcctgtcggaatggtgttgaaaaaaggaatgacgacagaggtattgcgaaggctgtgccaggttgccctgcaccgcgacggcccatccctgccccatcaggatcgcttcgcatcacgatgccgcgcgccaaaggcgcacccggcggggcgaaaggtaaaaatccgtgaattttccccctgtcggatcaatgtttcgcgtggtcgttccgataagggcgcacactttgcatggttatccgggttcggcttaccccgccgcgttttgcgcacggtgtcggacaatttgtcataactgcgacacaggagtttgcgatgaccctgaatatgatgctcgacgccgtcctcgcagtaccattgcaaccgactttacagcaagaagtgattctggcacgcatggaacaaattcttgccagtcgggctttatccgatgacgaacgcgcacagcttttatatgagcgcggagtgttgtatgatagtctcggtctgagggcattagcgcgaaatgatttttcacaagcgctggcaatccgacccgatatgcctgaagtattcaattacttaggcatttacttaacgcaggcaggcaattttgatgctgcctatgaagcgtttgattctgtacttgagcttgatcgccattgagctggcttcccgaccgcagggcggcacctgcctgaccctgcgtttcccgctgtttaacaccctgaccggaggtgaagcatgatccctgaatccgacccggacaccaccgtcagacgcttcgacctctctcagcagttcaccgccatgcagcggataagcgtggtgctgagccgggccaccgaggccagcaaaacgctgcaggaggtgctcagcgtattacacaacgatgcctttatgcagcacgggatgatctgcctgtacgacagcgagcaggagatcctcagtatcgaagcgctgcagcaaaccggccagcagcccctccccggcagcacgcagatccgctatcgccccggcgagggactggtggggaccgtgctggcccaggggcagtcgctggtgctgccccgggtcgccgacgatcagcgttttctcgaccgcctgagcctctacgattacgatctgccgtttatcgccgtaccgttgatggggcccaacgcccggccaataggggtgctggcggcccagccgatggcgcgccaggaagagcggctgccggcctgcacccgttttctcgaaaccgtc

SEQ ID NO: 115 - DNA, 1155 bp, synthetic construct; identical to SEQ ID NO: 111
  misc_feature 1..1155: note = Synthetic polynucleotide
  misc_feature 1..1155: note = glnE-delta-AR-2 36bp deletion
  source 1..1155: mol_type = other DNA

SEQ ID NO: 116 - DNA, 2155 bp, synthetic construct; identical to SEQ ID NO: 112
  misc_feature 1..2155: note = Synthetic polynucleotide
  misc_feature 1..2155: note = glnE-delta-AR-2 36bp deletion
  source 1..2155: mol_type = other DNA

SEQ ID NO: 117 - DNA, 413 bp, synthetic construct
  misc_feature 1..413: note = Synthetic polynucleotide
  misc_feature 1..413: note = delta-nifL::Prm6.2
  source 1..413: mol_type = other DNA
atgaccctgaatatgatgctcgagctaaagttctcggctaatcgctgataacatttgacgcaatgcgcaataaaagggcatcatttgatgccctttttgcacgctttcataccagaacctggctcatcagtgattttttttgtcataatcattgctgagacaggctctgaagagggcgtttatacaccaaaccattcgagcggtagcgcgacggcaagtcagcgttctcctttgcaatagcagggaagaggcgccagaaccgccagcgttgaagcagtttgaacgcgttcagtgtataatccgaaacttaatttcggtttggagccattgagctggcttcccgaccgcagggcggcacctgcctgaccctgcgtttcccgctgtttaacaccctgaccggaggtgaagcatga

SEQ ID NO: 118 - DNA, 1413 bp, synthetic construct
  misc_feature 1..1413: note = Synthetic polynucleotide
  misc_feature 1..1413: note = delta-nifL::Prm6.2 with 500bp flank
  source 1..1413: mol_type = other DNA
ggccgtcgcccagcgtcggcgtccccaacagcagggccgggtaggccagcaggtccgccagcgtggcgcggttaatattgaccggggcggcggcggcctcccccagctgcttgtggatcattttcgcgatcttgcgggttttaccggtatcggtaccaaagaaaatgccaatgttcgccatagtacgctcctgtcggaatggtgttgaaaaaaggaatgacgacagaggtattgcgaaggctgtgccaggttgccctgcaccgcgacggcccatccctgccccatcaggatcgcttcgcatcacgatgccgcgcgccaaaggcgcacccggcggggcgaaaggtaaaaatccgtgaattttccccctgtcggatcaatgtttcgcgtggtcgttccgataagggcgcacactttgcatggttatccgggttcggcttaccccgccgcgttttgcgcacggtgtcggacaatttgtcataactgcgacacaggagtttgcgatgaccctgaatatgatgctcgagctaaagttctcggctaatcgctgataacatttgacgcaatgcgcaataaaagggcatcatttgatgccctttttgcacgctttcataccagaacctggctcatcagtgattttttttgtcataatcattgctgagacaggctctgaagagggcgtttatacaccaaaccattcgagcggtagcgcgacggcaagtcagcgttctcctttgcaatagcagggaagaggcgccagaaccgccagcgttgaagcagtttgaacgcgttcagtgtataatccgaaacttaatttcggtttggagccattgagctggcttcccgaccgcagggcggcacctgcctgaccctgcgtttcccgctgtttaacaccctgaccggaggtgaagcatgatccctgaatccgacccggacaccaccgtcagacgcttcgacctctctcagcagttcaccgccatgcagcggataagcgtggtgctgagccgggccaccgaggccagcaaaacgctgcaggaggtgctcagcgtattacacaacgatgcctttatgcagcacgggatgatctgcctgtacgacagcgagcaggagatcctcagtatcgaagcgctgcagcaaaccggccagcagcccctccccggcagcacgcagatccgctatcgccccggcgagggactggtggggaccgtgctggcccaggggcagtcgctggtgctgccccgggtcgccgacgatcagcgttttctcgaccgcctgagcctctacgattacgatctgccgtttatcgccgtaccgttgatggggcccaacgcccggccaataggggtgctggcggcccagccgatggcgcgccaggaagagcggctgccggcctgcacccgttttctcgaaaccgtc

SEQ ID NO: 119 - DNA, 513 bp, synthetic construct
  misc_feature 1..513: note = Synthetic polynucleotide
  misc_feature 1..513: note = delta-nifL::Prm1.2
  source 1..513: mol_type = other DNA
atgaccctgaatatgatgctcgagcccgctgaccgaccagaacttccaccttggactcggctatacccttggcgtgacggcgcgcgataactgggactacatccccattccggtgatcttaccattggcgtcaataggttacggtccggcgactttccagatgacctatattcccggcacctacaataacggtaacgtttacttcgcctgggctcgtatacagttttaattcgctaagtcttagcaataaatgagataagcggtgtgtcttgtggaaaaacaaggactaaagcgttacccactaaaaaagatagcgacttttatcactttttagcaaagttgcactggacaaaaggtaccacaattggtgtactgatactcgacacagcattagtgtcgatttttcatataaaggtaattttggccattgagctggcttcccgaccgcagggcggcacctgcctgaccctgcgtttcccgctgtttaacaccctgaccggaggtgaagcatga

SEQ ID NO: 120 - DNA, 1513 bp, synthetic construct
  misc_feature 1..1513: note = Synthetic polynucleotide
  misc_feature 1..1513: note = delta-nifL::Prm1.2 with 500bp flank
  source 1..1513: mol_type = other DNA
ggccgtcgcccagcgtcggcgtccccaacagcagggccgggtaggccagcaggtccgccagcgtggcgcggttaatattgaccggggcggcggcggcctcccccagctgcttgtggatcattttcgcgatcttgcgggttttaccggtatcggtaccaaagaaaatgccaatgttcgccatagtacgctcctgtcggaatggtgttgaaaaaaggaatgacgacagaggtattgcgaaggctgtgccaggttgccctgcaccgcgacggcccatccctgccccatcaggatcgcttcgcatcacgatgccgcgcgccaaaggcgcacccggcggggcgaaaggtaaaaatccgtgaattttccccctgtcggatcaatgtttcgcgtggtcgttccgataagggcgcacactttgcatggttatccgggttcggcttaccccgccgcgttttgcgcacggtgtcggacaatttgtcataactgcgacacaggagtttgcgatgaccctgaatatgatgctcgagcccgctgaccgaccagaacttccaccttggactcggctatacccttggcgtgacggcgcgcgataactgggactacatccccattccggtgatcttaccattggcgtcaataggttacggtccggcgactttccagatgacctatattcccggcacctacaataacggtaacgtttacttcgcctgggctcgtatacagttttaattcgctaagtcttagcaataaatgagataagcggtgtgtcttgtggaaaaacaaggactaaagcgttacccactaaaaaagatagcgacttttatcactttttagcaaagttgcactggacaaaaggtaccacaattggtgtactgatactcgacacagcattagtgtcgatttttcatataaaggtaattttggccattgagctggcttcccgaccgcagggcggcacctgcctgaccctgcgtttcccgctgtttaacaccctgaccggaggtgaagcatgatccctgaatccgacccggacaccaccgtcagacgcttcgacctctctcagcagttcaccgccatgcagcggataagcgtggtgctgagccgggccaccgaggccagcaaaacgctgcaggaggtgctcagcgtattacacaacgatgcctttatgcagcacgggatgatctgcctgtacgacagcgagcaggagatcctcagtatcgaagcgctgcagcaaaccggccagcagcccctccccggcagcacgcagatccgctatcgccccggcgagggactggtggggaccgtgctggcccaggggcagtcgctggtgctgccccgggtcgccgacgatcagcgttttctcgaccgcctgagcctctacgattacgatctgccgtttatcgccgtaccgttgatggggcccaacgcccggccaataggggtgctggcggcccagccgatggcgcgccaggaagagcggctgccggcctgcacccgttttctcgaaaccgtc

SEQ ID NO: 121 - DNA, 1155 bp, synthetic construct; identical to SEQ ID NO: 111
  misc_feature 1..1155: note = Synthetic polynucleotide
  misc_feature 1..1155: note = glnE-delta-AR-2 36bp deletion
  source 1..1155: mol_type = other DNA

SEQ ID NO: 122 - DNA, 2155 bp, synthetic construct; identical to SEQ ID NO: 112
  misc_feature 1..2155: note = Synthetic polynucleotide
  misc_feature 1..2155: note = glnE-delta-AR-2 36bp deletion
  source 1..2155: mol_type = other DNA

SEQ ID NO: 123 - DNA, 613 bp, synthetic construct; identical to SEQ ID NO: 109
  misc_feature 1..613: note = Synthetic polynucleotide
  misc_feature 1..613: note = delta-nifL::PinfC
  source 1..613: mol_type = other DNA

SEQ ID NO: 124 - DNA, 1613 bp, synthetic construct; identical to SEQ ID NO: 110
  misc_feature 1..1613: note = Synthetic polynucleotide
  misc_feature 1..1613: note = delta-nifL::PinfC with 500bp flank
  source 1..1613: mol_type = other DNA

SEQ ID NO: 125 - DNA, 1191 bp, synthetic construct; identical to SEQ ID NO: 107
  misc_feature 1..1191: note = Synthetic polynucleotide
  misc_feature 1..1191: note = glnE-delta-AR-2
  source 1..1191: mol_type = other DNA

SEQ ID NO: 126 - DNA, 2191 bp, synthetic construct; identical to SEQ ID NO: 108
  misc_feature 1..2191: note = Synthetic polynucleotide
  misc_feature 1..2191: note = glnE-delta-AR-2 with 500bp flank
  source 1..2191: mol_type = other DNA

SEQ ID NO: 127 - DNA, 1188 bp, synthetic construct
  misc_feature 1..1188: note = Synthetic polynucleotide
  misc_feature 1..1188: note = glnE-delta-AR-2
  source 1..1188: mol_type = other DNA
atggcgctcaaacagttaatccgtctgtgtgccgcctcgccgatggtcgcgacacaacttgcacgtcatcctttattgctcgatgaactgctcgacccgcgcacgctttaccagccgattgagccgggcgcttaccgcgacgaactgcgtcagtatctgatgcgggtgccaacagaagacgaagaacagcagcttgaagccgtgcgccagttcaaacaggcccagcatttgcgtatcgcagccggggatatttccggggcattgccggtgatgaaagtcagtgaccatttaacctaccttgccgaggccattctcgatgtcgtggtgcagcatgcgtgggaacaaatggtcgtaaaatacgggcagcccgcgcatcttcagcaccgtgaggggcgcggttttgccgtggtcggttacgggaaactcggtggctgggagctgggttatagctcagatctggatctggtcttcctgctcgattgcgcgccggaggtgatgacggacggcgaacgcagcatcgacggacgtcagttttatcttcggctggcgcagcgcattatgcacttattcagcacccggacatcgtcaggcattctttacgaggttgatccgcgtctgcgaccttccggcgcatccggcatgctggtcagtaccattgaagcgtttgcagattatcaggccaatgaagcctggacgtgggagcatcaggcgctggttcgcgcgcgcgtggtttacggggatccgcaactgacacagcaatttaacgccacgcgtcgcgacattctttgccgccagcgcgatggcgacggcctgcgtaaggaggtccgtgaaatgcgcgagaaaatgtatgcccatctggggagtaaaaaagcccacgagtttgatctgaaagccgatccgggtggcatcacggatattgaattcattgcacaatacctggttctgcgtttcgcgcatgatgagccgaagctgacgcgctggtctgataacgtgcggatttttgaactgatggcacgatatgacatcatgccggaagaggaagcgcgccatctgacgcaggcttatgtgacgctgcgcgatgaaattcatcatctggcgttgcaggaacacagcgggaaagtggccgcggacagctttgctactgagcgcgcgcagatccgtgccagctgggcaaagtggctcggctga

SEQ ID NO: 128 - DNA, 2188 bp, synthetic construct
  misc_feature 1..2188: note = Synthetic polynucleotide
  misc_feature 1..2188: note = glnE-delta-AR-2 with 500bp flank
  source 1..2188: mol_type = other DNA
cggtactggaacagaaatcggcggatgcgcaggaaatttgttatgacacggcctgtctgaagtgcaagttagtgcttacttcctggctggcaacctcaggctggacgccgtttattgatgataaatctgcgaagaaactggacgcttccttcaaacgttttgctgacatcatgctcggtcgtaccgcagcggatctgaaagaagcctttgcgcagccactgacggaagaaggttatcgcgatcagctggcgcgcctgaaacgccagatcattaccttccatttgcttgccggtgcttaccctgaaaaagacgtcgatgcgtatattgccggctgggtggacctgcaacaggccatcgttcagcagcaacacgcctgggaggattcggcccgttctcacgcggtgatgatggatgctttctggttaaacgggcaacctcgttaactgactgactagcctgggcaaactgcccgggcttttttttgcaaggaatctgatttcatggcgctcaaacagttaatccgtctgtgtgccgcctcgccgatggtcgcgacacaacttgcacgtcatcctttattgctcgatgaactgctcgacccgcgcacgctttaccagccgattgagccgggcgcttaccgcgacgaactgcgtcagtatctgatgcgggtgccaacagaagacgaagaacagcagcttgaagccgtgcgccagttcaaacaggcccagcatttgcgtatcgcagccggggatatttccggggcattgccggtgatgaaagtcagtgaccatttaacctaccttgccgaggccattctcgatgtcgtggtgcagcatgcgtgggaacaaatggtcgtaaaatacgggcagcccgcgcatcttcagcaccgtgaggggcgcggttttgccgtggtcggttacgggaaactcggtggctgggagctgggttatagctcagatctggatctggtcttcctgctcgattgcgcgccggaggtgatgacggacggcgaacgcagcatcgacggacgtcagttttatcttcggctggcgcagcgcattatgcacttattcagcacccggacatcgtcaggcattctttacgaggttgatccgcgtctgcgaccttccggcgcatccggcatgctggtcagtaccattgaagcgtttgcagattatcaggccaatgaagcctggacgtgggagcatcaggcgctggttcgcgcgcgcgtggtttacggggatccgcaactgacacagcaatttaacgccacgcgtcgcgacattctttgccgccagcgcgatggcgacggcctgcgtaaggaggtccgtgaaatgcgcgagaaaatgtatgcccatctggggagtaaaaaagcccacgagtttgatctgaaagccgatccgggtggcatcacggatattgaattcattgcacaatacctggttctgcgtttcgcgcatgatgagccgaagctgacgcgctggtctgataacgtgcggatttttgaactgatggcacgatatgacatcatgccggaagaggaagcgcgccatctgacgcaggcttatgtgacgctgcgcgatgaaattcatcatctggcgttgcaggaacacagcgggaaagtggccgcggacagctttgctactgagcgcgcgcagatccgtgccagctgggcaaagtggctcggctgagggtttttattcggctaacaggcgcttgtgatattatccggcgcattgtatttacccgatttgatttatctgttttggagtcttgggatgaaagtgactttgcctgattttcaccgcgcaggtgtgctggttgtcggtgacgtaatgttagaccgttactggtatggcccgaccaatcgtatttctccggaagctccggtgccggtggtgaaggtcagtaccattgaagagcggcctggcggtgcagctaacgtggcgatgaacatttcatctctgggcgcctcttcctgtctgatcggcctgaccggcgtagacgacgctgcgcgtgccctcagtgagcgtctggcagaagtgaaagttaactgcgatttcgtcgcactatccacacatcctaccatcaccaaactgcgaattttgtcccgtaaccagcaactgatccgcctcgactttgaggaaggttttgaaggcgttgatctcgagccgatgctgaccaaaataga

SEQ ID NO: 129 - DNA, 635 bp, synthetic construct
  misc_feature 1..635: note = Synthetic polynucleotide
  misc_feature 1..635: note = delta-nifL::Prm6.1
  source 1..635: mol_type = other DNA
atgagcatcacggcgttatcagcatcatttcctgaggggaatatcgccagccgcttgtcgctgcaacatccttcactgttttataccgtggttgaacaatcttcggtggcgatttcgctgaccgatccgcaggcgcgcatttgttatgccaatccggcattctgccgccagacgggttttgcacttgagacacttttgggcgagaaccaccgtctgctggaattttttttcacaaagcgtagcgttattgaatcgcacattttaaactgttggccgctgtggaagcgaatattggtgaaaggtgcggttttaaggcctttttctttgactctctgtcgttacaaagttaatatgcgcgccctccgtctctgaagctctcggtgaacattgttgcgaggcaggatgcgagctggttgtgttttgacattaccgataatgtgccgcgtgaacgggtgcgttatgcccgcccggaagcggcgttttcccgtccggggaatggcatggagctgcgccttatccagacgctgatcgcccatcatcgcggttctttagatctctcggtccgccctgatggcggcaccttgctgacgttacgcctgccggtacagcaggttatcaccggaggcttaaaatga

SEQ ID NO: 130 - DNA, 1635 bp, synthetic construct
  misc_feature 1..1635: note = Synthetic polynucleotide
  misc_feature 1..1635: note = delta-nifL::Prm6.1 with 500bp flank
  source 1..1635: mol_type = other DNA
tgtttcgtctcgaggccgggcaactgagcggccccgttgaaaccgacctgggctggcatctgttgttgtgcgaacaaattcgcctgccgcaacccttgccgaaagccgaagccttaacgcgggtgcgtcagcaactgattgcccggcaacagaaacattatcagcgccagtggctgcaacaactgatcaacgcctgagcctgttctccttcttgttgatgcagacgggttaatgcccgttttgcacgaaaaatgcacataaattgcctgcgttgccttataacagcgcagggaaatcctgcctccggccttgtgccacaccgcgctttgcctggtttgtggtaaaaactggcccgctttgcatcctgatgcttaaaacaccccgttcagatcaacctttgggcagataagcccgcgaaaggcctgcaaattgcacggttattccgggtgagtatatgtgtgatttgggttccggcattgcgcaataaaggggagaaagacatgagcatcacggcgttatcagcatcatttcctgaggggaatatcgccagccgcttgtcgctgcaacatccttcactgttttataccgtggttgaacaatcttcggtggcgatttcgctgaccgatccgcaggcgcgcatttgttatgccaatccggcattctgccgccagacgggttttgcacttgagacacttttgggcgagaaccaccgtctgctggaattttttttcacaaagcgtagcgttattgaatcgcacattttaaactgttggccgctgtggaagcgaatattggtgaaaggtgcggttttaaggcctttttctttgactctctgtcgttacaaagttaatatgcgcgccctccgtctctgaagctctcggtgaacattgttgcgaggcaggatgcgagctggttgtgttttgacattaccgataatgtgccgcgtgaacgggtgcgttatgcccgcccggaagcggcgttttcccgtccggggaatggcatggagctgcgccttatccagacgctgatcgcccatcatcgcggttctttagatctctcggtccgccctgatggcggcaccttgctgacgttacgcctgccggtacagcaggttatcaccggaggcttaaaatgacccagttacctaccgcgggcccggttatccggcgctttgatatgtctgcccagtttacggcgctttatcgcatcagcgtggcgctgagtcaggaaagcaacaccgggcgcgcactggcggcgatcctcgaagtgcttcacgatcatgcatttatgcaatacggcatggtgtgtctgtttgataaagaacgcaatgcactctttgtggaatccctgcatggcatcgacggcgaaaggaaaaaagagacccgccatgtccgttaccgcatgggggaaggcgtgatcggcgcggtgatgagccagcgtcaggcgctggtgttaccgcgcatttcagacgatcagcgttttctcgaccgcctgaatatttacgattacagcctgccgttgattggcgtgccgatccccggtgcggataatcagccatcgggcgtgctggtggcacagccgatggcgttgcacgaagaccggctgactgccagtacgcggtttttagaaatggtc

SEQ ID NO: 131 - DNA, 635 bp, synthetic construct; identical to SEQ ID NO: 129
  misc_feature 1..635: note = Synthetic polynucleotide
  misc_feature 1..635: note = delta-nifL::Prm6.1
  source 1..635: mol_type = other DNA

SEQ ID NO: 132 - DNA, 1635 bp, synthetic construct; identical to SEQ ID NO: 130
  misc_feature 1..1635: note = Synthetic polynucleotide
  misc_feature 1..1635: note = delta-nifL::Prm6.1 with 500bp flank
  source 1..1635: mol_type = other DNA

SEQ ID NO: 133 - DNA, 786 bp, synthetic construct
  misc_feature 1..786: note = Synthetic polynucleotide
  misc_feature 1..786: note = delta-nifL::Prm7.1
  source 1..786: mol_type = other DNA
atgagcatcacggcgttatcagcatcatttcctgaggggaatatcgccagccgcttgtcgctgcaacatccttcactgttttataccgtggttgaacaatcttcggtggcgatttcgctgaccgatccgcaggcgcgcatttgttatgccaatccggcattctgccgccagacgggttttgcacttgagacacttttgggcgagaaccaccgtctgctggttaaaaacgtgaccacgagcattaataaacgccacgaaatgtggcgtttatttattcaaaaagtatcttctttcataaaaagtgctaaatgcagtagcagcaaaattgggataagtcccatggaatacggctgttttcgctgcaatttttaactttttcgtaaaaaaagatgtttctttgagcgaacgatcaaaatatagcgttaaccggcaaaaaattattctcattagaaaatagtttgtgtaatacttgtaacgctacatggagattaacttaatctagagggttttataccgtctctgaagctctcggtgaacattgttgcgaggcaggatgcgagctggttgtgttttgacattaccgataatgtgccgcgtgaacgggtgcgttatgcccgcccggaagcggcgttttcccgtccggggaatggcatggagctgcgccttatccagacgctgatcgcccatcatcgcggttctttagatctctcggtccgccctgatggcggcaccttgctgacgttacgcctgccggtacagcaggttatcaccggaggcttaaaatga

SEQ ID NO: 134 - DNA, 1786 bp, synthetic construct
  misc_feature 1..1786: note = Synthetic polynucleotide
  misc_feature 1..1786: note = delta-nifL::Prm7.1 with 500bp flank
  source 1..1786: mol_type = other DNA
gtttcgtctcgaggccgggcaactgagcggccccgttgaaaccgacctgggctggcatctgttgttgtgcgaacaaattcgcctgccgcaacccttgccgaaagccgaagccttaacgcgggtgcgtcagcaactgattgcccggcaacagaaacattatcagcgccagtggctgcaacaactgatcaacgcctgagcctgttctccttcttgttgatgcagacgggttaatgcccgttttgcacgaaaaatgcacataaattgcctgcgttgccttataacagcgcagggaaatcctgcctccggccttgtgccacaccgcgctttgcctggtttgtggtaaaaactggcccgctttgcatcctgatgcttaaaacaccccgttcagatcaacctttgggcagataagcccgcgaaaggcctgcaaattgcacggttattccgggtgagtatatgtgtgatttgggttccggcattgcgcaataaaggggagaaagacatgagcatcacggcgttatcagcatcatttcctgaggggaatatcgccagccgcttgtcgctgcaacatccttcactgttttataccgtggttgaacaatcttcggtggcgatttcgctgaccgatccgcaggcgcgcatttgttatgccaatccggcattctgccgccagacgggttttgcacttgagacacttttgggcgagaaccaccgtctgctggttaaaaacgtgaccacgagcattaataaacgccacgaaatgtggcgtttatttattcaaaaagtatcttctttcataaaaagtgctaaatgcagtagcagcaaaattgggataagtcccatggaatacggctgttttcgctgcaatttttaactttttcgtaaaaaaagatgtttctttgagcgaacgatcaaaatatagcgttaaccggcaaaaaattattctcattagaaaatagtttgtgtaatacttgtaacgctacatggagattaacttaatctagagggttttataccgtctctgaagctctcggtgaacattgttgcgaggcaggatgcgagctggttgtgttttgacattaccgataatgtgccgcgtgaacgggtgcgttatgcccgcccggaagcggcgttttcccgtccggggaatggcatggagctgcgccttatccagacgctgatcgcccatcatcgcggttctttagatctctcggtccgccctgatggcggcaccttgctgacgttacgcctgccggtacagcaggttatcaccggaggcttaaaatgacccagttacctaccgcgggcccggttatccggcgctttgatatgtctgcccagtttacggcgctttatcgcatcagcgtggcgctgagtcaggaaagcaacaccgggcgcgcactggcggcgatcctcgaagtgcttcacgatcatgcatttatgcaatacggcatggtgtgtctgtttgataaagaacgcaatgcactctttgtggaatccctgcatggcatcgacggcgaaaggaaaaaagagacccgccatgtccgttaccgcatgggggaaggcgtgatcggcgcggtgatgagccagcgtcaggcgctggtgttaccgcgcatttcagacgatcagcgttttctcgaccgcctgaatatttacgattacagcctgccgttgattggcgtgccgatccccggtgcggataatcagccatcgggcgtgctggtggcacagccgatggcgttgcacgaagaccggctgactgccagtacgcggtttttagaaatggtcg

SEQ ID NO: 135 - DNA, 993 bp, synthetic construct
  misc_feature 1..993: note = Synthetic polynucleotide
  misc_feature 1..993: note = delta-nifL::Prm1.2
  source 1..993: mol_type = other DNA
atgagcatcacggcgttatcagcatcatttcctgaggggaatatcgccagccgcttgtcgctgcaacatccttcactgttttataccgtggttgaacaatcttcggtggcgatttcgctgaccgatccgcaggcgcgcatttgttatgccaatccggcattctgccgccagacgggttttgcacttgagacacttttgggcgagaaccaccgtctgctggtgaacatcactgatgcacaagctacctatgtcgaagaattaactaaaaaactgcaagatgcaggcattcgcgttaaagccgacttgagaaatgagaagattggctttaaaattcgcgaacacacgctacgccgtgttccttatatgttagtttgtggcgataaagaggtcgaagcaggcaaagttgctgttcgtacccgccgcggcaaagacttaggaagcatggatgttagcgaagtcgttgacaaactgctggcggaaatccgcagcagaagtcttcatcaactggaggaataaagtattaaaggcggaaaacgagttcaaccggcgcgtcctaatcgcattaacaaagagattcgcgcgcaagaagttcgcctcacaggcgtcgatggcgagcagattggtattgtcagtctgaatgaagctcttgaaaaagctgaggaagcgggcgtcgatttagtagaaatcagtccgaatgccgagccgccagtttgtcgaatcccgtctctgaagctctcggtgaacattgttgcgaggcaggatgcgagctggttgtgttttgacattaccgataatgtgccgcgtgaacgggtgcgttatgcccgcccggaagcggcgttttcccgtccggggaatggcatggagctgcgccttatccagacgctgatcgcccatcatcgcggttctttagatctctcggtccgccctgatggcggcaccttgctgacgttacgcctgccggtacagcaggttatcaccggaggcttaaaatga

SEQ ID NO: 136 - DNA, 1993 bp, synthetic construct
  misc_feature 1..1993: note = Synthetic polynucleotide
  misc_feature 1..1993: note = delta-nifL::Prm1.2 with 500bp flank
  source 1..1993: mol_type = other DNA
tgtttcgtctcgaggccgggcaactgagcggccccgttgaaaccgacctgggctggcatctgttgttgtgcgaacaaattcgcctgccgcaacccttgccgaaagccgaagccttaacgcgggtgcgtcagcaactgattgcccggcaacagaaacattatcagcgccagtggctgcaacaactgatcaacgcctgagcctgttctccttcttgttgatgcagacgggttaatgcccgttttgcacgaaaaatgcacataaattgcctgcgttgccttataacagcgcagggaaatcctgcctccggccttgtgccacaccgcgctttgcctggtttgtggtaaaaactggcccgctttgcatcctgatgcttaaaacaccccgttcagatcaacctttgggcagataagcccgcgaaaggcctgcaaattgcacggttattccgggtgagtatatgtgtgatttgggttccggcattgcgcaataaaggggagaaagacatgagcatcacggcgttatcagcatcatttcctgaggggaatatcgccagccgcttgtcgctgcaacatccttcactgttttataccgtggttgaacaatcttcggtggcgatttcgctgaccgatccgcaggcgcgcatttgttatgccaatccggcattctgccgccagacgggttttgcacttgagacacttttgggcgagaaccaccgtctgctggtgaacatcactgatgcacaagctacctatgtcgaagaattaactaaaaaactgcaagatgcaggcattcgcgttaaagccgacttgagaaatgagaagattggctttaaaattcgcgaacacacgctacgccgtgttccttatatgttagtttgtggcgataaagaggtcgaagcaggcaaagttgctgttcgtacccgccgcggcaaagacttaggaagcatggatgttagcgaagtcgttgacaaactgctggcggaaatccgcagcagaagtcttcatcaactggaggaataaagtattaaaggcggaaaacgagttcaaccggcgcgtcctaatcgcattaacaaagagattcgcgcgcaagaagttcgcctcacaggcgtcgatggcgagcagattggtattgtcagtctgaatgaagctcttgaaaaagctgaggaagcgggcgtcgatttagtagaaatcagtccgaatgccgagccgccagtttgtcgaatcccgtctctgaagctctcggtgaacattgttgcgaggcaggatgcgagctggttgtgttttgacattaccgataatgtgccgcgtgaacgggtgcgttatgcccgcccggaagcggcgttttcccgtccggggaatggcatggagctgcgccttatccagacgctgatcgcccatcatcgcggttctttagatctctcggtccgccctgatggcggcaccttgctgacgttacgcctgccggtacagcaggttatcaccggaggcttaaaatgacccagttacctaccgcgggcccggttatccggcgctttgatatgtctgcccagtttacggcgctttatcgcatcagcgtggcgctgagtcaggaaagcaacaccgggcgcgcactggcggcgatcctcgaagtgcttcacgatcatgcatttatgcaatacggcatggtgtgtctgtttgataaagaacgcaatgcactctttgtggaatccctgcatggcatcgacggcgaaaggaaaaaagagacccgccatgtccgttaccgcatgggggaaggcgtgatcggcgcggtgatgagccagcgtcaggcgctggtgttaccgcgcatttcagacgatcagcgttttctcgaccgcctgaatatttacgattacagcctgccgttgattggcgtgccgatccccggtgcggataatcagccatcgggcgtgctggtggcacagccgatggcgttgcacgaagaccggctgactgccagtacgcggtttttagaaatggtc

SEQ ID NO: 137 - DNA, 993 bp, synthetic construct; identical to SEQ ID NO: 135
  misc_feature 1..993: note = Synthetic polynucleotide
  misc_feature 1..993: note = delta-nifL::Prm1.2
  source 1..993: mol_type = other DNA

SEQ ID NO: 138 - DNA, 1993 bp, synthetic construct; identical to SEQ ID NO: 136
  misc_feature 1..1993: note = Synthetic polynucleotide
  misc_feature 1..1993: note = delta-nifL::Prm1.2 with 500bp flank
  source 1..1993: mol_type = other DNA

SEQ ID NO: 139 - DNA, 1188 bp, synthetic construct; identical to SEQ ID NO: 127
  misc_feature 1..1188: note = Synthetic polynucleotide
  misc_feature 1..1188: note = glnE-delta-AR-2
  source 1..1188: mol_type = other DNA

SEQ ID NO: 140 - DNA, 2188 bp, synthetic construct; identical to SEQ ID NO: 128
  misc_feature 1..2188: note = Synthetic polynucleotide
  misc_feature 1..2188: note = glnE-delta-AR-2 with 500bp flank
  source 1..2188: mol_type = other DNA

SEQ ID NO: 141 - DNA, 663 bp, synthetic construct
  misc_feature 1..663: note = Synthetic polynucleotide
  misc_feature 1..663: note = delta-nifL::Prm3.1
  source 1..663: mol_type = other DNA
atgagcatcacggcgttatcagcatcatttcctgaggggaatatcgccagccgcttgtcgctgcaacatccttcactgttttataccgtggttgaacaatcttcggtggcgatttcgctgaccgatccgcaggcgcgcatttgttatgccaatccggcattctgccgccagacgggttttgcacttgagacacttttgggcgagaaccaccgtctgctggtacagtagcgcctctcaaaaatagataaacggctcatgtacgtgggccgtttattttttctacccataatcgggaaccggtgttataatgccgcgccctcatattgtggggatttcttaatgacctatcctgggtcctaaagttgtagttgacattagcggagcactaacccgtctctgaagctctcggtgaacattgttgcgaggcaggatgcgagctggttgtgttttgacattaccgataatgtgccgcgtgaacgggtgcgttatgcccgcccggaagcggcgttttcccgtccggggaatggcatggagctgcgccttatccagacgctgatcgcccatcatcgcggttctttagatctctcggtccgccctgatggcggcaccttgctgacgttacgcctgccggtacagcaggttatcaccggaggcttaaaatga

SEQ ID NO: 142 - DNA, 1663 bp, synthetic construct
  misc_feature 1..1663: note = Synthetic polynucleotide
  misc_feature 1..1663: note = delta-nifL::Prm3.1 with 500bp flank
  source 1..1663: mol_type = other DNA
tgtttcgtctcgaggccgggcaactgagcggccccgttgaaaccgacctgggctggcatctgttgttgtgcgaacaaattcgcctgccgcaacccttgccgaaagccgaagccttaacgcgggtgcgtcagcaactgattgcccggcaacagaaacattatcagcgccagtggctgcaacaactgatcaacgcctgagcctgttctccttcttgttgatgcagacgggttaatgcccgttttgcacgaaaaatgcacataaattgcctgcgttgccttataacagcgcagggaaatcctgcctccggccttgtgccacaccgcgctttgcctggtttgtggtaaaaactggcccgctttgcatcctgatgcttaaaacaccccgttcagatcaacctttgggcagataagcccgcgaaaggcctgcaaattgcacggttattccgggtgagtatatgtgtgatttgggttccggcattgcgcaataaaggggagaaagacatgagcatcacggcgttatcagcatcatttcctgaggggaatatcgccagccgcttgtcgctgcaacatccttcactgttttataccgtggttgaacaatcttcggtggcgatttcgctgaccgatccgcaggcgcgcatttgttatgccaatccggcattctgccgccagacgggttttgcacttgagacacttttgggcgagaaccaccgtctgctggtacagtagcgcctctcaaaaatagataaacggctcatgtacgtgggccgtttattttttctacccataatcgggaaccggtgttataatgccgcgccctcatattgtggggatttcttaatgacctatcctgggtcctaaagttgtagttgacattagcggagcactaacccgtctctgaagctctcggtgaacattgttgcgaggcaggatgcgagctggttgtgttttgacattaccgataatgtgccgcgtgaacgggtgcgttatgcccgcccggaagcggcgttttcccgtccggggaatggcatggagctgcgccttatccagacgctgatcgcccatcatcgcggttctttagatctctcggtccgccctgatggcggcaccttgctgacgttacgcctgccggtacagcaggttatcaccggaggcttaaaatgacccagttacctaccgcgggcccggttatccggcgctttgatatgtctgcccagtttacggcgctttatcgcatcagcgtggcgctgagtcaggaaagcaacaccgggcgcgcactggcggcgatcctcgaagtgcttcacgatcatgcatttatgcaatacggcatggtgtgtctgtttgataaagaacgcaatgcactctttgtggaatccctgcatggcatcgacggcgaaaggaaaaaagagacccgccatgtccgttaccgcatgggggaaggcgtgatcggcgcggtgatgagccagcgtcaggcgctggtgttaccgcgcatttcagacgatcagcgttttctcgaccgcctgaatatttacgattacagcctgccgttgattggcgtgccgatccccggtgcggataatcagccatcgggcgtgctggtggcacagccgatggcgttgcacgaagaccggctgactgccagtacgcggtttttagaaatggtc

SEQ ID NO: 143 - DNA, 1188 bp, synthetic construct; identical to SEQ ID NO: 127
  misc_feature 1..1188: note = Synthetic polynucleotide
  misc_feature 1..1188: note = glnE-delta-AR-2
  source 1..1188: mol_type = other DNA

SEQ ID NO: 144 - DNA, 2188 bp, synthetic construct; identical to SEQ ID NO: 128
  misc_feature 1..2188: note = Synthetic polynucleotide
  misc_feature 1..2188: note = glnE-delta-AR-2 with 500bp flank
  source 1..2188: mol_type = other DNA

SEQ ID NO: 145 - DNA, 613 bp, synthetic construct
  misc_feature 1..613: note = Synthetic polynucleotide
  misc_feature 1..613: note = delta-nifL::PinfC
  source 1..613: mol_type = other DNA
atgagcatcacggcgttatcagctgaatatcactgactcacaagctacctatgtcgaagaattaactaaaaaactgcaagatgcaggcattcgcgttaaagccgacttgagaaatgagaagattggctttaaaattcgcgaacacacgctacgccgtgttccttatatgttagtttgtggcgataaagaggtcgaagcaggcaaagttgctgttcgtactcgtcgcggcaaagacttaggaagcatggatgttagcgaagtcgttgacaaactgctggcggaaatccgcagcagaagtcatcatcaactggaggaataaagtattaaaggcggaaaacgagttcaaccggcgcgtcctaatcgcattaacaaagagattcgcgcgcaagaagttcgcctcaccggcgtcgatggcgagcagattggtattgtcagtctgaatgaagctcttgaaaaagctgaggaagcgggcgtcgatttagtagaaatcagtccgaatgccgagccgccagtttgtcgaatctctttagatctctcggtccgccctgatggcggcaccttgctgacgttacgcctgccggtacagcaggttatcaccggaggcttaaaatga

SEQ ID NO: 146 - DNA, 1613 bp, synthetic construct
  misc_feature 1..1613: note = Synthetic polynucleotide
  misc_feature 1..1613: note = delta-nifL::PinfC with 500bp flank
  source 1..1613: mol_type = other DNA
tgtttcgtctcgaagccgggcaactgagcagccccgttgaaaccgaactgggctggcatctgttgttgtgcgaacaaattcgcctgccgcaacccttgccgaaagccgaggccttaacgcgggtgcgtcagcaactgattgcccggcaacagaatcattatcagcgccagtggctgcaacaactgatcaacgcctgagcctgttctccttcttgttggtgcagacgggttaatgcccgttttgcacgaaaaatgcacataaactgccttcgctgccttataacagcgcatggaaatcctgcctcctgccttgtgccacgccgcgctttgcctggtttgtggtaaaaactggcccgctttgcatcctgatgtttaaaacaccccgttcagatcaacctttgggcagataagcccgcgaaaggcctgcaaattgcacggttattccgggtgagtatatgtgtgatttgggttccggcattgcgcaataaaggggagaaagacatgagcatcacggcgttatcagctgaatatcactgactcacaagctacctatgtcgaagaattaactaaaaaactgcaagatgcaggcattcgcgttaaagccgacttgagaaatgagaagattggctttaaaattcgcgaacacacgctacgccgtgttccttatatgttagtttgtggcgataaagaggtcgaagcaggcaaagttgctgttcgtactcgtcgcggcaaagacttaggaagcatggatgttagcgaagtcgttgacaaactgctggcggaaatccgcagcagaagtcatcatcaactggaggaataaagtattaaaggcggaaaacgagttcaaccggcgcgtcctaatcgcattaacaaagagattcgcgcgcaagaagttcgcctcaccggcgtcgatggcgagcagattggtattgtcagtctgaatgaagctcttgaaaaagctgaggaagcgggcgtcgatttagtagaaatcagtccgaatgccgagccgccagtttgtcgaatctctttagatctctcggtccgccctgatggcggcaccttgctgacgttacgcctgccggtacagcaggttatcaccggaggcttaaaatgacccagttacctaccgcgggcccggttatccggcgctttgatatgtctgcccagtttacggcgctttatcgcatcagcgtggcgctgagtcaggaaagcaataccgcgcgcgcactggcggcgatcctcgaagtgcttcacgatcatgcatttatgcaatacggcatggtgtgtctgttcgataaagaacgcaatgcactgtttgtggaatccctgcatggcatcgacggcgaaaggaaaaaagaaacccgccatgtccgttaccgcatgggggaaggcgtgatcggcgcggtgatgagccagcgtcaggcgctggtgttaccgcgcatttcagacgatcagcgttttctcgaccgcctgaatatttacgattacagcctgccgctgattggtgtgccgatccccggtgcggataatcagcctgcgggtgtgctggtggcacagccgatggcgttgcacgaagaccggctggctgccagtacgcggtttttagaaatggtc

SEQ ID NO: 147 - DNA, 426 bp, synthetic construct
  misc_feature 1..426: note = Synthetic polynucleotide
  misc_feature 1..426: note = delta-nifL::Prm5
  source 1..426: mol_type = other DNA
atgaccctgaatatgatgatggatgccggcggacatcatcgcgacaaacaatattaataccggcaaccacaccggcaatttacgagactgcgcaggcatcctttctcccgtcaatttctgtcaaataaagtaaaagaggcagtctacttgaattacccccggctggttgagcgtttgttgaaaaaaagtaactgaaaaatccgtagaatagcgccactctgatggttaattaacctattcaattaagaattatctggatgaatgtgccattaaatgcgcagcataatggtgcgttgtgcgggaaaactgcttttttttgaaagggttggtcagtagcggaaacaactcacttcacaccccgaagggggaagttgcctgaccctacgattcccgctatttcattcactgaccggaggttcaaaatga

SEQ ID NO: 148 - DNA, 1426 bp, synthetic construct
  misc_feature 1..1426: note = Synthetic polynucleotide
  misc_feature 1..1426: note = delta-nifL::Prm5 with 500bp flank
  source 1..1426: mol_type = other DNA
accggatacgagagaaaagtgtctacatcggttcggttgatattgaccggcgcatccgccagcccgcccagtttctggtggatctgtttggcgattttgcgggtcttgccggtgtcggtgccgaaaaaaataccaatatttgccataacacacgctcctgttgaaaaagagatcccgccgggaaatgcggtgaacgtgtctgatattgcgaagagtgtgccagttttggtcgcgggcaaaacctgcaccagtttggttattaatgcaccagtctggcgctttttttcgccgagtttctcctcgctaatgcccgccaggcgcggctttggcgctgatagcgcgctgaataccgatctggatcaaggttttgtcgggttatcagccaaaaggtgcactctttgcatggttatacgtgcctgacatgttgtccgggcgacaaacggcctggtggcacaaattgtcagaactacgacacgactaactgaccgcaggagtgtgcgatgaccctgaatatgatgatggatgccggcggacatcatcgcgacaaacaatattaataccggcaaccacaccggcaatttacgagactgcgcaggcatcctttctcccgtcaatttctgtcaaataaagtaaaagaggcagtctacttgaattacccccggctggttgagcgtttgttgaaaaaaagtaactgaaaaatccgtagaatagcgccactctgatggttaattaacctattcaattaagaattatctggatgaatgtgccattaaatgcgcagcataatggtgcgttgtgcgggaaaactgcttttttttgaaagggttggtcagtagcggaaacaactcacttcacaccccgaagggggaagttgcctgaccctacgattcccgctatttcattcactgaccggaggttcaaaatgacccagcgaaccgagtcgggtaataccgtctggcgcttcgatttgtcccagcagttcactgcgatgcagcgcataagcgtggtactcagccgggcgaccgaggtcgatcagacgctccagcaagtgctgtgcgtattgcacaatgacgcctttttgcagcacggcatgatctgtctgtacgacagccagcaggcgattttgaatattgaagcgttgcaggaagccgatcagcagttaatccccggcagctcgcaaatccgctatcgtccgggcgaagggctggtcgggacggtgctttcgcagggccaatcattagtgctggcgcgcgttgctgacgatcagcgctttcttgaccggctcgggttgtatgattacaacctgccgtttatcgccgtgccgctgatagggccagatgcgcagactttcggtgtgctgacggcacaacccatggcgcgttacgaagagcgattacccgcctgcacccgctttctggaaacggtc

SEQ ID NO: 149 - DNA, 461 bp, synthetic construct
  misc_feature 1..461: note = Synthetic polynucleotide
  misc_feature 1..461: note = delta-nifL::Prm1
  source 1..461: mol_type = other DNA
atgaccctgaatatgatgatggatgccggccgtcctgtaataataaccggacaattcggactgattaaaaaagcgcccttgtggcgctttttttatattcccgcctccatttaaaataaaaaatccaatcggatttcactatttaaactggccattatctaagatgaatccgatggaagctcgctgttttaacacgcgttttttaaccttttattgaaagtcggtgcttctttgagcgaacgatcaaatttaagtggattcccatcaaaaaaatattctcaacctaaaaaagtttgtgtaatacttgtaacgctacatggagattaactcaatctagagggtattaataatgaatcgtactaaactggtactgggcgcaactcacttcacaccccgaagggggaagttgcctgaccctacgattcccgctatttcattcactgaccggaggttcaaaatga

SEQ ID NO: 150 - DNA, 1461 bp, synthetic construct
  misc_feature 1..1461: note = Synthetic polynucleotide
  misc_feature 1..1461: note = delta-nifL::Prm1 with 500bp flank
  source 1..1461: mol_type = other DNA
accggatacgagagaaaagtgtctacatcggttcggttgatattgaccggcgcatccgccagcccgcccagtttctggtggatctgtttggcgattttgcgggtcttgccggtgtcggtgccgaaaaaaataccaatatttgccataacacacgctcctgttgaaaaagagatcccgccgggaaatgcggtgaacgtgtctgatattgcgaagagtgtgccagttttggtcgcgggcaaaacctgcaccagtttggttattaatgcaccagtctggcgctttttttcgccgagtttctcctcgctaatgcccgccaggcgcggctttggcgctgatagcgcgctgaataccgatctggatcaaggttttgtcgggttatcagccaaaaggtgcactctttgcatggttatacgtgcctgacatgttgtccgggcgacaaacggcctggtggcacaaattgtcagaactacgacacgactaactgaccgcaggagtgtgcgatgaccctgaatatgatgatggatgccggccgtcctgtaataataaccggacaattcggactgattaaaaaagcgcccttgtggcgctttttttatattcccgcctccatttaaaataaaaaatccaatcggatttcactatttaaactggccattatctaagatgaatccgatggaagctcgctgttttaacacgcgttttttaaccttttattgaaagtcggtgcttctttgagcgaacgatcaaatttaagtggattcccatcaaaaaaatattctcaacctaaaaaagtttgtgtaatacttgtaacgctacatggagattaactcaatctagagggtattaataatgaatcgtactaaactggtactgggcgcaactcacttcacaccccgaagggggaagttgcctgaccctacgattcccgctatttcattcactgaccggaggttcaaaatgacccagcgaaccgagtcgggtaataccgtctggcgcttcgatttgtcccagcagttcactgcgatgcagcgcataagcgtggtactcagccgggcgaccgaggtcgatcagacgctccagcaagtgctgtgcgtattgcacaatgacgcctttttgcagcacggcatgatctgtctgtacgacagccagcaggcgattttgaatattgaagcgttgcaggaagccgatcagcagttaatccccggcagctcgcaaatccgctatcgtccgggcgaagggctggtcgggacggtgctttcgcagggccaatcattagtgctggcgcgcgttgctgacgatcagcgctttcttgaccggctcgggttgtatgattacaacctgccgtttatcgccgtgccgctgatagggccagatgcgcagactttcggtgtgctgacggcacaacccatggcgcgttacgaagagcgattacccgcctgcacccgctttctggaaacggtc

SEQ ID NO: 151 - DNA, 461 bp, synthetic construct; identical to SEQ ID NO: 149
  misc_feature 1..461: note = Synthetic polynucleotide
  misc_feature 1..461: note = delta-nifL::Prm1
  source 1..461: mol_type = other DNA

SEQ ID NO: 152 - DNA, 1461 bp, synthetic construct; identical to SEQ ID NO: 150
  misc_feature 1..1461: note = Synthetic polynucleotide
  misc_feature 1..1461: note = delta-nifL::Prm1 with 500bp flank
  source 1..1461: mol_type = other DNA

SEQ ID NO: 153 - DNA, 1188 bp, synthetic construct
  misc_feature 1..1188: note = Synthetic polynucleotide
  misc_feature 1..1188: note = glnE-delta-AR-2
  source 1..1188: mol_type = other DNA
atggcactgaaacacctcatttccctgtgtgccgcgtcgccgatggttgccagtcagctggcgcgctacccgatcctgcttgatgaattgctcgacccgaatacgctctatcaaccgacggcgatgaatgcctatcgcgatgagctgcgccaatacctgctgcgcgtgccggaagatgatgaagagcaacagcttgaggcgctgcggcagtttaagcaggcgcagttgctgcgcgtggcggcggcggatattgccggtacgttgccagtaatgaaagtgagcgatcacttaacctggctggcggaagcgattattgatgcggtggtgcagcaagcctgggggcagatggtggcgcgttatggccagccaacgcatctgcacgatcgcgaagggcgcggttttgcggtggtcggttatggcaagctgggcggctgggagctgggttacagctccgatctggatctggtattcctgcacgactgcccgatggatgtgatgaccgatggcgagcgtgaaatcgatggtcgccagttctatttgcgtctcgcgcagcgcgtgatgcacctgtttagcacgcgcacgtcgtccggcatcctttatgaagttgatgcgcgtctgcgtccatctggcgctgcggggatgctggtcactactacggaatcgttcgccgattaccagcaaaacgaagcctggacgtgggaacatcaggcgctggcccgtgcgcgcgtggtgtacggcgatccgcaactgaccgccgaatttgacgccattcgccgcgatattctgatgacgcctcgcgacggcgcaacgctgcaaaccgacgtgcgagaaatgcgcgagaaaatgcgtgcccatcttggcaacaagcataaagaccgcttcgatctgaaagccgatgaaggcggtatcaccgacatcgagtttatcgcccaatatctggtgctgcgctttgcccatgacaagccgaaactgacgcgctggtcggataatgtgcgcattctcgaagggctggcgcaaaacggcatcatggaggagcaggaagcgcaggcattgacgctggcgtacaccacattgcgtgatgagctgcaccacctggcgctgcaagagttgccgggacatgtggcgctctcctgttttgtcgccgagcgtgcgcttattaaaaccagctgggacaagtggctggtggaa

SEQ ID NO: 154 - DNA, 2206 bp, synthetic construct
  misc_feature 1..2206: note = Synthetic polynucleotide
  misc_feature 1..2206: note = glnE-delta-AR-2 with 500bp flank
  source 1..2206: mol_type = other DNA
gcgcaaagcgagtgctcacttacgtgatctgttgacacaatctgaagcgaccataacttctgccgtttcagcgaatacggcggtgtggagcgcacaatcagccctggcgaagctggtgctcaccgagtggctagtgacgcagggctggcgaaccttccttgatgaaaaagcgcaggccaaattcgccgactcctttaaacgctttgctgacatccatctgtcacgcagcgccgccgagctgaaaaaagcctttgcccaaccgctgggcgacagctatcgcgaccagttgccgcgcctggcgcgtgatatcgactgcgcgttactgctggccgggcattacgatcgcgcgcgcgccgtggaatggctggaaaactggcaggggcttcagcacgccattgaaacgcgccagagagtcgaaatcgaacatttccgtaataccgcgattacccaggagccgttctggttgcacagcggaaaacgttaacgaaaggatatttcgcatggcactgaaacacctcatttccctgtgtgccgcgtcgccgatggttgccagtcagctggcgcgctacccgatcctgcttgatgaattgctcgacccgaatacgctctatcaaccgacggcgatgaatgcctatcgcgatgagctgcgccaatacctgctgcgcgtgccggaagatgatgaagagcaacagcttgaggcgctgcggcagtttaagcaggcgcagttgctgcgcgtggcggcggcggatattgccggtacgttgccagtaatgaaagtgagcgatcacttaacctggctggcggaagcgattattgatgcggtggtgcagcaagcctgggggcagatggtggcgcgttatggccagccaacgcatctgcacgatcgcgaagggcgcggttttgcggtggtcggttatggcaagctgggcggctgggagctgggttacagctccgatctggatctggtattcctgcacgactgcccgatggatgtgatgaccgatggcgagcgtgaaatcgatggtcgccagttctatttgcgtctcgcgcagcgcgtgatgcacctgtttagcacgcgcacgtcgtccggcatcctttatgaagttgatgcgcgtctgcgtccatctggcgctgcggggatgctggtcactactacggaatcgttcgccgattaccagcaaaacgaagcctggacgtgggaacatcaggcgctggcccgtgcgcgcgtggtgtacggcgatccgcaactgaccgccgaatttgacgccattcgccgcgatattctgatgacgcctcgcgacggcgcaacgctgcaaaccgacgtgcgagaaatgcgcgagaaaatgcgtgcccatcttggcaacaagcataaagaccgcttcgatctgaaagccgatgaaggcggtatcaccgacatcgagtttatcgcccaatatctggtgctgcgctttgcccatgacaagccgaaactgacgcgctggtcggataatgtgcgcattctcgaagggctggcgcaaaacggcatcatggaggagcaggaagcgcaggcattgacgctggcgtacaccacattgcgtgatgagctgcaccacctggcgctgcaagagttgccgggacatgtggcgctctcctgttttgtcgccgagcgtgcgcttattaaaaccagctgggacaagtggctggtggaaccgtgcgccccggcgtaagtgtggtatcatcgcgcgcaaattttgtatctctcaggagacaggaatgaaagtgacgctgccagagtttaagcaagccggtgtaatggtggtgggtgatgtgatgctggatcgttactggtatggcccaaccagccgtatctctccggaagcgccagtcccggttgttaaagtcgataccattgaagagcgtcctggcggcgcggcaaacgtggcgatgaatatcgcctcactgggcgccacggcgcgtctggttggcctgactggcattgacgatgcggcgcgcgcgctgagcaaagcgctggccgatgttaacgttaaatgtgacttcgtttctgttccgacgcatcccaccatcactaagctgcgcgtgctgtcgcgtaaccagcagctgattcgcctggactttgaagagggttttgaaggagtcgatccgcaaccgatgcatgaacgcatcagccaggcgcttggtaatattggcgcgctggtgctgtcggatt

SEQ ID NO: 155 - DNA, 1563 bp, synthetic construct
  misc_feature 1..1563: note = Synthetic polynucleotide
  misc_feature 1..1563: note = glnE-delta-AR-1
  source 1..1563: mol_type = other DNA
atgtttaacgatctgattggcgatgatgaaacggattcgccggaagatgcgctttctgagagctggcgcgaattgtggcaggatgcgttgcaggaggaggattccacgcccgtgctggcgcatctctcagaggacgatcgccgccgcgtggtggcgctgattgccgattttcgcaaagagttggataaacgcaccattggcccgcgagggcggcaggtactcgatcacttaatgccgcatctgctcagcgatgtatgctcgcgcgacgatgcgccagtaccgctgtcacgcctgacgccgctgctcaccggaattattacccgcaccacttaccttgagctgctaagtgaatttcccggcgcactgaaacacctcatttccctgtgtgccgcgtcgccgatggttgccagtcagctggcgcgctacccgatcctgcttgatgaattgctcgacccgaatacgctctatcaaccgacggcgatgaatgcctatcgcgatgagctgcgccaatacctgctgcgcgtgccggaagatgatgaagagcaacagcttgaggcgctgcggcagtttaagcaggcgcagttgctgcgcgtggcggcggcggatattgccggtacgttgccagtaatgaaagtgagcgatcacttaacctggctggcggaagcgattattgatgcggtggtgcagcaagcctgggggcagatggtggcgcgttatggccagccaacgcatctgcacgatcgcgaagggcgcggttttgcggtggtcggttatggcaagctgggcggctgggagctgggttacagctccgatctggatctggtattcctgcacgactgcccgatggatgtgatgaccgatggcgagcgtgaaatcgatggtcgccagttctatttgcgtctcgcgcagcgcgtgatgcacctgtttagcacgcgcacgtcgtccggcatcctttatgaagttgatgcgcgtctgcgtccatctggcgctgcggggatgctggtcactactacggaatcgttcgccgattaccagcaaaacgaagcctggacgtgggaacatcaggcgctggcccgtgcgcgcgtggtgtacggcgatccgcaactgaccgccgaatttgacgccattcgccgcgatattctgatgacgcctcgcgacggcgcaacgctgcaaaccgacgtgcgagaaatgcgcgagaaaatgcgtgcccatcttggcaacaagcataaagaccgcttcgatctgaaagccgatgaaggcggtatcaccgacatcgagtttatcgcccaatatctggtgctgcgctttgcccatgacaagccgaaactgacgcgctggtcggataatgtgcgcattctcgaagggctggcgcaaaacggcatcatggaggagcaggaagcgcaggcattgacgctggcgtacaccacattgcgtgatgagctgcaccacctggcgctgcaagagttgccgggacatgtggcgctctcctgttttgtcgccgagcgtgcgcttattaaaaccagctgggacaagtggctggtggaaccgtgcgccccggcgtaa

SEQ ID NO: 156 - DNA, 461 bp, synthetic construct; identical to SEQ ID NO: 149
  misc_feature 1..461: note = Synthetic polynucleotide
  misc_feature 1..461: note = delta-nifL::Prm1
  source 1..461: mol_type = other DNA

SEQ ID NO: 157 - DNA, 1461 bp, synthetic construct; identical to SEQ ID NO: 150
  misc_feature 1..1461: note = Synthetic polynucleotide
  misc_feature 1..1461: note = delta-nifL::Prm1 with 500bp flank
  source 1..1461: mol_type = other DNA

SEQ ID NO: 158 - DNA, 2563 bp, synthetic construct
  misc_feature 1..2563: note = Synthetic polynucleotide
  misc_feature 1..2563: note = glnE-delta-AR-1 with 500bp flank
  source 1..2563: mol_type = other DNA
gcgcaaagcgagtgctcacttacgtgatctgttgacacaatctgaagcgaccataacttctgccgtttcagcgaatacggcggtgtggagcgcacaatcagccctggcgaagctggtgctcaccgagtggctagtgacgcagggctggcgaaccttccttgatgaaaaagcgcaggccaaattcgccgactcctttaaacgctttgctgacatccatctgtcacgcagcgccgccgagctgaaaaaagcctttgcccaaccgctgggcgacagctatcgcgaccagttgccgcgcctggcgcgtgatatcgactgcgcgttactgctggccgggcattacgatcgcgcgcgcgccgtggaatggctggaaaactggcaggggcttcagcacgccattgaaacgcgccagagagtcgaaatcgaacatttccgtaataccgcgattacccaggagccgttctggttgcacagcggaaaacgttaacgaaaggatatttcgcatgtttaacgatctgattggcgatgatgaaacggattcgccggaagatgcgctttctgagagctggcgcgaattgtggcaggatgcgttgcaggaggaggattccacgcccgtgctggcgcatctctcagaggacgatcgccgccgcgtggtggcgctgattgccgattttcgcaaagagttggataaacgcaccattggcccgcgagggcggcaggtactcgatcacttaatgccgcatctgctcagcgatgtatgctcgcgcgacgatgcgccagtaccgctgtcacgcctgacgccgctgctcaccggaattattacccgcaccacttaccttgagctgctaagtgaatttcccggcgcactgaaacacctcatttccctgtgtgccgcgtcgccgatggttgccagtcagctggcgcgctacccgatcctgcttgatgaattgctcgacccgaatacgctctatcaaccgacggcgatgaatgcctatcgcgatgagctgcgccaatacctgctgcgcgtgccggaagatgatgaagagcaacagcttgaggcgctgcggcagtttaagcaggcgcagttgctgcgcgtggcggcggcggatattgccggtacgttgccagtaatgaaagtgagcgatcacttaacctggctggcggaagcgattattgatgcggtggtgcagcaagcctgggggcagatggtggcgcgttatggccagccaacgcatctgcacgatcgcgaagggcgcggttttgcggtggtcggttatggcaagctgggcggctgggagctgggttacagctccgatctggatctggtattcctgcacgactgcccgatggatgtgatgaccgatggcgagcgtgaaatcgatggtcgccagttctatttgcgtctcgcgcagcgcgtgatgcacctgtttagcacgcgcacgtcgtccggcatcctttatgaagttgatgcgcgtctgcgtccatctggcgctgcggggatgctggtcactactacggaatcgttcgccgattaccagcaaaacgaagcctggacgtgggaacatcaggcgctggcccgtgcgcgcgtggtgtacggcgatccgcaactgaccgccgaatttgacgccattcgccgcgatattctgatgacgcctcgcgacggcgcaacgctgcaaaccgacgtgcgagaaatgcgcgagaaaatgcgtgcccatcttggcaacaagcataaagaccgcttcgatctgaaagccgatgaaggcggtatcaccgacatcgagtttatcgcccaatatctggtgctgcgctttgcccatgacaagccgaaactgacgcgctggtcggataatgtgcgcattctcgaagggctggcgcaaaacggcatcatggaggagcaggaagcgcaggcattgacgctggcgtacaccacattgcgtgatgagctgcaccacctggcgctgcaagagttgccgggacatgtggcgctctcctgttttgtcgccgagcgtgcgcttattaaaaccagctgggacaagtggctggtggaaccgtgcgccccggcgtaagtgtggtatcatcgcgcgcaaattttgtatctctcaggagacaggaatgaaagtgacgctgccagagtttaagcaagccggtgtaatggtggtgggtgatgtgatgctggatcgttactggtatggcccaaccagccgtatctctccggaagcgccagtcccggttgttaaagtcgataccattgaagagcgtcctggcggcgcggcaaacgtggcgatgaatatcgcctcactgggcgccacggcgcgtctggttggcctgactggcattgacgatgcggcgcgcgcgctgagcaaagcgctggccgatgttaacgttaaatgtgacttcgtttctgttccgacgcatcccaccatcactaagctgcgcgtgctgtcgcgtaaccagcagctgattcgcctggactttgaagagggttttgaaggagtcgatccgcaaccgatgcatgaacgcatcagccaggcgcttggtaatattggcgcgctggtgctgtcggatt

SEQ ID NO: 159 - DNA, 1563 bp, synthetic construct; identical to SEQ ID NO: 155
  misc_feature 1..1563: note = Synthetic polynucleotide
  misc_feature 1..1563: note = glnE-delta-AR-1
  source 1..1563: mol_type = other DNA

SEQ ID NO: 160 - DNA, 2563 bp, synthetic construct; identical to SEQ ID NO: 158
  misc_feature 1..2563: note = Synthetic polynucleotide
  misc_feature 1..2563: note = glnE-delta-AR-1 with 500bp flank
  source 1..2563: mol_type = other DNA

SEQ ID NO: 161 - DNA, 426 bp, synthetic construct; identical to SEQ ID NO: 147
  misc_feature 1..426: note = Synthetic polynucleotide
  misc_feature 1..426: note = delta-nifL::Prm5
  source 1..426: mol_type = other DNA

SEQ ID NO: 162 - DNA, 1426 bp, synthetic construct; identical to SEQ ID NO: 148
  misc_feature 1..1426: note = Synthetic polynucleotide
  misc_feature 1..1426: note = delta-nifL::Prm5 with 500bp flank
  source 1..1426: mol_type = other DNA

SEQ ID NO: 163 - DNA, 461 bp, synthetic construct; identical to SEQ ID NO: 149
  misc_feature 1..461: note = Synthetic polynucleotide
  misc_feature 1..461: note = delta-nifL::Prm1
  source 1..461: mol_type = other DNA

SEQ ID NO: 164 - DNA, 1461 bp, synthetic construct; identical to SEQ ID NO: 150
  misc_feature 1..1461: note = Synthetic polynucleotide
  misc_feature 1..1461: note = delta-nifL::Prm1 with 500bp flank
  source 1..1461: mol_type = other DNA

SEQ ID NO: 165 - DNA, 1188 bp, synthetic construct; identical to SEQ ID NO: 153
  misc_feature 1..1188: note = Synthetic polynucleotide
  misc_feature 1..1188: note = glnE-delta-AR-2
  source 1..1188: mol_type = other DNA

SEQ ID NO: 166 - DNA, 2206 bp, synthetic construct; identical to SEQ ID NO: 154
  misc_feature 1..2206: note = Synthetic polynucleotide
  misc_feature 1..2206: note = glnE-delta-AR-2 with 500bp flank
  source 1..2206: mol_type = other DNA

SEQ ID NO: 167 - DNA, 199 bp, synthetic construct
  misc_feature 1..199: note = Synthetic polynucleotide
  misc_feature 1..199: note = delta-amtB
  source 1..199: mol_type = other DNA
tttcgctgaaggtgtgaccatgggccatcaggtgctggtgcagctggaaagtgttgccatcactatcgtgtggtctggcgtggtggcctttattggttacaaactggcggacatgacggtaggcctgcgcgtaccggaagaacaagaacgtgaagggctggatgtaaacagccacggcgaaaacgcctataacgcctga

SEQ ID NO: 168 - DNA, 1199 bp, synthetic construct
  misc_feature 1..1199: note = Synthetic polynucleotide
  misc_feature 1..1199: note = delta-amtB with 500bp flank
  source 1..1199: mol_type = other DNA
tttcctttctgactctgcccgtccgggcgcactaacggcctgaaatactccctcttttcattcctggcacaacgattgcaatgtctgttgcgtgttagctgcggccattatcgaattcgactggagggggatctatgaagctggttaccgtggtgattaagccattcaaacttgaagacgtgcgtgaagcgctttcttctattggtattcaagggttgaccgtaactgaagtgaaaggctttggccgtcagaagggtcacgctgagctgtaccgcggtgcggaatatagcgttaatttcctgccgaaagtgaaaattgatgtggcgatcgctgacgatcaactcgatgaagtaatcgatgtgatcagcaaagcggcctacaccggaaaaattggcgacggcaaaattttcgttgctgagctgcaacgcgtcattcgtattcgtaccggcgaagccgacgaagcggcactgtaatacaagacacacagtgatggggatcggtttcgctgaaggtgtgaccatgggccatcaggtgctggtgcagctggaaagtgttgccatcactatcgtgtggtctggcgtggtggcctttattggttacaaactggcggacatgacggtaggcctgcgcgtaccggaagaacaagaacgtgaagggctggatgtaaacagccacggcgaaaacgcctataacgcctgattgcgttgagttatctcctgagcataaaaaagcctccattcggaggcttttctttttttaagtttaaagcgcggttagttgcgattgcgcatgacgccttcctgcacgctggacgcgaccagcacaccctcttgcgtatagaactcgccgcgcacaaaaccgcgagcgctggaggctgacgtgctttccacactgtagagcagccattcgttcatattaaacgggcgatggaaccacatggagtggtcaatggtggcaacctgcataccgcgctcaaggaagcccacgccgtgcggctgaagtgcaaccggcaggaagttaaagtctgaggcatatccaagcagatattgatgtacgcgaaaatcgtccggcaccgtgccgtttgcgcggatccatacctggcgggtgggatcggcaacgtggcctttcagcgggttatgaaactcaaccgggcggatctccagtggtttatcactaagaaacttctctttggcctgcgg

SEQ ID NO: 169 - DNA, 1563 bp, synthetic construct; identical to SEQ ID NO: 155
  misc_feature 1..1563: note = Synthetic polynucleotide
  misc_feature 1..1563: note = glnE-delta-AR-1
  source 1..1563: mol_type = other DNA

SEQ ID NO: 170 - DNA, 2563 bp, synthetic construct; identical to SEQ ID NO: 158
  misc_feature 1..2563: note = Synthetic polynucleotide
  misc_feature 1..2563: note = glnE-delta-AR-1 with 500bp flank
  source 1..2563: mol_type = other DNA

SEQ ID NO: 171 - DNA, 461 bp, synthetic construct; identical to SEQ ID NO: 149
  misc_feature 1..461: note = Synthetic polynucleotide
  misc_feature 1..461: note = delta-nifL::Prm1
  source 1..461: mol_type = other DNA

SEQ ID NO: 172 - DNA, 1461 bp, synthetic construct; identical to SEQ ID NO: 150
  misc_feature 1..1461: note = Synthetic polynucleotide
  misc_feature 1..1461: note = delta-nifL::Prm1 with 500bp flank
  source 1..1461: mol_type = other DNA

SEQ ID NO: 173 - DNA, 199 bp, synthetic construct; identical to SEQ ID NO: 167
  misc_feature 1..199: note = Synthetic polynucleotide
  misc_feature 1..199: note = delta-amtB
  source 1..199: mol_type = other DNA

SEQ ID NO: 174 - DNA, 1199 bp, synthetic construct; identical to SEQ ID NO: 168
  misc_feature 1..1199: note = Synthetic polynucleotide
  misc_feature 1..258: note = delta-amtB with 500bp flank
  source 1..1199: mol_type = other DNA

SEQ ID NO: 175 - DNA, 607 bp, synthetic construct
  misc_feature 1..607: note = Synthetic polynucleotide
  misc_feature 1..607: note = delta-nifL::PinfC
  source 1..607: mol_type = other DNA
atgacctttaatatgatgcctggggtcactggagcgctttatcggcatcctgaccgaagaatttgccggtttcttcccgacctggctggcccctgttcaggttgtggtgatgaatatcactgattctcaagctgaatatgtcaacgaattgacccgtaaattgcaaaatgcgggcattcgtgtaaaagcggacttgagaaacgagaagattggctttaaaatccgcgagcacactttacgtcgtgtcccttatatgttggtctgtggtgataaagaggtggaagcaggcaaagtggccgttcgcacccgccgcggtaaagacctgggcagcctggacgtaagtgaagtgattgagaagctgcaacaagagattcgcagccgcagtcttcaacaactggaggaataaggtattaaaggcggaaaacgagttcaaacggcacgtccgaatcgtatcaatggcgagattcgcgcccaggaagttcgcttaactggtctggaaggtgagcagctgggtattgcaatagaactaactacccgccctgaaggcggtacctgcctgaccctgcgattcccgttatttcattcactgaccggaggcccacgatga

SEQ ID NO: 176 - DNA, 1607 bp, synthetic construct
  misc_feature 1..1607: note = Synthetic polynucleotide
  misc_feature 1..1607: note = delta-nifL::PinfC with 500bp flank
  source 1..1607: mol_type = other DNA
ggtacgacaaaaacgtctccagcgacgtgcggttaatattgactggcgcatccgccacatcccccagtttttgctggatcagtttggcgattttgcgggtttttcccgtgtcactgccaaaaaaaataccaatgttagccatgtcgcgctcctgttgagaaagaataaggccgcctgcaaacggcggatatcccttctcctgttgcgaaggctgtgccaggtttttttaaggccttctgtgcactgaaatgggtgaaaaaatgactcttttttgtgcaggcaccgtcctctctccgctatccagacctgctttgaaggcctctgagggccaaatcagggccaaaacacgaatcaggatcaatgtttcggcgcgttacctgttcgaaaggtgcactctttgcatggttaatcacacccaatcagggctgcggatgtcgggcgtttcacaacacaaaatgttgtaaatgcgacacagccgggcctgaaaccaggagcgtgtgatgacctttaatatgatgcctggggtcactggagcgctttatcggcatcctgaccgaagaatttgccggtttcttcccgacctggctggcccctgttcaggttgtggtgatgaatatcactgattctcaagctgaatatgtcaacgaattgacccgtaaattgcaaaatgcgggcattcgtgtaaaagcggacttgagaaacgagaagattggctttaaaatccgcgagcacactttacgtcgtgtcccttatatgttggtctgtggtgataaagaggtggaagcaggcaaagtggccgttcgcacccgccgcggtaaagacctgggcagcctggacgtaagtgaagtgattgagaagctgcaacaagagattcgcagccgcagtcttcaacaactggaggaataaggtattaaaggcggaaaacgagttcaaacggcacgtccgaatcgtatcaatggcgagattcgcgcccaggaagttcgcttaactggtctggaaggtgagcagctgggtattgcaatagaactaactacccgccctgaaggcggtacctgcctgaccctgcgattcccgttatttcattcactgaccggaggcccacgatgacccagcgacccgagtcgggcaccaccgtctggcgttttgatctctcacagcaatttaccgccatgcagcgcatcagcgtggtgttgagtcgcgcaaccgagataagccagacgctgcaggaggtgctgtgtgttctgcataatgacgcatttatgcaacacggcatgctgtgtctgtatgacaaccagcaggaaattctgagtattgaagccttgcaggaggcagaccaacatctgatccccggcagctcgcaaattcgctatcgccctggcgaagggctggtaggagccgtactgtcccagggacaatctcttgtgctgccgcgtgtcgccgacgatcaacgctttctcgacaggcttggcatctatgattacaacctgccgtttatcgccgtccccttaatggggccaggcgcgcagacgattggcgtgctcgccgcgcagccgatggcgcgtctggaggagcggcttccttcctgtacgcgctttctggaaaccgtc

SEQ ID NO: 177 - DNA, 1536 bp, Kosakonia sacchari
  gene 1..1536: note = 16S-1
  source 1..1536: mol_type = genomic DNA
ttgaagagtttgatcatggctcagattgaacgctggcggcaggcctaacacatgcaagtcgaacggtagcacagagagcttgctctcgggtgacgagtggcggacgggtgagtaatgtctgggaaactgcctgatggagggggataactactggaaacggtagctaataccgcataacgtcgcaagaccaaagagggggaccttcgggcctcttgccatcagatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttcgggttgtaaagcactttcagcggggaggaagggagtaaggttaataaccttattcattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcatccgaaactggcaggcttgagtctcgtagagggaggtagaattccaggtgtagcggtgaaatgcgtagagatctggaggaataccggtggcgaaggcggcctcctggacgaagactgacgctcaggtgcgaaagcgtggggagcaaacaggattagataccctggtagtccacgccgtaaacgatgtctatttggaggttgtgcccttgaggcgtggcttccggagctaacgcgttaaatagaccgcctggggagtacggccgcaaggttaaaactcaaatgaattgacgggggcccgcacaagcggtggagcatgtggtttaattcgatgcaacgcgaagaaccttacctggtcttgacatccacagaactttccagagatggattggtgccttcgggaactgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagtaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 178 - DNA, 1537 bp, Kosakonia sacchari
  source 1..1537: mol_type = genomic DNA
ttgaagagtttgatcatggctcagattgaacgctggcggcaggcctaacacatgcaagtcgaacggtagcacagagagcttgctctcgggtgacgagtggcggacgggtgagtaatgtctgggaaactgcctgatggagggggataactactggaaacggtagctaataccgcataacgtcgcaagaccaaagagggggaccttcgggcctcttgccatcagatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttcgggttgtaaagcactttcagcggggaggaaggnantanggttaataacctgtgttnattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcatccgaaactggcaggcttgagtctcgtagagggaggtagaattccaggtgtagcggtgaaatgcgtagagatctggaggaataccggtggcgaaggcggcctcctggacgaagactgacgctcaggtgcgaaagcgtggggagcaaacaggattagataccctggtagtccacgccgtaaacgatgtctatttggaggttgtgcccttgaggcgtggcttccggagctaacgcgttaaatagaccgcctggggagtacggccgcaaggttaaaactcaaatgaattgacgggggcccgcacaagcggtggagcatgtggtttaattcgatgcaacgcgaagaaccttacctggtcttgacatccacagaacttagcagagatgctttggtgccttcgggaactgtgagacaggtgctgcatggctgtcgtcagctcgtgttgtgaaatgttgggttaagtcccgcaacgagcgcaacccttatcctttgttgccagcggttaggccgggaactcaaaggagactgccagtgataaactggaggaaggtggggatgacgtcaagtcatcatggcccttacgaccagggctacacacgtgctacaatggcgcatacaaagagaagcgacctcgcgagagtaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 179 - DNA, 882 bp, Kosakonia sacchari
  gene 1..882: note = nifH
  source 1..882: mol_type = genomic DNA
atgaccatgcgtcaatgcgccatttacggcaaaggtgggatcggcaaatcgaccaccacacagaacctggtcgccgcgctggcggagatgggtaaaaaagtcatgattgtcggctgtgacccgaaagccgattccacgcgtttgatcctgcatgcgaaagcgcagaacaccattatggagatggctgctgaagtcggctccgtggaagacctggagttagaagacgtgctgcaaatcggttacggcggcgtgcgctgcgcagagtccggcggcccggagccaggcgtgggctgtgccggtcgcggggtgatcaccgcgattaacttcctcgaagaagaaggcgcttacgtgccggatctcgattttgttttctacgacgtgctgggcgacgtggtatgcggtggtttcgccatgccgattcgtgaaaacaaagcgcaggagatctacatcgtttgctctggcgaaatgatggcgatgtacgccgccaacaacatctccaaaggcatcgtgaaatacgccaaatccggtaaagtgcgcctcggcgggctgatttgtaactcgcgccagaccgaccgtgaagatgaactgatcattgcgctggcagaaaaactcggcacgcagatgatccactttgttccccgcgacaacattgtgcagcgtgcggaaatccgccgtatgacggttatcgaatatgacccgacctgcaatcaggcgaacgaatatcgcagccttgccagcaaaatcgtcaacaacaccaaaatggtggtgcccaccccctgcaccatggatgaactggaagaactgctgatggagttcggcattatggatgtggaagacaccagcatcattggtaaaaccgccgccgaagaaaacgccgtctga

SEQ ID NO: 180 - DNA, 1449 bp, Kosakonia sacchari
  gene 1..1449: note = nifD2
  source 1..1449: mol_type = genomic DNA
atgagcaatgcaacaggcgaacgcaacctggagataatcgagcaggtgctcgaggttttcccggagaagacgcgcaaagaacgcagaaaacacatgatggtgacggacccggagcaggaaagcgtcggtaagtgcatcatctctaaccgcaaatcgcagccaggcgtgatgaccgtgcgcggctgctcgtatgccggttcgaaaggggtggtatttgggccaatcaaggatatggcgcatatctcgcatggcccaatcggctgcggccaatactcccgcgccgggcggcggaactactacaccggcgtcagcggcgtggacagcttcggcacgctcaacttcacctccgattttcaggagcgcgacatcgtgtttggcggcgataaaaagctcgccaaactgattgaagagctggaagagctgttcccgctgaccaaaggcatttcgattcagtcggaatgcccggtcggcctgattggcgatgacattgaggccgtcgcgaacgccagccgcaaagccatcaacaaaccggttattccggtgcgttgcgaaggctttcgcggcgtgtcgcaatccctcggtcaccatattgccaacgatgtgatccgcgactgggtgctggataaccgcgaaggcaaaccgttcgaatccaccccttacgatgtggcgatcatcggcgattacaacatcggcggcgatgcctgggcttcgcgcattttgctcgaagagatgggcttgcgggtggtggcacagtggtctggcgacggtacgctggtggagatggaaaacacgccgttcgtcaaactgaacctggtgcattgttaccgctcaatgaactacatctcgcgccatatggaggagaagcacggtattccgtggatggaatacaacttctttggtccgacgaaaatcgcggaatcgctgcgcaaaatcgccgaccagtttgacgacaccattcgcgccaacgccgaagcggtgatcgccagataccaggcgcaaaacgacgccattatcgccaaatatcgcccgcgtctggaggggcgcaaagtgctgctttatatgggcgggctgcgtccgcgccatgtgattggcgcctatgaagacctgggaatggagatcatcgctgccggttatgagttcggtcataacgatgattacgaccgcaccttgccggatctgaaagagggcacgctgctgtttgatgatgccagcagttatgagctggaggcgttcgtcaacgcgctgaaaccggatctcatcggttccggcatcaaagagaagtacatctttcagaaaatgggcgtgccgtttcgccagatgcactcctgggattactccggcccgtaccacggctatgacggcttcgccatcttcgcccgcgatatggatatgacgctcaacaaccccgcgtggggccagttgaccgcgccgtggctgaaatccgcctga

SEQ ID NO: 181 - DNA, 1563 bp, Kosakonia sacchari
  gene 1..1563: note = nifK2
  source 1..1563: mol_type = genomic DNA
atgagccagactgctgagaaaatacagaattgccatcccctgtttgaacaggatgcttaccagacgctgtttgccggtaaacgggcactcgaagaggcgcactcgccggagcgggtgcaggaagtgtttcaatggaccactaccccggaatatgaagcgctgaactttaaacgcgaagcgctgactatcgacccggcaaaagcctgccagccgctgggcgcggtgctctgttcgctggggtttgccaataccctaccgtatgtgcacggttcacagggttgcgtggcctatttccgcacgtactttaaccgccactttaaagaaccggtggcctgcgtgtcggattcaatgacggaagacgcggcggtgttcggcgggaataacaacctcaacaccggcttacaaaacgccagcgcgctgtataaaccggagattatcgccgtctctaccacctgtatggcggaagtgatcggtgatgatttgcaggcctttatcgccaacgccaaaaaagatggttttctcgatgccgccatccccgtgccctacgcgcacacccccagttttatcggcagccatatcaccggctgggataacatgtttgaaggttttgcccggacctttacggcagaccatgaagctcagcccggcaaactttcacgcatcaacctggtgaccgggtttgaaacctatctcggcaatttccgcgtgctgaaacgcatgatggaacaaatggaggtgccggcgagtgtgctctccgatccgtcggaagtgctggatactcccgccaacgggcattaccagatgtacgcgggcgggacgacgcagcaagagatgcgcgaggcgccggatgctatcgacaccctgttgctgcagccctggcaactggtgaaaagcaaaaaagtggtgcaggagatgtggaatcagcccgccaccgaggtttctgttcccgttgggctggcaggaacagacgaactgttgatggcgattagccagttaaccggcaaggccattcccgattcactggcgctggagcgcgggcggctggtcgatatgatgctcgattcccacacctggttgcacggtaaaaaattcggcctgtttggcgatccggattttgtcatgggattgacccgtttcctgctggagctgggctgcgaaccgaccgttatcctctgccacaacggtaacaaacgctggcagaaagcaatgaagaaaatgcttgacgcctcgccgtacggccaggagagcgaagtgtttatcaactgcgatttgtggcatttccgctcgctgatgtttacccgccagccggattttatgattggcaactcgtacggcaagttcattcagcgcgacaccttagccaaaggcgagcagtttgaagttccgctgatccgcctcggttttcccctgttcgaccgccaccatctgcaccgccagaccacctggggctacgagggcgccatgagcattctcactacccttgtgaatgcggtactggagaaagtggacaaagagaccatcaagctcggcaaaaccgactacagcttcgatcttatccgttaa

SEQ ID NO: 182 - DNA, 1488 bp, Kosakonia sacchari
  gene 1..1488: note = nifL
  source 1..1488: mol_type = genomic DNA
atgaccctgaatatgatgatggatgccggcgcgcccgaggcaatcgccggtgcgctttcgcgacaccatcctgggctgttttttaccatcgttgaagaagcgcccgtcgccatttcgctgactgatgccgacgcacgcattgtctatgccaacccggctttctgccgccagaccggctatgaactagaagcgttgttgcagcaaaatccccgcctgcttgcaagtcgccaaaccccacgggaaatctatcaggatatgtggcacaccttgttacaacgccgaccgtggcgcgggcaattgattaaccgccaccgcgacggcagcctgtatctggtcgagatcgatatcaccccggtgattaacccgtttggcgaactggaacactacctggcaatgcagcgcgatatcagcgccagttatgcgctggagcagcggttgcgcaatcacatgacgctgaccgaagcggtgctgaataacattccggcggcggtggttgtagtggatgaacgcgatcatgtggttatggataaccttgcctacaaaacgttctgtgccgactgcggcggaaaagagctcctgagcgaactcaatttttcagcccgaaaagcggagctggcaaacggccaggtcttaccggtggtgctgcgcggtgaggtgcgctggttgtcggtgacctgctgggcgctgccgggcgtcagcgaagaagccagtcgctactttattgataacaggctgacgcgcacgctggtggtgatcaccgacgacacccaacaacgccagcagcaggaacagggccgacttgaccgccttaaacagcagatgaccaacggcaaactactggcagcgatccgcgaagcgcttgacgccgcgctgatccagcttaactgccccatcaatatgctggcggcggcgcgacgtttaaacggcagtgataacaacaatgtggcgctcgacgccgcgtggcgcgaaggtgaagaggcgatggcgcggctgaaacgttgccgcccgtcgctggaactggaaagtgcggccgtctggccgctgcaacccttttttgacgatctgcgcgcgctttatcacacccgctacgagcaggggaaaaatttgcaggtcacgctggattcccatcatctggtgggatttggtcagcgtacgcaactgttagcctgcctgagtctgtggctcgatcgcacgctggatattgccgccgggctgggtgatttcaccgcgcaaacgcagatttacgcccgcgaagaagagggctggctctctttgtatatcactgacaatgtgccgctgatcccgctgcgccacacccactcgccggatgcgcttaacgctccgggaaaaggcatggagctgcgcctgatccagacgctggtggcacaccaccacggcgcaatagaactcacttcacaccccgaagggggaagttgcctgaccctacgattcccgctatttcattcactgaccggaggttcaaaatga

SEQ ID NO: 183 - DNA, 1575 bp, Kosakonia sacchari
  gene 1..1575: note = nifA
  source 1..1575: mol_type = genomic DNA
atgacccagcgaaccgagtcgggtaataccgtctggcgcttcgatttgtcccagcagttcactgcgatgcagcgcataagcgtggtactcagccgggcgaccgaggtcgatcagacgctccagcaagtgctgtgcgtattgcacaatgacgcctttttgcagcacggcatgatctgtctgtacgacagccagcaggcgattttgaatattgaagcgttgcaggaagccgatcagcagttaatccccggcagctcgcaaatccgctatcgtccgggcgaagggctggtcgggacggtgctttcgcagggccaatcattagtgctggcgcgcgttgctgacgatcagcgctttcttgaccggctcgggttgtatgattacaacctgccgtttatcgccgtgccgctgatagggccagatgcgcagactttcggtgtgctgacggcacaacccatggcgcgttacgaagagcgattacccgcctgcacccgctttctggaaacggtcgctaacctggtcgcgcaaaccgtgcgtttgatggcaccaccggcagtgcgcccttccccgcgcgccgccataacacaggccgccagcccgaaatcctgcacggcctcacgcgcatttggttttgaaaatatggtcggtaacagtccggcgatgcgccagaccatggagattatccgtcaggtttcgcgctgggacaccaccgttctggtacgcggcgagagtggcaccggcaaggagctgattgccaacgccatccaccaccattcgccgcgtgccggtgcgccatttgtgaaattcaactgtgcggcgctgccggacacactgctggaaagcgaattgttcggtcacgagaaaggggcatttaccggcgcggtacgccagcgtaaaggccgttttgagctggccgatggcggcacgctgtttcttgacgagatcggcgagagtagcgcctcgtttcaggctaagctgctgcgcattttgcaggaaggcgaaatggaacgcgtcggcggcgacgagacattgcaagtgaatgtgcgcattattgccgcgacgaaccgcaatcttgaagatgaagtccggctggggcactttcgcgaagatctctattatcgcctgaatgtgatgcccatcgccctgccgccactacgcgaacgccaggaggacattgccgagctggcgcactttctggtgcgtaaaatcgcccataaccagagccgtacgctgcgcattagcgagggcgctatccgcctgctgatgagctacaactggcccggtaatgtgcgcgaactggaaaactgccttgagcgctcagcggtgatgtcggagaacggtctgatcgatcgggatgtgattttgtttaatcatcgcgaccagccagccaaaccgccagttatcagcgtctcgcatgatgataactggctcgataacaaccttgacgagcgccagcggctgattgcggcgctggaaaaagcgggatgggtacaagccaaagccgcgcgcttgctggggatgacgccgcgccaggtcgcctatcgtattcagacgatggatataaccctgccaaggctataa

SEQ ID NO: 184 - DNA, 2850 bp, Kosakonia sacchari
  gene 1..2850: note = glnE
  source 1..2850: mol_type = genomic DNA
atgccgcaccacgcaggattgtcgcagcactggcaaacggtattttctcgtctgccggaatcgctcaccgcgcagccattgagcgcgcaggcgcagtcagtgctcacttttagtgattttgttcaggacagcatcatcgcgcatcctgagtggctggcagagcttgaaagcgcgccgccgcctgcgaacgaatggcaacactatgcgcaatggctgcaagcggcgctggatggcgtcaccgatgaagcctcgctgatgcgcgcgctgcggctgtttcgccgtcgcatcatggtgcgcatcgcctggagccaggcgttacagttggtggcggaagaagatatcctgcaacagcttagcgtgctggcggaaaccctgatcgtcgccgcgcgcgactggctttatgaggcctgctgccgtgagtggggaacgccgagcaatccacaaggcgtggcgcagccgatgctggtactcggcatgggcaaactgggtggcggcgaactcaatttctcatccgatatcgatttgattttcgcctggccggaaaatggcgcaacgcgcggtggacgccgtgagctggataacgcgcaatttttcactcgccttggtcaacggctgattaaagtcctcgaccagccaacgcaggatggctttgtctaccgcgtcgatatgcgcttgcgcccgtttggcgacagcggcccgctggtgctgagctttgccgcgctggaagattactaccaggagcaggggcgcgattgggaacgctacgcgatggtgaaagcgcgcattatgggcgataacgacggcgaccatgcgcgggagttgcgcgcaatgctgcgcccgtttgttttccgccgttatatcgacttcagcgtgattcagtccctgcgtaacatgaaaggcatgattgcccgcgaagtgcgtcgccgtggcctgaaggacaacattaagctcggcgcgggcgggatccgcgaaatagaatttatcgtccaggttttccagctgattcgcggcggtcgcgagcctgcactgcaatcgcgttcactgttgccgacgcttgctgccatagatcaactgcatctgctgccggatggcgacgcaacccggctgcgcgaggcgtatttgtggctgcgacggctggagaacctgctgcaaagcatcaatgacgaacagacacagacgctgccgggcgatgaactgaatcgcgcgcgcctcgcctggggaatgggcaaagatagctgggaagcgctctgcgaaacgctggaagcgcatatgtcggcggtgcgtcagatatttaacgatctgattggcgatgatgaaacggattcgccggaagatgcgctttctgagagctggcgcgaattgtggcaggatgcgttgcaggaggaggattccacgcccgtgctggcgcatctctcagaggacgatcgccgccgcgtggtggcgctgattgccgattttcgcaaagagttggataaacgcaccattggcccgcgagggcggcaggtactcgatcacttaatgccgcatctgctcagcgatgtatgctcgcgcgacgatgcgccagtaccgctgtcacgcctgacgccgctgctcaccggaattattacccgcaccacttaccttgagctgctaagtgaatttcccggcgcactgaaacacctcatttccctgtgtgccgcgtcgccgatggttgccagtcagctggcgcgctacccgatcctgcttgatgaattgctcgacccgaatacgctctatcaaccgacggcgatgaatgcctatcgcgatgagctgcgccaatacctgctgcgcgtgccggaagatgatgaagagcaacagcttgaggcgctgcggcagtttaagcaggcgcagttgctgcgcgtggcggcggcggatattgccggtacgttgccagtaatgaaagtgagcgatcacttaacctggctggcggaagcgattattgatgcggtggtgcagcaagcctgggggcagatggtggcgcgttatggccagccaacgcatctgcacgatcgcgaagggcgcggttttgcggtggtcggttatggcaagctgggcggctgggagctgggttacagctccgatctggatctggtattcctgcacgactgcccgatggatgtgatgaccgatggcgagcgtgaaatcgatggtcgccagttctatttgcgtctcgcgcagcgcgtgatgcacctgtttagcacgcgcacgtcgtccggcatcctttatgaagttgatgcgcgtctgcgtccatctggcgctgcggggatgctggtcactactacggaatcgttcgccgattaccagcaaaacgaagcctggacgtgggaacatcaggcgctggcccgtgcgcgcgtggtgtacggcgatccgcaactgaccgccgaatttgacgccattcgccgcgatattctgatgacgcctcgcgacggcgcaacgctgcaaaccgacgtgcgagaaatgcgcgagaaaatgcgtgcccatcttggcaacaagcataaagaccgcttcgatctgaaagccgatgaaggcggtatcaccgacatcgagtttatcgcccaatatctggtgctgcgctttgcccatgacaagccgaaactgacgcgctggtcggataatgtgcgcattctcgaagggctggcgcaaaacggcatcatggaggagcaggaagcgcaggcattgacgctggcgtacaccacattgcgtgatgagctgcaccacctggcgctgcaagagttgccgggacatgtggcgctctcctgttttgtcgccgagcgtgcgcttattaaaaccagctgggacaagtggctggtggaaccgtgcgccccggcgtaa

SEQ ID NO: 185 - DNA, 1536 bp, Kosakonia sacchari
  gene 1..1536: note = 16S-3
  source 1..1536: mol_type = genomic DNA
attgaagagtttgatcatggctcagattgaacgctggcggcaggcctaacacatgcaagtcgaacggtagcacagagagcttgctctcgggtgacgagtggcggacgggtgagtaatgtctgggaaactgcctgatggagggggataactactggaaacggtagctaataccgcataacgtcgcaagaccaaagagggggaccttcgggcctcttgccatcagatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttcgggttgtaaagcactttcagcggggaggaagggagtaaggttaataaccttgctcattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcatccgaaactggcaggcttgagtctcgtagagggaggtagaattccaggtgtagcggtgaaatgcgtagagatctggaggaataccggtggcgaaggcggcctcctggacgaagactgacgctcaggtgcgaaagnnnnnnnnnnnaacaggattagataccctggtagtccatgccgtaaacgatgtctactagccgttggggcctttgaggctttagtggcgcagctaacgcgataagtagaccgcctggggagtacggtcgcaagactaaanctcaaatgaattgacgggggcccgcacaagcggtggagcatgtggtttaattcgatgcaacgcgaagaaccttacctggccttgacatagtaagaattttccagagatggattggtgccttcgggaacttacatacaggtgctgcatggctgtcgtcagctcgtgtcgtgagatgttgggttaagtcccgcaacgagcgcaacccttgtcattagttgctacatttagttgggcactctaatgagactgccggtgacaaaccggaggaaggtggggatgacgtcaagtcctcatggcccttataggtggggctacacacgtcatacaatggctggtacaaagggttgccaacccgcgagggggagctaatcccataaaaccagtcgtagtccggatcgcagtctgcaactcgactgcgtgaagtcggaatcgctagtaatcgtggatcagaatgtcacggtgaatacgttcccgggtcttgtacacaccgcccgtcacaccatgggagcgggttctgccagaagtagttagcttaaccgcaaggagggcgattaccacggcagggttcgtgactggggtgaagtcgtaacaaggtagccgtatcggaaggtgcggctggatcacctccttt

SEQ ID NO: 186 - DNA, 882 bp, Kosakonia sacchari; identical to SEQ ID NO: 179
  gene 1..882: note = nifD1
  source 1..882: mol_type = genomic DNA

SEQ ID NO: 187 - DNA, 1386 bp, Kosakonia sacchari
  gene 1..1386: note = nifK1
  source 1..1386: mol_type = genomic DNA
atggccgaaattctgcgcagtaaaaaaccgctggcggtcagcccgataaaaagcggccagccgctgggggcgatcctcgcaagcctgggtgtcgaacagtgcataccgctggtacacggcgcacagggatgtagcgcgttcgcgaaggtgttctttattcaacattttcacgatccgatcccgctgcaatcgacggcgatggatccgacttccaccattatgggcgccgatgaaaacatttttaccgcgctcaatgtgctctgccagcgcaacgccgcgaaagccattgtgctgctcagcaccgggctttcagaagcccagggcagcgacatttcgcgggtggtgcgccagtttcgtgatgattttccccggcataaaggcgttgcgctgctcaccgtcaacacacccgatttctacggctcgctggaaaacggctacagcgccgtgctggaaagcatgattgaacagtgggtacccgcacagcccgccgccagcctgcgcaaccgccgtgtcaacctgctggtcagccatttactgacaccaggcgatatcgaactgttgcgcagttatgttgaagccttcggcctgcaaccggtgattgtgccggatctgtcgctgtcgctggacgggcatctggcagacggtgatttttcgcctgttacccaagggggaacatcgctgcgcatgattgaacagatggggcaaaacctggccacctttgtgattggcgcctcgctgggccgtgcggcggcgttactggcgcagcgcagccgtggcgaggtgatcgccctgccgcatctgatgacgcttgcagcctgcgacacgtttattcatcgactgaaaaccctctccgggcgcgatgtccccgcgtggattgagcgccagcgcggccaagttcaggatgcgatgatcgattgccatatgtggctgcagggtgcggctatcgccatggcagcagaaggcgatcacctggcggcatggtgcgatttcgcccgcagccagggcatgatccccggcccgattgtcgcaccggtcagccagccggggttgcaaaatctgccggttgaaaccgtggttatcggcgatctggaagatatgcaggatcggctttgcgcgacgcccgccgcgttactggtggccaattctcatgccgccgatctcgccacgcagtttgatttgtcacttatccgcgccgggttcccggtgtatgaccggctgggggaatttcgtcgcctgcgccaggggtacagcggcattcgtgacacgctgtttgagctggcgaatgtgatgcgcgagcgccatcacccgcttgcaacctaccgctcgccgctgcgccagcacgccgacgacaacgttacgcctggagatctgtatgccgcatgttaa

SEQ ID NO: 188 - DNA, 1287 bp, Kosakonia sacchari
  gene 1..1287: note = amtB
  source 1..1287: mol_type = genomic DNA
atgaaaaacacaacattaaaaacagcgcttgcttcgctggcgttactgcctggcctggcgatggcggctcccgctgtggcggataaagccgacaacggctttatgatgatttgcaccgcgctggtgctgtttatgaccattccgggcattgcgctgttctacggcggtttgatccgcggtaaaaacgtgctgtcgatgctgacgcaggttgccgtcaccttcgcactggtgtgcattctgtgggtggtgtatggctactcgctggcatttggcgagggcaacagcttcttcgggagttttaactgggcgatgttgaaaaacatcgaactgaaagccgtgatgggcagcatttatcagtatatccacgtggcgttccagggttccttcgcctgtatcaccgttggcctgattgtcggtgcactggctgagcgtattcgcttctctgcggtgctgatttttgtggtggtatggctgacgctttcttacgtgccgattgcacacatggtgtggggcggcggtctgctggcaacccacggtgcgctggatttcgcaggcggtacggttgttcacatcaacgctgcgattgcaggtctggtgggggcttacctgattggcaaacgcgtgggctttggcaaagaagcattcaaaccgcataacctgccgatggtcttcactggcaccgctatcctgtatgttggctggtttggtttcaacgccggctccgcaagctcggcgaacgaaattgctgcgctggccttcgtgaacactgtcgttgccactgctgccgctattctggcgtgggtatttggcgaatgggcaatgcgcggcaagccgtctctgctcggtgcctgttctggtgccatcgcgggtctggttggtatcacccccgcctgtggttatgtgggtgtcggcggtgcgctgattgtgggtctgattgccggtctggctgggctgtggggcgttactgcgctgaaacgtatgttgcgtgtcgatgacccgtgtgacgtattcggtgtgcacggcgtgtgcggcatcgtgggctgtatcctgacgggtatcttcgcctctacgtcgctgggtggtgtcggtttcgctgaaggtgtgaccatgggccatcaggtgctggtgcagctggaaagtgttgccatcactatcgtgtggtctggcgtggtggcctttattggttacaaactggcggacatgacggtaggcctgcgcgtaccggaagaacaagaacgtgaagggctggatgtaaacagccacggcgaaaacgcctataacgcctga

SEQ ID NO: 189 - DNA, 348 bp, Kosakonia sacchari
  gene 1..348: note = Prm1
  source 1..348: mol_type = genomic DNA
cgtcctgtaataataaccggacaattcggactgattaaaaaagcgcccttgtggcgctttttttatattcccgcctccatttaaaataaaaaatccaatcggatttcactatttaaactggccattatctaagatgaatccgatggaagctcgctgttttaacacgcgttttttaaccttttattgaaagtcggtgcttctttgagcgaacgatcaaatttaagtggattcccatcaaaaaaatattctcaacctaaaaaagtttgtgtaatacttgtaacgctacatggagattaactcaatctagagggtattaataatgaatcgtactaaactggtactgggcgc

SEQ ID NO: 190 - DNA, 313 bp, Kosakonia sacchari
  gene 1..313: note = Prm5
  source 1..313: mol_type = genomic DNA
ggacatcatcgcgacaaacaatattaataccggcaaccacaccggcaatttacgagactgcgcaggcatcctttctcccgtcaatttctgtcaaataaagtaaaagaggcagtctacttgaattacccccggctggttgagcgtttgttgaaaaaaagtaactgaaaaatccgtagaatagcgccactctgatggttaattaacctattcaattaagaattatctggatgaatgtgccattaaatgcgcagcataatggtgcgttgtgcgggaaaactgcttttttttgaaagggttggtcagtagcggaaac

SEQ ID NO: 191 - DNA, 1485 bp, Rahnella aquatilis; identical to SEQ ID NO: 19
  gene 1..1485: note = nifL
  source 1..1485: mol_type = genomic DNA

SEQ ID NO: 192 - DNA, 1575 bp, Rahnella aquatilis; identical to SEQ ID NO: 20
  gene 1..1575: note = nifA
  source 1..1575: mol_type = genomic DNA

SEQ ID NO: 193 - DNA, 1540 bp, Rahnella aquatilis
  gene 1..1540: note = 16S-1
  source 1..1540: mol_type = genomic DNA
attgaagagtttgatcatggctcagattgaacgctggcggcaggcctaacacatgcaagtcgagcggcagcgggaagtagcttgctactttgccggcgagcggcggacgggtgagtaatgtctgggaaactgcctgatggagggggataactactggaaacggtagctaataccgcatgacctcgaaagagcaaagtgggggatcttcggacctcacgccatcggatgtgcccagatgggattagctagtaggtgaggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttagggttgtaaagcactttcagcgaggaggaaggcatcatacttaatacgtgtggtgattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgagcttaacttgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaatttgccagagatggcgaagtgccttcgggaactctgagacaggtgctgcatggctgtcgtcagctcgtgttgtgaaatgttgggttaagtcccgcaacgagcgcaacccttatcctttgttgccagcacgtaatggtgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 194 - DNA, 1540 bp, Rahnella aquatilis
  gene 1..1540: note = 16S-2
  source 1..1540: mol_type = genomic DNA
attgaagagtttgatcatggctcagattgaacgctggcggcaggcctaacacatgcaagtcgagcggcatcgggaagtagcttgctactttgccggcgagcggcggacgggtgagtaatgtctgggaaactgcctgatggagggggataactactggaaacggtagctaataccgcatgacctcgaaagagcaaagtgggggatcttcggacctcacgccatcggatgtgcccagatgggattagctagtaggtgaggtaatggctnacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttagggttgtaaagcactttcagcgaggaggaaggcatcatacttaatacgtgtggtgattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgagcttaacttgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaatttgccagagatggcgaagtgccttcgggaactctgagacaggtgctgcatggctgtcgtcagctcgtgttgtgaaatgttgggttaagtcccgcaacgagcgcaacccttatcctttgttgccagcacgtgatggtgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 195 - DNA, 1540 bp, Rahnella aquatilis
  gene 1..1540: note = 16S-3
  source 1..1540: mol_type = genomic DNA
attgaagagtttgatcatggctcagattgaacgctggcggcaggcctaacacatgcaagtcgagcggcagcgggaagtagcttgctactttgccggcgagcggcggacgggtgagtaatgtctgggaaactgcctgatggagggggataactactggaaacggtagctaataccgcatgacctcgaaagagcaaagtgggggatcttcggacctcacgccatcggatgtgcccagatgggattagctagtaggtgaggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttagggttgtaaagcactttcagcgaggaggaaggcancatacttaatacgtgtggtgattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgagcttaacttgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaatttgccagagatggcgaagtgccttcgggaactctgagacaggtgctgcatggctgtcgtcagctcgtgttgtgaaatgttgggttaagtcccgcaacgagcgcaacccttatcctttgttgccagcacgtgatggtgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 196 - DNA, 1540 bp, Rahnella aquatilis
  gene 1..1540: note = 16S-4
  source 1..1540: mol_type = genomic DNA
attgaagagtttgatcatggctcagattgaacgctggcggcaggcctaacacatgcaagtcgagcggcancgggaagtagcttgctactttgccggcgagcggcggacgggtgagtaatgtctgggaaactgcctgatggagggggataactactggaaacggtagctaataccgcatgacctcgaaagagcaaagtgggggatcttcggacctcacgccatcggatgtgcccagatgggattagctagtaggtgaggtaatggcttacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttagggttgtaaagcactttcagcgaggaggaaggcatcacacttaatacgtgtggtgattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgagcttaacttgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaatttgccagagatggcgaagtgccttcgggaactctgagacaggtgctgcatggctgtcgtcagctcgtgttgtgaaatgttgggttaagtcccgcaacgagcgcaacccttatcctttgttgccagcacgtgatggtgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 197 - DNA, 1540 bp, Rahnella aquatilis
  gene 1..1540: note = 16S-5
  source 1..1540: mol_type = genomic DNA
attgaagagtttgatcatggctcagattgaacgctggcggcaggcctaacacatgcaagtcgagcggcagcgggaagtagcttgctactttgccggcgagcggcggacgggtgagtaatgtctgggaaactgcctgatggagggggataactactggaaacggtagctaataccgcatgacctcgaaagagcaaagtgggggatcttcggacctcacgccatcggatgtgcccagatgggattagctagtaggtgaggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttagggttgtaaagcactttcagcgaggaggaaggcatcacacttaatacgtgtgttgattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgagcttaacttgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaatttgccagagatggcgaagtgccttcgggaactctgagacaggtgctgcatggctgtcgtcagctcgtgttgtgaaatgttgggttaagtcccgcaacgagcgcaacccttatcctttgttgccagcacgtgatggtgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 198 - DNA, 1009 bp, Rahnella aquatilis
  gene 1..1009: note = 16S-6
  source 1..1009: mol_type = genomic DNA
gtagctaataccgcatgacctcgaaagagcaaagtgggggatcttcggacctcacgccatcggatgtgcccagatgggattagctagtaggtgaggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttagggttgtaaagcactttcagcgaggaggaaggcatcanacttaatacgtgtgntgattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgagcttaacttgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaatttgccagagatggcgaagtgccttcgggaactctgagacaggtgctgcatggctgtcgtcagctcgtgttgtgaaatgttgggttaagtcccgcaacgagcgcaacccttatcctttgttgccagcacgtnatggtgggaactcaaaggagactgccggtgataaac

SEQ ID NO: 199 - DNA, 1519 bp, Rahnella aquatilis
  gene 1..1519: note = 16S-7
  source 1..1519: mol_type = genomic DNA
attgaagagtttgatcatggctcagattgaacgctggcggcaggcctaacacatgcaagtcgagcggcagcgggaagtagcttgctactttgccggcgagcggcggacgggtgagtaatgtcctgatggaggggataactactggaacggtagctaataccgcacctcgaaagagcaaagtgggggatcttcggacctcacgccatcggatgtgcccagatgggattagctagtaggtgaggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttagggttgtaaagcactttcagcgaggaggaaggcatcatacttaatacgtgtggtgattgacgttactcgcagaagaagcaccggctaactccgtgccagcagccgcggtaatacggagggtgcaagcttaatcggaattactgggcgtaaagcgcacgcaggcggttgttaagtcagatgtgaaatccccgagcttaacttgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattaataccctggtagtccacgctgtaacgatgtcgacttggaggttgtgccctgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaatttgccagagatggcgaagtgccttcgggaactctgagacaggtgctgcatggctgtcgtcagctcgtgttgtgaaatgttgggttaagtcccgcaacgagcgcaacccttatcctttgttgccagcacgtaatggtgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 200 - DNA, 882 bp, Rahnella aquatilis; identical to SEQ ID NO: 13
  gene 1..882: note = nifH1
  source 1..882: mol_type = genomic DNA

SEQ ID NO: 201 - DNA, 1113 bp, Rahnella aquatilis; identical to SEQ ID NO: 14
  gene 1..1113: note = nifH2
  source 1..1113: mol_type = genomic DNA

SEQ ID NO: 202 - DNA, 1374 bp, Rahnella aquatilis; identical to SEQ ID NO: 15
  gene 1..1374: note = nifD1
  source 1..1374: mol_type = genomic DNA

SEQ ID NO: 203 - DNA, 1449 bp, Rahnella aquatilis; identical to SEQ ID NO: 16
  gene 1..1449: note = nifD2
  source 1..1449: mol_type = genomic DNA

SEQ ID NO: 204 - DNA, 1386 bp, Rahnella aquatilis; identical to SEQ ID NO: 17
  gene 1..1386: note = nifK1
  source 1..1386: mol_type = genomic DNA

SEQ ID NO: 205 - DNA, 1563 bp, Rahnella aquatilis; identical to SEQ ID NO: 18
  gene 1..1563: note = nifK2
  source 1..1563: mol_type = genomic DNA

SEQ ID NO: 206 - DNA, 2838 bp, Rahnella aquatilis; identical to SEQ ID NO: 21
  gene 1..2838: note = glnE
  source 1..2838: mol_type = genomic DNA

SEQ ID NO: 207 - DNA, 449 bp, Rahnella aquatilis
  gene 1..449: note = Prm4
  source 1..449: mol_type = genomic DNA
agtctgaactcatcctgcggcagtcggtgagacgtatttttgaccaaagagtgatctacatcacggaattttgtggttgttgctgcttaaaagggcaaatctacccttagaatcaactgttatatcagggggattcagagagatattaggaatttgcacaagcgcacaatttaaccacatcatgataacgccatgtaaaacaaagataaaaaaacaaaatgcagtgacttacatcgcaagcaaggcattttcttatccaattgctcaaagtttggcctttcatatcgcaacgaaaatgcgtaatatacgcgcccttgcggacatcagtatggtcattcctagttcatgcgcatcggacaccaccagcttacaaattgcctgattgcggccccgatggccggtatcactgaccgaccatttcgtgccttatgtcatgcgatgggggctgg

SEQ ID NO: 208 - DNA, 500 bp, Rahnella aquatilis
  gene 1..500: note = Prm1.2
  source 1..500: mol_type = genomic DNA
tgaacatcactgatgcacaagctacctatgtcgaagaattaactaaaaaactgcaagatgcaggcattcgcgttaaagccgacttgagaaatgagaagattggctttaaaattcgcgaacacacgctacgccgtgttccttatatgttagtttgtggcgataaagaggtcgaagcaggcaaagttgctgttcgtacccgccgcggcaaagacttaggaagcatggatgttagcgaagtcgttgacaaactgctggcggaaatccgcagcagaagtcttcatcaactggaggaataaagtattaaaggcggaaaacgagttcaaccggcgcgtcctaatcgcattaacaaagagattcgcgcgcaagaagttcgcctcacaggcgtcgatggcgagcagattggtattgtcagtctgaatgaagctcttgaaaaagctgaggaagcgggcgtcgatttagtagaaatcagtccgaatgccgagccgccagtttgtcgaatc

SEQ ID NO: 209 - DNA, 170 bp, Rahnella aquatilis
  gene 1..170: note = Prm3.1
  source 1..170: mol_type = genomic DNA
tacagtagcgcctctcaaaaatagataaacggctcatgtacgtgggccgtttattttttctacccataatcgggaaccggtgttataatgccgcgccctcatattgtggggatttcttaatgacctatcctgggtcctaaagttgtagttgacattagcggagcactaac

SEQ ID NO: 210 - DNA, 142 bp, Rahnella aquatilis
  gene 1..142: note = Prm6.1
  source 1..142: mol_type = genomic DNA
aattttttttcacaaagcgtagcgttattgaatcgcacattttaaactgttggccgctgtggaagcgaatattggtgaaaggtgcggttttaaggcctttttctttgactctctgtcgttacaaagttaatatgcgcgccct

SEQ ID NO: 211 - DNA, 293 bp, Rahnella aquatilis
  gene 1..293: note = Prm7.1
  source 1..293: mol_type = genomic DNA
ttaaaaacgtgaccacgagcattaataaacgccacgaaatgtggcgtttatttattcaaaaagtatcttctttcataaaaagtgctaaatgcagtagcagcaaaattgggataagtcccatggaatacggctgttttcgctgcaatttttaactttttcgtaaaaaaagatgtttctttgagcgaacgatcaaaatatagcgttaaccggcaaaaaattattctcattagaaaatagtttgtgtaatacttgtaacgctacatggagattaacttaatctagagggttttata

SEQ ID NO: 212 - DNA, 1188 bp, synthetic construct; identical to SEQ ID NO: 127
  misc_feature 1..1188: note = Synthetic polynucleotide
  misc_feature 1..1188: note = glnE-delta-AR-2
  source 1..1188: mol_type = other DNA

SEQ ID NO: 213 - DNA, 2188 bp, synthetic construct; identical to SEQ ID NO: 128
  misc_feature 1..2188: note = Synthetic polynucleotide
  misc_feature 1..2188: note = glnE-delta-AR-2 with 500bp flank
  source 1..2188: mol_type = other DNA

SEQ ID NO: 214 - DNA, 524 bp, synthetic construct
  misc_feature 1..524: note = Synthetic polynucleotide
  misc_feature 1..524: note = delta-nifL::null-v1
  source 1..524: mol_type = other DNA
atgagcatcacggcgttatcagcatcatttcctgaggggaatatcgccagccgcttgtcgctgcaacatccttcactgttttataccgtggttgaacaatcttcggtggcgatttcgctgaccgatccgcaggcgcgcatttgttatgccaatccggcattctgccgccagacgggttttgcacttgagacacttttgggcgagaaccaccgtctgctggagtctgaactcatcctgcgatgggggctgggccgtctctgaagctctcggtgaacattgttgcgaggcaggatgcgagctggttgtgttttgacattaccgataatgtgccgcgtgaacgggtgcgttatgcccgcccggaagcggcgttttcccgtccggggaatggcatggagctgcgccttatccagacgctgatcgcccatcatcgcggttctttagatctctcggtccgccctgatggcggcaccttgctgacgttacgcctgccggtacagcaggttatcaccggaggcttaaaatga

SEQ ID NO: 215 - DNA, 1524 bp, synthetic construct
  misc_feature 1..1524: note = Synthetic polynucleotide
  misc_feature 1..1524: note = delta-nifL::null-v1 with 500bp flank
  source 1..1524: mol_type = other DNA
tgtttcgtctcgaggccgggcaactgagcggccccgttgaaaccgacctgggctggcatctgttgttgtgcgaacaaattcgcctgccgcaacccttgccgaaagccgaagccttaacgcgggtgcgtcagcaactgattgcccggcaacagaaacattatcagcgccagtggctgcaacaactgatcaacgcctgagcctgttctccttcttgttgatgcagacgggttaatgcccgttttgcacgaaaaatgcacataaattgcctgcgttgccttataacagcgcagggaaatcctgcctccggccttgtgccacaccgcgctttgcctggtttgtggtaaaaactggcccgctttgcatcctgatgcttaaaacaccccgttcagatcaacctttgggcagataagcccgcgaaaggcctgcaaattgcacggttattccgggtgagtatatgtgtgatttgggttccggcattgcgcaataaaggggagaaagacatgagcatcacggcgttatcagcatcatttcctgaggggaatatcgccagccgcttgtcgctgcaacatccttcactgttttataccgtggttgaacaatcttcggtggcgatttcgctgaccgatccgcaggcgcgcatttgttatgccaatccggcattctgccgccagacgggttttgcacttgagacacttttgggcgagaaccaccgtctgctggagtctgaactcatcctgcgatgggggctgggccgtctctgaagctctcggtgaacattgttgcgaggcaggatgcgagctggttgtgttttgacattaccgataatgtgccgcgtgaacgggtgcgttatgcccgcccggaagcggcgttttcccgtccggggaatggcatggagctgcgccttatccagacgctgatcgcccatcatcgcggttctttagatctctcggtccgccctgatggcggcaccttgctgacgttacgcctgccggtacagcaggttatcaccggaggcttaaaatgacccagttacctaccgcgggcccggttatccggcgctttgatatgtctgcccagtttacggcgctttatcgcatcagcgtggcgctgagtcaggaaagcaacaccgggcgcgcactggcggcgatcctcgaagtgcttcacgatcatgcatttatgcaatacggcatggtgtgtctgtttgataaagaacgcaatgcactctttgtggaatccctgcatggcatcgacggcgaaaggaaaaaagagacccgccatgtccgttaccgcatgggggaaggcgtgatcggcgcggtgatgagccagcgtcaggcgctggtgttaccgcgcatttcagacgatcagcgttttctcgaccgcctgaatatttacgattacagcctgccgttgattggcgtgccgatccccggtgcggataatcagccatcgggcgtgctggtggcacagccgatggcgttgcacgaagaccggctgactgccagtacgcggtttttagaaatggtc

SEQ ID NO: 216 - DNA, 266 bp, synthetic construct
  misc_feature 1..266: note = Synthetic polynucleotide
  misc_feature 1..266: note = delta-nifL::null-v2
  source 1..266: mol_type = other DNA
atgagcatcacggcgttatcagcatcatttcctgaggggaatatcgccagccgcttgtcgctgcaacatccttcactgttttataccgtggttgaacaatcttcggtggcgatttcgctgaccgatccgcaggcgcgcatttgttatgccaatccggcattctgccgccagacgggttttgcacttgagacacttttgggcgagaaccaccgtctgctggttaaagcctgccggtacagcaggttatcaccggaggcttaaaatga

SEQ ID NO: 217 - DNA, 1266 bp, synthetic construct
  misc_feature 1..1266: note = Synthetic polynucleotide
  misc_feature 1..1266: note = delta-nifL::null-v2 with 500bp flank
  source 1..1266: mol_type = other DNA
tgtttcgtctcgaggccgggcaactgagcggccccgttgaaaccgacctgggctggcatctgttgttgtgcgaacaaattcgcctgccgcaacccttgccgaaagccgaagccttaacgcgggtgcgtcagcaactgattgcccggcaacagaaacattatcagcgccagtggctgcaacaactgatcaacgcctgagcctgttctccttcttgttgatgcagacgggttaatgcccgttttgcacgaaaaatgcacataaattgcctgcgttgccttataacagcgcagggaaatcctgcctccggccttgtgccacaccgcgctttgcctggtttgtggtaaaaactggcccgctttgcatcctgatgcttaaaacaccccgttcagatcaacctttgggcagataagcccgcgaaaggcctgcaaattgcacggttattccgggtgagtatatgtgtgatttgggttccggcattgcgcaataaaggggagaaagacatgagcatcacggcgttatcagcatcatttcctgaggggaatatcgccagccgcttgtcgctgcaacatccttcactgttttataccgtggttgaacaatcttcggtggcgatttcgctgaccgatccgcaggcgcgcatttgttatgccaatccggcattctgccgccagacgggttttgcacttgagacacttttgggcgagaaccaccgtctgctggttaaagcctgccggtacagcaggttatcaccggaggcttaaaatgacccagttacctaccgcgggcccggttatccggcgctttgatatgtctgcccagtttacggcgctttatcgcatcagcgtggcgctgagtcaggaaagcaacaccgggcgcgcactggcggcgatcctcgaagtgcttcacgatcatgcatttatgcaatacggcatggtgtgtctgtttgataaagaacgcaatgcactctttgtggaatccctgcatggcatcgacggcgaaaggaaaaaagagacccgccatgtccgttaccgcatgggggaaggcgtgatcggcgcggtgatgagccagcgtcaggcgctggtgttaccgcgcatttcagacgatcagcgttttctcgaccgcctgaatatttacgattacagcctgccgttgattggcgtgccgatccccggtgcggataatcagccatcgggcgtgctggtggcacagccgatggcgttgcacgaagaccggctgactgccagtacgcggtttttagaaatggtc

SEQ ID NO: 218 - DNA, 943 bp, synthetic construct
  misc_feature 1..943: note = Synthetic polynucleotide
  misc_feature 1..943: note = delta-nifL::Prm4
  source 1..943: mol_type = other DNA
atgagcatcacggcgttatcagcatcatttcctgaggggaatatcgccagccgcttgtcgctgcaacatccttcactgttttataccgtggttgaacaatcttcggtggcgatttcgctgaccgatccgcaggcgcgcatttgttatgccaatccggcattctgccgccagacgggttttgcacttgagacacttttgggcgagaaccaccgtctgctggagtctgaactcatcctgcggcagtcggtgagacgtatttttgaccaaagagtgatctacatcacggaattttgtggttgttgctgcttaaaagggcaaatctacccttagaatcaactgttatatcagggggattcagagagatattaggaatttgcacaagcgcacaatttaaccacatcatgataacgccatgtaaaacaaagataaaaaaacaaaatgcagtgacttacatcgcaagcaaggcattttcttatccaattgctcaaagtttggcctttcatatcgcaacgaaaatgcgtaatatacgcgcccttgcggacatcagtatggtcattcctagttcatgcgcatcggacaccaccagcttacaaattgcctgattgcggccccgatggccggtatcactgaccgaccatttcgtgccttatgtcatgcgatgggggctgggccgtctctgaagctctcggtgaacattgttgcgaggcaggatgcgagctggttgtgttttgacattaccgataatgtgccgcgtgaacgggtgcgttatgcccgcccggaagcggcgttttcccgtccggggaatggcatggagctgcgccttatccagacgctgatcgcccatcatcgcggttctttagatctctcggtccgccctgatggcggcaccttgctgacgttacgcctgccggtacagcaggttatcaccggaggcttaaaatga

SEQ ID NO: 219 - DNA, 1943 bp, synthetic construct
  misc_feature 1..1943: note = Synthetic polynucleotide
  misc_feature 1..1943: note = delta-nifL::Prm4 with 500bp flank
  source 1..1943: mol_type = other DNA
tgtttcgtctcgaggccgggcaactgagcggccccgttgaaaccgacctgggctggcatctgttgttgtgcgaacaaattcgcctgccgcaacccttgccgaaagccgaagccttaacgcgggtgcgtcagcaactgattgcccggcaacagaaacattatcagcgccagtggctgcaacaactgatcaacgcctgagcctgttctccttcttgttgatgcagacgggttaatgcccgttttgcacgaaaaatgcacataaattgcctgcgttgccttataacagcgcagggaaatcctgcctccggccttgtgccacaccgcgctttgcctggtttgtggtaaaaactggcccgctttgcatcctgatgcttaaaacaccccgttcagatcaacctttgggcagataagcccgcgaaaggcctgcaaattgcacggttattccgggtgagtatatgtgtgatttgggttccggcattgcgcaataaaggggagaaagacatgagcatcacggcgttatcagcatcatttcctgaggggaatatcgccagccgcttgtcgctgcaacatccttcactgttttataccgtggttgaacaatcttcggtggcgatttcgctgaccgatccgcaggcgcgcatttgttatgccaatccggcattctgccgccagacgggttttgcacttgagacacttttgggcgagaaccaccgtctgctggagtctgaactcatcctgcggcagtcggtgagacgtatttttgaccaaagagtgatctacatcacggaattttgtggttgttgctgcttaaaagggcaaatctacccttagaatcaactgttatatcagggggattcagagagatattaggaatttgcacaagcgcacaatttaaccacatcatgataacgccatgtaaaacaaagataaaaaaacaaaatgcagtgacttacatcgcaagcaaggcattttcttatccaattgctcaaagtttggcctttcatatcgcaacgaaaatgcgtaatatacgcgcccttgcggacatcagtatggtcattcctagttcatgcgcatcggacaccaccagcttacaaattgcctgattgcggccccgatggccggtatcactgaccgaccatttcgtgccttatgtcatgcgatgggggctgggccgtctctgaagctctcggtgaacattgttgcgaggcaggatgcgagctggttgtgttttgacattaccgataatgtgccgcgtgaacgggtgcgttatgcccgcccggaagcggcgttttcccgtccggggaatggcatggagctgcgccttatccagacgctgatcgcccatcatcgcggttctttagatctctcggtccgccctgatggcggcaccttgctgacgttacgcctgccggtacagcaggttatcaccggaggcttaaaatgacccagttacctaccgcgggcccggttatccggcgctttgatatgtctgcccagtttacggcgctttatcgcatcagcgtggcgctgagtcaggaaagcaacaccgggcgcgcactggcggcgatcctcgaagtgcttcacgatcatgcatttatgcaatacggcatggtgtgtctgtttgataaagaacgcaatgcactctttgtggaatccctgcatggcatcgacggcgaaaggaaaaaagagacccgccatgtccgttaccgcatgggggaaggcgtgatcggcgcggtgatgagccagcgtcaggcgctggtgttaccgcgcatttcagacgatcagcgttttctcgaccgcctgaatatttacgattacagcctgccgttgattggcgtgccgatccccggtgcggataatcagccatcgggcgtgctggtggcacagccgatggcgttgcacgaagaccggctgactgccagtacgcggtttttagaaatggtc

SEQ ID NO: 220 - DNA, 2864 bp, Kosakonia sacchari
  gene 1..2864: note = glnD with flanking sequences
  source 1..2864: mol_type = genomic DNA
ttctgacattagcaaacgcgataaacggtccgctctgcgggccgttttttttaccgctcttccctgcagcgtcatacatataaacgacttttcatttttcattatctcttttaagttgttttgctatgcatatgcactccagacgggtggcccataatgagcaacatcacttcagaacaatttgtcagcgcggtacaacccacccttcccggccagcctgaatatccaggggtttggtcacaagacgaattccagtgcgccaatatcaaagcgcatattgaggctttccagcagtggctcgggcacgtctttgacgccggaatttcagccgagcaattaattgaagcgcgcaccgaatttattgatcaacttctgcaacgcttatggctggagtacggttttggcgacgtcagtgataccgcgctggttgcggtaggcggttacggtcgcggcgaactgcatccgctatcggatattgatttactgatcctcagccgcagcaaactttccgacgaacaagcgcagaagattggcgaactgctcacgctgctgtgggatgtcaaactcgaagtgggacacagcgttcgtacgctggatgagtgcttacaggaagggttatcggatctcactgtcgcgaccaacttaattgaatcacgtctgctgattggcgacgtcgcgctgtttctggaactgcaaaagcatatttttagcgacagcttctggccatcggaaacttttttcccggcaaaagtggcggaacaaaacgagcgccaccagcgttatcacggcaccagttataacctggaacccgacataaaaagtagccctggtggtctgcgcgatattcacaccctgcaatgggttgctcgccgtcactttggtgccacatcgctggatgaaatggtgggctttggctttctgaccgaagccgagcgcaatgagctgaacgagtgtctgcacctgctttggcgtatccgcttcgcgctgcatctggaagtgagccgctacgacaaccgcctgctgttcgaccgccaactgagcgtagcgcagcgtctgaattatgtcggtgaaggcaaccagcctgtcgaacatatgatgaaggatttcttccgcgttacccgccgcgtcgcggagctgaaccagatgctgttacagctctttgatgaagcgattctggcgctgaccaccgacgagaaaccgcgcccgattgacgatgaattccaactgcgcggttcgcttatcgatctgcgtgacgaaacgctgtttatccgtgagccgcaggccatcctgcgcatgttctatatcatggtgcgcaacagcaccattaccggcatctactccaccacgctgcgccacttgcgccacgcccgccgccatctgacgcagccgctgtgttacatcgcggaagcacgctctattttcctcagcatgttgcgccatccaggcgccgtcagccgcggtttgctgccgatgcaccgccacagcgtgttgtgggcttatatgccacagtggtcgcacatcgtcggtcagatgcagtttgacctgttccacgcctataccgtggatgagcacaccattcgcgtattgctgaagctggaaagttttgccaaagaagagacgcgcaataagcaccctctatgcgtggaactgtggccgcgtttgacccacccggagctgatcctgatcgcggcgttgttccatgatatcgccaaaggacgcggcggcgaccactcggtgctgggtgcgcaggatgtgctgaagttcgctgaattacacgggcttaactcacgcgaaacccagctggttgcctggctggtgcgccatcatctgttgatgtcggtcaccgcccagcgccgtgatattcaggatccggaagtcatcaagcagtttgccgaagaggtgcaaacggaaaaccgtctgcgcttcctcgtctgtctgaccgtggcggatatctgtgccaccaacgaaaccctgtggaacagttggaagcagagcttgctgcgcgagctctatttcgcgactgaaaaacagctgcgtcgcggcatgcaaaacacgccggatatgcgcgaacgcgtgcgccatcaccagcttcaggcgctggcgttgctgcgtatggataatattgatgaagagcagttgcatcagatttgggcgcgctgtcgggcgaactattttgttcgccatagcccaaaccagcttgcctggcatgcgcgccacctgctgcaacatgatctgagcaagccgatgattttgctcagcccacaggctacgcgcggcggcaccgagatctttatctggagcccggatcgcccgtatctgtttgcagcggtgtgcgccgagcttgatcgtcgcaacctgagcgttcacgacgcgcagatttttaccacccgcgacggcatggcaatggatacctttattgtgctggaaccggacggaagcccgctctccgccgatcgccacgacgcgatttgtttcgggctggagcaggccattacgcaacacacctggcagccgccgcaaccgcgccgtcagcctgccaaattgcgccactttaccgtcgataccgaagtgaatttcctgccaacccataccgacagaaaatcgtttcttgaattgattgcccttgaccagccagggctgcttgcccgcgtcgggcaggtgtttgcggatctgggaatttcgctgcacggggcgagaattacaaccattggtgagcgagtagaagatttatttataatcgcgactgccgaccggcgtggccttaataatgcgctgcaaaaggaggtacaacaacggttgacagaggccctcaatccaaacgataaagggtgattatttttttacacagatggaaagagtaaaca

SEQ ID NO: 221 - DNA, 426 bp, synthetic construct; identical to SEQ ID NO: 147
  misc_feature 1..426: note = delta-nifL::Prm5
  source 1..426: mol_type = other DNA

SEQ ID NO: 222 - DNA, 1563 bp, synthetic construct; identical to SEQ ID NO: 155
  misc_feature 1..1563: note = glnE-delta-AR
  source 1..1563: mol_type = other DNA

SEQ ID NO: 223 - DNA, 188 bp, synthetic construct
  misc_feature 1..188: note = delta-glnD
  source 1..188: mol_type = other DNA
ttctgacattagcaaacgcgataaacggtccgctctgcgggccgttttttttaccgctcttccctgcagcgtcatacatataaacgacttttcatttttcattatctcttttaagttgttttgctatgcatatgcactccagacgggtggcccatattatttttttacacagatggaaagagtaaaca

(19) United States
(12) Patent Application Publication
(10) Pub. No.: US 2025/0145544 A1
(43) Pub. Date: May 8, 2025

(54) DRY FORMULATED NITROGEN-FIXING MICROBE PACKAGED IN WATER-SOLUBLE FILM FOR RAPID AND SAFE DISPERSAL IN AQUEOUS MIXTURES

(71) Applicant: Pivot Bio, Inc., Berkeley, CA (US)

(72) Inventors: Scott Alan STROBEL, Emeryville, CA (US); Farzaneh REZAEI, Cary, NC (US); Ernest SANDERS, Wright City, MO (US); Richard BROGLIE, Landenberg, PA (US)

(73) Assignee: Pivot Bio, Inc., Berkeley, CA (US)

(21) Appl. No.: 18/836,847

(22) PCT Filed: Feb. 9, 2023
(86) PCT No.: PCT/US2023/062299

§ 371 Date: Aug. 8, 2024

Related U.S. Application Data
(60) Provisional application No. 63/308,308, filed on Feb. 9, 2022.

(51) Int. Cl. C05F 11/08; C05G 5/16; C12N 1/04; C12N 1/20; C12R 1/22

(52) U.S. Cl. CPC C05F 11/08; C05G 5/16; C12N 1/04; C12N 1/205; C12R 2001/22

(57) ABSTRACT

The present disclosure provides water-soluble film packages composed of a water-soluble film, where the packages include: one or more compartment(s) including dehydrated microbes. The disclosure further provides methods of production of the water-soluble film packages, and methods of application thereof. The water-soluble packages of the disclosure enable convenient and accurate application of microbial dry powders to plants, promote uniform dispersion of powdered microbes, enhance microbial shelf life, and enhance the survival of microbes on seed, while being environmentally sustainable.

### CROSS REFERENCE TO RELATED APPLICATIONS

The present application claims the benefit of priority to U.S. Provisional Application No. 63/308,308, filed on Feb. 9, 2022, the contents of which are hereby incorporated by reference in their entirety.

### REFERENCE TO AN ELECTRONIC SEQUENCE LISTING

The contents of the electronic sequence listing (PIVO_021_02WO_SeqList_ST26.xml; Size: 613,217 bytes; and Date of Creation: Feb. 7, 2023) are herein incorporated by reference in its entirety.

### FIELD

The present disclosure relates to water-soluble packages comprising dehydrated microorganisms, methods of preparing thereof, and methods of use thereof. The water-soluble packages disclosed herein may be used to improve yield and productivity, and have other beneficial effects on agricultural crops.

### BACKGROUND

Application of plant beneficial microbes, such as nitrogen-fixing bacteria, to crops can increase agricultural yield, while potentially decreasing the use of fertilizers. These beneficial microbes must be cultured and transplanted to the soil near the root structure of the plant. Formulations that contain nitrogen-fixing bacteria are typically in the form of dry powders, which are often cumbersome to apply in a safe and consistent manner. Liquid formulations of plant beneficial microbes have short shelf life and therefore, are not preferred.

Thus, there continues to be a need for safe, convenient and eco-friendly packaging solutions for agricultural formulations containing microbes, particularly for dry microbial powder. Further, these packaging solutions should maintain the viability of the microbes by protecting them from excess drying and moisture until use.

### SUMMARY

The disclosure provides water-soluble film packages composed of a water-soluble film, the packages comprising: one or more compartment(s), wherein at least one of the one or more compartments comprising dehydrated microbes. In some embodiments, the dehydrated microbes are nitrogen-fixing microbes. In some embodiments, the nitrogen fixing microbes are diazotrophic bacteria. In some embodiments, the dehydrated microbes are in granular form. In some embodiments, the dehydrated microbes are powdered microbes.

The disclosure further provides methods for supplying nitrogen to a plant, the method comprising: a) contacting any of the water-soluble film packages disclosed herein with a liquid to produce a dispersion of live microbes; and b) applying the dispersion of live microbes to a locus comprising the plant, thereby colonizing the locus with the microbes; wherein the microbes fix atmospheric nitrogen, thereby supplying nitrogen to the plant. The disclosure also provides methods for coating a seed with nitrogen-fixing microbes, the method comprising: a) contacting any of the water-soluble film packages disclosed herein with a liquid to produce a dispersion of live microbes; and b) coating the seed with the dispersion of live microbes.

The disclosure provides methods of producing a dispersible formulation of dehydrated microbes, comprising: encapsulating dehydrated microbes in a water-soluble film, thereby producing a water-soluble film package comprising one or more compartment(s). The disclosure also provides method of producing a dispersible formulation of dehydrated microbes, comprising: a) providing microbes and a water-soluble film; b) culturing the microbes in growth media to produce a microbial culture liquid, c) admixing the microbial culture liquid with an ingredient selected from the group consisting of a bulking agent, an anticaking agent, a microbial stabilizer, a physical stabilizer, a dispersant, and/or milk solids nonfat; d) dehydrating the microbial culture liquid to produce dried material, wherein the dried material comprises dehydrated microbes, e) encapsulating the dried material in the water-soluble film, thereby producing a water-soluble film package.

### BRIEF DESCRIPTION OF THE FIGURES

FIG. 1 shows the number of colony forming units (CFUs) obtained upon plating each of the dispersions as indicated, relative to the weight of the microbial powder dissolved, at 0 h or 24 h after dispersion.

FIGS. 2A and 2B show the concentration (CFU/mL) of viable Kosakonia sacchari 6-5687 cells (FIG. 2A) or viable Klebsiella variicola 137-2253 cells (FIG. 2B) obtained after rehydration of dry microbial powder either in 1×PBS alone, or in 1×PBS in the presence of 2% (w/v) of a commercial grade, water-soluble MonoSol® film (either M7031, M8310 or M8534) and incubated at 21° C. for 1 hour.

FIG. 3 shows the amount of acetylene (average mmol of ethylene/CFU/h) produced in the acetylene reduction assay (ARA) in the presence of ammonium chloride (AmCl) by Kosakonia sacchari designated with the strain ID 6-5687 or Klebsiella variicola designated with the strain ID 137-2253 upon growth as a liquid suspension culture, or upon rehydration of freeze dried powder in 1×PBS alone, or in 1×PBS in the presence of 2% (w/v) of a water-soluble film MonoSol® M8534.

### DETAILED DESCRIPTION

The present disclosure provides water-soluble film packages composed of a water-soluble film, the packages comprising: one or more compartment(s) comprising dehydrated microbes, methods of producing and using thereof.

### Definitions

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context.

The disclosure of any ranges will be understood to include the recited endpoints. Thus a range of 1-5 will include values 1 and 5.

The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

“Plant tissues,” as used herein, refers to any part of the plant during any aspect of the growing cycle, including seeds, seedlings, plants, or plant parts. Plant parts include leaves, roots, root hairs, rhizomes, stems, seed, ovules, pollen, flowers, fruit, cuttings, tubers, bulbs, etc. An agricultural plant tissue “comprising” a dispersion of live microbes disclosed herein includes agricultural plant tissues to which the dispersion of live microbes has been applied by any of the means set forth herein, e.g., spraying, in-furrow application, seed treatment, etc.

“Plant productivity” refers generally to any aspect of growth or development of a plant that is a reason for which the plant is grown. For food crops, such as grains or vegetables, “plant productivity” can refer to the yield of grain or fruit harvested from a particular crop. As used herein, improved plant productivity refers broadly to improvements in yield of grain, fruit, flowers, or other plant parts harvested for various purposes, improvements in growth of plant parts, including stems, leaves and roots, promotion of plant growth, maintenance of high chlorophyll content in leaves, increasing fruit or seed numbers, increasing fruit or seed unit weight, and similar improvements of the growth and development of plants. In some embodiments, plant productivity is determined by comparing the productivity (e.g., yield) of a treated plant (e.g., via in furrow application of dispersion of live microbes disclosed herein), vs. a plant to which the dispersion of live microbes has not been applied, and no additional fertilizer beyond what is provided to the treated plant. Thus, in some embodiments, the dispersion of live microbes result in reductions in NO₂ emission due to reduced nitrogen fertilizer usage.

Microbes in and around food crops can influence the traits of those crops. Plant traits that may be influenced by microbes include: yield (e.g., grain production, biomass generation, fruit development, flower set); nutrition (e.g., nitrogen, phosphorus, potassium, iron, micronutrient acquisition); abiotic stress management (e.g., drought tolerance, salt tolerance, heat tolerance); and biotic stress management (e.g., pest, weeds, insects, fungi, and bacteria). Strategies for altering crop traits include: increasing key metabolite concentrations; changing temporal dynamics of microbe influence on key metabolites; linking microbial metabolite production/degradation to new environmental cues; reducing negative metabolites; and improving the balance of metabolites or underlying proteins.

As used herein, “in planta” may refer to in the plant, on the plant, or intimately associated with the plant, depending upon context of usage (e.g. endophytic, epiphytic, or rhizospheric associations). As used herein, the term “plant” can include plant parts, tissue, leaves, roots, root hairs, rhizomes, stems, seeds, ovules, pollen, flowers, fruit, etc. Thus, when the disclosure discusses providing a plurality of corn plants to a particular locus, it is understood that this may entail planting a corn seed at a particular locus.

Fertilizers and exogenous nitrogen of the present disclosure may comprise the following nitrogen-containing molecules: ammonium, nitrate, nitrite, ammonia, glutamine, etc. Nitrogen sources of the present disclosure may include anhydrous ammonia, ammonia sulfate, urea, diammonium phosphate, urea-form, monoammonium phosphate, ammonium nitrate, nitrogen solutions, calcium nitrate, potassium nitrate, sodium nitrate, etc.

As used herein, “exogenous nitrogen” refers to non-atmospheric nitrogen readily available in the soil, field, or growth medium that is present under non-nitrogen limiting conditions, including ammonia, ammonium, nitrate, nitrite, urea, uric acid, ammonium acids, etc.

As used herein, “non-nitrogen limiting conditions” refers to non-atmospheric nitrogen available in the soil, field, media at concentrations greater than about 4 mM nitrogen, as disclosed by Kant et al. (2010. J. Exp. Biol. 62(4):1499-1509), which is incorporated herein by reference for all purposes.

A “wild type microbe,” e.g., a “wild type bacterium,” as used herein refers to a microbe that has not been genetically modified. Wild type microbes may be isolated and cultivated from a natural source. Wild type microbes may be selected for specific naturally occurring traits.

In some embodiments, the bacteria of the present disclosure have been modified such that they are not naturally occurring bacteria.

A “diazotroph” is a microbe that fixes atmospheric nitrogen gas into a more usable form, such as ammonia. A diazotroph is a microorganism that is able to grow without external sources of fixed nitrogen. All diazotrophs contain iron-molybdenum or -vanadium nitrogenase systems.

In some embodiments, the increase of nitrogen fixation and/or the production of 1% or more of the nitrogen in the plant are measured relative to control plants, which have not been exposed to the bacteria of the present disclosure. All increases or decreases in bacteria are measured relative to control bacteria. All increases or decreases in plants are measured relative to control plants.

As used herein, a “water-soluble film package”, interchangeably used herein with “water-soluble package” refers to an encasement that is capable of disintegrating upon contact with a liquid, and is composed of a water-soluble film. In some embodiments, the liquid is water or an aqueous solution. In some embodiments, one or more components of the water-soluble film package are water-soluble, such as, for example, a water-soluble polymer. In some embodiments, one or more components of the water-soluble film package are water-insoluble, such as, for example, a water-insoluble polymer. In some embodiments, the water-insoluble component of the disclosed packages may be a net (e.g., a plastic net) that is embedded in a water-soluble film. Water insoluble portions of the water-soluble package would not be water tight without the water-soluble portions of the package.

As used herein, a “water-soluble film” refers to a film that is capable of disintegrating upon contact with a liquid. In some embodiments, the liquid is water or an aqueous solution. In some embodiments, one or more components of the water-soluble film are water-soluble, such as, for example, a water-soluble polymer. In some embodiments, the water-soluble film is “fully” soluble, meaning that all ingredients in the film are capable of fully dissolving in liquid such as water. In some embodiments, the water-soluble film further comprises one or more water-insoluble components, that still lose cohesion when exposed to a liquid, such as water. For example, in some embodiments, the water-soluble film may contain granules, strips, netting, or other non-water-soluble ingredients that are held together to form a pouch with water-soluble ingredients, such that exposure of the film to water or another liquid still causes the film to lose integrity and dissipate, even if some components are not fully solvated in the liquid.

As used herein, the terms “agricultural composition”, “agricultural formulation”, or “agricultural biological” refers to a dry composition, comprising dehydrated microbes, encapsulated within one or more compartments of the water-soluble packages. In some embodiments, the agricultural composition comprises one or more additives in addition to the dehydrated microbes, as described herein. In some embodiments, all the components of the agricultural composition, such as the microbes and additives, may be contained within a water-soluble package comprising a single compartment. In some embodiments, the agricultural compositions of the present disclosure may be split into more than one compartment within the water-soluble pouch. In some embodiments, the compartments within the water-soluble pouch have the same composition (i.e., have the same ingredients). In some embodiments, the agricultural compositions of the present disclosure are split across more than one compartment, such that at least two compartments comprise different ingredients (e.g., with one having the dehydrated microbes an microbial stabilizer and the other having fertilizer for the plant). Thus, in some embodiments, the term “agricultural composition” refers to all the ingredients contained within the water-soluble pouch, whether they are mixed together, or spread across different compartments.

As used herein, a “dispersion of live microbes” or “microbial dispersion” refers to the composition obtained by contacting any one of the water-soluble film packages disclosed herein with a liquid. In some embodiments, the liquid is water or an aqueous solution. In some embodiments, the dispersion of live microbes comprises the liquid, the microbes that were contained in the water-soluble package, and one or more of the components of the water-soluble package. In some embodiments, one or more of the components of the water-soluble package that is in the dispersion of live microbes is polyvinyl alcohol. In some embodiments, the microbes in the dispersion are rehydrated.

As used herein, an “intergeneric microorganism” is a microorganism that is formed by the deliberate combination of genetic material originally isolated from organisms of different taxonomic genera. An “intergeneric mutant” can be used interchangeably with “intergeneric microorganism”. An exemplary “intergeneric microorganism” includes a microorganism containing a mobile genetic element which was first identified in a microorganism in a genus different from the recipient microorganism. Further explanation can be found, inter alia, in 40 C.F.R. § 725.3. In aspects, microbes taught herein are “non-intergeneric,” which means that the microbes are not intergeneric.

As used herein, an “intrageneric microorganism” is a microorganism that is formed by the deliberate combination of genetic material originally isolated from organisms of the same taxonomic genera. An “intrageneric mutant” can be used interchangeably with “intrageneric microorganism.”

As used herein, an “intragenic” microorganism, is a microorganism that is engineered to comprise a genetic edit, or genetic modification, or genetic element, or genetic material (e.g. a nucleic acid sequence), that has been sourced from within the organism's own genome.

As used herein, a “transgenic” microorganism, is a microorganism that is engineered to comprise a genetic edit, or genetic modification, or genetic element, or genetic material (e.g. a nucleic acid sequence), that has been sourced from outside the organism's own species.

As used herein, in the context of non-intergeneric microorganisms, the term “remodeled” is used synonymously with the term “engineered”. Consequently, a “non-intergeneric remodeled microorganism” has a synonymous meaning to “non-intergeneric engineered microorganism,” and will be utilized interchangeably.

As used herein, “applying,” “coating,” and “treating” agricultural plant tissues or the environs thereof with the dispersion of live microbes includes any means by which the plant tissues or the environs thereof are made to come into contact (i.e. exposed) to a dispersion of live microbes. In some embodiments, “applying” refers to placing or distributing the dispersion of live microbes onto an area, volume, or quantity of agricultural plant tissues or the environs thereof. Consequently, “applying” includes any of the following means of exposure to a dispersion of live microbes: spraying, dripping, submerging, hand broadcast, machine spreading, brushing, machine broadcasting, irrigating, top dressing vehicle, and the like, onto agricultural plant tissues or the environs thereof, applying as a seed coat, applying to a field that will then be planted with seed, applying to a field already planted with seed, etc.

As used herein, “the environs” of agricultural plant tissues include the elements of the vicinity around the agricultural plant tissues that come into contact with the agricultural plant tissues. For example, application to the environs of agricultural plant tissues would include soil application and in-furrow application means.

As used herein the terms “microorganism” or “microbe” should be taken broadly. These terms, used interchangeably, include but are not limited to, the two prokaryotic domains, Bacteria and Archaea. The term may also encompass eukaryotic fungi and protists.

As used herein, when the disclosure discuses a particular microbial deposit by accession number, it is understood that the disclosure also contemplates a microbial strain having all of the identifying characteristics of said deposited microbe, and/or a mutant thereof.

In certain aspects of the disclosure, the isolated microbes exist as “isolated and biologically pure cultures.” It will be appreciated by one of skill in the art, that an isolated and biologically pure culture of a particular microbe, denotes that said culture is substantially free of other living organisms and contains only the individual microbe in question. The culture can contain varying concentrations of said microbe. The present disclosure notes that isolated and biologically pure microbes often “necessarily differ from less pure or impure materials.” See, e.g. In re Bergstrom, 427 F.2d 1394, (CCPA 1970)(discussing purified prostaglandins), see also, In re Bergy, 596 F.2d 952 (CCPA 1979)(discussing purified microbes), see also, Parke-Davis & Co. v. H.K. Mulford & Co., 189 F. 95 (S.D.N.Y. 1911) (Learned Hand discussing purified adrenaline), aff'd in part, rev'd in part, 196 F. 496 (2d Cir. 1912), each of which are incorporated herein by reference. Furthermore, in some aspects, the disclosure provides for certain quantitative measures of the concentration, or purity limitations, that must be found within an isolated and biologically pure microbial culture. The presence of these purity values, in certain embodiments, is a further attribute that distinguishes the presently disclosed microbes from those microbes existing in a natural state. See, e.g., Merck & Co. v. Olin Mathieson Chemical Corp., 253 F.2d 156 (4th Cir. 1958) (discussing purity limitations for vitamin B12 produced by microbes), incorporated herein by reference.

Microbes of the present disclosure may include spores and/or vegetative cells. In some embodiments, microbes of the present disclosure include microbes in a viable but non-culturable (VBNC) state. As used herein, “spore” or “spores” refer to structures produced by bacteria and fungi that are adapted for survival and dispersal. Spores are generally characterized as dormant structures; however, spores are capable of differentiation through the process of germination. Germination is the differentiation of spores into vegetative cells that are capable of metabolic activity, growth, and reproduction. The germination of a single spore results in a single fungal or bacterial vegetative cell. Fungal spores are units of asexual reproduction, and In some embodiments are necessary structures in fungal life cycles. Bacterial spores are structures for surviving conditions that may ordinarily be nonconducive to the survival or growth of vegetative cells.

As used herein, the term “agronomically stable” refers to a composition (such as, the agricultural compositions within the water-soluble packages disclosed herein) that maintains the viability of the microorganisms over time. The agricultural compositions disclosed herein may exhibit a decline in the bacterial concentration over time, but at a reduced rate compared to traditional liquid formulations or dry microbial powders not encapsulated in the water-soluble packages disclosed herein. Loss of live bacterial concentration may be measured in log loss of CFU per unit over time.

As used herein, a “seed treatment” refers to a substance (e.g., a dispersion of live microbes disclosed herein) that may be applied to agricultural seeds. The seed treatment may provide one or more benefits to the seed and/or plant resulting from the seed. Without limitation, seed treatments may include the dispersion of live microbes disclosed herein, pesticides, herbicides, insecticides, nematicides, plant-growth promoting factors, fertilizers, and the like.

The term “colony forming unit” or “CFU” as used herein is a unit used to estimate the number of viable microbial cells in a sample. Viable is defined as the ability to multiply under the controlled conditions. Counting colony-forming units requires culturing the microbes and counting only viable cells, in contrast with microscopic examination which counts all cells, living or dead. The visual appearance of a colony in a cell culture requires significant growth and may result from the growth of individual or multiple viable cells.

As used herein, a “buffering agent,” “buffer solution,” or “buffer,” also known as a “pH buffer” or “hydrogen ion buffer,” consists of a mixture of a weak acid and its conjugate base, or a weak base and its conjugate acid. Its pH changes very little when a small amount of strong acid or base is added to it. Buffering agents are used as a means of keeping pH at a nearly constant value, or within a certain pH range, over a period of time. Herein, “a buffering agent” may refer to either a chemical compound used to buffer a formulation or to a buffering system comprising a combination of acids, bases, and/or salts.

As used herein, a “dispersing agent” or “dispersant” is a substance that, when added to a dispersion of solid or liquid particles in a liquid, is capable of promoting the separation of the dispersed particles and thus, prevent clumping or settling of the dispersed particles. In some embodiments, a dispersing agent added to a dispersion of live microbes disclosed herein can improve and/or stabilize the dispersion by promoting the separation of the microbes, and preventing the clumping or settling of the microbes. In some embodiments, addition of a dispersing agent to a dispersion of live microbes can promote rehydration, viability, and/or shelf-life of the microbes. In some embodiments, the dispersing agent is a biologically compatible dispersing agent, such as, for example, non-ionic, anionic, amphoteric, or cationic dispersing and emulsifying agents. In some embodiments, the dispersing agent is comprised within the water-soluble pouch, so that it is included in the dispersion of live microbes after the pouch is dissolved in a liquid.

### Current Dry Microbe Application Technologies

Plant beneficial microbes, such as nitrogen-fixing bacteria, must be cultured and transplanted to the soil near the root structure of the plant. In order to be active, beneficial microbes are usually stored in a dehydrated form, and then rehydrated and/or cultured just prior to, or after being applied to plants. Since dry microbial formulations (such as, powders or granules) typically have a longer shelf compared to liquid microbial formulations, dry powder formulations are a preferred mode of transporting and storing beneficial microbes until use.

Dry microbial formulations however, have several disadvantages, as described below.

There may be real and perceived safety concerns over the direct handling of dry powders containing microbes. For instance, farmers and treatment applicators may be concerned about potential inhalation of the microbial powder or contact of the microbial powder with skin or eye. It is also challenging for end-users to apply accurate and reproducible doses of dry formulations, at least in part, because of potential weight variability due to differing moisture content of the products in different climates, or volume fluctuations with particle aggregation. Moreover, the packaging of microbial dry powder for individual field doses can result in excess plastic and non-degradable waste.

Beneficial microbes are sometimes administered in the form of seed coatings. This approach however, is also not without its potential drawbacks. For example, the long-term viability of the microbes coated on seed surfaces is often reduced by excess drying due to seed processing methods used or storage conditions. Furthermore, some microbial seed coatings may be incompatible with other types of chemical seed treatments, and most seed treatment facilities are not equipped to apply dry microbial powder to seeds.

To overcome the challenges of using dry microbial powder, while harnessing the beneficial effects of these microbes and the shelf stability of dry microbial compositions, the present disclosure provides safe, convenient and eco-friendly water-soluble packaging solutions for agricultural formulations containing dehydrated microbes, particularly for dry microbial powder, as described in detail herein.

### Water-Soluble Film Packaging Comprising Dehydrated Microbes

The disclosure provides water-soluble film packages, comprising agricultural compositions, which comprise microbes, for example, dehydrated microbes. In some embodiments, the packages are composed of a water-soluble film. In some embodiments, the package comprises one or more compartment(s). In some embodiments, the one or more compartment(s) comprises agricultural compositions, comprising dehydrated microbes.

When the water-soluble packages disclosed herein are brought into contact with a liquid (such as, water or an aqueous solution), the package disintegrates, releasing the agricultural composition contained therein into the liquid, thereby forming a “dispersion of live microbes” that can be applied to plants or any plant part, such as seeds, as described in detail herein. The use of water-soluble packages disclosed herein enable convenient and accurate application of microbes to soil and plants and offer the following advantages.

First, the use of the water-soluble packages disclosed herein obviates the need for direct handling of the dry microbial powder by the end-user, such as a seed treater or a farmer, thus eliminating any real or perceived safety concerns due to the potential inhalation or contact of the microbial powder with skin or eye. Second, the water-soluble packages disclosed herein can be designed to contain a standardized unit of microbes for simplified dosing. Third, the use of the water-soluble packages disclosed herein can promote uniform dispersion of the powdered microbe. For instance, in some embodiments, the components of the water-soluble packages help stabilize the microbes during dry storage and improve dispersion of the microbe in liquids. Therefore, the use of the water-soluble packages promotes consistency in application results.

Fourth, the water-soluble packages disclosed herein enhance the shelf life of the microbes contained therein, since they provide an effective barrier between the microbial powder, and moisture and/or oxygen. Fifth, the water-soluble packages are environment-friendly and reduce packaging waste, while having the potential to be aesthetically pleasing. Sixth, they allow for single dose administration of multiple components that may not be amenable to comingling during storage, or which benefit from different administration timings. Finally, one or more components of the water-soluble packages disclosed herein (such as, polyvinyl alcohols) are released into the dispersion of live microbes upon contact with the liquid. Such components may enhance the survival of the microbes in the dispersion and/or on seed, when the dispersion is applied as a seed treatment.

### I. Compartments of the Water-Soluble Package

In some embodiments, the water-soluble film package comprises one compartment. In some embodiments, the water-soluble film package comprises more than one compartment, such as, for example, 1, 2, 3, 4, 5, 6, 7, 8, 9, or 10 compartments. In some embodiments, the package comprises 2 compartments. In some embodiments, package comprises 3 compartments. In some embodiments, the package comprises 4 compartments. In some embodiments, the package comprises 5 compartments. In some embodiments, the one or more compartments are placed adjacent to each other in the water-soluble package. In some embodiments, one or more compartments may be within one or more other compartments of the water-soluble package.

In some embodiments, the package comprises more than one compartment, in which at least one of the compartments contains an agricultural composition, comprising dehydrated microbes. In some embodiments, one or more additives disclosed herein or known in the art are also contained in a compartment containing the dehydrated microbes. In some embodiments, one or more additives disclosed herein or known in the art are contained in a compartment that does not contain dehydrated microbes.

The water-soluble packages disclosed herein enable the co-administration of components (such as, microbes and one or more additives) that may not be amenable to being in contact with each other prior to the time of administration, during storage, and/or for long periods of time. Furthermore, when the disclosed agricultural components are brought in contact with a liquid to form a dispersion that can be applied to plants or plant parts, it may be desirable to bring different components in contact with each other in a timed and/or ordered fashion. This kind of regulation of the contact (as well as the timing and/or the order of the contact) between the components can be achieved using the compartmentalization of the disclosed water-soluble packages, as described below.

In some embodiments, contact between one or more dehydrated microbes and one or more additives is prevented until use by placing them in separate compartments of the water-soluble package. In some embodiments, contact among one or more of the dehydrated microbes is prevented until use by placing the one or more microbes in separate compartments of the disclosed packages. In some embodiments, contact among one or more of the additives until use is prevented by placing the one or more additives in separate compartments of the disclosed packages.

In some embodiments, the compartmentalization of the water-soluble package is used to regulate the timing of the contact among one or more microbes; between one or more microbes and one or more of the additives; and/or among the one or more additives. In some embodiments, the compartmentalization of the water-soluble package is used to regulate the timing of the contact between one additive and another additive. The timing of contact between one or more components of the water-soluble package may be regulated by regulating the dissolution kinetics of the water-soluble film in a spatial manner. That is, different compartments of the disclosed packages may be enveloped by water-soluble films that differ in their dissolution kinetics, such that a compartment enveloped by a water-soluble film having faster dissolution kinetics disintegrates faster than a compartment enveloped by a water-soluble film having slower dissolution kinetics.

The dissolution kinetics of the water-soluble film may be influenced or determined by the physical properties of the film (such as, density, thickness, porosity, etc.), its structure (such as whether a compartment is internal, or faces the outside environment), and/or the chemical properties of the film (such as, the composition of the film). Therefore, in some embodiments, the water-soluble packages comprise one or more compartments, wherein the one or more compartments differ from each other based on the physical properties of the water-soluble film that envelopes them (that is, they may have different density, thickness, porosity, etc.) and/or chemical properties of the water-soluble film that envelopes them (that is, they may comprise different components, such as different polymers).

In some embodiments, the package comprises a first and a second compartment, and wherein the dehydrated microbes are in the first compartment. In some embodiments, the first and/or the second compartment comprises any one or more additives disclosed herein, or known in the art to be used for this purpose.

In some embodiments, one or more compartment(s) of the water-soluble film package comprise a carbon source capable of enhancing growth of the dehydrated microbes. In some embodiments, the first or second compartment comprises a carbon source capable of enhancing growth of the dehydrated microbes. In some embodiments, the one or more compartment(s) comprise a dispersing agent. In some embodiments, the first or second compartment comprises a dispersing agent. In some embodiments, the one or more compartment(s) comprise a fertilizer. In some embodiments, the first or second compartment comprises a fertilizer. In some embodiments, the one or more compartment(s) comprise a plant growth hormone. In some embodiments, the first or second compartment comprises a plant growth hormone.

In some embodiments, the one or more compartment(s) (for instance, the first or second compartment) comprises an agent selected from the group consisting of a bulking agent, an anticaking agent, a microbial stabilizer, a physical stabilizer, and a dispersant. In some embodiments, the one or more compartment(s) (for instance, the first or second compartment) comprises a microbial stabilizer selected from the list consisting of a monosaccharide, a disaccharide, a polysaccharide, a pentose, a hexose, an oligosaccharide, an oligofructose, a sugar alcohol, an amino acid, a protein or protein hydrolysate, glucose, fructose, trehalose, sucrose, lactose, melibiose, inulin, lactulose and a polymer. In some embodiments, the one or more compartment(s) (for instance, the first or second compartment) comprises a physical stabilizer selected from the list consisting of maltodextrin, polyethylene glycol (PEG), xanthan gum, pectin, alginates, microcrystalline cellulose, and dextran.

### II. Water-Soluble Film

In some embodiments, the present disclosure provides water-soluble packages, comprising a water-soluble film. In some embodiments, the water-soluble package comprises one or more sheets of the water-soluble film. In some embodiments, when the packages are brought in contact with a liquid (such as, water or an aqueous solution), the package comprising the water-soluble film disintegrates, and releases its contents into the liquid forming a dispersion of live microbes.

In some embodiments, the water-soluble film comprises a water-soluble polymer. As used herein the phrase “water-soluble polymer” and variants thereof refer to a polymer that is at least partially, predominantly, or fully soluble in water. In some embodiments, the water-soluble film comprises a water-soluble polymer and one or more water-insoluble components, such as a water-insoluble polymer. In some embodiments, when the water-soluble polymers dissolve in the liquid, the water-soluble film loses integrity and the contents of the disclosed packages are released into the liquid. In some embodiments, when the water-soluble polymers dissolve in the liquid, the water-insoluble components dissociate from the structure of the package.

In some embodiments, the water-soluble film and/or the water-soluble package is biodegradable. As used herein, “biodegradable” refers to a substance that is capable of being decomposed by living organisms, such as bacteria. In some embodiments, water-soluble film comprises one or more biodegradable polymers, copolymers, block polymers and any combinations thereof. In some embodiments, biodegradable polymers are natural or synthetically made, and may comprise of ester, amide, and ether functional groups. In some embodiments, the biodegradable polymers are polysaccharides, proteins, biopolyesters such as polyhydroxybutyrate and polylactic acid, and any combinations thereof. In some embodiments, the disclosed packages may comprise any other biodegradable polymers known in the art, such as, those disclosed in Vroman I, Tighzert L., Biodegradable Polymers, Materials, 2009; 2(2):307-344, the contents of which are incorporated herein by reference in its entirety for all purposes. In some embodiments, the water-soluble film and/or the water-soluble package is biocompatible. As used herein, the term “biocompatible” refers to a substance or material that is not harmful living tissue or living organisms.

In some embodiments, the water-soluble film comprises a polymer selected from the group consisting of cellulose, a cellulose derivative, pullulan, polyvinyl pyrrolidone, polyvinyl alcohol, polyethylene glycol, carboxyvinyl copolymers, hydroxypropylmethyl cellulose, hydroxyethyl cellulose, hydroxypropyl cellulose, carboxymethyl cellulose, sodium alginate, xanthan gum, tragacanth gum, guar gum, acacia gum, arabic gum, polyacrylic acid, methylmethacrylate copolymer, carboxyvinyl copolymers, starch, gelatin, and combinations thereof. In some embodiments, the water-soluble film further comprises a polyethylene oxide.

In some embodiments, the water-soluble film further comprises a water-insoluble polymer selected from the group consisting of ethylcellulose, hydroxypropyl ethyl cellulose, cellulose acetate phthalate, hydroxypropyl methyl cellulose phthalate, polyvinylacetatephthalates, phthalated gelatin, crosslinked gelatin, poly(lactic acid)/poly(glycolic acid)/polyethyleneglycol copolymers, polycaprolactone and combinations thereof.

In some embodiments, the water-soluble film further comprises a polymer selected from the group consisting of methylmethacrylate copolymer, polyacrylic acid polymer, poly(glycolic acid) (PGA), poly(lactic acid) (PLA), poly(lactic acid)/poly(glycolic acid)/polyethyleneglycol copolymers, polydioxanones, polyoxalates, poly(\acute{α}-esters), polyanhydrides, polyacetates, polycaprolactones, poly(orthoesters), polyamino acids, polyaminocarbonates, polyurethanes, polycarbonates, polyamides, poly(alkyl cyanoacrylates), and mixtures and copolymers thereof. In some embodiments, the water-soluble film comprises polyvinyl alcohol.

In some embodiments, the water-soluble film further comprises a polymer selected from the group consisting of stereopolymers of L- and D-lactic acid, copolymers of bis(p-carboxyphe-noxy) propane acid and sebacic acid, sebacic acid copolymers, copolymers of caprolactone, poly(lactic acid)/poly (glycolic acid)/polyethyleneglycol copolymers, copolymers of polyurethane and (poly(lactic acid), copolymers of poly-urethane and poly(lactic acid), copolymers of a-amino acids, copolymers of a-amino acids and caproic acid, copolymers of a-benzyl glutamate and polyethylene glycol, copolymers of succinate and poly(glycols), polyphosp-hazene, polyhydroxy-alkanoates and mixtures thereof. In some embodiments, the polymer is marketed under the Medisorb and Biodel trademarks. The Medisorb materials are marketed by the Dupont Company of Wilmington, Del. and are generically identified as a “lactide/glycolide co-polymer” containing “propanoic acid, 2-hydroxy-polymer with hydroxy-polymer with hydroxyacetic acid.” Four such polymers include lactide/glycolide 100 L; lactide/glycolide 100 L; lactide/glycolide 85/15; and lactide/glycolide 50/50. The Biodel materials represent a family of various polyanhydrides which differ chemically and generally regarded as biocompatible.

In some embodiments, the water-soluble film comprises a water-soluble polymer, a water swellable polymer, a water insoluble polymer, or any combination thereof. As used herein, water swellable polymer refers to a polymer that absorbs water.

In some embodiments, the water-soluble film comprises one or more crosslinkers, such as salts of calcium, sodium and potassium. In some embodiments, the water-soluble film comprises higher molecular weight polymers and polysaccharides and gums, which include without limitation, alginate, carageenan, hydroxypropyl methyl cellulose, locust bean gum, guar gum, xanthan gum, dextran, gum arabic, gellan gum, and any combination thereof. In some embodiments, the water-soluble film comprises a plasticizer. In some embodiments, the water-soluble film does not comprise a plasticizer. The plasticizer can be, without limitation, at least one of polyethylene oxide, polypropylene glycol, polyethylene glycol, glycerin, edible polyols, glycerol, polyols, maltitol, isomalt, and reduced sugars. In some embodiments, the water-soluble film comprises hydroxypropyl cellulose (HPC), hydroxypropyl methylcellulose (HPMC), or a combination thereof.

In some embodiments, the water-soluble film comprises a coloring agent. The use of titanium dioxide will create a white product. Other edible pigments may be used, such as Colorcon Red #40. The coloring can also be multilayered by taking advantage of the rheological differences of the layers of the film relative to the solubility and/or density of the coloring material.

In some embodiments, the water-soluble film is a “controlled release” or “regulated release” film. As used herein, the term “controlled release” refers to the release of the agricultural composition comprising the dehydrated microbes at a desired release rate upon contact with the liquid. In some embodiments, the release rate is reflected by the time period between the contact of the package with the liquid and the release of the contents of the disclosed package into the liquid. The desired release rate may be any rate depending on the application. In some embodiments, the release of the microbes into the liquid may occur immediately upon contact with the liquid. In some embodiments, the time period between the contact of the package with the liquid and the release of the contents of the disclosed package into the liquid may be in the range of about 1 second to about an hour, for example about 10s, about 30s, about 1 min, about 2 mins, about 3 mins, about 4 mins, about 5 mins, about 10 mins, about 15 mins, about 20 mins, about 25 mins, about 30 mins, or about 1 hour, including all values and sub ranges that lie there between.

In some embodiments, combinations of release patterns are contemplated, such that there is an initial fast release of some contents of the disclosed packages followed by a slower release of other contents from the same water-soluble package. This type of combination of release patterns may be achieved varying the dissolution kinetics of different compartments of the water-soluble packages, wherein one or more compartments disintegrate faster than others, as described in detail elsewhere in this application. For instance, in some embodiments, the microbes and/or additives in one compartment are released faster followed by the release of microbes and/or additives from another compartment.

In some embodiments, the release rate of the water-soluble film may be regulated by also varying the ratio of water-soluble polymers to water-insoluble polymers in the film. For instance, packages or package compartments comprising a higher ratio of water-insoluble polymers to water-soluble polymers may disintegrate and release their contents at a slower rate, compared to packages or package compartments comprising a lower ratio of water-insoluble polymers to water-soluble polymers. In some embodiments, the package or package compartment may comprise a water-soluble film having a particular release rate, which is further coated by a second water-soluble film with a different release rate. In this manner, the release of the contents from the package or package compartments may be regulated with time.

Further details on the components of the water-soluble film and water-soluble packages disclosed herein are provided in U.S. Pat. Nos. 7,357,891, 8,617,589, 11,028,352, 6,484,879, 7,642,226, 11,407,866, 11,453,754, 11,352,468, 7,897,080, 7,824,588, 9,834,354, 10,604,318, 10,472,476, 11,168,289, 10,526,479, 10,899,518, 10,562,701, 9,873,558, 10,183,794, 10,913,832, 10,815,346, 11,473,039, 9,073,294, 10,443,024, 11,193,092, 10,087,401, 10,611,555, 10,696,460, 6,821,590, 7,005,168, 9,796,833, 9,908,675, 9,670,440, 10,829,621, 7,803,872, 10,907,117, 9,150,782, 10,202,227, 10,208,422, 11,104,497, 10,808,210, 9,670,437, 11,459,433, 9,828,154, 8,268,914, 10,844,183, 8,728,449, 9,394,092, 10,513,588, 11,492,190, 10,428,297, 11,473,040, and WO 2014/202412, the contents of each of which are herein incorporated by reference in its entirety for all purposes.

### III. Dehydrated Microbes

The water-soluble packages disclosed herein comprise agricultural compositions comprising dehydrated microbes, such as plant beneficial bacteria. In some embodiments, the dehydrated microbes are nitrogen-fixing microbes. In some embodiments, the nitrogen fixing microbes are diazotrophic bacteria. In some embodiments, the dehydrated microbes are in granular form. In some embodiments, the dehydrated microbes are in the form of a dry microbial powder. In some embodiments, the disclosure provides the water-soluble packages described in any one of: U.S. Pat. Nos. 7,357,891, 8,617,589, WO 2014/202412, WO 2014/202412, WO 2010/0088112, EP 1375637, EP 1394065, and US 2001/0033883, the packages comprising any one or more of the agricultural compositions, comprising the dehydrated microbes disclosed herein.

In some embodiments, the microbes are obtained from any source. In some embodiments, microbes are bacteria, archaea, protozoa, algae, or fungi. In some embodiments, the microbes of this disclosure are nitrogen fixing microbes, for example nitrogen fixing bacteria, nitrogen fixing archaea, nitrogen fixing fungi, nitrogen fixing yeast, nitrogen fixing algae, or nitrogen fixing protozoa. In some embodiments, microbes are spore forming microbes, for example spore forming bacteria. In some embodiments, bacteria disclosed herein are Gram positive bacteria or Gram negative bacteria. In some embodiments, the bacteria are endospore forming bacteria of the Firmicute phylum. In some embodiments, the bacteria are diazotrophs. In some embodiments, the bacteria are not diazotrophs.

In some embodiments, the microbes are an archaea, such as, for example, Methanothermobacter thermoautotrophicus, Methanosarcina barkeri, Methanospirillum hungatei, Methanobacterium bryantii, Methanococcus thermolithotrophicus, and Methanococcus maripaludis.

In some embodiments, bacteria include, but are not limited to, Agrobacterium radiobacter, Bacillus acidocaldarius, Bacillus acidoterrestris, Bacillus agri, Bacillus aizawai, Bacillus albolactis, Bacillus alcalophilus, Bacillus alvei, Bacillus aminoglucosidicus, Bacillus aminovorans, Bacillus amylolyticus (also known as Paenibacillus amylolyticus) Bacillus amyloliquefaciens, Bacillus aneurinolyticus, Bacillus atrophaeus, Bacillus azotoformans, Bacillus badius, Bacillus cereus (synonyms: Bacillus endorhythmos, Bacillus medusa), Bacillus chitinosporus, Bacillus circulans, Bacillus coagulans, Bacillus endoparasiticus Bacillus fastidiosus, Bacillus firmus, Bacillus kurstaki, Bacillus lacticola, Bacillus lactimorbus, Bacillus lactis, Bacillus laterosporus (also known as Brevibacillus laterosporus), Bacillus lautus, Bacillus lentimorbus, Bacillus lentus, Bacillus licheniformis, Bacillus maroccanus, Bacillus megaterium, Bacillus metiens, Bacillus mycoides, Bacillus natto, Bacillus nematocida, Bacillus nigrificans, Bacillus nigrum, Bacillus pantothenticus, Bacillus popillae, Bacillus psychrosaccharolyticus, Bacillus pumilus, Bacillus siamensis, Bacillus smithii, Bacillus sphaericus, Bacillus subtilis, Bacillus thuringiensis, Bacillus uniflagellatus, Bradyrhizobium japonicum, Brevibacillus brevis, Brevibacillus laterosporus (formerly Bacillus laterosporus), Chromobacterium subtsugae, Delftia acidovorans, Lactobacillus acidophilus, Lysobacter antibioticus, Lysobacter enzymogenes, Paenibacillus alvei, Paenibacillus polymyxa, Paenibacillus popilliae (formerly Bacillus popilliae), Pantoea agglomerans, Pasteuria penetrans (formerly Bacillus penetrans), Pasteuria usgae, Pectobacterium carotovorum (formerly Erwinia carotovora), Pseudomonas aeruginosa, Pseudomonas aureofaciens, Pseudomonas cepacia (formerly known as Burkholderia cepacia), Pseudomonas chlororaphis, Pseudomonas fluorescens, Pseudomonas proradix, Pseudomonas putida, Pseudomonas syringae, Serratia entomophila, Serratia marcescens, Streptomyces colombiensis, Streptomyces galbus, Streptomyces goshikiensis, Streptomyces griseoviridis, Streptomyces lavendulae, Streptomyces prasinus, Streptomyces saraceticus, Streptomyces venezuelae, Xanthomonas campestris, Xenorhabdus luminescens, Xenorhabdus nematophila, Rhodococcus globerulus AQ719 (NRRL Accession No. B-21663), Bacillus sp. AQ175 (ATCC Accession No. 55608), Bacillus sp. AQ 177 (ATCC Accession No. 55609), Bacillus sp. AQ178 (ATCC Accession No. 53522), and Streptomyces sp. strain NRRL Accession No. B-30145. In some embodiments, the bacterium is Azotobacter chroococcum, Methanosarcina barkeri, Klebsiella pneumoniae, Azotobacter vinelandii, Rhodobacter spharoides, Rhodobacter capsulatus, Rhodobacter palustris, Rhodospirillum rubrum, Rhizobium leguminosarum or Rhizobium etli.

In some embodiments, the bacterium is a species of Clostridium, for example Clostridium pasteurianum, Clostridium beijerinckii, Clostridium perfringens, Clostridium tetani, Clostridium acetobutylicum.

In some embodiments, the bacteria are cyanobacteria. Examples of cyanobacterial genuses include Anabaena (for example Anabaena sp. PCC7120), Nostoc (for example Nostoc punctiforme), or Synechocystis (for example Synechocystis sp. PCC6803). In some embodiments, the bacteria belong to the phylum Chlorobi, for example Chlorobium tepidum.

In some embodiments, the microbes comprise a gene homologous to a known NifH gene. Sequences of known NifH genes may be found in, for example, the Zehr lab NifH database, (wwwzehr.pmc.ucsc.edu/nifH_Database_Public/, Apr. 4, 2014), or the Buckley lab NifH database (www.css.cornell.edu/faculty/buckley/nifh.htm, and Gaby, John Christian, and Daniel H. Buckley. “A comprehensive aligned nifH gene database: a multipurpose tool for studies of nitrogen-fixing bacteria.” Database 2014 (2014): bau001.). In some embodiments, microbes comprise a sequence which encodes a polypeptide with at least 60%, 70%, 80%, 85%, 90%, 95%, 96%, 96%, 98%, 99% or more than 99% sequence identity to a sequence from the Zehr lab NifH database, (wwwzehr.pmc.ucsc.edu/nifH_Database_Public/, Apr. 4, 2014). In some embodiments, the microbes comprise a sequence which encodes a polypeptide with at least 60%, 70%, 80%, 85%, 90%, 95%, 96%, 96%, 98%, 99% or more than 99% sequence identity to a sequence from the Buckley lab NifH database, (Gaby, John Christian, and Daniel H. Buckley. “A comprehensive aligned nifH gene database: a multipurpose tool for studies of nitrogen-fixing bacteria.” Database 2014 (2014): bau001.).

In some embodiments, the bacteria are able to self-propagate efficiently on the leaf surface, root surface, or inside plant tissues without inducing a damaging plant defense reaction, or bacteria that are resistant to plant defense responses. In some embodiments, the bacteria described herein are isolated by culturing a plant tissue extract or leaf surface wash in a medium with no added nitrogen.

In some embodiments, the bacteria described herein is an endophyte or an epiphyte or a bacterium inhabiting the plant rhizosphere (rhizospheric bacteria). Endophytes are organisms that enter the interior of plants without causing disease symptoms or eliciting the formation of symbiotic structures, and are of agronomic interest because they can enhance plant growth and improve the nutrition of plants (e.g., through nitrogen fixation). The bacteria can be a seed-borne endophyte. Seed-borne endophytes include bacteria associated with or derived from the seed of a grass or plant, such as a seed-borne bacterial endophyte found in mature, dry, undamaged (e.g., no cracks, visible fungal infection, or prematurely germinated) seeds. The seed-borne bacterial endophyte can be associated with or derived from the surface of the seed; alternatively, or in addition, it can be associated with or derived from the interior seed compartment (e.g., of a surface-sterilized seed). In some embodiments, a seed-borne bacterial endophyte is capable of replicating within the plant tissue, for example, the interior of the seed.

Also, in some embodiments, the seed-borne bacterial endophyte is capable of surviving desiccation.

In some embodiments, the bacteria can comprise a plurality of different bacterial taxa in combination to form a bacterial consoritium. By way of example, the bacteria may include Proteobacteria (such as Pseudomonas, Enterobacter, Stenotrophomonas, Burkholderia, Rhizobium, Herbaspirillum, Pantoea, Serratia, Rahnella, Azospirillum, Azorhizobium, Azotobacter, Duganella, Delftia, Bradyrhizobium, Sinorhizobium and Halomonas), Firmicutes (such as Bacillus, Paenibacillus, Lactobacillus. Mycoplasma, and Acetobacterium),and Actinobacteria (such as Streptomyces, Rhodococcus, Microbacterium, and Curtobacterium). In some embodiments, the bacteria of this disclosure may include nitrogen fixing bacterial consortia of two or more species. In some embodiments, one or more bacterial species of the bacterial consortia may be capable of fixing nitrogen. In some embodiments, one or more species of the bacterial consortia facilitate or enhance the ability of other bacteria to fix nitrogen. The bacteria which fix nitrogen and the bacteria which enhance the ability of other bacteria to fix nitrogen may be the same or different. In some embodiments, a bacterial strain is able to fix nitrogen when in combination with a different bacterial strain, or in a certain bacterial consortia, but may be unable to fix nitrogen in a monoculture. Examples of bacterial genera which may be found in a nitrogen fixing bacterial consortia include, but are not limited to, Herbaspirillum, Azospirillum, Enterobacter, and Bacillus.

Bacteria that can be used in the agricultural compositions and methods disclosed herein include Azotobacter sp., Bradyrhizobium sp., Klebsiella sp., and Sinorhizobium sp. In some embodiments, the bacteria are selected from the group consisting of: Azotobacter vinelandii, Bradyrhizobium japonicum, Klebsiella pneumoniae, and Sinorhizobium meliloti. In some embodiments, the bacteria are of the genus Enterobacter or Rahnella. In some embodiments, the bacteria are of the genus Frankia, or Clostridium. Examples of bacteria of the genus Clostridium include, but are not limited to, Clostridium acetobutylicum, Clostridium pasteurianum, Clostridium beijerinckii, Clostridium perfringens, and Clostridium tetani. In some embodiments, the bacteria are of the genus Paenibacillus, for example Paenibacillus azotofixans, Paenibacillus borealis, Paenibacillus durus, Paenibacillus macerans, Paenibacillus polymyxa, Paenibacillus alvei, Paenibacillus amylolyticus, Paenibacillus campinasensis, Paenibacillus chibensis, Paenibacillus glucanolyticus, Paenibacillus illinoisensis, Paenibacillus larvae subsp. larvae, Paenibacillus larvae subsp. pulvifaciens, Paenibacillus lautus, Paenibacillus macerans, Paenibacillus macquariensis, Paenibacillus macquariensis, Paenibacillus pabuli, Paenibacillus peoriae, or Paenibacillus polymyxa.

In some embodiments, bacteria for use in the present compositions and methods can be a member of one or more of the following taxa: Achromobacter, Acidithiobacillus, Acidovorax, Acidovorax, Acinetobacter, Actinoplanes, Adlercreutzia, Aerococcus, Aeromonas, Afipia, Agromyces, Ancylobacter, Arthrobacter, Atopostipes, Azospirillum, Bacillus, Bdellovibrio, Beijerinckia, Bosea, Bradyrhizobium, Brevibacillus, Brevundimonas, Burkholderia, Candidatus Haloredivirus, Caulobacter, Cellulomonas, Cellvibrio, Chryseobacterium, Citrobacter, Clostridium, Coraliomargarita, Corynebacterium, Cupriavidus, Curtobacterium, Curvibacter, Deinococcus, Delftia, Desemzia, Devosia, Dokdonella, Dyella, Enhydrobacter, Enterobacter, Enterococcus, Erwinia, Escherichia, Escherichia/Shigella, Exiguobacterium, Ferroglobus, Filimonas, Finegoldia, Flavisolibacter, Flavobacterium, Frigoribacterium, Gluconacetobacter, Hafnia, Halobaculum, Halomonas, Halosimplex, Herbaspirillum, Hymenobacter, Klebsiella, Kocuria, Kosakonia, Lactobacillus, Leclercia, Lentzea, Luteibacter, Luteimonas, Massilia, Mesorhizobium, Methylobacterium, Microbacterium, Micrococcus, Microvirga, Mycobacterium, Neisseria, Nocardia, Oceanibaculum, Ochrobactrum, Okibacterium, Oligotropha, Oryzihumus, Oxalophagus, Paenibacillus, Panteoa, Pantoea, Pelomonas, Perlucidibaca, Plantibacter, Polynucleobacter, Propionibacterium, Propioniciclava, Pseudoclavibacter, Pseudomonas, Pseudonocardia, Pseudoxanthomonas, Psychrobacter, Rahnella, Ralstonia, Rheinheimera, Rhizobium, Rhodococcus, Rhodopseudomonas, Roseateles, Ruminococcus, Sebaldella, Sediminibacillus, Sediminibacterium, Serratia, Shigella, Shinella, Sinorhizobium, Sinosporangium, Sphingobacterium, Sphingomonas, Sphingopyxis, Sphingosinicella, Staphylococcus, Stenotrophomonas, Strenotrophomonas, Streptococcus, Streptomyces, Stygiolobus, Sulfurisphaera, Tatumella, Tepidimonas, Thermomonas, Thiobacillus, Variovorax, WPS-2 genera incertae sedis, Xanthomonas, and Zimmermannella.

In some embodiments, the bacteria are gram-negative bacteria of a genus selected from the following list: Acetobacter, Achromobacter, Aerobacter, Anabaena, Azoarcus, Azomonas, Azorhizobium, Azospirillum, Azotobacter, Beijernickia, Bradyrhizobium, Burkholderia, Citrobacter, Derxia, Enterobacter, Herbaspirillum, Klebsiella, Kluyvera, Kosakonia, Nostoc, Mesorhizobium, Rahnella, Rhizobium, Rhodobacter, Rhodopseudomonas, Rhodospirillum, Serratia Sinorhizobium, Spirillum, Trichodesmium, and Xanthomonas.

In some embodiments, the nitrogen fixing microbes are of a species selected from the group consisting of: Achromobacter marplatensis, Achromobacter spiritinus, Azospirillum lipoferum, Enterobacter sacchari, Herbaspirillum aquaticum, Klebsiella variicola, Kluyvera intermedia, Kosakonia pseudosacchari, Kosakonia sacchari, Metakosakonia intestini, Paraburkholderia tropica, Rahnella aquatilis, and combinations thereof.

In some embodiments, a bacterial species selected from at least one of the following genera are utilized: Enterobacter, Klebsiella, Kosakonia, and Rahnella. In some embodiments, a combination of bacterial species from the following genera are utilized: Enterobacter, Klebsiella, Kosakonia, and Rahnella In some embodiments, the species utilized can be one or more of: Enterobacter sacchari. Klebsiella variicola, Kosakonia sacchari, and Rahnella aquatilis.

In some embodiments, a Gram positive microbe may have a Molybdenum-Iron nitrogenase system comprising: nifH, nifD, nifK, nifB, nifE, nifN, nifX, hesA, nifV, nifW, nifU, nifS, nifl1, and nifl2. In some embodiments, a Gram positive microbe may have a vanadium nitrogenase system comprising: vnfDG, vnfK, vnfE, vnfN, vupC, vupB, vupA, vnfV, viR1, vnfH, vnfR2, vnfA (transcriptional regulator). In some embodiments, a Gram positive microbe may have an iron-only nitrogenase system comprising: anfK, anfG, anfD, anfH, anfA (transcriptional regulator). In some embodiments, a Gram positive microbe may have a nitrogenase system comprising glnB, and glnK (nitrogen signaling proteins). Some examples of enzymes involved in nitrogen metabolism in Gram positive microbes include glnA (glutamine synthetase), gdh (glutamate dehydrogenase), bdh (3-hydroxybutyrate dehydrogenase), glutaminase, gltAB/gltB/gltS (glutamate synthase), asnA/asnB (aspartate-ammonia ligase/asparagine synthetase), and ansA/ansZ (asparaginase). Some examples of proteins involved in nitrogen transport in Gram positive microbes include amtB (ammonium transporter), glnK (regulator of ammonium transport), glnPHQ/glnQHMP (ATP-dependent glutamine/glutamate transporters), glnT/alsT/yrbD/yflA (glutamine-like proton symport transporters), and gltP/gltT/yhcl/nqt (glutamate-like proton symport transporters).

Examples of Gram positive microbes for use within the present agricultural compositions include Paenibacillus polymixa, Paenibacillus riograndensis, Paenibacillus sp., Frankia sp., Heliobacterium sp., Heliobacterium chlorum, Heliobacillus sp., Heliophilum sp., Heliorestis sp., Clostridium acetobutylicum, Clostridium sp., Methanobacterium sp., Micrococcus sp., Mycobacterium flavum, Mycobacterium sp., Arthrobacter sp., Agromyces sp., Corynebacterium autitrophicum, Corynebacterium sp., Micromonospora sp., Propionibacteria sp., Streptomyces sp., and Microbacterium sp.

In some embodiments, the amount of microbes present in the agricultural composition is in the range of about 10⁴ to about 10¹² CFU/g of the agricultural composition for example, about 10⁴ CFU/g, about 10⁵ CFU/g, about 10⁶ CFU/g, about 10⁷ CFU/g, about 10⁸ CFU/g, about 10⁹ CFU/g, about 10¹⁰ CFU/g, about 10¹¹ CFU/g, or about 10¹² CFU/g, including all values and subranges that lie therebetween. In some embodiments, the microorganisms are present in the agricultural composition disclosed herein at a concentration in the range of about 10⁹ CFU/g to about 10¹¹ CFU/g. In some embodiments, the microorganisms are present in the agricultural composition disclosed herein at a concentration of about 3×10¹⁰ CFU/g.

In some embodiments, the microorganisms are present in the dispersion of live microbes disclosed herein at a concentration in the range of about 10⁴ to about 10¹² CFU/ml, for example, about 10⁴ CFU/ml, about 10⁵ CFU/ml, about 10⁶ CFU/ml, about 10⁷ CFU/ml, about 10⁸ CFU/ml, about 10⁹ CFU/ml, about 10¹⁰ CFU/ml, about 10¹¹ CFU/ml, or about 10¹² CFU/mL, including all values and subranges that lie therebetween. For instance, in some embodiments, the microorganisms are present in the dispersion of live microbes disclosed herein at a concentration in the range of about 10⁷ CFU/ml to about 10¹¹ CFU/ml. In some embodiments, the microorganisms are present in the dispersion of live microbes disclosed herein at a concentration in the range of about 10⁸ CFU/mL to about 10¹⁰ CFU/mL.

a. Genetic Alterations

In some embodiments, the water-soluble packages disclosed herein comprise a microbe capable of fixing nitrogen. In some embodiments, the microbe can naturally fix nitrogen. In some embodiments the microbe is genetically modified to fix nitrogen. In some embodiments, the organism is genetically modified to have improved nitrogen fixation capabilities. Thus, in some embodiments, the microbes comprise one or more genetic variations introduced into one or more genes regulating nitrogen fixation. The genetic variation may be introduced into a gene selected from the group consisting of nifA, nifL, ntrB, ntrC, glutamine synthetase, glnA, glnB, glnK, draT, amtB, glutaminase, glnD, glnE, nifJ, nifH, nifD, nifK, nifY, nifE, nifN, nifU, nifS, nifV, nifW, nifZ, nifM, nifF, nifB, and nifQ. The genetic variation may be a variation in a gene encoding a protein with functionality selected from the group consisting of: glutamine synthetase, glutaminase, glutamine synthetase adenylyltransferase, transcriptional activator, anti-transcriptional activator, pyruvate flavodoxin oxidoreductase, flavodoxin, and NAD+-dinitrogen-reductase aDP-D-ribosyltransferase. The genetic variation may be a mutation that results in one or more of: increased expression or activity of nifA or glutaminase; decreased expression or activity of nifL, ntrB, glutamine synthetase, glnB, glnK, draT, amtB; decreased adenylyl-removing activity of GlnE; decreased expression of GlnD; or decreased uridylyl-removing activity of GlnD. The genetic variation may be a variation in a gene selected from the group consisting of: bcsii, bcsiii, yjbE, fhaB, pehA, otsB, treZ, glsA2, and combinations thereof.

In some embodiments, the microbe has a disrupted (e.g., deleted or partially deleted) nifL gene. In some embodiments, the microbe has a nifL gene that has been disrupted with the introduction of a promoter sequence that acts on the nifA gene. In some embodiments, e.g., when the microbe is a strain of K. variicola, the promoter is a K. variicola PinfC promoter. In some embodiments, e.g., when the microbe is a strain of K. sacchari, the promoter is a K. sacchari Prm5 promoter. In some embodiments, the microbe has a glnE gene that has been altered to remove the adenylyl-removing (AR) domain, while leaving the coding region for the adenyltransferase (AT) domain, which is functionally expressed. In some embodiments, the microbe has a deletion of the glnD gene.

The genetic variation introduced into one or more bacteria of the agricultural compositions disclosed herein may be a knock-out mutation or it may abolish a regulatory sequence of a target gene, or it may comprise insertion of a heterologous regulatory sequence, for example, insertion of a regulatory sequence found within the genome of the same bacterial species or genus. The regulatory sequence can be chosen based on the expression level of a gene in a bacterial culture or within plant tissue. The genetic variation may be produced by chemical mutagenesis. The plants grown may be exposed to biotic or abiotic stressors. However, in some embodiments, the methods disclosed herein also envision altering the impact of ATP or O₂ on the circuitry, or replacing the circuitry with other regulatory cascades in the cell, or altering genetic circuits other than nitrogen fixation. Gene clusters can be re-engineered to generate functional products under the control of a heterologous regulatory system. By eliminating native regulatory elements outside of, and within, coding sequences of gene clusters, and replacing them with alternative regulatory systems, the functional products of complex genetic operons and other gene clusters can be controlled and/or moved to heterologous cells, including cells of different species other than the species from which the native genes were derived. Once re-engineered, the synthetic gene clusters can be controlled by genetic circuits or other inducible regulatory systems, thereby controlling the products' expression as desired. The expression cassettes can be designed to act as logic gates, pulse generators, oscillators, switches, or memory devices. The controlling expression cassette can be linked to a promoter such that the expression cassette functions as an environmental sensor, such as an oxygen, temperature, touch, osmotic stress, membrane stress, or redox sensor.

As an example, the nifL, nifA, nifT, and nifX genes can be eliminated from the nif gene cluster. Synthetic genes can be designed by codon randomizing the DNA encoding each amino acid sequence. Codon selection is performed, specifying that codon usage be as divergent as possible from the codon usage in the native gene. Proposed sequences are scanned for any undesired features, such as restriction enzyme recognition sites, transposon recognition sites, repetitive sequences, sigma 54 and sigma 70 promoters, cryptic ribosome binding sites, and rho independent terminators. Synthetic ribosome binding sites are chosen to match the strength of each corresponding native ribosome binding site, such as by constructing a fluorescent reporter plasmid in which the 150 bp surrounding a gene's start codon (from −60 to +90) is fused to a fluorescent gene. This chimera can be expressed under control of the Ptac promoter, and fluorescence measured via flow cytometry. To generate synthetic ribosome binding sites, a library of reporter plasmids using 150 bp (−60 to +90) of a synthetic expression cassette is generated. Briefly, a synthetic expression cassette can consist of a random DNA spacer, a degenerate sequence encoding an RBS library, and the coding sequence for each synthetic gene. Multiple clones are screened to identify the synthetic ribosome binding site that best matched the native ribosome binding site. Synthetic operons that consist of the same genes as the native operons are thus constructed and tested for functional complementation. A further exemplary description of synthetic operons is provided in US20140329326.

Some examples of genetic alterations which may be made in Gram positive microbes include: deleting glnR to remove negative regulation of BNF in the presence of environmental nitrogen, inserting different promoters directly upstream of the nif cluster to eliminate regulation by GlnR in response to environmental nitrogen, mutating glnA to reduce the rate of ammonium assimilation by the GS-GOGAT pathway, deleting amtB to reduce uptake of ammonium from the media, mutating glnA so it is constitutively in the feedback-inhibited (FBI-GS) state, to reduce ammonium assimilation by the GS-GOGAT pathway.

In some embodiments, glnR is the main regulator of N metabolism and fixation in, e.g., Paenibacillus species. In some embodiments, the genome of a Paenibacillus species does not contain a gene to produce glnR. In some embodiments, the genome of a Paenibacillus species does not contain a gene to produce glnE or glnD. In some embodiments, the genome of a Paenibacillus species does contain a gene to produce glnB or glnK. For example, Paenibacillus sp. WLY78 doesn't contain a gene for glnB, or its homologs found in the archaeon Methanococcus maripaludis, nifI1 and nifI2. In some embodiments, the genomes of Paenibacillus species are variable. For example, Paenibacillus polymixa E681 lacks glnK and gdh, has several nitrogen compound transporters, but only amtB appears to be controlled by GlnR. In another example, Paenibacillus sp. JDR2 has glnK, gdh and most other central nitrogen metabolism genes, has many fewer nitrogen compound transporters, but does have glnPHQ controlled by GlnR. Paenibacillus riograndensis SBR5 contains a standard glnRA operon, an fdx gene, a main nif operon, a secondary nif operon, and an anf operon (encoding iron-only nitrogenase). Putative glnR/tnrA sites were found upstream of each of these operons. GlnR may regulate all of the above operons, except the anf operon. GlnR may bind to each of these regulatory sequences as a dimer.

Paenibacillus N-fixing strains may fall into two subgroups: Subgroup I, which contains only a minimal nif gene cluster and subgroup II, which contains a minimal cluster, plus an uncharacterized gene between nifX and hesA, and often other clusters duplicating some of the nif genes, such as nifH, nifHDK, nifBEN, or clusters encoding vanadium nitrogenase (vnf) or iron-only nitrogenase (anf) genes.

In some embodiments, the genome of a Paenibacillus species may not contain a gene to produce glnB or glnK. In some embodiments, the genome of a Paenibacillus species may contain a minimal nif cluster with 9 genes transcribed from a sigma-70 promoter. In some embodiments, a Paenibacillus nif cluster is negatively regulated by nitrogen or oxygen. In some embodiments, the genome of a Paenibacillus species does not contain a gene to produce sigma-54. For example, Paenibacillus sp. WLY78 does not contain a gene for sigma-54. In some embodiments, a nif cluster is regulated by glnR, and/or TnrA. In some embodiments, activity of a nif cluster is altered by altering activity of glnR, and/or TnrA.

In Bacilli, glutamine synthetase (GS) is feedback-inhibited by high concentrations of intracellular glutamine, causing a shift in confirmation (referred to as FBI-GS). Nif clusters contain distinct binding sites for the regulators GlnR and TnrA in several Bacilli species. GlnR binds and represses gene expression in the presence of excess intracellular glutamine and AMP. A role of GlnR may be to prevent the influx and intracellular production of glutamine and ammonium under conditions of high nitrogen availability. TnrA may bind and/or activate (or repress) gene expression in the presence of limiting intracellular glutamine, and/or in the presence of FBI-GS. In some embodiments, the activity of a Bacilli nif cluster is altered by altering the activity of GlnR.

Feedback-inhibited glutamine synthetase (FBI-GS) may bind GlnR and stabilize binding of GlnR to recognition sequences. Several bacterial species have a GlnR/TnrA binding site upstream of the nif cluster. Altering the binding of FBI-GS and GlnR may alter the activity of the nif pathway.

Additional genetic modifications suitable for the microbes of the present disclosure may be found in International Patent Application No. PCT/US2019/039528, the contents of which are herein incorporated by reference in their entirety.

b. Sources of Microbes

Microbes of the present disclosure can be obtained from any source, including environmental and commercial sources. The bacteria (or any microbe according to the disclosure) may be obtained from any general terrestrial environment, including its soils, plants, fungi, animals (including invertebrates) and other biota, including the sediments, water and biota of lakes and rivers; from the marine environment, its biota and sediments (for example, sea water, marine muds, marine plants, marine invertebrates (for example, sponges), marine vertebrates (for example, fish)); the terrestrial and marine geosphere (regolith and rock, for example, crushed subterranean rocks, sand and clays); the cryosphere and its meltwater; the atmosphere (for example, filtered aerial dusts, cloud and rain droplets); urban, industrial and other man-made environments (for example, accumulated organic and mineral matter on concrete, roadside gutters, roof surfaces, and road surfaces).

The plants from which the bacteria (or any microbe according to the disclosure) are obtained may be a plant having one or more desirable traits, for example a plant which naturally grows in a particular environment or under certain conditions of interest. By way of example, a certain plant may naturally grow in sandy soil or sand of high salinity, or under extreme temperatures, or with little water, or it may be resistant to certain pests or disease present in the environment, and it may be desirable for a commercial crop to be grown in such conditions, particularly if they are, for example, the only conditions available in a particular geographic location. By way of further example, the bacteria may be collected from commercial crops grown in such environments, or more specifically from individual crop plants best displaying a trait of interest amongst a crop grown in any specific environment: for example the fastest-growing plants amongst a crop grown in saline-limiting soils, or the least damaged plants in crops exposed to severe insect damage or disease epidemic, or plants having desired quantities of certain metabolites and other compounds, including fiber content, oil content, and the like, or plants displaying desirable colors, taste or smell. The bacteria may be collected from a plant of interest or any material occurring in the environment of interest, including fungi and other animal and plant biota, soil, water, sediments, and other elements of the environment as referred to previously.

The bacteria (or any microbe according to the disclosure) may be isolated from plant tissue. This isolation can occur from any appropriate tissue in the plant, including for example root, stem and leaves, and plant reproductive tissues. Non-limiting examples of plant tissues include a seed, seedling, leaf, cutting, plant, bulb, tuber, root, and rhizomes. In some embodiments, microorganisms are isolated from a seed. In some embodiments, microorganisms are isolated from a root.

Persons having skill in the art will be familiar with techniques for recovering microbes from various environmental sources. For example, microbes useful in the methods and agricultural compositions disclosed herein can be obtained by extracting microbes from surfaces or tissues of native plants; grinding seeds to isolate microbes; planting seeds in diverse soil samples and recovering microbes from tissues; or inoculating plants with exogenous microbes and determining which microbes appear in plant tissues. The parameters for processing samples may be varied to isolate different types of associative microbes, such as rhizospheric, epiphytes, or endophytes. By way of example, some methods for isolation from plants include the sterile excision of the plant material of interest (e.g. root or stem lengths, leaves), surface sterilization with an appropriate solution (e.g. 2% sodium hypochlorite), after which the plant material is placed on nutrient medium for microbial growth. Alternatively, the surface-sterilized plant material can be crushed in a sterile liquid (usually water) and the liquid suspension, including small pieces of the crushed plant material spread over the surface of a suitable solid agar medium, or media, which may or may not be selective (e.g. contain only phytic acid as a source of phosphorus). This approach is especially useful for bacteria which form isolated colonies and can be picked off individually to separate plates of nutrient medium, and further purified to a single species by well-known methods. Alternatively, the plant root or foliage samples may not be surface sterilized but only washed gently thus including surface-dwelling epiphytic microorganisms in the isolation process, or the epiphytic microbes can be isolated separately, by imprinting and lifting off pieces of plant roots, stem or leaves onto the surface of an agar medium and then isolating individual colonies as above. This approach is especially useful for bacteria, for example. Alternatively, the roots may be processed without washing off small quantities of soil attached to the roots, thus including microbes that colonize the plant rhizosphere. Otherwise, soil adhering to the roots can be removed, diluted and spread out onto agar of suitable selective and non-selective media to isolate individual colonies of rhizospheric bacteria.

Microbes may also be sourced from a repository, such as environmental strain collections, instead of initially isolating from a first plant. The microbes can be genotyped and phenotyped, via sequencing the genomes of isolated microbes; profiling the composition of communities in planta; characterizing the transcriptomic functionality of communities or isolated microbes; or screening microbial features using selective or phenotypic media (e.g., nitrogen fixation or phosphate solubilization phenotypes). Selected candidate strains or populations can be obtained via sequence data; phenotype data; plant data (e.g., genome, phenotype, and/or yield data); soil data (e.g., pH, N/P/K content, and/or bulk soil biotic communities); or any combination of these.

c. Budapest Treaty on the International Recognition of the Deposit of Microorganisms for the Purpose of Patent Procedures

The microbial deposits of the present disclosure were made under the provisions of the Budapest Treaty on the International Recognition of the Deposit of Microorganisms for the Purpose of Patent Procedure (Budapest Treaty).

Applicants state that pursuant to 37 C.F.R. § 1.808(a)(2) “all restrictions imposed by the depositor on the availability to the public of the deposited material will be irrevocably removed upon the granting of the patent.” This statement is subject to paragraph (b) of this section (i.e. 37 C.F.R. § 1.808(b)).

The Enterobacter sacchari has now been reclassified as Kosakonia sacchari, the name for the organism may be used interchangeably throughout the present disclosure.

Some microbes of the present disclosure are derived from two wild-type strains. Strain CI006 is a bacterial species previously classified in the genus Enterobacter (see aforementioned reclassification into Kosakonia). Strain CI019 is a bacterial species classified in the genus Rahnella. The deposit information for the C1006 Kosakonia wild type (WT) and CI019 Rahnella WT are found in Table 1.

Some microorganisms described in this application were deposited on Jan. 6, 2017 or Aug. 11, 2017 with the Bigelow National Center for Marine Algae and Microbiota (NCMA), located at 60 Bigelow Drive, East Boothbay, Maine 04544, USA. As aforementioned, all deposits were made under the terms of the Budapest Treaty on the International Recognition of the Deposit of Microorganisms for the Purposes of Patent Procedure. The Bigelow National Center for Marine Algae and Microbiota accession numbers and dates of deposit for the aforementioned Budapest Treaty deposits are provided in Table 1.

Biologically pure cultures of Kosakonia sacchari (WT), Rahnella aquatilis (WT), and a variant/remodeled Kosakonia sacchari strain were deposited on Jan. 6, 2017 with the Bigelow National Center for Marine Algae and Microbiota (NCMA), located at 60 Bigelow Drive, East Boothbay, Maine 04544, USA, and assigned NCMA Patent Deposit Designation numbers 201701001, 201701003, and 201701002, respectively. The applicable deposit information is found below in Table 1.

Biologically pure cultures of variant/remodeled Kosakonia sacchari strains were deposited on Aug. 11, 2017 with the Bigelow National Center for Marine Algae and Microbiota (NCMA), located at 60 Bigelow Drive, East Boothbay, Maine 04544, USA, and assigned NCMA Patent Deposit Designation numbers 201708004, 201708003, and 201708002, respectively. The applicable deposit information is found below in Table 1.

A biologically pure culture of Klebsiella variicola (WT) was deposited on Aug. 11, 2017 with the Bigelow National Center for Marine Algae and Microbiota (NCMA), located at 60 Bigelow Drive, East Boothbay, Maine 04544, USA, and assigned NCMA Patent Deposit Designation number 201708001. Biologically pure cultures of two Klebsiella variicola variants/remodeled strains were deposited on Dec. 20, 2017 with the Bigelow National Center for Marine Algae and Microbiota (NCMA), located at 60 Bigelow Drive, East Boothbay, Maine 04544, USA, and assigned NCMA Patent Deposit Designation numbers 201712001 and 201712002, respectively. The applicable deposit information is found below in Table 1.

Biologically pure cultures of two Kosakonia sacchari variants/remodeled strains were deposited on Dec. 23, 2019 with the American Type Culture Collection (ATCC), located at 10801 University Boulevard, Manassas, Virginia 20110-2209, USA and assigned ATCC Patent Deposit Numbers PTA-126575 and PTA-126576. Biologically pure cultures of four Klebsiella variicola variants/remodeled strains were deposited on Dec. 23, 2019 with the American Type Culture Collection (ATCC), located at 10801 University Boulevard, Manassas, Virginia 20110-2209, USA and assigned ATCC Patent Deposit Numbers PTA-126577, PTA-126578, PTA-126579 and PTA-126580. A biologically pure culture of a Paenibacillus polymyxa (WT) strain was deposited on Dec. 23, 2019 with the American Type Culture Collection (ATCC), located at 10801 University Boulevard, Manassas, Virginia 20110-2209, USA and assigned ATCC Patent Deposit Number PTA-126581. A biologically pure culture of a Paraburkholderia tropica (WT) strain was deposited on Dec. 23, 2019 with the American Type Culture Collection (ATCC), located at 10801 University Boulevard, Manassas, Virginia 20110-2209, USA and assigned ATCC Patent Deposit Number PTA-126582. A biologically pure culture of a Herbaspirillum aquaticum (WT) strain was deposited on Dec. 23, 2019 with the American Type Culture Collection (ATCC), located at 10801 University Boulevard, Manassas, Virginia 20110-2209, USA and assigned ATCC Patent Deposit Number PTA-126583. Biologically pure cultures of four Metakosakonia intestini variants/remodeled strains were deposited on Dec. 23, 2019 with the American Type Culture Collection (ATCC), located at 10801 University Boulevard, Manassas, Virginia 20110-2209, USA and assigned ATCC Patent Deposit Numbers PTA-126584, PTA-126586, PTA-126587 and PTA-126588. A biologically pure culture of a Metakosakonia intestini (W7) strain was deposited on Dec. 23, 2019 with the American Type Culture Collection (ATCC), located at 10801 University Boulevard, Manassas, Virginia 20110-2209, USA and assigned ATCC Patent Deposit Number PTA-126585. A biologically pure culture of a Klebsiella variicola variant/remodeled strain was deposited on Mar. 25, 2020 with the American Type Culture Collection (ATCC), located at 10801 University Boulevard, Manassas, Virginia 20110-2209, USA and assigned ATCC Patent Deposit Number PTA-126740. A biologically pure culture of a Kosakonia sacchari variant/remodeled strain was deposited on Mar. 25, 2020 with the American Type Culture Collection (ATCC), located at 10801 University Boulevard, Manassas, Virginia 20110-2209, USA and assigned ATCC Patent Deposit Number PTA-126743. The applicable deposit information is found below in Table 1.

TABLE 1
Microorganisms Deposited under the Budapest Treaty

| | Pivot Strain | | | |
|---|---|---|---|---|
| | Designation (some strains | | | |
| Depository | have multiple designations) | Taxonomy | Accession Number | Date of Deposit |
| NCMA | CI006, | Kosakonia sacchari (WT) | 201701001 | Jan. 06, 2017 |
| | PBC6.1, 6 | | | |
| NCMA | CI019, 19 | Rahnella aquatilis (WT) | 201701003 | Jan. 06, 2017 |
| NCMA | CM029, 6-412 | Kosakonia sacchari | 201701002 | Jan. 06, 2017 |
| NCMA | 6-403 CM037 | Kosakonia sacchari | 201708004 | Aug. 11, 2017 |
| NCMA | 6-404, CM38, | Kosakonia sacchari | 201708003 | Aug. 11, 2017 |
| | PBC6.38 | | | |
| NCMA | CM094, 6-881, | Kosakonia sacchari | 201708002 | Aug. 11, 2017 |
| NCMA | CI137, 137, | Klebsiella variicola (WT) | 201708001 | Aug. 11, 2017 |
| | PB137 | | | |
| NCMA | 137-1034 | Klebsiella variicola | 201712001 | Dec. 20, 2017 |
| NCMA | 137-1036 | Klebsiella variicola | 201712002 | Dec. 20, 2017 |
| ATCC | 6-2425 | Kosakonia sacchari | PTA-126575 | Dec. 23, 2019 |
| ATCC | 6-2634 | Kosakonia sacchari | PTA-126576 | Dec. 23, 2019 |
| ATCC | 137-1968 | Klebsiella variicola | PTA-126577 | Dec. 23, 2019 |
| ATCC | 137-2219 | Klebsiella variicola | PTA-126578 | Dec. 23, 2019 |
| ATCC | 137-2237 | Klebsiella variicola | PTA-126579 | Dec. 23, 2019 |
| ATCC | 137-2285 | Klebsiella variicola | PTA-126580 | Dec. 23, 2019 |
| ATCC | 41 | Paenibacillus polymyxa | PTA-126581 | Dec. 23, 2019 |
| | | (WT) | | |
| ATCC | 8 | Paraburkholderia tropica | PTA-126582 | Dec. 23, 2019 |
| | | (WT) | | |
| ATCC | 3069 | Herbaspirillum | PTA-126583 | Dec. 23, 2019 |
| | | aquaticum (WT) | | |
| ATCC | 910-3655 | Metakosakonia intestini | PTA-126584 | Dec. 23, 2019 |
| ATCC | 910 | Metakosakonia intestini | PTA-126585 | Dec. 23, 2019 |
| | | (WT) | | |
| ATCC | 910-3963 | Metakosakonia intestini | PTA-126586 | Dec. 23, 2019 |
| ATCC | 910-3961 | Metakosakonia intestini | PTA-126587 | Dec. 23, 2019 |
| ATCC | 910-3994 | Metakosakonia intestini | PTA-126588 | Dec. 23, 2019 |
| ATCC | 6-5687 | Kosakonia sacchari | PTA-126743 | Mar. 25, 2020 |
| ATCC | 137-2253 | Klebsiella variicola | PTA-126740 | Mar. 25, 2020 |

In some aspects, the isolated and biologically pure microorganisms of the disclosure are those from Table 1. In other aspects, the isolated and biologically pure microorganisms of the disclosure are derived from a microorganism of Table 1. For example, a strain, child, mutant, or derivative, of a microorganism from Table 1 are provided herein. The disclosure contemplates all possible combinations of microbes listed in Table 1, said combinations sometimes forming a microbial consortia. The microbes from Table 1, either individually or in any combination, can be combined with any plant, active molecule (synthetic, organic, etc.), adjuvant, carrier, supplement, or biological, mentioned in the disclosure.

d. Remodeled Microbes

In some embodiments, the microbes are non-intergeneric remodeled microbes. The term “non-intergeneric” indicates that the genetic variations introduced into the host do not contain nucleic acid sequences from outside the host genus (e.g., no transgenic DNA). Therefore, in some embodiments, the microbes are not transgenic. For example, for non-transgenic microbes with varied promoters, promoters for promoter swapping are selected from within the microbe's genome, or genus.

Exemplary non-intergeneric genetic variations include a mutation in the gene of interest that may improve the function of the protein encoded by the gene; a constitutionally active promoter that can replace the endogenous promoter of the gene of interest to increase the expression of the gene; a mutation that will inactivate the gene of interest; the insertion of a promoter from within the host's genome into a heterologous location, e.g. insertion of the promoter into a gene that results in inactivation of said gene and upregulation of a downstream gene; and the like. The mutations can be point mutations, insertions, and/or deletions (full or partial deletion of the gene). For example, in some embodiments, to improve the nitrogen fixation activity of the host microbe, a genetic variation may comprise an inactivating mutation of the nifL gene (negative regulator of nitrogen fixation pathway) and/or comprise replacing the endogenous promoter of the nifA and/or nifH gene (nitrogenase iron protein that catalyzes a key reaction to fix atmospheric nitrogen) with a constitutionally active promoter that will drive the expression of the nifA and/or nifH gene constitutively. Additional genetic variations of interest are described further in the foregoing “Genetic alterations” section.

Exemplary microbes for use in the agricultural compositions and methods of the present disclosure are provided in Tables 4-5.

TABLE 4
Microbes of the disclosure

| | Strain | SEQ ID | | |
|---|---|---|---|---|
| Strain | ID | NO | Genotype | Description |
| CI63; | 63 | SEQ ID | 16S | N/A |
| CI063 | | NO 1 | | |
| CI63; | 63 | SEQ ID | nifH | N/A |
| CI063 | | NO 2 | | |
| CI63; | 63 | SEQ ID | nifD1 | 1 of 2 unique genes annotated as nifD in 63 genome |
| CI063 | | NO 3 | | |
| CI63; | 63 | SEQ ID | nifD2 | 2 of 2 unique genes annotated as nifD in 63 genome |
| CI063 | | NO 4 | | |
| CI63; | 63 | SEQ ID | nifK1 | 1 of 2 unique genes annotated as nifK in 63 genome |
| CI063 | | NO 5 | | |
| CI63; | 63 | SEQ ID | nifK2 | 2 of 2 unique genes annotated as nifK in 63 genome |
| CI063 | | NO 6 | | |
| CI63; | 63 | SEQ ID | nifL | N/A |
| CI063 | | NO 7 | | |
| CI63; | 63 | SEQ ID | nifA | N/A |
| CI063 | | NO 8 | | |
| CI63; | 63 | SEQ ID | glnE | N/A |
| CI063 | | NO 9 | | |
| CI63; | 63 | SEQ ID | amtB | N/A |
| CI063 | | NO 10 | | |
| CI63; | 63 | SEQ ID | PinfC | 500 bp immediately upstream of the ATG start codon |
| CI063 | | NO 11 | | of the infC gene |
| CI137 | 137 | SEQ ID | 16S | N/A |
| | | NO 12 | | |
| CI137 | 137 | SEQ ID | nifH1 | 1 of 2 unique genes annotated as nifH in 137 genome |
| | | NO 13 | | |
| CI137 | 137 | SEQ ID | nifH2 | 2 of 2 unique genes annotated as nifH in 137 genome |
| | | NO 14 | | |
| CI137 | 137 | SEQ ID | nifD1 | 1 of 2 unique genes annotated as nifD in 137 genome |
| | | NO 15 | | |
| CI137 | 137 | SEQ ID | nifD2 | 2 of 2 unique genes annotated as nifD in 137 genome |
| | | NO 16 | | |
| CI137 | 137 | SEQ ID | nifK1 | 1 of 2 unique genes annotated as nifK in 137 genome |
| | | NO 17 | | |
| CI137 | 137 | SEQ ID | nifK2 | 2 of 2 unique genes annotated as nifK in 137 genome |
| | | NO 18 | | |
| CI137 | 137 | SEQ ID | nifL | N/A |
| | | NO 19 | | |
| CI137 | 137 | SEQ ID | nifA | N/A |
| | | NO 20 | | |
| CI137 | 137 | SEQ ID | glnE | N/A |
| | | NO 21 | | |
| CI137 | 137 | SEQ ID | PinfC | 500 bp immediately upstream of the TTG start codon of |
| | | NO 22 | | infC |
| CI137 | 137 | SEQ ID | amtB | N/A |
| | | NO 23 | | |
| CI137 | 137 | SEQ ID | Prm8.2 | internal promoter located in nlpl gene; 299 bp starting |
| | | NO 24 | | at 81 bp after the A of the ATG of the nlpI gene |
| CI137 | 137 | SEQ ID | Prm6.2 | 300 bp upstream of the secE gene starting at 57 bp |
| | | NO 25 | | upstream of the A of the ATG of sccE |
| CI137 | 137 | SEQ ID | Prm1.2 | 400 bp immediately upstream of the ATG of cspE gene |
| | | NO 26 | | |
| none | 728 | SEQ ID | 16S | N/A |
| | | NO 27 | | |
| none | 728 | SEQ ID | nifH | N/A |
| | | NO 28 | | |
| none | 728 | SEQ ID | nifD1 | 1 of 2 unique genes annotated as nifD in 728 genome |
| | | NO 29 | | |
| none | 728 | SEQ ID | nifD2 | 2 of 2 unique genes annotated as nifD in 728 genome |
| | | NO 30 | | |
| none | 728 | SEQ ID | nifK1 | 1 of 2 unique genes annotated as nifK in 728 genome |
| | | NO 31 | | |
| none | 728 | SEQ ID | nifK2 | 2 of 2 unique genes annotated as nifK in 728 genome |
| | | NO 32 | | |
| none | 728 | SEQ ID | nifL | N/A |
| | | NO 33 | | |
| none | 728 | SEQ ID | nifA | N/A |
| | | NO 34 | | |
| none | 728 | SEQ ID | glnE | N/A |
| | | NO 35 | | |
| none | 728 | SEQ ID | amtB | N/A |
| | | NO 36 | | |
| none | 850 | SEQ ID | 16S | N/A |
| | | NO 37 | | |
| none | 852 | SEQ ID | 16S | N/A |
| | | NO 38 | | |
| none | 853 | SEQ ID | 16S | N/A |
| | | NO 39 | | |
| none | 910 | SEQ ID | 16S | N/A |
| | | NO 40 | | |
| none | 910 | SEQ ID | nifH | N/A |
| | | NO 41 | | |
| none | 910 | SEQ ID | Dinitrogenase | N/A |
| | | NO 42 | iron-molybdenum | |
| | | | cofactor CDS | |
| none | 910 | SEQ ID | nifD1 | N/A |
| | | NO 43 | | |
| none | 910 | SEQ ID | nifD2 | N/A |
| | | NO 44 | | |
| none | 910 | SEQ ID | nifK1 | N/A |
| | | NO 45 | | |
| none | 910 | SEQ ID | nifK2 | N/A |
| | | NO 46 | | |
| none | 910 | SEQ ID | nifL | N/A |
| | | NO 47 | | |
| none | 910 | SEQ ID | nifA | N/A |
| | | NO 48 | | |
| none | 910 | SEQ ID | glnE | N/A |
| | | NO 49 | | |
| none | 910 | SEQ ID | amtB | N/A |
| | | NO 50 | | |
| none | 910 | SEQ ID | PinfC | 498 bp immediately upstream of the ATG of the infC |
| | | NO 51 | | gene |
| none | 1021 | SEQ ID | 16S | N/A |
| | | NO 52 | | |
| none | 1021 | SEQ ID | nifH | N/A |
| | | NO 53 | | |
| none | 1021 | SEQ ID | nifD1 | 1 of 2 unique genes annotated as nifD in 910 genome |
| | | NO 54 | | |
| none | 1021 | SEQ ID | nifD2 | 2 of 2 unique genes annotated as nifD in 910 genome |
| | | NO 55 | | |
| none | 1021 | SEQ ID | nifK1 | 1 of 2 unique genes annotated as nifK in 910 genome |
| | | NO 56 | | |
| none | 1021 | SEQ ID | nifK2 | 2 of 2 unique genes annotated as nifK in 910 genome |
| | | NO 57 | | |
| none | 1021 | SEQ ID | nifL | N/A |
| | | NO 58 | | |
| none | 1021 | SEQ ID | nifA | N/A |
| | | NO 59 | | |
| none | 1021 | SEQ ID | glnE | N/A |
| | | NO 60 | | |
| none | 1021 | SEQ ID | amtB | N/A |
| | | NO 61 | | |
| none | 1021 | SEQ ID | PinfC | 500 bp immediately upstream of the ATG start codon |
| | | NO 62 | | of the infC gene |
| none | 1021 | SEQ ID | Prm1 | 348 bp includes the 319 bp immediately upstream of the |
| | | NO 63 | | ATG start codon of the lpp gene and the first 29 bp of |
| | | | | the lpp gene |
| none | 1021 | SEQ ID | Prm7 | 339 bp upstream of the sspA gene, ending at 46 bp |
| | | NO 64 | | upstream of the ATG of the sspA gene |
| none | 1113 | SEQ ID | 16S | N/A |
| | | NO 65 | | |
| none | 1113 | SEQ ID | nifH | N/A |
| | | NO 66 | | |
| none | 1113 | SEQ ID | nifD1 | 1 of 2 unique genes annotated as nifD in 1113 genome |
| | | NO 67 | | |
| none | 1113 | SEQ ID | nifD2 | 2 of 2 unique genes annotated as nifD in 1113 genome |
| | | NO 68 | | |
| none | 1113 | SEQ ID | nifK | N/A |
| | | NO 69 | | |
| none | 1113 | SEQ ID | nifL | N/A |
| | | NO 70 | | |
| none | 1113 | SEQ ID | nifA partial gene | due to a gap in the sequence assembly, we can only |
| | | NO 71 | | identify a partial gene from the 1113 genome |
| none | 1113 | SEQ ID | glnE | N/A |
| | | NO 72 | | |
| none | 1116 | SEQ ID | 16S | |
| | | NO 73 | | |
| none | 1116 | SEQ ID | nifH | |
| | | NO 74 | | |
| none | 1116 | SEQ ID | nifD1 | 1 of 2 unique genes annotated as nifD in 1116 genome |
| | | NO 75 | | |
| none | 1116 | SEQ ID | nifD2 | 2 of 2 unique genes annotated as nifD in 1116 genome |
| | | NO 76 | | |
| none | 1116 | SEQ ID | nifK1 | 1 of 2 unique genes annotated as nifK in 1116 genome |
| | | NO 77 | | |
| none | 1116 | SEQ ID | nifK2 | 2 of 2 unique genes annotated as nifK in 1116 genome |
| | | NO 78 | | |
| none | 1116 | SEQ ID | nifL | N/A |
| | | NO 79 | | |
| none | 1116 | SEQ ID | nifA | N/A |
| | | NO 80 | | |
| none | 1116 | SEQ ID | glnE | N/A |
| | | NO 81 | | |
| none | 1116 | SEQ ID | amtB | N/A |
| | | NO 82 | | |
| none | 1293 | SEQ ID | 16S | N/A |
| | | NO 83 | | |
| none | 1293 | SEQ ID | nifH | N/A |
| | | NO 84 | | |
| none | 1293 | SEQ ID | nifD1 | 1 of 2 unique genes annotated as nifD in 1293 genome |
| | | NO 85 | | |
| none | 1293 | SEQ ID | nifD2 | 2 of 2 unique genes annotated as nifD in 1293 genome |
| | | NO 86 | | |
| none | 1293 | SEQ ID | nifK | 1 of 2 unique genes annotated as nifK in 1293 genome |
| | | NO 87 | | |
| none | 1293 | SEQ ID | nifK1 | 2 of 2 unique genes annotated as nifK in 1293 genome |
| | | NO 88 | | |
| none | 1293 | SEQ ID | nifA | N/A |
| | | NO 89 | | |
| none | 1293 | SEQ ID | glnE | N/A |
| | | NO 90 | | |
| none | 1293 | SEQ ID | amtB1 | 1 of 2 unique genes annotated as amtB in 1293 genome |
| | | NO 91 | | |
| none | 1293 | SEQ ID | amtB2 | 2 of 2 unique genes annotated as amtB in 1293 genome |
| | | NO 92 | | |
| none | 1021- | SEQ ID | ΔnifL::PinfC | starting at 24 bp after the A of the ATG start codon, |
| | 1612 | NO 93 | | 1375 bp of nifL have been deleted and replaced with |
| | | | | the 1021 PinfC promoter sequence |
| none | 1021- | SEQ ID | ΔnifL::PinfC with | starting at 24 bp after the A of the ATG start codon, |
| | 1612 | NO 94 | 500 bp flank | 1375 bp of nifL have been deleted and replaced with |
| | | | | the 1021 PinfC promoter sequence; 500 bp flanking the |
| | | | | nifL gene upstream and downstream are included |
| none | 1021- | SEQ ID | glnEΔAR-2 | glnE gene with 1673 bp immediately downstream of the |
| | 1612 | NO 95 | | ATG start codon deleted, resulting in a truncated glnE |
| | | | | protein lacking the adenylyl-removing (AR) domain |
| none | 1021- | SEQ ID | glnEΔAR-2 with | glnE gene with 1673 bp immediately downstream of the |
| | 1612 | NO 96 | 500 bp flank | ATG start codon deleted, resulting in a truncated glnE |
| | | | | protein lacking the adenylyl-removing (AR) domain; |
| | | | | 500 bp flanking the glnE gene upstream and |
| | | | | downstream are included |
| none | 1021- | SEQ ID | ΔnifL::Prm1 | starting at 24 bp after the A of the ATG start codon, |
| | 1615 | NO 97 | | 1375 bp of nifL have been deleted and replaced with |
| | | | | the 1021 Prm1 promoter sequence |
| none | 1021- | SEQ ID | ΔnifL::Prm1 with | starting at 24 bp after the A of the ATG start codon, |
| | 1615 | NO 98 | 500 bp flank | 1375 bp of nifL have been deleted and replaced with |
| | | | | the 1021 rml promoter sequence; 500 bp flanking the |
| | | | | nifL gene upstream and downstream are included |
| none | 1021- | SEQ ID | glnEΔAR-2 | glnE gene with 1673 bp immediately downstream of the |
| | 1615 | NO 99 | | ATG start codon deleted, resulting in a truncated glnE |
| | | | | protein lacking the adenylyl-removing (AR) domain |
| none | 1021- | SEQ ID | glnEΔAR-2 with | glnE gene with 1673 bp immediately downstream of the |
| | 1615 | NO 100 | 500 bp flank | ATG start codon deleted, resulting in a truncated glnE |
| | | | | protein lacking the adenylyl-removing (AR) domain; |
| | | | | 500 bp flanking the glnE gene upstream and |
| | | | | downstream are included |
| none | 1021- | SEQ ID | ΔnifL::Prm1 | starting at 24 bp after the A of the ATG start codon, |
| | 1619 | NO 101 | | 1375 bp of nifL have been deleted and replaced with |
| | | | | the 1021 Prm1 promoter sequence |
| none | 1021- | SEQ ID | ΔnifL::Prm1 with | starting at 24 bp after the A of the ATG start codon, |
| | 1619 | NO 102 | 500 bp flank | 1375 bp of nifL have been deleted and replaced with |
| | | | | the 1021 rm1 promoter sequence; 500 bp flanking the |
| | | | | nifL gene upstream and downstream are included |
| none | 1021- | SEQ ID | glnEΔAR-2 | glnE gene with 1673 bp immediately downstream of the |
| | 1623 | NO 103 | | ATG start codon deleted, resulting in a truncated glnE |
| | | | | protein lacking the adenylyl-removing (AR) domain |
| none | 1021- | SEQ ID | glnEΔAR-2 with | glnE gene with 1673 bp immediately downstream of the |
| | 1623 | NO 104 | 500 bp flank | ATG start codon deleted, resulting in a truncated glnE |
| | | | | protein lacking the adenylyl-removing (AR) domain; |
| | | | | 500 bp flanking the glnE gene upstream and |
| | | | | downstream are included |
| none | 1021- | SEQ ID | ΔnifL::Prm7 | starting at 24 bp after the A of the ATG start codon, |
| | 1623 | NO 105 | | 1375 bp of nifL have been deleted and replaced with |
| | | | | the 1021 Prm7 promoter sequence |
| none | 1021- | SEQ ID | ΔnifL::Prm7 with | starting at 24 bp after the A of the ATG start codon, |
| | 1623 | NO 106 | 500 bp flank | 1375 bp of nifL have been deleted and replaced with |
| | | | | the 1021 rm7 promoter sequence; 500 bp flanking the |
| | | | | nifL gene upstream and downstream are included |
| none | 137- | SEQ ID | glnEΔAR-2 | glnE gene with 1290 bp immediately downstream of the |
| | 1034 | NO 107 | | ATG start codon deleted, resulting in a truncated glnE |
| | | | | protein lacking the adenylyl-removing (AR) domain |
| none | 137- | SEQ ID | glnEΔAR-2 with | glnE gene with 1290 bp immediately downstream of the |
| | 1034 | NO 108 | 500 bp flank | ATG start codon deleted, resulting in a truncated glnE |
| | | | | protein lacking the adenylyl-removing (AR) domain; |
| | | | | 500 bp flanking the glnE gene upstream and |
| | | | | downstream are included |
| none | 137- | SEQ ID | ΔnifL::PinfC | starting at 24 bp after the A of the ATG start codon, |
| | 1036 | NO 109 | | 1372 bp of nifL have been deleted and replaced with |
| | | | | the 137 PinfC promoter sequence |
| none | 137- | SEQ ID | ΔnifL::PinfC with | starting at 24 bp after the A of the ATG start codon, |
| | 1036 | NO 110 | 500 bp flank | 1372 bp of nifL have been deleted and replaced with |
| | | | | the 137 PinfC promoter sequence; 500 bp flanking the |
| | | | | nifL gene upstream and downstream are included |
| none | 137- | SEQ ID | glnEΔAR-236 bp | glnE gene with 1290 bp immediately downstream of the |
| | 1314 | NO 111 | deletion | ATG start codon deleted AND 36 bp deleted beginning |
| | | | | at 1472 bp downstream of the start codon, resulting in a |
| | | | | truncated glnE protein lacking the adenylyl-removing |
| | | | | (AR) domain |
| none | 137- | SEQ ID | glnEΔAR-236 bp | glnE gene with 1290 bp immediately downstream of the |
| | 1314 | NO 112 | deletion | ATG start codon deleted AND 36 bp deleted beginning |
| | | | | at 1472 bp downstream of the start codon, resulting in a |
| | | | | truncated glnE protein lacking the adenylyl-removing |
| | | | | (AR) domain; 500 bp flanking the nifL gene upstream |
| | | | | and downstream are included |
| none | 137- | SEQ ID | ΔnifL::Prm8.2 | starting at 24 bp after the A of the ATG start codon, |
| | 1314 | NO 113 | | 1372 bp of nifL have been deleted and replaced with |
| | | | | the 137 Prm8.2 promoter sequence |
| none | 137- | SEQ ID | ΔnifL::Prm8.2 with | starting at 24 bp after the A of the ATG start codon, |
| | 1314 | NO 114 | 500 bp flank | 1372 bp of nifL have been deleted and replaced with |
| | | | | the 137 Prm8.2 promoter sequence; 500 bp flanking the |
| | | | | nifL gene upstream and downstream are included |
| none | 137- | SEQ ID | glnEΔAR-236 bp | glnE gene with 1290 bp immediately downstream of the |
| | 1329 | NO 115 | deletion | ATG start codon deleted AND 36 bp deleted beginning |
| | | | | at 1472 bp downstream of the start codon, resulting in a |
| | | | | truncated glnE protein lacking the adenylyl-removing |
| | | | | (AR) domain |
| none | 137- | SEQ ID | glnEΔAR-236 bp | glnE gene with 1290 bp immediately downstream of the |
| | 1329 | NO 116 | deletion | ATG start codon deleted AND 36 bp deleted beginning |
| | | | | at 1472 bp downstream of the start codon, resulting in a |
| | | | | truncated glnE protein lacking the adenylyl-removing |
| | | | | (AR) domain; 500 bp flanking the nifL gene upstream |
| | | | | and downstream are included |
| none | 137- | SEQ ID | ΔnifL::Prm6.2 | starting at 24 bp after the A of the ATG start codon, |
| | 1329 | NO 117 | | 1372 bp of nifL have been deleted and replaced with |
| | | | | the 137 Prm6.2 promoter sequence |
| none | 137- | SEQ ID | ΔnifL::Prm6.2 with | starting at 24 bp after the A of the ATG start codon, |
| | 1329 | NO 118 | 500 bp flank | 1372 bp of nifL have been deleted and replaced with |
| | | | | the 137 Prm6.2 promoter sequence; 500 bp flanking the |
| | | | | nifL gene upstream and downstream are included |
| none | 137- | SEQ ID | ΔnifL::Prm1.2 | starting at 24 bp after the A of the ATG start codon, |
| | 1382 | NO 119 | | 1372 bp of nifL have been deleted and replaced with |
| | | | | the 137 Prm1.2 promoter sequence |
| none | 137- | SEQ ID | ΔnifL::Prm1.2 with | starting at 24 bp after the A of the ATG start codon, |
| | 1382 | NO 120 | 500 bp flank | 1372 bp of nifL have been deleted and replaced with |
| | | | | the 137 Prm1.2 promoter sequence; 500 bp flanking the |
| | | | | nifL gene upstream and downstream are included |
| none | 137. | SEQ ID | glnEΔAR-236 bp | glnE gene with 1290 bp immediately downstream of the |
| | 1382 | NO 121 | deletion | ATG start codon deleted AND 36 bp deleted beginning |
| | | | | at 1472 bp downstream of the start codon, resulting in a |
| | | | | truncated glnE protein lacking the adenylyl-removing |
| | | | | (AR) domain |
| none | 137- | SEQ ID | glnEΔAR-236 bp | glnE gene with 1290 bp immediately downstream of the |
| | 1382 | NO 122 | deletion | ATG start codon deleted AND 36 bp deleted beginning |
| | | | | at 1472 bp downstream of the start codon, resulting in a |
| | | | | truncated glnE protein lacking the adenylyl-removing |
| | | | | (AR) domain; 500 bp flanking the nifL gene upstream |
| | | | | and downstream are included |
| none | 137- | SEQ ID | ΔnifL::PinfC | starting at 24 bp after the A of the ATG start codon, |
| | 1586 | NO 123 | | 1372 bp of nifL have been deleted and replaced with |
| | | | | the 137 PinfC promoter sequence |
| none | 137- | SEQ ID | ΔnifL::PinfC with | starting at 24 bp after the A of the ATG start codon, |
| | 1586 | NO 124 | 500 bp flank | 1372 bp of nifL have been deleted and replaced with |
| | | | | the 137 PinfC promoter sequence; 500 bp flanking the |
| | | | | nifL gene upstream and downstream are included |
| none | 137- | SEQ ID | glnEΔAR-2 | glnE gene with 1290 bp immediately downstream of the |
| | 1586 | NO 125 | | ATG start codon deleted, resulting in a truncated glnE |
| | | | | protein lacking the adenylyl-removing (AR) domain |
| none | 137- | SEQ ID | glnEΔAR-2 with | glnE gene with 1290 bp immediately downstream of the |
| | 1586 | NO 126 | 500 bp flank | ATG start codon deleted, resulting in a truncated glnE |
| | | | | protein lacking the adenylyl-removing (AR) domain; |
| | | | | 500 bp flanking the glnE gene upstream and |
| | | | | downstream are included |
| none | 19-594 | SEQ ID | glnEΔAR-2 | glnE gene with 1650 bp immediately downstream of the |
| | | NO 127 | | ATG start codon deleted, resulting in a truncated glnE |
| | | | | protein lacking the adenylyl-removing (AR) domain |
| none | 19-594 | SEQ ID | glnEΔAR-2 with | glnE gene with 1650 bp immediately downstream of the |
| | | NO 128 | 500 bp flank | ATG start codon deleted, resulting in a truncated glnE |
| | | | | protein lacking the adenylyl-removing (AR) domain; |
| | | | | 500 bp flanking the glnE gene upstream and |
| | | | | downstream are included |
| none | 19-594 | SEQ ID | ΔnifL::Prm6.1 | starting at 221 bp after the A of the ATG start codon, |
| | | NO 129 | | 845 bp of nifL have been deleted and replaced with the |
| | | | | CI019 Prm6.1 promoter sequence |
| none | 19-594 | SEQ ID | ΔnifL::Prm6.1 with | starting at 221 bp after the A of the ATG start codon, |
| | | NO 130 | 500 bp flank | 84 5 bp of nifL have been deleted and replaced with the |
| | | | | CI019 Prm6.Ipromoter sequence; 500 bp flanking the |
| | | | | nifL gene upstream and downstream are included |
| none | 19-714 | SEQ ID | ΔnifL::Prm6.1 | starting at 221 bp after the A of the ATG start codon, |
| | | NO 131 | | 845 bp of nifL have been deleted and replaced with the |
| | | | | CI019 Prm6.1 promoter sequence |
| none | 19-714 | SEQ ID | ΔnifL::Prm6.1 with | starting at 221 bp after the A of the ATG start codon, |
| | | NO 132 | 500 bp flank | 845 bp of nifL have been deleted and replaced with the |
| | | | | CI019 Prm6. Ipromoter sequence; 500 bp flanking the |
| | | | | nifL gene upstream and downstream are included |
| none | 19-715 | SEQ ID | ΔnifL:Prm7.1 | starting at 221 bp after the A of the ATG start codon, |
| | | NO 133 | | 845 bp of nifL have been deleted and replaced with the |
| | | | | CI019 Prm7.1 promoter sequence |
| none | 19-715 | SEQ ID | ΔnifL::Prm7.1 with | starting at 221 bp after the A of the ATG start codon, |
| | | NO 134 | 500 bp flank | 845 bp of nifL have been deleted and replaced with the |
| | | | | CI019 Prm76.1promoter sequence; 500 bp flanking the |
| | | | | nifL gene upstream and downstream are included |
| 19-713 | 19-750 | SEQ ID | ΔnifL::Prm1.2 | starting at 221 bp after the A of the ATG start codon, |
| | | NO 135 | | 84 S bp of nifL have been deleted and replaced with the |
| | | | | CI019 Prm1.2 promoter sequence |
| 19-713 | 19-750 | SEQ ID | ΔnifL::Prm1.2 with | starting at 221 bp after the A of the ATG start codon, |
| | | NO 136 | 500 bp flank | 845 bp of nifL have been deleted and replaced with the |
| | | | | CI019 Prm1.2 promoter sequence; 500 bp flanking the |
| | | | | nifL gene upstream and downstream are included |
| 17-724 | 19-804 | SEQ ID | ΔnifL::Prm1.2 | starting at 221 bp after the A of the ATG start codon, |
| | | NO 137 | | 845 bp of nifL have been deleted and replaced with the |
| | | | | CI019 Prm1.2 promoter sequence |
| 17-724 | 19-804 | SEQ ID | ΔnifL::Prm1.2 with | starting at 221 bp after the A of the ATG start codon, |
| | | NO 138 | 500 bp flank | 845 bp of nifL have been deleted and replaced with the |
| | | | | CI019 Prm1.2 promoter sequence; 500 bp flanking the |
| | | | | nifL gene upstream and downstream are included |
| 17-724 | 19-804 | SEQ ID | glnEΔAR-2 | glnE gene with 1650 bp immediately downstream of the |
| | | NO 139 | | ATG start codon deleted, resulting in a truncated glnE |
| | | | | protein lacking the adenylyl-removing (AR) domain |
| 17-724 | 19-804 | SEQ ID | glnEΔAR-2 with | glnE gene with 1650 bp immediately downstream of the |
| | | NO 140 | 500 bp flank | ATG start codon deleted, resulting in a truncated glnE |
| | | | | protein lacking the adenylyl-removing (AR) domain; |
| | | | | 500 bp flanking the glnE gene upstream and |
| | | | | downstream are included |
| 19-590 | 19-806 | SEQ ID | ΔnifL::Prm3.1 | starting at 221 bp after the A of the ATG start codon, |
| | | NO 141 | | 845 bp of nifL have been deleted and replaced with the |
| | | | | CI019 Prm3.1 promoter sequence |
| 19-590 | 19-806 | SEQ ID | ΔnifL::Prm3.1 with | starting at 221 bp after the A of the ATG start codon, |
| | | NO 142 | 500 bp flank | 845 bp of nifL have been deleted and replaced with the |
| | | | | CI019 Prm3.1 promoter sequence; 500 bp flanking the |
| | | | | nifL gene upstream and downstream are included |
| 19-590 | 19-806 | SEQ ID | glnEΔAR-2 | glnE gene with 1650 bp immediately downstream of the |
| | | NO 143 | | ATG start codon deleted, resulting in a truncated glnE |
| | | | | protein lacking the adenylyl-removing (AR) domain |
| 19-590 | 19-806 | SEQ ID | glnEΔAR-2 with | glnE gene with 1650 bp immediately downstream of the |
| | | NO 144 | 500 bp flank | ATG start codon deleted, resulting in a truncated glnE |
| | | | | protein lacking the adenylyl-removing (AR) domain; |
| | | | | 500 bp flanking the glnE gene upstream and |
| | | | | downstream are included |
| none | 63- | SEQ ID | ΔnifL:PinfC | starting at 24 bp after the A of the ATG start codon, |
| | 1146 | NO 145 | | 1375 bp of nifL have been deleted and replaced with |
| | | | | the 63 PinfC promoter sequence |
| none | 63- | SEQ ID | ΔnifL::PinfC with | starting at 24 bp after the A of the ATG start codon, |
| | 1146 | NO 146 | 500 bp flank | 1375 bp of nifL have been deleted and replaced with |
| | | | | the 63 PinfC promoter sequence; 500 bp flanking the |
| | | | | nifL gene upstream and downstream are included |
| CM015; | 6-397 | SEQ ID | ΔnifL::Prm5 | starting at 31 bp after the A of the ATG start codon, |
| PBC6.15 | | NO 147 | | 1375 bp of nifL have been deleted and replaced with |
| | | | | the CI006 Prm5 promoter sequence |
| CM015; | 6-397 | SEQ ID | ΔnifL::Prm5 with | starting at 31 bp after the A of the ATG start codon, |
| PBC6.15 | | NO 148 | 500 bp flank | 1375 bp of nifL have been deleted and replaced with |
| | | | | the CI006 Prm5 promoter sequence; 500 bp flanking |
| | | | | the nifL gene upstream and downstream are included |
| CM014 | 6-400 | SEQ ID | ΔnifL::Prm1 | starting at 31 bp after the A of the ATG start codon, |
| | | NO 149 | | 1375 bp of nifL have been deleted and replaced with |
| | | | | the CI006 Prm1 promoter sequence |
| CM014 | 6-400 | SEQ ID | ΔnifL::Prm1 with | starting at 31 bp after the A of the ATG start codon, |
| | | NO 150 | 500 bp flank | 1375 bp of nifL have been deleted and replaced with |
| | | | | the CI006 Prm1 promoter sequence; 500 bp flanking |
| | | | | the nifL gene upstream and downstream are included |
| CM037; | 6-403 | SEQ ID | ΔnifL::Prm1 | starting at 31 bp after the A of the ATG start codon, |
| PBC6.37 | | NO 151 | | 1375 bp of nifL have been deleted and replaced with |
| | | | | the CI006 Prm1 promoter sequence |
| CM037; | 6-403 | SEQ ID | ΔnifL:Prm1 with | starting at 31 bp after the A of the ATG start codon, |
| PBC6.38 | | NO 152 | 500 bp flank | 1375 bp of nifL have been deleted and replaced with |
| | | | | the CI006 Prm1 promoter sequence; 500 bp flanking |
| | | | | the nifL gene upstream and downstream are included |
| CM037; | 6-403 | SEQ ID | glnEΔAR-2 | glnE gene with 1644 bp immediately downstream of the |
| PBC6.39 | | NO 153 | | ATG start codon deleted, resulting in a truncated glnE |
| | | | | protein lacking the adenylyl-removing (AR) domain |
| CM037; | 6-403 | SEQ ID | glnEΔAR-2 with | glnE gene with 1644 bp immediately downstream of the |
| PBC6.40 | | NO 154 | 500 bp flank | ATG start codon deleted, resulting in a truncated glnE |
| | | | | protein lacking the adenylyl-removing (AR) domain; |
| | | | | 500 bp flanking the glnE gene upstream and |
| | | | | downstream are included |
| CM038; | 6-404 | SEQ ID | glnEΔAR-1 | glnE gene with 1287 bp immediately downstream of the |
| PBC6.38 | | NO 155 | | ATG start codon deleted, resulting in a truncated glnE |
| | | | | protein lacking the adenylyl-removing (AR) domain |
| CM038; | 6-404 | SEQ ID | ΔnifL::Prm1 | starting at 31 bp after the A of the ATG start codon, |
| PBC6.38 | | NO 156 | | 1375 bp of nifL have been deleted and replaced with |
| | | | | the CI006 Pril promoter sequence |
| CM038; | 6-404 | SEQ ID | ΔnifL::Prm 1 with | starting at 31 bp after the A of the ATG start codon, |
| PBC6.38 | | NO 157 | 500 bp flank | 1375 bp of nifL have been deleted and replaced with |
| | | | | the CI006 Prm1 promoter sequence; 500 bp flanking |
| | | | | the nifL gene upstream and downstream are included |
| CM038; | 6-404 | SEQ ID | glnEΔAR-1 with | glnE gene with 1287 bp immediately downstream of the |
| PBC6.38 | | NO 158 | 500 bp flank | ATG start codon deleted, resulting in a truncated glnE |
| | | | | protein lacking the adenylyl-removing (AR) domain; |
| | | | | 500 bp flanking the glnE gene upstream and |
| | | | | downstream are included |
| CM029; | 6-412 | SEQ ID | glnEΔAR-1 | glnE gene with 1287 bp immediately downstream of the |
| PBC6.29 | | NO 159 | | ATG start codon deleted, resulting in a truncated glnE |
| | | | | protein lacking the adenylyl-removing (AR) domain |
| CM029; | 6-412 | SEQ ID | glnEΔAR-1 with | glnE gene with 1287 bp immediately downstream of the |
| PBC6.29 | | NO 160 | 500 bp flank | ATG start codon deleted, resulting in a truncated glnE |
| | | | | protein lacking the adenylyl-removing (AR) domain; |
| | | | | 500 bp flanking the glnE gene upstream and |
| | | | | downstream are included |
| CM029; | 6-412 | SEQ ID | ΔnifL::Prm5 | starting at 31 bp after the A of the ATG start codon, |
| PBC6.29 | | NO 161 | | 1375 bp of nifL have been deleted and replaced with |
| | | | | the CI006 Prm5 promoter sequence |
| CM029; | 6-412 | SEQ ID | ΔnifL::Prm5 with | starting at 31 bp after the A of the ATG start codon, |
| PBC6.29 | | NO 162 | 500 bp flank | 1375 bp of nifL have been deleted and replaced with |
| | | | | the CI006 Prm5 promoter sequence; 500 bp flanking |
| | | | | the nifL gene upstream and downstream are included |
| CM093; | 6-848 | SEQ ID | ΔnifL::Prm1 | starting at 31 bp after the A of the ATG start codon, |
| PBC6.93 | | NO 163 | | 1375 bp of nifL have been deleted and replaced with |
| | | | | the CI006 Prm1 promoter sequence |
| CM093; | 6-848 | SEQ ID | ΔnifL::Prm1 with | starting at 31 bp after the A of the ATG start codon, |
| PBC6.93 | | NO 164 | 500 bp flank | 1375 bp of nifL have been deleted and replaced with |
| | | | | the CI006 Prm1 promoter sequence; 500 bp flanking |
| | | | | the nifL gene upstream and downstream are included |
| CM093; | 6-848 | SEQ ID | glnEΔAR-2 | glnE gene with 1644 bp immediately downstream of the |
| PBC6.93 | | NO 165 | | ATG start codon deleted, resulting in a truncated glnE |
| | | | | protein lacking the adenylyl-removing (AR) domain |
| CM093; | 6-848 | SEQ ID | glnEΔAR-2 with | glnE gene with 1644 bp immediately downstream of the |
| PBC6.93 | | NO 166 | 500 bp flank | ATG start codon deleted, resulting in a truncated glnE |
| | | | | protein lacking the adenylyl-removing (AR) domain; |
| | | | | 500 bp flanking the glnE gene upstream and |
| | | | | downstream are included |
| CM093; | 6-848 | SEQ ID | ΔamtB | First 1088 bp of amtB gene and 4 bp upstream of start |
| PBC6.93 | | NO 167 | | codon deleted; 199 bp of gene remaining lacks a start |
| | | | | codon; no amtB protein is translated |
| CM093; | 6-848 | SEQ ID | ΔamtB with 500 bp | First 1088 bp of amtB gene and 4 bp upstream of start |
| PBC6.93 | | NO 168 | flank | codon deleted; 199 bp of gene remaining lacks a start |
| | | | | codon; no amtB protein is translated |
| CM094; | 6-881 | SEQ ID | glnEΔAR-1 | glnE gene with 1287 bp immediately downstream of the |
| PBC6.94 | | NO 169 | | ATG start codon deleted, resulting in a truncated glnE |
| | | | | protein lacking the adenylyl-removing (AR) domain |
| CM094; | 6-881 | SEQ ID | glnEΔAR-1 with | glnE gene with 1287 bp immediately downstream of the |
| PBC6.94 | | NO 170 | 500 bp flank | ATG start codon deleted, resulting in a truncated glnE |
| | | | | protein lacking the adenylyl-removing (AR) domain; |
| | | | | 500 bp flanking the glnE gene upstream and |
| | | | | downstream are included |
| CM094; | 6-881 | SEQ ID | ΔnifL::Prm1 | starting at 31 bp after the A of the ATG start codon, |
| PBC6.94 | | NO 171 | | 1375 bp of nifL have been deleted and replaced with |
| | | | | the CI006 Prm1 promoter sequence |
| CM094; | 6-881 | SEQ ID | ΔnifL::Prm1 with | starting at 31 bp after the A of the ATG start codon, |
| PBC6.94 | | NO 172 | 500 bp flank | 1375 bp of nifL have been deleted and replaced with |
| | | | | the CI006 Prm1 promoter sequence; 500 bp flanking |
| | | | | the nifL gene upstream and downstream are included |
| CM094; | 6-881 | SEQ ID | ΔamtB | First 1088 bp of amtB gene and 4 bp upstream of start |
| PBC6.94 | | NO 173 | | codon deleted; 199 bp of gene remaining lacks a start |
| | | | | codon; no amtB protein is translated |
| CM094; | 6-881 | SEQ ID | ΔamtB with 500 bp | First 1088 bp of amtB gene and 4 bp upstream of start |
| PBC6.94 | | NO 174 | flank | codon deleted; 199 bp of gene remaining lacks a start |
| | | | | codon; no amtB protein is translated |
| none | 910- | SEQ ID | ΔnifL::PinfC | starting at 20 bp after the A of the ATG start codon, |
| | 1246 | NO 175 | | 1379 bp of nifL have been deleted and replaced with |
| | | | | the 910 PinfC promoter sequence |
| none | 910- | SEQ ID | ΔnifL::PinfC with | starting at 20 bp after the A of the ATG start codon, |
| | 1246 | NO 176 | 500 bp flank | 1379 bp of nifL have been deleted and replaced with |
| | | | | the 910 PinfC promoter sequence; 500 bp flanking the |
| | | | | nifL gene upstream and downstream are included |
| PBC6.1, | CI006 | SEQ ID | 16S-1 | 1 of 3 unique 16S rDNA genes in the CI006 genome |
| 6, CI6 | | NO 177 | | |
| PBC6.1, | CI006 | SEQ ID | 16S-2 | 2 of 3 unique 16S rDNA genes in the CI006 genome |
| 6, CI6 | | NO 178 | | |
| PBC6.1, | CI006 | SEQ ID | nifH | N/A |
| 6, CI6 | | NO 179 | | |
| PBC6.1, | CI006 | SEQ ID | nifD2 | 2 of 2 unique genes annotated as nifD in CI006 |
| 6, CI6 | | NO 180 | | genome |
| PBC6.1, | CI006 | SEQ ID | nifK2 | 2 of 2 unique genes annotated as nifK in CI006 |
| 6, C16 | | NO 181 | | genome |
| PBC6.1, | CI006 | SEQ ID | nifL | N/A |
| 6, CI6 | | NO 182 | | |
| PBC6.1, | CI006 | SEQ ID | nifA | N/A |
| 6, CI6 | | NO 183 | | |
| PBC6.1, | CI006 | SEQ ID | glnE | N/A |
| 6, CI6 | | NO 184 | | |
| PBC6.1, | CI006 | SEQ ID | 16S-3 | 3 of 3 unique 16S rDNA genes in the CI006 genome |
| 6, CI6 | | NO 185 | | |
| PBC6.1, | CI006 | SEQ ID | nifD1 | 1 of 2 unique genes annotated as nifD in CI006 |
| 6, CI6 | | NO 186 | | genome |
| PBC6.1, | CI006 | SEQ ID | nifK1 | 1 of 2 unique genes annotated as nifK in CI006 |
| 6, CI6 | | NO 187 | | genome |
| PBC6.1, | CI006 | SEQ ID | amtB | N/A |
| 6, CI6 | | NO 188 | | |
| PBC6.1, | CI006 | SEQ ID | Prm1 | 348 bp includes the 319 bp immediately upstream of the |
| 6, CI6 | | NO 189 | | ATG start codon of the Ipp gene and the first 29 bp of |
| | | | | the Ipp gene |
| PBC6.1, | CI006 | SEQ ID | Prm5 | 313 bp starting at 432 bp upstream of the ATG start |
| 6, CI6 | | NO 190 | | codon of the ompX gene and ending 119 bp upstream |
| | | | | of the ATG start codon of the ompX gene |
| 19, CI19 | CI019 | SEQ ID | nifL | N/A |
| | | NO 191 | | |
| 19, CI19 | CI019 | SEQ ID | nifA | N/A |
| | | NO 192 | | |
| 19, CI19 | CI019 | SEQ ID | 16S-1 | 1 of 7 unique 16S rDNA genes in the CI019 genome |
| | | NO 193 | | |
| 19, CI19 | CI019 | SEQ ID | 16S-2 | 2 of 7 unique 16S rDNA genes in the CI019 genome |
| | | NO 194 | | |
| | | NO 195 | | |
| 19, CI19 | CI019 | SEQ ID | 16S-4 | 4 of 7 unique 16S rDNA genes in the CI019 genome |
| | | NO 196 | | |
| 19. CI19 | CI019 | SEQ ID | 16S-5 | 5 of 7 unique 16S rDNA genes in the CI019 genome |
| | | NO 197 | | |
| 19, CI19 | CI019 | SEQ ID | 16S-6 | 6 of 7 unique 168 rDNA genes in the CI019 genome |
| | | NO 198 | | |
| 19, CI19 | CI019 | SEQ ID | 16S-7 | 7 of 7 unique 16S rDNA genes in the CI019 genome |
| | | NO 199 | | |
| 19, CI19 | CI019 | SEQ ID | nifH1 | 1 of 2 unique genes annotated as nifH in CI019 |
| | | NO 200 | | genome |
| 19, CI19 | CI019 | SEQ ID | nifH2 | 2 of 2 unique genes annotated as nifH in CI019 |
| | | NO 201 | | genome |
| 19, CI19 | CI019 | SEQ ID | nifD1 | 1 of 2 unique genes annotated as nifD in CI019 |
| | | NO 202 | | genome |
| 19, CI19 | CI019 | SEQ ID | nifD2 | 2 of 2 unique genes annotated as nifD in CI019 |
| | | NO 203 | | genome |
| 19, CI19 | CI019 | SEQ ID | nifK1 | 1 of 2 unique genes annotated as nifK in CI019 |
| | | NO 204 | | genome |
| 19, CI19 | CI019 | SEQ ID | nifK2 | 2 of 2 unique genes annotated as nifK in CI019 |
| | | NO 205 | | genome |
| 19. CI19 | CI019 | SEQ ID | glnE | N/A |
| | | NO 206 | | |
| 19, CI19 | CI019 | SEQ ID | Prm4 | 449 bp immediately upstream of the ATG of the dscC 2 |
| | | NO 207 | | gene |
| 19, CI19 | CI019 | SEQ ID | Prm1.2 | 500 bp immediately upstream of the TTG start codon of |
| | | NO 208 | | the infC gene |
| 19, CI19 | CI019 | SEQ ID | Prm3.1 | 170 bp immediately upstream of the ATG start codon |
| | | NO 209 | | of the rplN gene |
| 19, CI20 | CI020 | SEQ ID | Prm6.1 | 142 bp immediately upstream of the ATG of a highly- |
| | | NO 210 | | expressed hypothetical protein (annotated as |
| | | | | PROKKA_00662 in CI019 assembly 82) |
| 19, CI21 | CI021 | SEQ ID | Prm7.1 | 293 bp immediately upstream of the ATG of the lpp |
| | | NO 211 | | gene |
| 19-375, | CM67 | SEQ ID | glnEΔAR-2 | glnE gene with 1650 bp immediately downstream of the |
| 19-417, | | NO 212 | | ATG start codon deleted, resulting in a truncated glnE |
| CM067 | | | | protein lacking the adenylyl-removing (AR) domain |
| 19-375, | CM67 | SEQ ID | glnEΔAR-2 with | glnE gene with 1650 bp immediately downstream of the |
| 19-417, | | NO 213 | 500 bp flank | ATG start codon deleted, resulting in a truncated glnE |
| CM067 | | | | protein lacking the adenylyl-removing (AR) domain; |
| | | | | 500 bp flanking the glnE gene upstream and |
| | | | | downstream are included |
| 19-375, | CM67 | SEQ ID | ΔnifL::null-v1 | starting at 221 bp after the A of the ATG start codon, |
| 19-417, | | NO 214 | | 845 bp of nifL have been deleted and replaced with the |
| CM067 | | | | 31 bp sequence |
| | | | | “GGAGTCTGAACTCATCCTGCGATGGGGGCTG” |
| 19-375, | CM67 | SEQ ID | ΔnifL::null-v1 with | starting at 221 bp after the A of the ATG start codon, |
| 19-417, | | NO 215 | 500 bp flank | 845 bp of nifL have been deleted and replaced with the |
| CM067 | | | | 31 bp sequence |
| | | | | “GGAGTCTGAACTCATCCTGCGATGGGGGCTG”; |
| | | | | 500 bp flanking the nifL gene upstream and |
| | | | | downstream are included |
| 19-377, | CM69 | SEQ ID | ΔnifL::null-v2 | starting at 221 bp after the A of the ATG start codon, |
| CM069 | | NO 216 | | 845 bp of nifL have been deleted and replaced with the |
| | | | | 5 bp sequence “TTAAA” |
| 19-377, | CM69 | SEQ ID | ΔnifL::null-v2 with | starting at 221 bp after the A of the ATG start codon, |
| CM069 | | NO 217 | 500 bp flank | 845 bp of nifL have been deleted and replaced with the |
| | | | | 5 bp sequence “TTAAA”; 500 bp flanking the nifL gene |
| | | | | upstream and downstream are included |
| 19-389, | CM81 | SEQ ID | ΔnifL::Prm4 | starting at 221 bp after the A of the ATG start codon, |
| 19-418, | | NO 218 | | 845 bp of nifL have been deleted and replaced with the |
| CM081 | | | | CI19 Prm4 sequence |
| 19-389, | CM81 | SEQ ID | ΔnifL::Prm4 with | starting at 221 bp after the A of the ATG start codon, |
| 19-418, | | NO 219 | 500 bp flank | 845 bp of nifL have been deleted and replaced with the |
| CM081 | | | | CI19 Prm4 sequence; 500 bp flanking the nifL gene |
| | | | | upstream and downstream are included |
| | CI006 | SEQ ID | glnD with flanking | glnD gene with upstream and downstream flanking |
| | | NO 220 | sequences | sequences included |
| | 6-5687 | SEQ ID | ΔnifL::Prm5 | Retaining the first 30 bp and the last 83 bp, the middle |
| | | NO 221 | | region of nifL is deleted and replaced with the CI006 |
| | | | | Prm5 promoter sequence. |
| | 6-5687 | SEQ ID | glnEΔAR | Deletion of the first 1287 bp after the ATG start codon |
| | | NO 222 | | of the ginE gene. Resulting GinE protein lacks AR |
| | | | | domain, but expresses ATase domain. |
| | 6-5687 | SEQ ID | ΔglnD with flanking | Deletion of glnD gene by removing all 2676 |
| | | NO 223 | sequences | nucleotides, including start and stop codons. Upstream |
| | | | | and downstream flanking sequences are included. |
| | 137- | | ΔnifL::PinfC | Deletion of the nifL gene from 20 bp after the ATG |
| | 1036 | | | (start) to 87 bp before the TGA (stop) of the gene. A |
| | | | | 500 bp fragment of the region upstream of the infC |
| | | | | gene was inserted (PinfC) upstream of nifA replacing |
| | | | | the deleted portion. |
| | 137- | | ΔglnE_{AR}-KO2 | Deletion of 1647 bp after the start codon of the glnE |
| | 1034 | | | gene. |
| | 137- | | ΔnifL::PinfC | Deletion of the nif gene from 20 bp after the ATG |
| | 2249 | | | (start) to 87 bp before the TGA (stop) of the gene. A |
| | | | | 500 bp fragment of the region upstream of the infC |
| | | | | gene was inserted (PinfC) upstream of nifA replacing |
| | | | | the deleted portion. |
| | 137- | | ΔglnE_{AR}-DxD | Modification of the “GAT” found 513 bp after the start |
| | 2249 | | | codon of glnE to a “GCG” codon. |
| | 137- | | ΔnifL::Prm8.2 | Deletion of the nif gene from 20 bp after the ATG |
| | 1968 | | | (start) to 87 bp before the TGA (stop) of the gene. A |
| | | | | 299 bp fragment (Prm8.2), found 77 bp after the start |
| | | | | codon of nipl to 376 bp after the start codon of nipI was |
| | | | | inserted upstream of nifA replacing the deleted portion. |
| | 137- | | ΔglnE_{AR}-KO2 | Deletion of 1647 bp after the start codon of the glnE |
| | 1968 | | | gene. |
| | 137- | | ΔnifL::PinfC | Deletion of the nifL gene from 20 bp after the ATG |
| | 1586 | | | (start) to 87 bp before the TGA (stop) of the gene. A |
| | | | | 500 bp fragment of the region upstream of the infC |
| | | | | gene was inserted (PinfC) upstream of nifA replacing |
| | | | | the deleted portion. |
| | 137- | | ΔglnE_{AR}-KO2 | Deletion of 1647 bp after the start codon of the glnE |
| | 1586 | | | gene. |
| | 137- | | ΔnifL::Prm1.2 | Deletion of the nifL gene from 20 bp after the ATG |
| | 2084 | | | (start) to 87 bp before the TGA (stop) of the gene. A |
| | | | | 400 bp fragment from the region upstream of the cspE |
| | | | | gene was inserted (Prm1.2) upstream of nifA replacing |
| | | | | the deleted portion. |
| | 137- | | ΔglnE_{AR}-KO2 | Deletion of 1647 bp after the start codon of the ginE |
| | 2084 | | | gene. |
| | 137- | | ΔnifL::Prm1.2 | Deletion of the nifL gene from 20 bp after the ATG |
| | 2251 | | | (start) to 87 bp before the TGA (stop) of the gene. A |
| | | | | 400 bp fragment from the region upstream of the cspE |
| | | | | gene was inserted (Prm1.2) upstream of nifA replacing |
| | | | | the deleted portion. |
| | 137- | | ΔglnE_{AR}-KO2 | Deletion of 1647 bp after the start codon of the ginE |
| | 2251 | | | gene. |
| | 137- | | rpoN-Prm8.2 | Deletion of the 47 bp between ibtB2 and rpoN and |
| | 2251 | | | insertion of a fragment (Prm8.2), found 77 bp after the |
| | | | | start codon of nlpI to 376 bp after the start codon of |
| | | | | nlpI, directly upstream of rpoN. |
| | 137- | | ΔnifL::Prm1.2 | Deletion of the nifL gene from 20 bp after the ATG |
| | 2219 | | | (start) to 87 bp before the TGA (stop) of the gene. A |
| | | | | 400 bp fragment from the region upstream of the cspE |
| | | | | gene was inserted (Prm1.2) upstream of nifA replacing |
| | | | | the deleted portion. |
| | 137- | | ΔglnE_{AR}-KO2 | Deletion of 1647 bp after the start codon of the glnE |
| | 2219 | | | gene. |
| | 137- | | ΔglnD_{ACT1/2} | Deletion of the 546 bp before the stop codon of the |
| | 2219 | | | glnD gene. |

TABLE 5
Further Descriptions of Strains from the Disclosure

| Strain | | | | Accession |
|---|---|---|---|---|
| ID | Lineage | Mutagenic DNA Description | Genotype | Number |
| CI006 | CI006 | Wildtype parent Kosakonia sacchari | WT | 201701001 |
| CI137 | CI137 | Wildtype parent Klebsiella variicola | WT | 201708001 |
| CI910 | CI910 | Wildtype parent Metakosakonia | WT | PTA-126585 |
| | | intestine | | |
| CI8 | CI8 | Wildtype parent Paraburkholderia | WT | PTA-126582 |
| | | tropica | | |
| CI41 | CI41 | Wildtype parent Paenibacillus | WT | PTA-126581 |
| | | polymyxa | | |
| CI3069 | CI3069 | Wildtype parent Herbaspirillum | WT | PTA-126583 |
| | | aquaticum | | |
| 6-403 | Mutant of | Disruption of nifL gene with a | ΔnifL::Prml, ΔglnE- | 201708004 |
| | CI006 | fragment of the region upstream of the | AR_KO2 | |
| | | lpp gene inserted (Prm1) upstream of | | |
| | | nifA. Deletion of the 1647 bp after the | | |
| | | start codon of the glnE gene containing | | |
| | | the adenylyl-removing domain of | | |
| | | glutamate-ammonia-ligase | | |
| | | adenylyltransferase (ΔglnE-AR_KO2). | | |
| 6-2425 | Mutant of | Disruption of nifL gene with a | ΔnifL::Prm1, | PTA-126575 |
| | CI006 | fragment of the region upstream of the | ΔglnD_UT_truncation | |
| | | lpp gene inserted (Prml) upstream of | | |
| | | nifA. Deletion of the 987 bp after the | | |
| | | start codon of the glnD gene containing | | |
| | | the uridylyltransferase (UT) domain of | | |
| | | the bifunctional | | |
| | | uridylyltransferase/uridylyl-removing | | |
| | | enzyme (ΔglnD-UT_truncation) | | |
| 6-2634 | Mutant of | Disruption of nifL gene with a | ΔnifL:Prm1, | PTA-126576 |
| | CI006 | fragment of the region upstream of the | ΔglnD_UT_deactivation | |
| | | lpp gene inserted (Prml) upstream of | | |
| | | nifA. Deactivation of the | | |
| | | uridyly ltransferase (UT) domain of the | | |
| | | bifunctional | | |
| | | uridylyltransferase/uridylyl-removing | | |
| | | enzyme, glnD, by mutating amino acid | | |
| | | residues 90 and 91 from GG to DV as | | |
| | | well as residue 104 from D to A. | | |
| 137- | Mutant of | Deletion of the 1647 bp after the start | ΔglnE-AR_KO2 | 201712001 |
| 1034 | CI137 | codon of the glnE gene containing the | | |
| | | adenylyl-removing domain of | | |
| | | glutamate-ammonia-ligase | | |
| | | adenylyltransferase (ΔglnE-AR_KO2). | | |
| 137- | Mutant of | Disruption of nifL gene with a | ΔnifL::Prm1.2 ΔglnE- | |
| 2084 | CI137 | fragment of the region upstream of the | AR_KO2 | |
| | | cspE gene inserted (Prm1.2) upstream | | |
| | | of nifA. Deletion of the 1647 bp after | | |
| | | the start codon of the glnE gene | | |
| | | containing the adenylyl-removing | | |
| | | domain of glutamate-ammonia-ligase | | |
| | | adenylyltransferase (ΔglnE-AR_KO2). | | |
| 137- | | Deletion of the native dctA1 promoter | ΔnifL::P8.2, ΔglnE- | PTA-126577 |
| 1968 | | and insertion of a fragment (Prm8.2), | AR_KO2 | |
| | | found 77 bp after the start codon of nlpI | | |
| | | to 376 bp after the start codon of nlpI, | | |
| | | directly upstream of dctA1. Deletion of | | |
| | | the 1647 bp after the start codon of the | | |
| | | glnE gene containing the adenylyl- | | |
| | | removing domain of glutamate- | | |
| | | ammonia-ligase adenylyltransferase | | |
| | | (ΔglnE-AR_KO2). | | |
| 137- | Mutant of | Disruption of nifL gene with a | ΔnifL::Prm1.2 ΔglnE- | PTA-126578 |
| 2219 | CI137 | fragment of the region upstream of the | AR_KO2, | |
| | | cspE gene inserted (Prm1.2) upstream | ΔglnD_ACT12_truncation | |
| | | of nifA. Deletion of the 1647 bp after | | |
| | | the start codon of the glnE gene | | |
| | | containing the adenylyl-removing | | |
| | | domain of glutamate-ammonia-ligase | | |
| | | adenylyltransferase (ΔglnE-AR_KO2). | | |
| | | Deletion of the 546 bp before the stop | | |
| | | codon of the glnD gene containing the | | |
| | | ACT1/2 domain of the bifunctional | | |
| | | uridylyltransferase/uridylyl-removing | | |
| | | enzyme (ΔglnD-ACT12_truncation) | | |
| 137- | Mutant of | Disruption of nifL gene with a | ΔnifL::Prm1.2, ΔglnE- | PTA-126579 |
| 2237 | CI137 | fragment of the region upstream of the | AR_KO2, glsA2::Prm1.2 | |
| | | cspE gene inserted (Prm1.2) upstream | | |
| | | of nifA. Deletion of the 1647 bp after | | |
| | | the start codon of the glnE gene | | |
| | | containing the adenylyl-removing | | |
| | | domain of glutamate-ammonia-ligase | | |
| | | adenylyltransferase (ΔglnE-AR_KO2). | | |
| | | Deletion of the native glsA2 promoter | | |
| | | and insertion of a fragment (Prm 1.2) | | |
| | | directly upstream of the glsA2 CDS. | | |
| 137- | Mutant of | Disruption of nifL gene with a | ΔnifL::Prm1.2, ΔglnE- | PTA-126580 |
| 2285 | CI137 | fragment of the region upstream of the | AR_KO2, rpoN::Prm1.2 | |
| | | cspE gene inserted (Prm1.2) upstream | | |
| | | of nifA. Deletion of the 1647 bp after | | |
| | | the start codon of the glnE gene | | |
| | | containing the adenylyl-removing | | |
| | | domain of glutamate-ammonia-ligase | | |
| | | adenylyltransferase (ΔglnE-AR_KO2). | | |
| | | Deletion of the native rpoN promoter | | |
| | | and insertion of a fragment (Prm1.2) | | |
| | | directly upstream of the rpoN CDS. | | |
| 910- | Mutant of | Disruption of nifL gene with a | ΔnifL::Prm2.1, ΔglnE- | PTA-126588 |
| 3994 | CI910 | fragment of the region upstream of the | AR_KO2, glsA2::Prm1.1 | |
| | | rmF gene inserted (Prm2.1) upstream | | |
| | | of nifA. Deletion of the 1647 bp after | | |
| | | the start codon of the glnE gene | | |
| | | containing the adenylyl-removing | | |
| | | domain of glutamate-ammonia-ligase | | |
| | | adenylyltransferase (ΔglnE-AR_KO2). | | |
| | | Deletion of the native glsA2 promoter | | |
| | | and insertion of a fragment upstream of | | |
| | | the csrA gene (Prm1.1) directly | | |
| | | upstream of the glsA2 CDS. | | |
| 910- | Mutant of | Disruption of nifL gene with a | ΔnifL::Prm2.1, ΔglnE- | PTA-126586 |
| 3963 | CI910 | fragment of the region upstream of the | AR_KO2, | |
| | | rmF gene inserted (Prm2.1) upstream | ΔglnD_UT_truncation | |
| | | of nifA. Deletion of the 1647 bp after | | |
| | | the start codon of the glnE gene | | |
| | | containing the adenylyl-removing | | |
| | | domain of glutamate-ammonia-ligase | | |
| | | adenylyltransferase (ΔglnE-AR_KO2). | | |
| | | Deletion of the 987 bp after the start | | |
| | | codon of the glnD gene containing the | | |
| | | uridylyltransferase (UT) domain of the | | |
| | | bifunctional | | |
| | | uridylyltransferase/uridylyl-removing | | |
| | | enzyme (ΔglnD-UT_truncation) | | |
| 910- | Mutant of | Disruption of nifL gene with a | ΔnifL::Prm2.1, ΔglnE- | PTA-126584 |
| 3655 | CI910 | fragment of the region upstream of the | AR_KO2 | |
| | | rmF gene inserted (Prm2.1) upstream | | |
| | | of nifA. Deletion of the 1647 bp after | | |
| | | the start codon of the glnE gene | | |
| | | containing the adenylyl-removing | | |
| | | domain of glutamate-ammonia-ligase | | |
| | | adenylyltransferase (ΔglnE-AR_KO2). | | |
| 910- | Mutant of | Disruption of nifL gene with a | ΔnifL::Prm2.1, | PTA-126587 |
| 3961 | CI910 | fragment of the region upstream of the | ΔglnD_UT_truncation | |
| | | rmF gene inserted (Prm2.1) upstream | | |
| | | of nifA. Deletion of the 987 bp after the | | |
| | | start codon of the glnD gene containing | | |
| | | the uridylyltransferase (UT) domain of | | |
| | | the bifunctional | | |
| | | uridylyltransferase/uridylyl-removing | | |
| | | enzyme (ΔglnD-UT_truncation). | | |
| 6-5687 | Mutant of | Disruption of nifL gene with a | ΔnifL::Prm5, glnEΔAR, | PTA-126743 |
| | CI006 | promoter that natively drives ompX | ΔglnD | |
| | | (Prm5) inserted upstream of nifA. | | |
| | | Deletion of the 1287 bp after the start | | |
| | | codon of the glnE gene containing the | | |
| | | adenylyl-removing domain of | | |
| | | glutamate-ammonia-ligase | | |
| | | adenylyltransferase Deletion of entire | | |
| | | glnD gene. | | |
| 137- | | | ΔnifL::PinfC, glnD- | PTA-126740 |
| 2253 | | | ΔUTase | |

### IV. Additives to Dehydrated Microbes in the Water-Soluble Film Package

In some embodiments, the one or more compartment(s) of the water-soluble package comprises an additive that enhances the shelf stability, uniformity, flow attributes, dissolution kinetics, or any combination thereof, of the dehydrated microbe. In some embodiments, the dehydrated microbes comprise a blend of dehydrated microbes with one or more hygroscopic salt(s). In some embodiments, the dehydrated microbes comprise a blend of dehydrated microbes with at least about 1% of one or more hygroscopic salt(s), for example, at least about 5%, at least about 10%, at least about 15%, at least about 20%, at least about 25%, at least about 30%, at least about 35%, including any values or sub ranges that lie there between. In some embodiments, the dehydrated microbes comprise a blend of dehydrated microbes with at least about 25% of one or more hygroscopic salt(s). In some embodiments, the dehydrated microbes comprise sucrose and milk solids nonfat. In some embodiments, the dehydrated microbes comprise a controlled release composition coating. In some embodiments, the dehydrated microbes comprise a rapid dissolution formulation of the microbes. In some embodiments, the one or more additives in the water-soluble package may be present in the same compartment as the dehydrated microbes, or in compartments other than the one with the dehydrated microbes. Additional formulations that are compatible for use within the water-soluble films of the present disclosure are provided in US 2022/0106238 and WO 2022/261433, both of which are incorporated by reference in their entireties for all purposes.

a. Buffering Agents

In some embodiments, the one or more compartment(s) of the water-soluble package comprises a buffering agent. In some embodiments, the buffering agent is not in the same compartment as the dehydrated microbes. When the water-soluble packages disintegrate upon contact with liquid, then the buffering agent is released from its compartment, and controls the pH of the dispersion of live microbes. In some embodiments, the buffering agent prevents fluctuations in the pH of the dispersion of live microbes. In some embodiments, the buffering agent prevents toxic levels of acidity or basicity in the dispersion of live microbes.

In some embodiments, the buffering agent maintains the pH of the dispersion of live microbes in the pH range of pH 5-9, pH 5-8, pH 5-7, pH 5-6, pH 6-9, pH 6-8, pH 6-7, pH 7-9, or pH 7-8. In some embodiments, the buffering agent maintains the pH of the dispersion of live microbes in the pH range of pH 6-8.

In some embodiments, the dispersion of live microbes is buffered to the desired pH using conventional buffering agents. Non-limiting examples of buffering agents include sodium citrate, ascorbate, succinate, lactate, citric acid, boric acid, borax, hydrochloric acid, disodium hydrogen phosphate, acetic acid, formic acid, glycine, bicarbonate, phosphate, tartaric acid, Tris-glycine, Tris-NaCl, Tris-ethylenediamine tetraacetic acid (“EDTA”), Tris-borate, Tris-borate-EDTA, Tris-acetate-EDTA (“TAB”), Tris-buffered saline, 4-(2-hydroxyethyl)-1-piperazineethanesulfonic acid (“HEPES”), 3-(N-morpholino) propanesulfonic acid (“MOPS”), piperazine-1,4-bis(2-ethanesulfonic acid) (“PIPES”), 2-(N-morpholino)ethanesulfonic acid (“MES”), and phosphate buffered saline (“PBS”). Table 2 also provides exemplary buffering agents as well as their pKa values and useful pH ranges.

TABLE 2
Exemplary buffering agents

| | pKa at | Useful pH |
|---|---|---|
| Common name and/or chemical name | 25° C. | range |
| ACES | 6.78 | 6.1-7.5 |
| Acetic acid (Ethanoic acid) | 4.8 | 3.8-5.8 |
| ADA | 6.59 | 6.0-7.2 |
| AMP | 9.7 | 9.0-10.5 |
| AMPD | 8.8 | 7.8-9.7 |
| AMPSO | 9 | 8.3-9.7 |
| BES | 7.09 | 6.4-7.8 |
| Bicine (2-(bis(2-hydroxyethyl)amino)acetic | 8.35 | 7.6-9.0 |
| acid) | | |
| Bis-Tris | 6.5 | 5.8-7.2 |
| Bis-Tris Propane | 6.8, 9.0 | 6.3-9.5 |
| Boric acid | 9.24 | 8.25-10.25 |
| CABS | 10.7 | 10.0-11.4 |
| Cacodylate (dimethylarsenic acid) | 6.27 | 5.0-7.4 |
| CAPS | 10.4 | 9.7-11.1 |
| CAPSO | 9.6 | 8.9-10.3 |
| CHES (N-Cyclohexyl-2-aminoethanesulfonic | 9.3 | 8.3-10.3 |
| acid) | | |
| Citric acid (2-Hydroxypropane-1,2,3- | 3.13, | 2.1-7.4 |
| tricarboxylic acid) | 4.76, 6.40 | |
| DIPSO | 7.6 | 7.0-8.2 |
| EPPS | 8 | 7.3-8.7 |
| Gly-Gly | 8.2 | 7.5-8.9 |
| HEPBS | 8.3 | 7.6-9.0 |
| HEPES (4-(2-hydroxyethyl)-1- | 7.48 | 2.5-3.5 or |
| piperazineethanesulfonic acid) | | 6.8-8.2 |
| HEPPSO | 7.8 | 7.1-8.5 |
| KH2PO4 (Monopotassium phosphate) | 7.2 | 6.2-8.2 |
| MES (2-(N-morpholino)ethanesulfonic acid) | 6.15 | 5.5-6.7 |
| MOBS | 7.6 | 6.9-8.3 |
| MOPS (3-(N-morpholino)propanesulfonic | 7.2 | 6.5-7.9 |
| acid) | | |
| MOPSO | 6.9 | 6.2-7.6 |
| PBS or high buffering capacity PBS | | 5.8-8.0 |
| PIPES | 6.76 | 6.1-7.5 |
| PIPES (piperazine-N,N'-bis(2-cthanesulfonic | 6.76 | 6.1-7.5 |
| acid)) | | |
| POPSO | 7.8 | 7.2-8.5 |
| TABS | 8.9 | 8.2-9.6 |
| TAPS ([tris(hydroxymethyl)methylamino] | 8.43 | 7.7-9.1 |
| propanesulfonic acid) | | |
| TAPSO (3-[N-tris(hydroxymethyl) | 7.635 | 7.0-8.2 |
| methylamino]-2-hydroxypropanesulfonic | | |
| acid) | | |
| TEA | 7.8 | 7.3-8.3 |
| TES (2-[1,3-dihydroxy-2-(hydroxymethyl) | 7.4 | 6.8-8.2 |
| propan-2-yl]aminoJethanesulfonic acid) | | |
| Tricine (N-[tris(hydroxymethyl)methyl] | 8.05 | 7.4-8.8 |
| glycine) | | |
| Tris (tris(hydroxymethyl)aminomethane) or | 8.07 | 7.1-9.1 |
| (2-amino-2-(hydroxymethyl)propane-1,3- | | |
| diol) | | |

Additional buffers and instructions on how to prepare them can be found in, e.g., “Common Buffers and Stock Solutions” (2011) Current Protocols in Nucleic Acid Chemistry, A.2A.1-A.2A.14 and in the Sigma Aldrich “Buffer Reference Center” www.sigmaaldrich.com/life-science/core-bioreagents/biological-buffers/learning-center/buffer-reference-center.html, the contents of each of which are incorporated herein in their entirety.

In some embodiments, the buffering agent is one with a high buffering capacity. In some embodiments, the buffering agent is a modified, high buffering capacity version of any one of the buffering agents disclosed herein. In some embodiments, the buffering agent is PBS.

b. Microbial Stabilizers

In some embodiments, the one or more compartment(s) of the water-soluble package comprises microbial stabilizer. In some embodiments, the microbial stabilizer is in the same compartment as the dehydrated microbes. A microbial stabilizer is an agent that acts to stabilize the microorganism population within the agricultural composition. In some embodiments, the microbial stabilizer decreases or slows the decay rate of the microbial population. In some embodiments, the microbial stabilizer accomplishes this change in the decay rate by maintaining the microorganisms in a semi-dormant state. In a semi-dormant state, microorganisms do not respond to environmental conditions as rapidly as they would in an active state.

In some embodiments, the microbial stabilizer improves microbial survival rate, decreases microbial decay, improves microbial metabolic activity, improves microbial catabolic gene expression, improves the microbial colonization rate, or decreases toxin accumulation within the agricultural composition after 1-6 months of storage compared to an agricultural composition without the microbial stabilizer.

In some embodiments, the microbial stabilizer increases the survival rate of microbial cells comprised by the agricultural composition after storage, e.g., after 1, 2, 3, 4, 5, or 6 months of storage. In some embodiments, the log loss of CFU/mL of microbes after the storage period is less than 1. In some embodiments, the log loss is less than 1, 0.9, 0.8, 0.7, 0.6, 0.5, 0.4, 0.3, or 0.2.

In some embodiments, the microbial stabilizer improves the metabolic activity and/or catabolic gene expression of the microorganisms comprised by the agricultural composition after the storage period. In some embodiments, the microbes are more metabolically and/or catabolically active than microbes from the agricultural composition without the microbial stabilizer.

In some embodiments, the microbial stabilizer improves the colonization rate of the microorganisms in the agricultural plant after the storage period compared to the agricultural composition minus the microbial stabilizer. In some embodiments, microbial colonization is unaffected by the storage period for the agricultural composition comprising the microbial stabilizer.

In some embodiments, the microbial stabilizer decreases toxin accumulation. In some embodiments, the toxin is a direct product or byproduct of nitrogen fixation. In some embodiments, the toxin is ammonia or ammonium. In some embodiments, the toxin is produced during cell growth/division.

In some embodiments, the microbial stabilizer decreases toxin accumulation at least two-fold over the target time period, e.g., three months, compared to the agricultural composition without the microbial stabilizer. In some embodiments, the microbial stabilizer decreases toxin accumulation at least two-fold to at least ten-fold compared to a comparable agricultural composition without the microbial stabilizer. In some embodiments, the microbial stabilizer decreases toxin accumulation at least two-fold, at least three-fold, at least four-fold, at least five-fold, at least six-fold, at least seven-fold, at least eight-fold, at least nine-fold, or at least ten-fold. In some embodiments, the microbial stabilizer decreases toxin accumulation about two-fold, about three-fold, about four-fold, about five-fold, about six-fold, about seven-fold, about eight-fold, about nine-fold, or about ten-fold.

In some embodiments, the microbial stabilizer is a sugar. In some embodiments, the microbial stabilizer is a non-reducing sugar. In some embodiments, the microbial stabilizer is a monosaccharide. Monosaccharides suitable for use include, but are not limited to, glucose and fructose. In some embodiments, the microbial stabilizer is fructose. In some embodiments, the microbial stabilizer is a disaccharide. Monosaccharides suitable for use include, but are not limited to, trehalose, sucrose, lactose, melibiose, and lactulose. In some embodiments, the microbial stabilizer is trehalose. In some embodiments, the microbial stabilizer is a polysaccharide. Polysaccharides suitable for use include, but are not limited to, maltodextrin, microcrystalline cellulose, and dextran. Additional carbohydrates suitable for use as microbial stabilizers within the agricultural compositions in the water-soluble packages of the present disclosure include, but are not limited to, pentoses (e.g., ribose, xylose), hexoses (e.g., mannose, sorbose), oligosaccharides (e.g., raffinose), and oligofructoses. In some embodiments, the microbial stabilizer is a sugar alcohol. Sugar alcohols suitable for use include, but are not limited to, glycerol, mannitol, and sorbitol.

In some embodiments, the microbial stabilizer is an amino acid. In some embodiments, the microbial stabilizer is glycine, proline, glutamate, or cysteine. In some embodiments, the microbial stabilizer is a protein or protein hydrolysate. Proteins or protein hydrolysates suitable for use as microbial stabilizers within the agricultural composition of the present disclosure include, but are not limited to, malt extract, milk powder, casein, whey powder, and yeast extract. In some embodiments, the microbial stabilizer is skimmed milk, starch, humic acid, chitosan, CMC, corn steep liquor, molasses, paraffin, pinolene, NFSM, MgSO₄, liquid growth medium, horse serum, or Ficoll.

In some embodiments, the microbial stabilizer is a desiccant. As used herein, a “desiccant” can include any compound or mixture of compounds that can be classified as a desiccant regardless of whether the compound or compounds are used in such concentrations that they in fact have a desiccating effect on the liquid inoculant. Such desiccants are ideally compatible with the microbial population used, and should promote the ability of the microbial population to survive application on the agricultural plant tissues or the environs thereof and to survive desiccation. Examples of suitable desiccants include one or more of trehalose, sucrose, glycerol, and methylene glycol. Other suitable desiccants include, but are not limited to, non-reducing sugars and sugar alcohols (e.g., mannitol or sorbitol).

In some embodiments, the microbial stabilizer comprised by the agricultural composition also acts as a physical stabilizer. In some embodiments, the substance acting as a microbial stabilizer within the agricultural composition has properties of a thickening agent and therefore also acts as a physical stabilizer. In some embodiments, an agricultural composition of the present disclosure comprising both a physical and a microbial stabilizer does so by comprising the same agent that has characteristics of both types of stabilizer.

In some embodiments, the concentration of microbial stabilizer in the dispersion of live microbes is in the range from about 0.1% w/v to about 20% w/v. In some embodiments, the concentration of microbial stabilizer in the dispersion of live microbes is in the range of 0.1-1.0% w/v, 1.0-5.0% w/v, 5.0-10% w/v, or 10-20% w/v. In some embodiments, the microbial stabilizer is present in the dispersion of live microbes at a concentration of about 0.5-10% w/v. In some embodiments, the microbial stabilizer is present in the dispersion of live microbes at a concentration of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0% w/v. In some embodiments, the microbial stabilizer is present at a concentration in the dispersion of live microbes of about 1.0% w/v. In some embodiments, the microbial stabilizer is present in the dispersion of live microbes at a concentration of about 1.3% w/v. In some embodiments, the microbial stabilizer is present in the dispersion of live microbes at a concentration of about 2.5% w/v.

c. Physical Stabilizers

In some embodiments, the one or more compartment(s) of the water-soluble package comprises a physical stabilizer. As used herein, a “physical stabilizer” refers to a substance that improves the homogeneity of the agricultural composition, such that the microbial cells are at a similar density throughout the liquid composition. By increasing homogeneity, the physical stabilizer prevents high concentrations of cells and/or toxins from accumulating in any one sub-volume of the dispersion of live microbes.

In some embodiments, the physical stabilizer increases the viscosity of the dispersion of live microbes. In some embodiments, the physical stabilizer is a thickening agent. In some embodiments, the physical stabilizer is an anti-settling agent. In some embodiments, the physical stabilizer is a suspension aid. In some embodiments, the physical stabilizer acts to maintain microbial cells in suspension, improving the cell's resistance to settle statically and flow under shear or rheological shear-thinning. In some embodiments, a physical stabilizer may also have properties of a microbial stabilizer and vice versa.

In some embodiments, the physical stabilizer is a polysaccharide. Polysaccharides suitable for use as physical stabilizers include, but are not limited to, polyethylene glycol (PEG), xanthan gum, pectin, and alginates. In some embodiments, the physical stabilizer is xanthan gum. In some embodiments, the physical stabilizer is a protein or protein hydrolysate. Proteins or protein hydrolysates suitable for use as physical stabilizers include, but are not limited to, gluten, collagen, gelatin, elastin, keratin, and albumin. In some embodiments, the physical stabilizer is a polymer. Polymers suitable for use as physical stabilizers include, but are not limited to, Carbopol® (CBP) polymers, methylene glycol, polyvinyl alcohol (PVA), polyvinylpyrrolidone (PVP), polyacrylate, hydroxyethyl cellulose, or hydroxypropyl methylcellulose. In some embodiments, the physical stabilizer is a gum or its derivative. Gums and their derivatives suitable for use as physical stabilizers include, but are not limited to, guar gum, gum Arabic, gum tragacanth, xanthan gum, derivitized guar, hydroxypropyl guar, and polysaccharide gums. In some embodiments, the physical stabilizer is a CBP polymer.

In some embodiments, the physical stabilizer is a suspension aid. Suitable suspension aids for use as physical stabilizers include, but are not limited to, water-soluble polymers such as acrylamide homo- and copolymers, acrylic acid homo- and copolymer, cellulose, methyl cellulose, ethyl cellulose, carboxymethyl cellulose (sodium and other salts), carboxymethyl hydroxyethyl cellulose, hydroxyethyl cellulose, hydrophobically modified hydroxyethyl cellulose, hydroxypropyl cellulose, hydroxypropyl methylcellulose, water-soluble cellulose ethers, carboxy-vinyl copolymers, alginic acid, polyacrylic acid, sodium polyacrylate, partially and fully hydrolyzed polyvinyl alcohols, partially neutralized polyacrylic acid, polyalkylene glycol, polyvinylpyrrolidone and derivatives, starch and its derivatives, vinylpyrrolidone homo- and copolymers, polyacrylamide, attapulgite, montmorillonite, organically modified montmorillonite clays, alumina, precipitated silica, or any mixture thereof.

In some embodiments, the concentration of physical stabilizer in the dispersion of live microbes ranges from about 0.01% w/v to about 30% w/v. In some embodiments, the concentration of physical stabilizer in the dispersion of live microbes is in the range of 0.01-0.1% w/v, 0.1-1.0% w/v, 1.0-5.0% w/v, 5.0-10% w/v, 10-15%, 15-20%, 20-25%, or 25-30% w/v. In some embodiments, the physical stabilizer is present in the dispersion of live microbes at a concentration of about 0.01-2.0% w/v. In some embodiments, the physical stabilizer is present in the dispersion of live microbes at a concentration of about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0% w/v. In some embodiments, the physical stabilizer is present in the dispersion of live microbes at a concentration of about 0.1% w/v. In some embodiments, the physical stabilizer in the dispersion of live microbes is present at a concentration of about 0.2% w/v.

d. Additional Agricultural Composition Components

In some embodiments, the one or more compartment(s) of the water-soluble package comprises additional components. These additional components may include protectants and beneficial ingredients including but not limited to animal and bird repellants, attractants, baits, herbicides, herbicide safeners, antidessicants, antitranspirants, frost prevention aids, inoculants, dyes, brighteners, markers, synergists, pigments, UV protectants, antioxidants, leaf polish, pigmentation stimulants and inhibitors, surfactants, moisture retention aids, humic acids and humates, lignins and lignates, bitter flavors, irritants, malodorous ingredients, molluscicides (e.g., slugs and snails), nematicides, rodenticides, defoliants, desiccants, sticky traps, IPM (integrated pest management) lures, chemosterilants, plant defense boosters (harpin protein and chitosan), and other beneficial or detrimental agents applied to the surface of the plant tissue or the environs thereof. In some embodiments, multiple active agents are readily formulated within a given agricultural composition, for example, multiple active agents may include two or more of any of the following fungicides, fertilizers, pesticides, herbicides, and any type of active ingredient or class of active ingredient.

Suitable additional ingredients for the agricultural compositions in the water-soluble packages of the present disclosure include, but are not limited to, the following:

Insecticides: A1) the class of carbamates consisting of aldicarb, alanycarb, benfuracarb, carbaryl, carbofuran, carbosulfan, methiocarb, methomyl, oxamyl, pirimicarb, propoxur and thiodicarb; A2) the class of organophosphates consisting of acephate, azinphos-ethyl, azinphos-methyl, chlorfenvinphos, chlorpyrifos, chlorpyrifos-methyl, demeton-S-methyl, diazinon, dichlorvos/DDVP, dicrotophos, dimethoate, disulfoton, ethion, fenitrothion, fenthion, isoxathion, malathion, methamidophos, methidathion, mevinphos, monocrotophos, oxymethoate, oxydemeton-methyl, parathion, parathion-methyl, phenthoate, phorate, phosalone, phosmet, phosphamidon, pirimiphos-methyl, quinalphos, terbufos, tetrachlorvinphos, triazophos and trichlorfon; A3) the class of cyclodiene organochlorine compounds such as endosulfan; A4) the class of fiproles consisting of ethiprole, fipronil, pyrafluprole and pyriprole; A5) the class of neonicotinoids consisting of acetamiprid, chlothianidin, dinotefuran, imidacloprid, nitenpyram, thiacloprid and thiamethoxam; A6) the class of spinosyns such as spinosad and spinetoram; A7) chloride channel activators from the class of mectins consisting of abamectin, emamectin benzoate, ivermectin, lepimectin and milbemectin; A8) juvenile hormone mimics such as hydroprene, kinoprene, methoprene, fenoxycarb and pyriproxyfen; A9) selective homopteran feeding blockers such as pymetrozine, flonicamid and pyrifluquinazon; A 10) mite growth inhibitors such as clofentezine, hexythiazox and etoxazole; A11) inhibitors of mitochondrial ATP synthase such as diafenthiuron, fenbutatin oxide and propargite; uncouplers of oxidative phosphorylation such as chlorfenapyr; A12) nicotinic acetylcholine receptor channel blockers such as bensultap, cartap hydrochloride, thiocyclam and thiosultap sodium; A13) inhibitors of the chitin biosynthesis type 0 from the benzoylurea class consisting of bistrifluron, diflubenzuron, flufenoxuron, hexaflumuron, lufenuron, novaluron and teflubenzuron; A14) inhibitors of the chitin biosynthesis type 1 such as buprofezin; A15) moulting disruptors such as cyromazine; A16) ecdyson receptor agonists such as methoxyfenozide, tebufenozide, halofenozide and chromafenozide; A17) octopamin receptor agonists such as amitraz; A18) mitochondrial complex electron transport inhibitors pyridaben, tebufenpyrad, tolfenpyrad, flufenerim, cyenopyrafen, cyflumetofen, hydramethylnon, acequinocyl or fluacrypyrim; A19) voltage-dependent sodium channel blockers such as indoxacarb and metaflumizone; A20) inhibitors of the lipid synthesis such as spirodiclofen, spiromesifen and spirotetramat; A21) ryanodine receptor-modulators from the class of diamides consisting of flubendiamide, the phthalamide compounds (R)-3-Chlor-N1-{2-methyl-4-[1,2,2,2-tetrafluor-1-(trifluormethyl)ethyl]phenyl}-N2-(1-methyl-2-methyl sulfonylethyl)phthalamid and (S)-3-Chlor-N1-{2-methyl-4-[1,2,2,2-tetrafluor-1-(trifluormethyl)ethyl]phenyl}-N2-(1-methyl-2-methylsulfonylethyl)phthalamid, chlorantraniliprole and cyantraniliprole; A22) compounds of unknown or uncertain mode of action such as azadirachtin, amidoflumet, bifenazate, fluensulfone, piperonyl butoxide, pyridalyl, sulfoxaflor; or A23) sodium channel modulators from the class of pyrethroids consisting of acrinathrin, allethrin, bifenthrin, cyfluthrin, gamma-cyhalothrin, lambda-cyhalothrin, cypermethrin, alpha-cypermethrin, beta-cypermethrin, zeta-cypermethrin, deltamethrin, esfenvalerate, etofenprox, fenpropathrin, fenvalerate, flucythrinate, tau-fluvalinate, permethrin, silafluofen, tefluthrin and tralomethrin and any suitable combinations thereof.

Fungicides: B1) azoles selected from the group consisting of bitertanol, bromuconazole, cyproconazole, difenoconazole, diniconazole, enilconazole, epoxiconazole, fluquinconazole, fenbuconazole, flusilazole, flutriafol, hexaconazole, imibenconazole, ipconazole, metconazole, myclobutanil, penconazole, propiconazole, prothioconazole, simeconazole, triadimefon, triadimenol, tebuconazole, tetraconazole, triticonazole, prochloraz, pefurazoate, imazalil, triflumizole, cyazofamid, benomyl, carbendazim, thiabendazole, fuberidazole, ethaboxam, etridiazole and hymexazole, azaconazole, diniconazole-M, oxpoconazol, paclobutrazol, uniconazol, 1-(4-chloro-phenyl)-2-([1,2,4]triazol-1-yl)-cycloheptanol and imazalilsulfphate; B2) strobilurins selected from the group consisting of azoxystrobin, dimoxystrobin, enestroburin, fluoxastrobin, kresoxim-methyl, metominostrobin, orysastrobin, picoxystrobin, pyraclostrobin, trifloxystrobin, methyl (2-chloro-5-[1-(3-methylbenzyloxyimino)ethyl]benzyl)carbamate, methyl (2-chloro-5-[1-(6-methylpyridin-2-ylmethoxyimino)ethyl]benzyl)carbamate and methyl 2-(ortho-(2,5-dimethylphenyloxymethylene)-phenyl)-3-methoxyacrylate, 2-(2-(6-(3-chloro-2-methyl-phenoxy)-5-fluoro-pyrimidin-4-yloxy)-phenyl)-2-methoxyimino-N-methyl-acetamide and 3-methoxy-2-(2-(N-(4-methoxy-phenyl)-cyclopropanecarboximidoylsulfanylmethyl)-phenyl)-acrylic acid methyl ester; B3) carboxamides selected from the group consisting of carboxin, benalaxyl, benalaxyl-M, fenhexamid, flutolanil, furametpyr, mepronil, metalaxyl, mefenoxam, ofurace, oxadixyl, oxycarboxin, penthiopyrad, isopyrazam, thifluzamide, tiadinil, 3,4-dichloro-N-(2-cyanophenyl)isothiazole-5-carboxamide, dimethomorph, flumorph, flumetover, fluopicolide (picobenzamid), zoxamide, carpropamid, diclocymet, mandipropamid, N-(2-(443-(4-chlorophenyl)prop-2-ynyloxy]-3-methoxyphenyl)ethyl)-2-methanesulfonyl-amino-3-methylbutyramide, N-(2-(4-[3-(4-chloro-phenyl)prop-2-ynyloxy]-3-methoxy-phenyl)ethyl)-2-ethanesulfonylamino-3-methylbutyramide, methyl 3-(4-chlorophenyl)-3-(2-isopropoxycarbonyl-amino-3-methyl-butyrylamino)propionate, N-(4′-bromobiphenyl-2-yl)-4-difluoromethylA-methylthiazole-6-carboxamide, N-(4′-trifluoromethyl-biphenyl-2-yl)-4-difluoromethyl-2-methylthiazole-5-carboxamide, N-(4′-chloro-3′-fluorobiphenyl-2-yl)-4-difluoromethyl-2-methyl-thiazole-5-carboxamide, N-(3′,4′-dichloro-4-fluorobiphenyl-2-yl)-3-difluoro-methyl-1-methyl-pyrazole-4-carboxamide, N-(3′,4′-dichloro-5-fluorobiphenyl-2-yl)-3-difluoromethyl-1-methylpyrazole-4-carboxamide, N-(2-cyano-phenyl)-3,4-dichloroisothiazole-5-carboxamide, 2-amino-4-methyl-thiazole-5-carboxanilide, 2-chloro-N-(1,1,3-trimethyl-indan-4-yl)-nicotinamide, N-(2-(1,3-dimethylbutyl)-phenyl)-1,3-dimethyl-5-fluoro-1H-pyrazole-4-carboxamide, N-(4′-chloro-3′,5-difluoro-biphenyl-2-yl)-3-difluoromethyl-1-methyl-1H-pyrazole-4-carboxamide, N-(4′-chloro-3′, 5-difluoro-biphenyl-2-yl)-3-trifluoromethyl-1-methyl-1H-pyrazole-4-carboxamide, N-(3′, 4′-dichloro-5-fluoro-biphenyl-2-yl)-3-trifluoromethyl-1-methyl-1H-pyrazole-4-carboxamide, N-(3′, 5-difluoro-4′-methyl-biphenyl-2-yl)-3-difluoromethyl-1-methyl-1H-pyrazole-4-carboxamide, N-(3′, 5-difluoro-4′-methyl-biphenyl-2-yl)-3-trifluoromethyl-1-methyl-1H-pyrazole-4-carboxamide, N-(cis-2-bicyclopropyl-2-yl-phenyl)-3-difluoromethyl-1-methyl-1H-pyrazole-4-carboxamide, N-(trans-2-bicyclopropyl-2-yl-phenyl)-3-difluoro-methyl-1-methyl-1H-pyrazole-4-carboxamide, fluopyram, N-(3-ethyl-3,5-5-trimethyl-cyclohexyl)-3-formylamino-2-hydroxy-benzamide, oxytetracyclin, silthiofam, N-(6-methoxy-pyridin-3-yl) cyclopropanecarboxamide, 2-iodo-N-phenyl-benzamide, N-(2-bicyclo-propyl-2-yl-phenyl)-3-difluoromethyl-1-methylpyrazol-4-ylcarboxamide, N-(3′,4′,5′-trifluorobiphenyl-2-yl)-1,3-dimethylpyrazol-4-ylcarboxamide, N-(3′,4′,5′-trifluorobiphenyl-2-yl)-1,3-dimethyl-5-fluoropyrazol-4-yl-carboxamide, N-(3′,4′,5′-trifluorobiphenyl-2-yl)-5-chloro-1,3-dimethyl-pyrazol-4-ylcarboxamide, N-(3′,4′,5′-trifluorobiphenyl-2-yl)-3-fluoromethyl-1-methylpyrazol-4-ylcarboxamide, N-(3′,4′,5′-trifluorobiphenyl-2-yl)-3-(chlorofluoromethyl)-1-methylpyrazol-4-ylcarboxamide, N-(3′,4′,5′-trifluorobiphenyl-2-yl)-3-difluoromethyl-1-methylpyrazol-4-ylcarboxamide, N-(3′,4′,5′-trifluorobiphenyl-2-yl)-3-difluoromethyl-5-fluoro-1-methylpyrazol-4-ylcarboxamide, N-(3′,4′,5′-trifluorobiphenyl-2-yl)-5-chloro-3-difluoromethyl-1-methylpyrazol-4-ylcarboxamide, N-(3′,4′,5′-trifluorobiphenyl-2-yl)-3-(chlorodifluoromethyl)-1-methylpyrazol-4-ylcarboxamide, N-(3′,4′,5′-trifluorobiphenyl-2-yl)-1-methyl-3-trifluoromethylpyrazol-4-ylcarboxamide, N-(3′,4′,5′-trifluorobiphenyl-2-yl)-5-fluoro-1-methyl-3-trifluoromethylpyrazol-4-ylcarboxamide, N-(3′,4′,5′-trifluorobiphenyl-2-yl)-5-chloro-1-methyl-3-trifluoromethylpyrazol-4-ylcarboxamide, N-(2′,4′,5′-trifluorobiphenyl-2-yl)-1,3-dimethylpyrazol-4-ylcarboxamide, N-(2′,4′,5′-trifluorobiphenyl-2-yl)-1,3-dimethyl-5-fluoropyrazol-4-ylcarboxamide, N-(2′,4′,5′-trifluorobiphenyl-2-yl)-5-chloro-1,3-dimethylpyrazol-4-ylcarboxamide, N-(2′,4′,5′-trifluorobiphenyl-2-yl)-3-fluoromethyl-1-methylpyrazol-4-ylcarboxamide, N-(2′,4′,5′-trifluorobiphenyl-2-yl)-3-(chlorofluoromethyl)-1-methylpyrazol-4-ylcarboxamide, N-(2′,4′,5′-trifluorobiphenyl-2-yl)-3-difluoromethyl-1-methylpyrazol-4-ylcarboxamide, N-(2′,4′,5′-trifluorobiphenyl-2-yl)-3-difluoromethyl-5-fluoro-1-methylpyrazol-4-ylcarboxamide, N-(2′,4′,5′-trifluorobiphenyl-2-yl)-5-chloro-3-difluoromethyl-1-methylpyrazol-4-ylcarboxamide, N-(2′,4′,5′-trifluorobiphenyl-2-yl)-3-(chlorodifluoromethyl)-1-methylpyrazol-4-ylcarboxamide, N-(2′,4′,5′-trifluorobiphenyl-2-yl)-1-methyl-3-trifluoromethylpyrazol-4-ylcarboxamide, N-(2′,4′,5′-trifluorobiphenyl-2-yl)-5-fluoro-1-methyl-3-trifluoromethylpyrazol-4-ylcarboxamide, N-(2′,4′,5′-trifluorobiphenyl-2-yl)-5-chloro-1-methyl-3-trifluoromethylpyrazol-4-ylcarboxamide, N-(3′,4′-dichloro-3-fluorobiphenyl-2-yl)-1-methyl-3-trifluoromethyl-1H-pyrazole-4-carboxamide, N-(3′,4′-dichloro-3-fluorobiphenyl-2-yl)-1-methyl-3-difluoromethyl-1H-pyrazole-4-carboxamide, N-(3′,4′-difluoro-3-fluorobiphenyl-2-yl)-1-methyl-3-trifluoromethyl-1H-pyrazole-4-carboxamide, N-(3′,4′-difluoro-3-fluorobiphenyl-2-yl)-1-methyl-S-difluoromethyl-1H-pyrazole-4-carboxamide, N-(3′-chloro-4′-fluoro-3-fluorobiphenyl-2-yl)-1-methyl-3-difluoromethyl-1H-pyrazole-4-carboxamide, N-(3′,4′-dichloro-4-fluorobiphenyl-2-yl)-1-methyl-3-trifluoromethyl-1H-pyrazole-4-carboxamide, N-(3′,4′-difluoro-4-fluorobiphenyl-2-yl)-1-methyl-S-trifluoromethyl-1H-pyrazole-4-carboxamide, N-(3′,4′-dichloro-4-fluorobiphenyl-2-yl)-1-methyl-3-difluoromethyl-1H-pyrazole-4-carboxamide, N-(3′,4′-difluoro-4-fluorobiphenyl-2-yl)-1-methyl-3-difluoromethyl-1H-pyrazole-4-carboxamide, N-(3′-chloro-4′-fluoro-4-fluorobiphenyl-2-yl)-1-methyl-S-difluoromethyl-1H-pyrazole-4-carboxamide, N-(3′,4′-dichloro-5-fluorobiphenyl-2-yl)-1-methyl-3-trifluoromethyl-1H-pyrazole-4-carboxamide, N-(3′,4′-difluoro-5-fluorobiphenyl-2-yl)-1-methyl-3-trifluoromethyl-1H-pyrazole-4-carboxamide, N-(3′,4′-dichloro-5-fluorobiphenyl-2-yl)-1-methyl-S-difluoromethyl-1H-pyrazole-carboxamide, N-(3′,4′-difluoro-5-fluorobiphenyl-2-yl)-1-methyl-3-difluoromethyl-1H-pyrazole-4-carboxamide, N-(3′,4′-dichloro-5-fluorobiphenyl-2-yl)-1,3-dimethyl-1H-pyrazole-4-carboxamide, N-(3′-chloro-4′-fluoro-5-fluorobiphenyl-2-yl)-1-methyl-3-difluoromethyl-1H-pyrazole-4-carboxamide, N-(4′-fluoro-4-fluorobiphenyl-2-yl)-1-methyl-3-trifluoromethyl-1H-pyrazole-4-carboxamide, N-(4′-fluoro-5-fluorobiphenyl-2-yl)-1-methyl-3-trifluoromethyl-1H-pyrazole-4-carboxamide, N-(4′-chloro-5-fluorobiphenyl-2-yl)-1-methyl-3-trifluoromethyl-1H-pyrazole-4-carboxamide, N-(4′-methyl-5-fluorobiphenyl-2-yl)-1-methyl-3-trifluoromethyl-1H-pyrazole-4-carboxamide, N-(4′-fluoro-5-fluorobiphenyl-2-yl)-1,3-dimethyl-1H-pyrazole-4-carboxamide, N-(4′-chloro-5-fluorobiphenyl-2-yl)-1,3-dimethyl-1H-pyrazole-4-carboxamide, N-(4′-methyl-5-fluorobiphenyl-2-yl)-1,3-dimethyl-1H-pyrazole-4-carboxamide, N-(4′-fluoro-6-fluorobiphenyl-2-yl)-1-methyl-3-trifluoromethyl-1H-pyrazole-4-carboxamide, N-(4′-chloro-6-fluorobiphenyl-2-yl)-1-methyl-3-trifluoromethyl-1H-pyrazole-4-carboxamide, N-[2-(1,1,2,3,3,3-hexafluoropropoxy)-phenyl]-3-difluoromethyl-1-methyl-1H-pyrazole-4-carboxamide, N-[4′-(trifluoromethylthio)-biphenyl-2-yl]-3-difluoromethyl-1-methyl-1H-pyrazole-4-carboxamide and N44′-(trifluoromethylthio)-biphenyl-2-yl]-1-methyl-3-trifluoromethyl-1-methyl-1H-pyrazole-4-carboxamide; B4) heterocyclic compounds selected from the group consisting of fluazinam, pyrifenox, bupirimate, cyprodinil, fenarimol, ferimzone, mepanipyrim, nuarimol, pyrimethanil, triforine, fenpiclonil, fludioxonil, aldimorph, dodemorph, fenpropimorph, tridemorph, fenpropidin, iprodione, procymidone, vinclozolin, famoxadone, fenamidone, octhilinone, proben-azole, 5-chloro-7-(4-methyl-piperidin-1-yl)-6-(2,4,6-trifluorophenyl)41,2,4]triazolo[1,5-a]pyrimidine, anilazine, diclomezine, pyroquilon, proquinazid, tricyclazole, 2-butoxy-6-iodo-3-propylchromen-4-one, acibenzolar-S-methyl, captafol, captan, dazomet, folpet, fenoxanil, quinoxyfen, N,N-dimethyl-3-(3-bromo-6-fluoro-2-methylindole-1-sulfonyl)-[1,2,4]triazole-1-sulfonamide, 5-ethyl-6-octyl41,2,4]triazolo[1 2,3,5,6-tetrachloro-4-methanesulfonyl-pyridine, 3,4,5-trichloro-pyridine-2,6-di-carbonitrile, N-(1-(5-bromo-3-chloro-pyridin-2-yl)-ethyl)-2,4-dichloro-nicotinamide, N-((5-bromo-3-chloro pyridin-2-yl)-methyl)-2,4-dichloro-nicotinamide, diflumetorim, nitrapyrin, dodemorphacetate, fluoroimid, blasticidin-S, chinomethionat, debacarb, difenzoquat, difenzoquat-methylsulphat, oxolinic acid and piperalin; B5) carbamates selected from the group consisting of mancozeb, maneb, metam, methasulphocarb, metiram, ferbam, propineb, thiram, zineb, ziram, diethofencarb, iprovalicarb, benthiavalicarb, propamocarb, propamocarb hydrochlorid, 4-fluorophenyl N-(1-(1-(4-cyanophenyl)-ethanesulfonyl)but-2-yl)carbamate, methyl 3-(4-chloro-phenyl)-3-(2-isopropoxycarbonylamino-3-methyl-butyrylamino)propanoate; or B6) other fungicides selected from the group consisting of guanidine, dodine, dodine free base, iminoctadine, guazatine, antibiotics: kasugamycin, streptomycin, polyoxin, validamycin A, nitrophenyl derivatives: binapacryl, dinocap, dinobuton, sulfur-containing heterocyclyl compounds: dithianon, isoprothiolane, organometallic compounds: fentin salts, organophosphorus compounds: edifenphos, iprobenfos, fosetyl, fosetyl-aluminum, phosphorous acid and its salts, pyrazophos, tolclofos-methyl, organochlorine compounds: dichlofluanid, flusulfamide, hexachloro-benzene, phthalide, pencycuron, quintozene, thiophanate-methyl, tolylfluanid, others: cyflufenamid, cymoxanil, dimethirimol, ethirimol, furalaxyl, metrafenone and spiroxamine, guazatine-acetate, iminoc-tadine-triacetate, iminoctadine-tris(albesilate), kasugamycin hydrochloride hydrate, dichlorophen, pentachlorophenol and its salts, N-(4-chloro-2-nitro-phenyl)-N-ethyl-4-methyl-benzenesulfonamide, did nitrothal-isopropyl, tecnazen, biphenyl, bronopol, diphenylamine, mildiomycin, oxincopper, prohexadione calcium, N-(cyclopropylmethoxyimino-(6-difluoromethoxy-2,3-difluoro-phenyl)-methyl)-2-phenyl acetamide, N′-(4-(4-chloro-3-trifluoromethyl-phenoxy)-2,5-dimethyl-phenyl)-N-ethyl-N-methyl formamidine, N′-(4-(4-fluoro-3-trifluoromethyl-phenoxy)-2,5-dimethyl-phenyl)-N-ethyl-N-methyl formamidine, N′-(2-methyl-5-trifluormethyl-4-(3-trimethylsilanyl-propoxy)-phenyl)-N-ethyl-N-methylformamidine and N′-(5-difluoromethyl-2-methyl-4-(3-trimethylsilanyl-propoxy)-phenyl)-N-ethyl-N-methyl formamidine, and any combinations thereof.

Herbicides: C1) acetyl-CoA carboxylase inhibitors (ACC), for example cyclohexenone oxime ethers, such as alloxydim, clethodim, cloproxydim, cycloxydim, sethoxydim, tralkoxydim, butroxydim, clefoxydim or tepraloxydim; phenoxyphenoxypropionic esters, such as clodinafop-propargyl, cyhalofop-butyl, diclofop-methyl, fenoxaprop-ethyl, fenoxaprop-P-ethyl, fenthiapropethyl, fluazifop-butyl, fluazifop-P-butyl, haloxyfop-ethoxyethyl, haloxyfop-methyl, haloxyfop-P-methyl, isoxapyrifop, propaquizafop, quizalofop-ethyl, quizalofop-P-ethyl or quizalofop-tefuryl; or arylaminopropionic acids, such as flamprop-methyl or flamprop-isopropyl; C2 acetolactate synthase inhibitors (ALS), for example imidazolinones, such as imazapyr, imazaquin, imazamethabenz-methyl (imazame), imazamox, imazapic or imazethapyr; pyrimidyl ethers, such as pyrithiobac-acid, pyrithiobac-sodium, bispyribac-sodium. KIH-6127 or pyribenzoxym; sulfonamides, such as florasulam, flumetsulam or metosulam; or sulfonylureas, such as amidosulfuron, azimsulfuron, bensulfuron-methyl, chlorimuron-ethyl, chlorsulfuron, cinosulfuron, cyclosulfamuron, ethametsulfuron-methyl, ethoxysulfuron, flazasulfuron, halosulfuron-methyl, imazosulfuron, metsulfuron-methyl, nicosulfuron, primisulfuron-methyl, prosulfuron, pyrazosulfuron-ethyl, rimsulfuron, sulfometuron-methyl, thifensulfuron-methyl, triasulfuron, tribenuron-methyl, triflusulfuron-methyl, tritosulfuron, sulfosulfuron, foramsulfuron or iodosulfuron; C3) amides, for example allidochlor (CDAA), benzoylprop-ethyl, bromobutide, chiorthiamid. diphenamid, etobenzanidibenzchlomet), fluthiamide, fosamin or monalide; C4) auxin herbicides, for example pyridinecarboxylic acids, such as clopyralid or picloram; or 2,4-D or benazolin; C5) auxin transport inhibitors, for example naptalame or diflufenzopyr; C6) carotenoid biosynthesis inhibitors, for example benzofenap, clomazone (dimethazone), diflufenican, fluorochloridone, fluridone, pyrazolynate, pyrazoxyfen, isoxaflutole, isoxachlortole, mesotrione, sulcotrione (chlormesulone), ketospiradox, flurtamone, norflurazon or amitrol; C7) enolpyruvylshikimate-3-phosphate synthase inhibitors (EPSPS), for example glyphosate or sulfosate; C8) glutamine synthetase inhibitors, for example bilanafos (bialaphos) or glufosinate-ammonium; C9) lipid biosynthesis inhibitors, for example anilides, such as anilofos or mefenacet; chloroacetanilides, such as dimethenamid, S-dimethenamid, acetochlor, alachlor, butachlor, butenachlor, diethatyl-ethyl, dimethachlor, metazachlor, metolachlor, S-metolachlor, pretilachlor, propachlor, prynachlor, terbuchlor, thenylchlor or xylachlor; thioureas, such as butylate, cycloate, di-allate, dimepiperate, EPTC. esprocarb, molinate, pebulate, prosulfocarb, thiobencarb (benthiocarb), tri-allate or vemolate; or benfuresate or perfluidone; C10) mitosis inhibitors, for example carbamates, such as asulam, carbetamid, chlorpropham, orbencarb, pronamid (propyzamid), propham or tiocarbazil; dinitroanilines, such as benefin, butralin, dinitramin, ethalfluralin, fluchloralin, oryzalin, pendimethalin, prodiamine or trifluralin; pyridines, such as dithiopyr or thiazopyr; or butamifos, chlorthal-dimethyl (DCPA) or maleic hydrazide; C11) protoporphyrinogen IX oxidase inhibitors, for example diphenyl ethers, such as acifluorfen, acifluorfen-sodium, aclonifen, bifenox, chlomitrofen (CNP), ethoxyfen, fluorodifen, fluoroglycofen-ethyl, fomesafen, furyloxyfen, lactofen, nitrofen, nitrofluorfen or oxyfluorfen; oxadiazoles, such as oxadiargyl or oxadiazon; cyclic imides, such as azafenidin, butafenacil, carfentrazone-ethyl, cinidon-ethyl, flumiclorac-pentyl, flumioxazin, flumipropyn, flupropacil, fluthiacet-methyl, sulfentrazone or thidiazimin; or pyrazoles, such as ET-751.JV 485 or nipyraclofen; C12) photosynthesis inhibitors, for example propanil, pyridate or pyridafol; benzothiadiazinones, such as bentazone; dinitrophenols, for example bromofenoxim, dinoseb, dinoseb-acetate, dinoterb or DNOC; dipyridylenes, such as cyperquat-chloride, difenzoquat-methyl sulfate, diquat or paraquat-dichloride; ureas, such as chlorbromuron, chlorotoluron, difenoxuron, dimefuron, diuron, ethidimuron, fenuron, fluometuron, isoproturonisouron, linuron, methabenzthiazuron, methazole, metobenzuron, metoxuron, monolinuron, neburon, siduron or tebuthiuron; phenols, such as bromoxynil or ioxynil; chloridazon; triazines, such as ametryn, atrazine, cyanazine, desmein, dimethamethryn, hexazinone, prometon, prometryn, propazine, simazine, simetryn, terbumeton, terbutryn, terbutylazine or trietazine; triazinones, such as metamitron or metribuzin; uracils, such as bromacil, lenacil or terbacil; or biscarbamates, such as desmedipham or phenmedipham; C13) synergists, for example oxiranes, such as tridiphane; C14) CIS cell wall synthesis inhibitors, for example isoxaben or dichlobenil; C16) various other herbicides, for example dichloropropionic acids, such as dalapon; dihydrobenzofurans, such as ethofumesate; phenylacetic acids, such as chlorfenac (fenac); or aziprotryn, barban, bensulide, benzthiazuron, benzofluor, buminafos, buthidazole, buturon, cafenstrole, chlorbufam, chlorfenprop-methyl, chloroxuron, cinmethylin, cumyluron, cycluron, cyprazine, cyprazole, dibenzyluron, dipropetryn, dymron, eglinazin-ethyl, endothall, ethiozin, flucabazone, fluorbentranil, flupoxam, isocarbamid, isopropalin, karbutilate, mefluidide, monuron, napropamide, napropanilide, nitralin, oxaciclomefone, phenisopham, piperophos, procyazine, profluralin, pyributicarb, secbumeton, sulfallate (CDEC), terbucarb, triaziflam, triazofenamid or trimeturon; or their environmentally compatible salts or combinations thereof.

Nematicides: Benomyl, cloethocarb, aldoxycarb, tirpate, diamidafos, fenamiphos, cadusafos, dichlofenthion, ethoprophos, fensulfothion, fosthiazate, heterophos, isamidofof, isazofos, phosphocarb, thionazin, imicyafos, mecarphon, acetoprole, benclothiaz, chloropicrin, dazomet, fluensulfone, oxamyl, terbufos and suitable combinations thereof.

Plant Growth Regulators or Hormones: D1) Antiauxins, such as clofibric acid, 2,3,5-triiodobenzoic acid; D2) Auxins such as 4-CPA, 2,4-D, 2,4-DB, 2,4-DEP, dichlorprop, fenoprop, IAA, IBA, naphthaleneacetamide, α-naphthaleneacetic acids, 1-naphthol, naphthoxyacetic acids, potassium naphthenate, sodium naphthenate, 2,4,5-T; D3) cytokinins, such as 21P, benzyladenine, 4-hydroxyphenethyl alcohol, kinetin, zeatin; D4) defoliants, such as calcium cyanamide, dimethipin, endothal, ethephon, merphos, metoxuron, pentachlorophenol, thidiazuron, tribufos; D5) ethylene inhibitors, such as aviglycine, 1-methylcyclopropene; D6) ethylene releasers, such as ACC, etacelasil, ethephon, glyoxime; D7) gametocides, such as fenridazon, maleic hydrazide; D8) gibberellins, such as gibberellins, gibberellic acid; D9) growth inhibitors, such as abscisic acid, ancymidol, butralin, carbaryl, chlorphonium, chlorpropham, dikegulac, flumetralin, fluoridamid, fosamine, glyphosine, isopyrimol, jasmonic acid, maleic hydrazide, mepiquat, piproctanyl, prohydrojasmon, propham, tiaojiean, 2,3,5-tri-iodobenzoic acid; D10) morphactins, such as chlorfluren, chlorflurenol, dichlorflurenol, flurenol; D11) growth retardants, such as chlormequat, daminozide, flurprimidol, mefluidide, paclobutrazol, tetcyclacis, uniconazole; D12) growth stimulators, such as brassinolide, brassinolide-ethyl, DCPTA, forchlorfenuron, hymexazol, prosuler, triacontanol; D13) unclassified plant growth regulators, such as bachmedesh, benzofluor, buminafos, carvone, choline chloride, ciobutide, clofencet, cyanamide, cyclanilide, cycloheximide, cyprosulfamide, epocholeone, ethychlozate, ethylene, fuphenthiourea, furalane, heptopargil, holosulf, inabenfide, karetazan, lead arsenate, methasulfocarb, prohexadione, pydanon, sintofen, triapenthenol, trinexapac.

In some embodiments, one or more compartments of the disclosed water-soluble packages comprise one or more microbes disclosed herein along with one or more of a fertilizer, nitrogen stabilizer, or urease inhibitor. Fertilizers include anhydrous ammonia, urea, ammonium nitrate, and urea-ammonium nitrate (UAN) compositions, among many others. In some embodiments, pop-up fertilization and/or starter fertilization is used in combination with the methods and bacteria of the present disclosure.

In some embodiments, nitrogen stabilizers are used in combination with the methods and bacteria of the present disclosure. Nitrogen stabilizers include nitrapyrin, 2-chloro-6-(trichloromethyl) pyridine, N-SERVE 24, INSTINCT, dicyandiamide (DCD). Urease inhibitors include N-(n-butyl)-thiophosphoric triamide (NBPT), AGROTAIN, AGROTAIN PLUS, and AGROTAIN PLUS SC. Further, the disclosure contemplates utilization of AGROTAIN ADVANCED 1.0, AGROTAIN DRI-MAXX, and AGROTAIN ULTRA.

In some embodiments, stabilized forms of fertilizer can be used. For example, a stabilized form of fertilizer is SUPER U, containing 46% nitrogen in a stabilized, urea-based granule, SUPERU contains urease and nitrification inhibitors to guard from denitrification, leaching, and volatilization. Stabilized and targeted foliar fertilizer such as NITAMIN may also be used herein.

Pop-up fertilizers are commonly used in corn fields. Pop-up fertilization comprises applying a few pounds of nutrients with the seed at planting. Pop-up fertilization is used to increase seedling vigor.

Slow- or controlled-release fertilizer that may be used herein entails: A fertilizer containing a plant nutrient in a form which delays its availability for plant uptake and use after application, or which extends its availability to the plant significantly longer than a reference ‘rapidly available nutrient fertilizer’ such as ammonium nitrate or urea, ammonium phosphate or potassium chloride. Such delay of initial availability or extended time of continued availability may occur by a variety of mechanisms. These include controlled water solubility of the material by semi-permeable coatings, occlusion, protein materials, or other chemical forms, by slow hydrolysis of water-soluble low molecular weight compounds, or by other unknown means.

Stabilized nitrogen fertilizer that may be used herein entails: A fertilizer to which a nitrogen stabilizer has been added. A nitrogen stabilizer is a substance added to a fertilizer which extends the time the nitrogen component of the fertilizer remains in the soil in the urea-N or ammoniacal-N form.

Nitrification inhibitor that may be used herein entails: A substance that inhibits the biological oxidation of ammoniacal-N to nitrate-N. Some examples include: (1) 2-chloro-6-(trichloromethyl-pyridine), common name Nitrapyrin, manufactured by Dow Chemical; (2) 4-amino-1,2,4-6-triazole-HCl, common name ATC, manufactured by Ishihada Industries; (3) 2,4-diamino-6-trichloro-methyltriazine, common name CI-1580, manufactured by American Cyanamid; (4) Dicyandiamide, common name DCD, manufactured by Showa Denko; (5) Thiourea, common name TU, manufactured by Nitto Ryuso; (6) 1-mercapto-1,2,4-triazole, common name MT, manufactured by Nippon; (7) 2-amino-4-chloro-6-methyl-pyramidine, common name AM, manufactured by Mitsui Toatsu; (8) 3,4-dimethylpyrazole phosphate (DMPP), from BASF; (9) 1-amide-2-thiourea (ASU), from Nitto Chemical Ind.; (10) Ammoniumthiosulphate (ATS); (11) 1H-1,2,4-triazole (HPLC); (12) 5-ethylene oxide-3-trichloro-methyl 1,2,4-thiodiazole (Terrazole), from Olin Mathieson; (13) 3-methylpyrazole (3-MP); (14) 1-carbamoyle-3-methyl-pyrazole (CMP); (15) Neem; and (16) DMPP.

Urease inhibitor that may be used herein entails: A substance that inhibits hydrolytic action on urea by the enzyme urease. Thousands of chemicals have been evaluated as soil urease inhibitors (Kiss and Simihaian, 2002). However, only a few of the many compounds tested meet the necessary requirements of being nontoxic, effective at low concentration, stable, and compatible with urea (solid and solutions), degradable in the soil and inexpensive. They can be classified according to their structures and their assumed interaction with the enzyme urease (Watson, 2000, 2005). Four main classes of urease inhibitors have been proposed: (a) reagents which interact with the sulphydryl groups (sulphydryl reagents), (b) hydroxamates, (c) agricultural crop protection chemicals, and (d) structural analogues of urea and related compounds. N-(n-Butyl) thiophosphoric triamide (NBPT), phenylphosphorodiamidate (PPD/PPDA), and hydroquinone are probably the most thoroughly studied urease inhibitors (Kiss and Simihaian, 2002). Research and practical testing has also been carried out with N-(2-nitrophenyl) phosphoric acid triamide (2-NPT) and ammonium thiosulphate (ATS). The organo-phosphorus compounds are structural analogues of urea and are some of the most effective inhibitors of urease activity, blocking the active site of the enzyme (Watson, 2005).

In some embodiments, the agricultural compositions in the water-soluble packages disclosed herein may comprise trace metal ions, such as molybdenum ions, iron ions, manganese ions, or combinations of these ions. The concentration of ions in examples of compositions as described herein may between about 0.1 mM and about 50 mM. In some embodiments, the agricultural compositions in the water-soluble packages disclosed herein may comprise additional carriers, besides those involved in the formulation process. Additional carriers may include beta-glucan, carboxylmethyl cellulose (CMC), bacterial extracellular polymeric substance (EPS), sugar, trehalose, maltose, animal milk, milk powder, or other suitable carriers. In some embodiments, peat or planting materials can be used as a carrier, or biopolymers in which a composition is entrapped in the biopolymer can be used as a carrier.

In some embodiments, agricultural compositions in the water-soluble packages described herein may include additional agriculturally acceptable carriers, in addition to the microbial stabilizers, physical stabilizers, and/or buffering agents included in the formulation process. Additional ingredients useful for these embodiments may include at least one member selected from the group consisting of a tackifier, a fungicide, an antibacterial agent, a preservative, a stabilizer, a surfactant, an anti-complex agent, an herbicide, a nematicide, an insecticide, a plant growth regulator, a fertilizer, a rodenticide, a desiccant, a bactericide, a nutrient, or any combination thereof, as described below.

For example, any of the agricultural compositions in the water-soluble packages described herein can include an agriculturally acceptable carrier (e.g., one or more of a fertilizer such as a non-naturally occurring fertilizer, an adhesion agent such as a non-naturally occurring adhesion agent, and a pesticide such as a non-naturally occurring pesticide). A non-naturally occurring adhesion agent can be, for example, a polymer, copolymer, or synthetic wax. For example, any of the coated plant tissues or the environs thereof described herein can contain such an agriculturally acceptable carrier in their coating. In any of the agricultural compositions in the water-soluble packages described herein, an agriculturally acceptable carrier can be or can include a non-naturally occurring compound (e.g., a non-naturally occurring fertilizer, a non-naturally occurring adhesion agent such as a polymer, copolymer, or synthetic wax, or a non-naturally occurring pesticide). Non-limiting examples of agriculturally acceptable carriers are described below. Additional examples of agriculturally acceptable carriers are known in the art.

In some cases, the agricultural compositions in the water-soluble pouches comprise an additional agriculturally acceptable carrier. The carrier can be a solid carrier or liquid carrier, and in various forms including microspheres, powders, emulsions and the like. The carrier may be any one or more of a number of carriers that confer a variety of properties, such as increased stability, wettability, or dispersability. Wetting agents such as natural or synthetic surfactants, which can be nonionic or ionic surfactants, or a combination thereof can be included in the agricultural composition. Suitable formulations that may be prepared include wettable powders, granules, gels, agar strips or pellets, thickeners, and the like, microencapsulated particles, and the like, liquids such as aqueous flowables, aqueous suspensions, water-in-oil emulsions, etc. The formulation may include grain or legume products, for example, ground grain or beans, broth or flour derived from grain or beans, starch, sugar, or oil.

In some embodiments, the agricultural carrier is a soil or a plant growth medium. Other agricultural carriers that may be used include water, fertilizers, plant-based oils, humectants, or combinations thereof. Alternatively, the agricultural carrier may be a solid, such as diatomaceous earth, loam, silica, alginate, clay, bentonite, vermiculite, seed cases, other plant and animal products, or combinations, including granules, pellets, or suspensions. Mixtures of any of the aforementioned ingredients are also contemplated as carriers, such as but not limited to, pesta (flour and kaolin clay), agar or flour-based pellets in loam, sand, or clay, etc.

Agricultural compositions in the water-soluble packages described herein may include food sources for the plant, such as barley, rice, or other biological materials such as seed, plant parts, sugar cane bagasse, hulls or stalks from grain processing, ground plant material or wood from building site refuse, sawdust or small fibers from recycling of paper, fabric, or wood. For example, a fertilizer can be used to help promote the growth or provide nutrients to a plant tissue, e.g., a seed, seedling, or plant. Non-limiting examples of fertilizers include nitrogen, phosphorous, potassium, calcium, sulfur, magnesium, boron, chloride, manganese, iron, zinc, copper, molybdenum, and selenium (or a salt thereof). Additional examples of fertilizers include one or more amino acids, salts, carbohydrates, vitamins, glucose, NaCl, yeast extract, NH₄H₂PO₄, (NH₄)₂SO₄, glycerol, valine, L-leucine, lactic acid, propionic acid, succinic acid, malic acid, citric acid, KH tartrate, xylose, lyxose, and lecithin.

Some examples of plant nutrients can be selected from the group consisting of a nitrogen fertilizer including, but not limited to Urea, Ammonium nitrate, Ammonium sulfate, Non-pressure nitrogen solutions, Aqua ammonia, Anhydrous ammonia, Ammonium thiosulfate, Sulfur-coated urea, Urea-formaldehydes, IBDU, Polymer-coated urea, Calcium nitrate, Ureaform, and Methylene urea, phosphorous fertilizers such as Diammonium phosphate, Monoammonium phosphate, Ammonium polyphosphate, Concentrated superphosphate and Triple superphosphate, and potassium fertilizers such as Potassium chloride, Potassium sulfate, Potassium-magnesium sulfate, Potassium nitrate. Such compositions can exist as free salts or ions within the agricultural composition. Alternatively, nutrients/fertilizers can be complexed or chelated to provide sustained release over time.

In one embodiment, the agricultural composition can include a tackifier or adherent (referred to as an adhesive agent) to help bind other active agents to a substance (e.g., a surface of a plant tissue or the environs thereof). Such agents are useful for combining microbes with carriers that can contain other compounds (e.g., control agents that are not biologic), to yield a coating composition. Such compositions help create coatings around the plant tissues or the environs thereof to maintain contact between the microbe and other agents with the plant tissues or the environs thereof. In one embodiment, adhesives are selected from the group consisting of: alginate, gums, starches, lecithins, formononetin, polyvinyl alcohol, alkali formononetinate, hesperetin, polyvinyl acetate, cephalins, Gum Arabic, Xanthan Gum, Mineral Oil, Polyethylene Glycol (PEG), Polyvinyl pyrrolidone (PVP), Arabino-galactan, Methyl Cellulose, PEG 400, Chitosan, Polyacrylamide, Polyacrylate, Polyacrylonitrile, Glycerol, Triethylene glycol, Vinyl Acetate, Gellan Gum, Polystyrene, Polyvinyl, Carboxymethyl cellulose, Gum Ghatti, and polyoxyethylene-polyoxybutylene block copolymers.

In some embodiments, the adhesives can be, e.g. a wax such as carnauba wax, beeswax, Chinese wax, shellac wax, spermaceti wax, candelilla wax, castor wax, ouricury wax, and rice bran wax, a polysaccharide (e.g., starch, dextrins, maltodextrins, alginate, and chitosans), a fat, oil, a protein (e.g., gelatin and zeins), gum ambles, and shellacs. Adhesive agents can be non-naturally occurring compounds, e.g., polymers, copolymers, and waxes. For example, non-limiting examples of polymers that can be used as an adhesive agent include: polyvinyl acetates, polyvinyl acetate copolymers, ethylene vinyl acetate (EVA) copolymers, polyvinyl alcohols, polyvinyl alcohol copolymers, celluloses (e.g., ethylcelluloses, methylcelluloses, hydroxymethylcelluloses, hydroxypropylcelluloses, and carboxymethylcelluloses), polyvinylpyrolidones, vinyl chloride, vinylidene chloride copolymers, calcium lignosulfonates, acrylic copolymers, polyvinylacrylates, polyethylene oxide, acylamide polymers and copolymers, polyhydroxyethyl acrylate, methylacrylamide monomers, and polychloroprene.

In some embodiments, one or more of the adhesion agents, anti-fungal agents, growth regulation agents, and pesticides (e.g., insecticide) are non-naturally occurring compounds (e.g., in any combination). Additional examples of agriculturally acceptable carriers include dispersants (e.g., polyvinylpyrrolidone/vinyl acetate PVPIVA S-630), surfactants, binders, and filler agents.

In some embodiments, the agricultural composition in the water-soluble packages contains a surfactant. Non-limiting examples of surfactants include nitrogen-surfactant blends such as Prefer 28 (Cenex), Surf-N(US), Inhance (Brandt), P-28 (Wilfarm) and Patrol (Helena); esterified seed oils include Sun-It II (AmCy), MSO (UAP), Scoil (Agsco), Hasten (Wilfarm) and Mes-100 (Drexel); and organo-silicone surfactants include Silwet L77 (UAP), Silikin (Terra), Dyne-Amic (Helena), Kinetic (Helena), Sylgard 309 (Wilbur-Ellis) and Century (Precision). In one embodiment, the surfactant is present at a concentration of between 0.01% v/v to 10% v/v in the dispersion of live microbes. In another embodiment, the surfactant is present at a concentration of between 0.1% v/v to 1% v/v in the dispersion of live microbes.

In some embodiments, a fungicide includes a compound or agent, whether chemical or biological, that can inhibit the growth of a fungus or kill a fungus. In some embodiments, a fungicide includes compounds that may be fungistatic or fungicidal. In some embodiments, a fungicide is a protectant, or agent that is effective predominantly on the surface of plant tissues or the environs thereof. In some embodiments, a fungicide is a protectant, or agent that is effective predominantly on the seed surface, providing protection against seed surface-borne pathogens and providing some level of control of soil-borne pathogens. Non-limiting examples of protectant fungicides include captan, maneb, thiram, or fludioxonil.

In some embodiments, fungicide can be a systemic fungicide, which can be absorbed into the emerging seedling and inhibit or kill the fungus inside host plant tissues. Systemic fungicides used for agricultural treatment include, but are not limited to the following: azoxystrobin, carboxin, mefenoxam, metalaxyl, thiabendazole, trifloxystrobin, and various triazole fungicides, including difenoconazole, ipconazole, tebuconazole, and triticonazole. Mefenoxam and metalaxyl are primarily used to target the water mold fungi Pythium and Phytophthora. Some fungicides are preferred over others, depending on the plant species, either because of subtle differences in sensitivity of the pathogenic fungal species, or because of the differences in the fungicide distribution or sensitivity of the plants. In some embodiments, fungicide can be a biological control agent, such as a bacterium or fungus. Such organisms may be parasitic to the pathogenic fungi, or secrete toxins or other substances which can kill or otherwise prevent the growth of fungi. Any type of fungicide, particularly ones that are commonly used on plants, can be used as a control agent in an agricultural composition.

In some embodiments, the agricultural composition in the water-soluble package comprises a control agent which has antibacterial properties. In one embodiment, the control agent with antibacterial properties is selected from the compounds described herein elsewhere. In another embodiment, the compound is Streptomycin, oxytetracycline, oxolinic acid, or gentamicin. Other examples of antibacterial compounds which can be used as part of an agricultural composition include those based on dichlorophene and benzylalcohol hemi formal (Proxel® from ICI or Acticide® RS from Thor Chemie and Kathon® MK 25 from Rohm & Haas) and isothiazolinone derivatives such as alkylisothiazolinones and benzisothiazolinones (Acticide® MBS from Thor Chemie).

In some embodiments, the plant growth regulator is selected from the group consisting of: Abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat (mepiquat chloride), naphthaleneacetic acid, N-6-benzyladenine, paclobutrazol, prohexadione phosphorotrithioate, 2,3,5-tri-iodobenzoic acid, trinexapac-ethyl and uniconazole. Additional non-limiting examples of growth regulators include brassinosteroids, cytokinines (e.g., kinetin and zeatin), auxins (e.g., indolylacetic acid and indolylacetyl aspartate), flavonoids and isoflavanoids (e.g., formononetin and diosmetin), phytoaixins (e.g., glyceolline), and phytoalexin-inducing oligosaccharides (e.g., pectin, chitin, chitosan, polygalacuronic acid, and oligogalacturonic acid), and gibellerins. Such agents are ideally compatible with the agricultural plant tissues or the environs thereof onto which the agricultural composition is applied (e.g., it should not be deleterious to the growth or health of the plant). Furthermore, the agent is ideally one which does not cause safety concerns for human, animal or industrial use (e.g., no safety issues, or the compound is sufficiently labile that the commodity plant product derived from the plant contains negligible amounts of the compound).

Some examples of nematode-antagonistic biocontrol agents include ARF18; 30 Arthrobotrys spp.; Chaetomium spp.; Cylindrocarpon spp.; Exophilia spp.; Fusarium spp.; Gliocladium spp.; Hirsutella spp.; Lecanicillium spp.; Monacrosporium spp.; Myrothecium spp.; Neocosmospora spp.; Paecilomyces spp.; Pochonia spp.; Stagonospora spp.; vesicular-arbuscular mycorrhizal fungi, Burkholderia spp.; Pasteuria spp., Brevibacillus spp.; Pseudomonas spp.; and Rhizobacteria. Particularly preferred nematode-antagonistic biocontrol agents include ARF18, Arthrobotrys oligospora, Arthrobotrys dactyloides, Chaetomium globosum, Cylindrocarpon heteronema, Exophilia jeanselmei, Exophilia pisciphila, Fusarium aspergilus, Fusarium solani, Gliocladium catenulatum, Gliocladium roseum, Gliocladium vixens, Hirsutella rhossiliensis, Hirsutella minnesotensis, Lecanicillium lecanii, Monacrosporium drechsleri, Monacrosporium gephyropagum, Myrothecium verrucaria, Neocosmospora vasinfecta, Paecilomyces lilacinus, Pochonia chlamydosporia, Stagonospora heteroderae, Stagonospora phaseoli, vesicular-arbuscular mycorrhizal fungi, Burkholderia cepacia, Pasteuria penetrans, Pasteuria thornei, Pasteuria nishizawae, Pasteuria ramosa, Pastrueia usage, Brevibacillus laterosporus strain G4, Pseudomonas fluorescens and Rhizobacteria.

Some examples of rodenticides may include selected from the group of substances consisting of 2-isovalerylindan-1,3-dione, 4-(quinoxalin-2-ylamino) benzenesulfonamide, alpha-chlorohydrin, aluminum phosphide, antu, arsenous oxide, barium carbonate, bisthiosemi, brodifacoum, bromadiolone, bromethalin, calcium cyanide, chloralose, chlorophacinone, cholecalciferol, coumachlor, coumafuryl, coumatetralyl, crimidine, difenacoum, difethialone, diphacinone, ergocalciferol, flocoumafen, fluoroacetamide, flupropadine, flupropadine hydrochloride, hydrogen cyanide, iodomethane, lindane, magnesium phosphide, methyl bromide, norbormide, phosacetim, phosphine, phosphorus, pindone, potassium arsenite, pyrinuron, scilliroside, sodium arsenite, sodium cyanide, sodium fluoroacetate, strychnine, thallium sulfate, warfarin and zinc phosphide.

In some embodiments, the agricultural composition in the water-soluble package comprises mineral carriers such as kaolin clay, pyrophyllite, bentonite, montmorillonite, diatomaceous earth, acid white soil, vermiculite, and pearlite, and inorganic salts such as ammonium sulfate, ammonium phosphate, ammonium nitrate, urea, ammonium chloride, and calcium carbonate. Also, organic fine powders such as wheat flour, wheat bran, and rice bran may be used.

Additional agricultural composition components for inclusion in the compositions disclosed herein may be found in International Patent Publication No. WO202000664, the contents of which are herein incorporated by reference in their entirety for all purposes.

e. Cross-Linked Alginate Microcapsules Comprising Dehydrated Microbes

In some embodiments, the dehydrated microbes in one or more compartments of the water-soluble packages disclosed herein are encapsulated within cross-linked alginate microcapsules (CLAMS). A detailed description of CLAMS, and methods of producing them is found in U.S. Pat. No. 9,700,519, the contents of which are hereby incorporated by reference in their entirety for all purposes.

Thus, in some embodiments, the one or more compartments of the water-soluble package comprises CLAMs comprising the dehydrated microbes encapsulated therein. In some embodiments, the CLAMs comprising the dehydrated microbes are suspended in a non-aqueous liquid. In some embodiments, the non-aqueous liquid is an oil. Thus, in some embodiments, the one or more compartments of the water-soluble package comprises a composition comprising: a) a non-aqueous liquid; and b) cross-linked alginate microcapsules (CLAMs) suspended in the non-aqueous liquid and comprising a diazotrophic bacterium encapsulated therein. In some embodiments, the non-aqeuous liquid is a plant-derived oil. In some embodiments, the non-aqueous liquid is a vegetable oil.

In some embodiments, the plant-derived oil is a soybean oil, canola oil, cottonseed oil, coconut oil, corn oil, olive oil, palm oil, peanut oil, rapeseed oil, safflower oil, sesame oil, soybean oil, or sunflower oil. In some embodiments, the plant-derived oil is a corn oil. In some embodiments, the composition comprising CLAMs and the dehydrated microbes further comprises one or more additional ingredients selected from the group consisting of: trehalose, milk powder, maltose, a pesticide, an insecticide, a fungicide, an herbicide, a fertilizer, a nematicide, a bio-stimulant, a biological, zinc, a plant growth promoter, a polymer latex, a wax emulsion, a surfactant, a polygalacturonate, chitosan, collagen, a soy protein, a whey protein, dicalcium phosphate, calcium carbonate, calcium oxalate, calcium phosphate, calcium meta-silicate, calcium tartrate, adipic acid, acrylic acid, glutaric acid, succinic acid, ascorbic acid, gallic acid, caffeic acid, and combinations thereof.

### Methods of Producing Water-Soluble Film Packaging Comprising Dehydrated Microbes

The disclosure provides methods of producing water-soluble film packages disclosed herein comprising one or more compartments, the methods comprising providing two sheets of water-soluble film, and sealing the film to form seal areas around one or more compartments. In some embodiments, a single sheet of water-soluble film is folded over to produce the packaging.

In some embodiments, the method comprises thermoforming the film. In some embodiments, thermoforming comprises a process in which a first sheet of film is subjected to a moulding process to form recesses in the film. The process involves heating the film to soften it and also the application of vacuum to hold the film in the moulds. The recesses are then filled with the agricultural composition. The packages are completed by overlaying a second sheet over the filled recesses and sealing it to the first sheet of film around the edges of the recesses to form a flat seal area. Relaxation of the first film typically then causes the applied second sheet to bulge out when the vacuum is released from the first sheet of film in the mould. The packages are cut apart to leave part of the flat seal area as a peripheral “skirt” around each capsule when it is removed from the mould. In some embodiments, water-soluble packages with more than one compartment may be produced by assembling two thermoformed compartments to form a multi-compartment package.

In some embodiments, the method comprises generating one or more compartments of the water-soluble package using a vertical form fill seal process, as described in detail in US 2001/0033883, the contents of which is incorporated herein by reference in its entirety for all purposes. Sealing can be done by any suitable method for example heat-sealing, solvent sealing or UV sealing. Particularly preferred is water-sealing. Water sealing may be carried out by applying moisture to the second sheet of film before it is sealed to the first sheet of film to form the seal areas. Further details of methods of producing water-soluble packages with one or more compartments are provided in WO 2014/202412, WO 2010/0088112, EP 1375637 and EP 1394065, the contents of each of which is incorporated herein by reference in its entirety for all purposes.

In some embodiments, the water-soluble film may be produced using any method known in the art. In some embodiments, the method of producing the water-soluble film comprises depositing a fluid composition including a film forming material and at least one other component immiscible with the film forming material and having a density different than the film forming material into a single layer, and drying the single layer such that the at least one other component has a predetermined non-uniform distribution in the thickness direction of the single layer after drying. Methods of producing water-soluble films are described further in U.S. Pat. Nos. 7,357,891 and 8,617,589, the contents of each of which is incorporated herein by reference in its entirety for all purposes.

The disclosure provides methods of producing a dispersible formulation of dehydrated microbes, comprising: encapsulating an agricultural biological in a water-soluble film, thereby producing a water-soluble film package comprising one or more compartment(s) comprising the agricultural biological; wherein said agricultural biological comprises dehydrated microbes.

In some embodiments, the method comprises fermentation of the microbe to produce a high cell density culture liquid. In some embodiments, bioreactors such as continuous stirred tank reactors, or batch reactors may be used. In some embodiments, the method comprises concentrating the culture liquid to further increase cell density. In some embodiments, cell density is measured using “spread plating”, which refers to the plating of the culture liquid or the microbes contained therein on an agar plate, and counting the number of colony forming units per volume of culture liquid. In some embodiments, the cell density may be in the range of about 10⁸ CFU/ml to about 10¹⁶ CFU/ml, for example about 10⁸ CFU/ml, 10⁹ CFU/ml, 10¹⁰ CFU/ml, 10¹¹ CFU/ml, 10¹² CFU/ml, 10¹³ CFU/ml, 10¹⁴ CFU/ml, 10¹⁵ CFU/ml, or 10¹⁶ CFU/ml, including all values and subranges that lie therebetween. In some embodiments, the cell density may be in the range of about 10¹⁰ CFU/ml to about 10¹² CFU/ml. Cell density may vary based on the microbes and concentration method employed. In some embodiments, the concentrating step comprises centrifugation, tangential flow filtration (TFF), or a combination thereof.

In some embodiments, the method comprises combining the culture liquid with one or more excipients. In some embodiments, the one or more excipients comprises sucrose, milk solids nonfat, or a combination thereof. Without being bound by a theory, it is thought that addition of the excipients might protect the microbes during bioprocessing steps and/or stabilize it during dry storage, thereby enabling long term storage with minimal decrease in viable microbial cell population upon rehydration. In some embodiments, the method comprises dehydrating the culture liquid to produce dried material, wherein the dried material comprises dehydrated microbes.

In some embodiments, the processing of the culture liquid to produce dried material comprises freeze drying, spray drying, fluidized bed drying, extrusion, drying, or any combination thereof. In some embodiments, the method comprises processing of the dried material, thereby reducing the size of the dried material. In some embodiments, the processing of the dried material produces a dry microbial powder. In some embodiments, the processing comprises milling, sieving, or a combination thereof. In some embodiments, the processing of the dried material comprises agglomeration of the dry microbial powder to produce granules.

Without being bound by a theory, it is thought that modifying the particle size of the dried material may enhance shelf stability of the microbes and further, enhance dissolution kinetics. That is, in some embodiments, modifying the particle size of the dried material may enhance the speed of dissolution upon rehydration. In some embodiments, the processing of the dried material instantizes the dried material. As used herein, “instantization” refers to processing steps that make a product have good reconstituting properties such that it disperses or dissolves quickly when added to a liquid. In some embodiments, the formation of granules instantizes the microbes. In some embodiments, the production of dry microbial powder instantizes the microbes.

In some embodiments, the method comprises blending the dry microbial powder with one or more excipients. In some embodiments, the one or more excipients comprises bulking agents, anticaking agents, dispersants, or any combination thereof. In some embodiments, the method comprises blending the dry microbial powder with at least 25% of one or more hygroscopic salt(s). Without being bound by a theory, it is thought that blending the dry microbial powder with one or more excipients might improve one or more physical properties of the dry microbial powder. For instance, addition of the excipient may reduce or prevent caking or clumping, and/or promote rapid dissolution.

As used herein, “encapsulating” refers to enclosing the agricultural composition within water-soluble film package, such that the agricultural composition is within the one or more sealed compartments formed by the water-soluble film. Encapsulation of the agricultural composition within the package can be done by any method known in the art for the purpose, or any method that can be conceived to result in the agricultural composition being encapsulated within the package.

In some embodiments, encapsulation comprises filling the dried material into one or more compartments of the package. As used herein, “filling” refers to the addition of the dried material to an existing water-soluble package comprising one or more compartments. The compartments are sealed in such a way as to allow the transfer of the dried material into the compartments. In some embodiments, the dried material is placed on a first sheet of water-soluble film, and a second sheet of water-soluble film is placed on top of the dried material so to cover it. The edges are sealed as described herein to produce the package.

In some embodiments, the water-soluble film package contains a unit dose of the dehydrated microbes. In some embodiments, the water-soluble package is sealed. In some embodiments, the water-soluble package is stored in a hermetic vessel until use. In some embodiments, the water-soluble package is the only barrier between the microbes contained therein, and moisture and/or oxygen. In some embodiments, the water-soluble package is further encased within additional protective packaging, such as a second airtight packaging with modified internal atmosphere. In some embodiments, one or more water-soluble packages disclosed herein are further encased within a container, such as, for example, a carboy. In some embodiments, the carboy comprises an oxygen absorber substance that is capable of increasing the stability of the microbes. In some embodiments, one or more water-soluble packages disclosed herein are further encased within another package, such as, for example, a sealed foil pack. In some embodiments, foil pack comprises an oxygen absorber substance that is capable of increasing the stability of the microbes.

In some embodiments, the water-soluble film package comprises a first and a second compartment, wherein the dry microbial powder or granules is filled into the first and/or second compartment of the water-soluble film package. In some embodiments, the method comprises filling the one or more compartments with any one or more of the additives that enhances the shelf stability, uniformity, flow attributes, dissolution kinetics, or any combination thereof, of the dehydrated microbes. In some embodiments, the method comprises filling the first and/or second compartment with an additive that enhances the shelf stability, uniformity, flow attributes, dissolution kinetics, or any combination thereof, of the dehydrated microbes.

In some embodiments, the method comprises filling the one or more compartments with a carbon source capable of enhancing growth of the dehydrated microbes. In some embodiments, the method comprises filling the first and/or second compartment with a carbon source capable of enhancing growth of the dehydrated microbes. In some embodiments, the method comprises filling the one or more compartments with a dispersing agent. In some embodiments, the method comprises filling the first and/or second compartment with a dispersing agent. In some embodiments, the method comprises filling the one or more compartments with a fertilizer. In some embodiments, the method comprises filling the first and/or second compartment with a fertilizer. In some embodiments, the method comprises filling the one or more compartments with a plant growth hormone. In some embodiments, the method comprises filling the first and/or second compartment with a plant growth hormone.

In some embodiments, the method comprises filling the one or more compartments with an agent selected from the group consisting of a bulking agent, an anticaking agent, a microbial stabilizer, a physical stabilizer, a buffering agent, and a dispersant. In some embodiments, the method comprises filling the first and/or second compartment with an agent selected from the group consisting of a bulking agent, an anticaking agent, a microbial stabilizer, a physical stabilizer, a buffering agent, and a dispersant.

The disclosure provides methods of producing a dispersible formulation of dehydrated microbes, comprising: encapsulating an agricultural biological in a water-soluble film, thereby producing a water-soluble film package comprising one or more compartment(s) comprising the agricultural biological; wherein said agricultural biological comprises dehydrated microbes, wherein the method comprises one or more of the following steps: fermentation of microbes to produce a high cell density culture liquid, combining the culture liquid with sucrose, milk solids nonfat, or a combination thereof, dehydrating the culture liquid to produce dried material, wherein the dried material comprises dehydrated microbes, and filling the dried material into one or more compartment(s) of the water-soluble film package. In some embodiments, the method comprises concentrating the culture liquid to further increase cell density. In some embodiments, the method comprises processing of the dried material to produce a dry microbial powder. In some embodiments, the method comprises agglomerating the dry microbial powder to produce granules. In some embodiments, the method comprises blending the dry microbial powder with bulking agents, anticaking agents, dispersants, or any combination thereof.

In some embodiments, the methods of producing water-soluble film package comprising dehydrated microbes comprise steps of varying and/or optimizing the contents of the water-soluble package along different parameters. In some embodiments, these parameters include the selection of an initial microbial cell density, the choice of microbial stabilizer, the choice of physical stabilizer, and the choice of buffering agent, each of which components is described in detail in its respective section.

In some embodiments, the different parameters are optimized to enhance the viability of the microbes upon contact of the water-soluble package with the liquid, and rehydration of the microbes. In some embodiments, enhancing the viability of the microbes refers to increasing the time interval for which the dehydrated microbes may be stored before use. In some embodiments, enhancing the viability of the microbes refers to increasing the proportion of microbes that are viable upon rehydration relative to the starting population of dehydrated microbes that was encased in the water-soluble package.

In some embodiments, the selection of the initial cell density with an acceptable decay rate, the selection of the buffering agent, the selection of the microbial stabilizer, and the selection of the physical stabilizer are performed in any order. In some embodiments, these selection steps are performed in parallel. In some embodiments, these selection steps are performed serially. In some embodiments, the selection of the initial cell density with an acceptable decay rate is performed first.

In some embodiments, the selections of the buffering agent, microbial stabilizer, and physical stabilizer are performed in tandem comprising different combinations. In some embodiments, the selections of any two of the buffering agent, microbial stabilizer, and physical stabilizer are performed in tandem. In some embodiments, when tested in tandem, the method comprises selecting combinations that have an additive or synergistic effect on microbial stability. In some embodiments, the selections of the buffering agent, microbial stabilizer, and physical stabilizer are performed separately varying each parameter individually. In some embodiments, the method comprises varying each parameter by assaying two or more possible components of each type. In some embodiments, the method comprises varying each parameter by assaying two or more concentrations of each component. In some embodiments, the method comprises comparing possible components and/or concentrations of components against each other.

In some embodiments, one or more screening assays are used to select the buffering agent, microbial stabilizer, and physical stabilizer for inclusion in the water-soluble package. The one or more screening assays may measure the viability of the microbes upon rehydration, and/or toxin accumulation in the water-soluble package, as described below. In some embodiments, microbial viability is measured in CFU/mL via a standard plating assay. In some embodiments, microbial viability is evaluated by measuring colonization potential. In some embodiments, the colonization potential is measured in log 10 copies per gram of fresh weight via a root colonization assay. In some embodiments, the contents of the water-soluble package are screened for toxin accumulation. In some embodiments, the contents of the water-soluble package are screened for toxin concentrations at a given time point, e.g., the target shelf life time point.

In terms of selection of the buffering agent, in some embodiments, a screening assay compares different buffering agents, different pH levels of buffering agents, different buffering capacities of a given buffering agent, and/or different molarities of buffering agents. In terms of selection of the microbial stabilizer, in some embodiments, a screening assay compares different microbial stabilizers and/or different concentrations of a given microbial stabilizer. In terms of selection of the physical stabilizer, in some embodiments, a screening assay compares different physical stabilizers and/or different concentrations of a given physical stabilizer. In some embodiments, the physical stabilizers and microbial stabilizers are assayed in tandem.

In some embodiments, the water-soluble packages disclosed herein comprise an initial microbial cell density to provide an acceptable rate of microbial decay. In some embodiments, the initial cell density is varied to identify an initial cell density that lowers the rate of decay compared to an existing formulation. In some embodiments, the initial cell density is varied to identify an initial cell density that minimizes the rate of decay while maximizing the cell density. In some embodiments, the water-soluble packages disclosed herein comprise an initial cell density with an acceptable rate of microbial decay, a rate of decay that is lower than existing formulations, or a rate of decay that is minimized while maximizing cell density.

In some embodiments, the initial cell density is selected to provide an acceptable rate of decay based on a target cell density at a later time point. For example, for an agricultural composition that is targeted to have at least a three-month shelf life, an acceptable rate of decay would be one that results in the three-month old agricultural composition comprising a microbial density above the target threshold given the value of the initial cell density.

To provide a non-limiting illustrative example of a calculation to identify an acceptable rate of decay, suppose the initial cell density is 1E10 CFU/mL; the acceptable threshold for cell density for the purposes of application to agricultural plant tissues or the environs thereof is 1E9 CFU/mL; and the target shelf life is at least three months. Then an acceptable rate of decay would be one that resulted in a composition having the cell density of 1E9 CFU/mL at the three month time point. Assuming that decay is approximately linear for the log of the cell density, this would be a decay rate that was less than or equal to the decay rate r that satisfied the equation log₁₀ T_{f}=log₁₀ Tᵢ−r×t, where T_{f} is the final cell density threshold in CFU/mL at the target shelf-life time point, Tᵢ is the initial cell density in CFU/mL, r is the decay rate in log₁₀ loss of CFU/mL per day, and t is the number of days at the target shelf-life time point. For this example, that would translate to: log₁₀ 1E9=log₁₀ 1E10-r×90, which is satisfied when r is 1/90≈0.011 log₁₀ loss of CFU/mL per day.

In some embodiments, the method comprises testing multiple initial cell densities and monitoring microbial viability over a period of time. In some embodiments, this comprises generating a titration curve of initial cell density versus microbial decay rate. In some embodiments, the initial cell density is selected to be one associated with an acceptable rate of decay. In some embodiments, the parameter of initial cell density is varied within the method and selected separately from the other parameters. In some embodiments, the parameter of initial cell density is varied at the same time as one or more other parameters, such as microbial stabilizer, physical stabilizer, or buffering agent.

### Methods of Use of Water-Soluble Film Packaging Comprising Dehydrated Microbes

The present disclosure provides methods for improving one or more aspects of agricultural plant characteristics through the use of the water-soluble packages disclosed herein, comprising a) contacting any one of the water-soluble film packages disclosed herein with a liquid to produce a dispersion of live microbes; and b) applying the dispersion of live microbes to a locus comprising the plant, thereby colonizing the locus with the microbes. In some embodiments, the one or more aspects of the agricultural plant characteristics comprises health, yield, yield variance, stress resistance, growth, or agronomic characteristics of the plant. In some embodiments, the present methods are used to increase agricultural plant crop yield and/or decrease agricultural plant crop yield variance.

In some embodiments, the methods are used to supply nitrogen to the plant. That is, the disclosure provides method for supplying nitrogen to a plant, the method comprising: a) contacting any one of the water-soluble film packages disclosed herein with a liquid to produce a dispersion of live nitrogen-fixing microbes; and b) applying the dispersion of live microbes to a locus comprising the plant, thereby colonizing the locus with the microbes, thereby supplying nitrogen to the plant.

In some embodiments, the dispersion of live microbes is applied to the agricultural plant tissues, any plant part, or the environs thereof. In some embodiments, the dispersion of live microbes is applied in-furrow. In some embodiments, the dispersion of live microbes is applied as a seed coat on a seed. In some embodiments, the dispersion of live microbes is applied to the roots of a plant. In some embodiments, the dispersion of live microbes is applied to the surface of a seedling, plant, plant part, or the environs thereof. In some embodiments, the dispersion of live microbes is applied as a layer above a surface of a seed, seedling, plant, plant part, or the environs thereof. In some embodiments, the dispersion of live microbes is applied to a seed, seedling, plant, plant part, or the environs thereof by spraying, immersing, dipping, rolling, shaking, immersing, flowing, misting, painting, brushing, washing, coating, sprinkling, and/or encapsulating the seed, seedling, plant, plant part, or the environs thereof with the dispersion of live microbes. Non-limiting examples of plant tissues include a seed, seedling, leaf, cutting, plant, bulb, tuber, root, and rhizomes.

In some embodiments, the methods provide an effective amount of the dispersion of live microbes to plant tissues or the environs thereof. In general, an effective amount is an amount sufficient to result in plants with improved traits (e.g. a desired level of nitrogen fixation). An effective amount of the dispersion of live microbes can be used to populate the sub-soil region around seeds, seedlings, plants, or plant parts with viable bacterial growth, or populate the seeds, seedlings, plants, or plant parts with viable bacterial growth. In some embodiments, the present methods result in a higher concentration of microbes surviving through storage, delivery, and/or transport until planting.

The disclosure provides methods of coating a seed with nitrogen-fixing microbes, the method comprising: a) contacting any one of the water-soluble film packages disclosed herein with a liquid to produce a dispersion of live microbes; and b) coating the seed with the dispersion of live microbes. In some embodiments, the water-soluble film package comprises polyvinyl alcohols. In some embodiments, the dispersion of live microbes comprises polyvinyl alcohols. In some embodiments, the viability of the seed-coated microbes in the presence of polyvinyl alcohols is greater than the viability of control seed-coated microbes in the absence of polyvinyl alcohols.

In some embodiments, the one or more water-soluble packages disclosed herein may be mixed with an extender at a site of use, such as a seed treatment facility. As used herein, an “extender” refers to a liquid that may be co-blended with the microbes before use. In some embodiments, the extender is mixed with microbes before application to seeds. Without being bound to a theory, it is thought that an extender can help the microbes adhere to a seed, extend the shelf life and/or viability of the microbes, or a combination thereof. In some embodiments, the one or more water-soluble packages disclosed herein may be added to a tank comprising a liquid, and optionally, mixed with an extender. In some embodiments, the tank is an IF tank, or an EnlighteN tank. In some embodiments, the extender is a liquid extender 3-1274. In some embodiments, the one or more water-soluble packages disclosed herein comprises Klebsiella variicola 137-2253 and Kosakonia sacchari 6-5687, and a liquid extender 3-1274. Further details are provided in the International Patent Publication Nos. WO2022/140656, WO2020/092940, WO2020/118111, WO2021/222643, and WO2022/260676 the contents of which are incorporated herein by reference in their entirety.

### Improvement of Shelf Stability

As used herein, the “shelf life” of an agricultural composition refers to the period of time over which the agricultural composition can be stored and still retain a desired level of efficacy for its intended purpose, e.g., retention of the viability of the microbes contained in the agricultural compositions disclosed herein. In some embodiments, the shelf life is the period of time over which an agricultural composition can be stored at room temperature and experience less than log loss CFU/mL of 1. In some embodiments, the shelf life is the period of time over which an agricultural composition can be stored at room temperature and experience less than log loss CFU/mL of 0.5-2. In some embodiments, the shelf life is the period of time over which an agricultural composition can be stored and experience less than about 50% loss of cell density in CFU/mL. In some embodiments, the shelf life is the period of time over which an agricultural composition can be stored and experience less than 90% loss of cell density in CFU/mL. In some embodiments, the shelf life is measured at room temperature. In some embodiments, the shelf life is measured at 4° C. In some embodiments, the temperature varies over the course of the period of storage over which shelf life is measured.

In some embodiments, the water-soluble packages disclosed herein improve the shelf stability of the microbes contained therein. In some embodiments, the microbes in the water-soluble packages disclosed herein have improved shelf stability relative to comparable liquid formulations, dry powders or granules comprising the same microbes which are not encapsulated by the water-soluble packages disclosed herein.

In some embodiments, this shelf stability is determined by measuring microbial viability as a function of time. In some embodiments, the microbes in the water-soluble packages disclosed are shelf stable for a period of about 1 hour to about 5 years, for example about 2 hours, about 10 hours, about 24 hours, about 2 days, about 4 days, about 1 week, about 2 weeks, about 10 days, about 1 month, about 3 months, about 6 months, about 9 months, about 1 year, about 2 years, about 3 years, or about 4 years, including all values and subranges that lie there between. In some embodiments, the microbes in the water-soluble packages disclosed are shelf stable for a period of about 1 year or 12 months. In some embodiments, the microbes in the water-soluble packages disclosed are shelf stable for a period of about 2 years or 24 months. In some embodiments, the microbes in the water-soluble packages disclosed are shelf stable for a period of at least three months. In some embodiments, the microbes in the water-soluble packages disclosed are shelf stable for a period of at least four months. In some embodiments, the microbes in the water-soluble packages disclosed are shelf stable for a period of at least five months. In some embodiments, the microbes in the water-soluble packages disclosed are shelf stable for a period of at least six months.

In some embodiments, the microbes in the water-soluble packages disclosed are shelf stable at room temperature for a period of about 1 hour to about 5 years, for example about 2 hours, about 10 hours, about 24 hours, about 2 days, about 4 days, about 1 week, about 2 weeks, about 10 days, about 1 month, about 3 months, about 6 months, about 9 months, about 1 year, about 2 years, about 3 years, or about 4 years, including all values and subranges that lie there between. In some embodiments, the microbes in the water-soluble packages disclosed are shelf stable at room temperature for a period of about 1 year or 12 months. In some embodiments, the microbes in the water-soluble packages disclosed are shelf stable at room temperature for a period of about 2 years or 24 months. In some embodiments, the water-dissolvable pouches and further encased in foil pouches along with an oxygen absorber.

### Concentrations and Rates of Application of the Dispersion of Live Microbes

In some embodiments, the dispersion of live microbes may be applied as a treatment to a seed, seedling, plant, plant part, or environs thereof in a variety of concentrations. For example, the microbes may be at a CFU concentration per seed of: 1×10¹, 1×10², 1×10³, 1×10⁴, 1×10⁵, 1×10⁶, 1×10⁷, 1×10⁸, 1×10⁹, 1×10¹⁰, or more. In particular aspects, the dispersion of live microbes comprises microbes at a concentration of about 1×10⁴ to about 1×10⁸ CFU per seed. In other particular aspects, the dispersion of live microbes comprises about 1×10⁵ to about 1×10⁷ CFU per seed. In other aspects, the dispersion of live microbes comprises about 1×10⁶ CFU per seed.

In the United States, about 10 of corn acreage is planted at a seed density of above about 36,000 seeds per acre; ⅓ of the corn acreage is planted at a seed density of between about 33,000 to 36,000 seeds per acre; ⅓ of the corn acreage is planted at a seed density of between about 30,000 to 33,000 seeds per acre, and the remainder of the acreage is variable. See, “Corn Seeding Rate Considerations,” written by Steve Butzen, available at: www.pioneer.com/home/site/us/agronomy/library/corn-seeding-rate-considerations/

Table 3 below utilizes various CFU concentrations per seed in a contemplated seed treatment embodiment (rows across) and various seed acreage planting densities (1^{st} column: 15K-41K) to calculate the total amount of CFU per acre, which would be utilized in various agricultural scenarios (i.e. seed treatment concentration per seed×seed density planted per acre). Thus, if one were to utilize a seed treatment with 1×10⁶ CFU per seed and plant 30,000 seeds per acre, then the total CFU content per acre would be 3×10¹⁰ (i.e. 30K*1×10⁶).

TABLE 3
Total CFU Per Acre Calculation for Seed Treatment Embodiments

| Corn Population | | | | | | | | |
|---|---|---|---|---|---|---|---|---|
| (i.e. seeds per acre) | 1.00E+02 | 1.00E+03 | 1.00E+04 | 1.00E+05 | 1.00E+06 | 1.00E+07 | 1.00E+08 | 1.00E+09 |
| 15,000 | 1.50E+06 | 1.50E+07 | 1.50E+08 | 1.50E+09 | 1.50E+10 | 1.50E+11 | 1.50E+12 | 1.50E+13 |
| 16,000 | 1.60E+06 | 1.60E+07 | 1.60E+08 | 1.60E+09 | 1.60E+10 | 1.60E+11 | 1.60E+12 | 1.60E+13 |
| 17,000 | 1.70E+06 | 1.70E+07 | 1.70E+08 | 1.70E+09 | 1.70E+10 | 1.70E+11 | 1.70E+12 | 1.70E+13 |
| 18,000 | 1.80E+06 | 1.80E+07 | 1.80E+08 | 1.80E+09 | 1.80E+10 | 1.80E+11 | 1.80E+12 | 1.80E+13 |
| 19,000 | 1.90E+06 | 1.90E+07 | 1.90E+08 | 1.90E+09 | 1.90E+10 | 1.90E+11 | 1.90E+12 | 1.90E+13 |
| 20,000 | 2.00E+06 | 2.00E+07 | 2.00E+08 | 2.00E+09 | 2.00E+10 | 2.00E+11 | 2.00E+12 | 2.00E+13 |
| 21,000 | 2.10E+06 | 2.10E+07 | 2.10E+08 | 2.10E+09 | 2.10E+10 | 2.10E+11 | 2.10E+12 | 2.10E+13 |
| 22,000 | 2.20E+06 | 2.20E+07 | 2.20E+08 | 2.20E+09 | 2.20E+10 | 2.20E+11 | 2.20E+12 | 2.20E+13 |
| 23,000 | 2.30E+06 | 2.30E+07 | 2.30E+08 | 2.30E+09 | 2.30E+10 | 2.30E+11 | 2.30E+12 | 2.30E+13 |
| 24,000 | 2.40E+06 | 2.40E+07 | 2.40E+08 | 2.40E+09 | 2.40E+10 | 2.40E+11 | 2.40E+12 | 2.40E+13 |
| 25,000 | 2.50E+06 | 2.50E+07 | 2.50E+08 | 2.50E+09 | 2.50E+10 | 2.50E+11 | 2.50E+12 | 2.50E+13 |
| 26,000 | 2.60E+06 | 2.60E+07 | 2.60E+08 | 2.60E+09 | 2.60E+10 | 2.60E+11 | 2.60E+12 | 2.60E+13 |
| 27,000 | 2.70E+06 | 2.70E+07 | 2.70E+08 | 2.70E+09 | 2.70E+10 | 2.70E+11 | 2.70E+12 | 2.70E+13 |
| 28,000 | 2.80E+06 | 2.80E+07 | 2.80E+08 | 2.80E+09 | 2.80E+10 | 2.80E+11 | 2.80E+12 | 2.80E+13 |
| 29,000 | 2.90E+06 | 2.90E+07 | 2.90E+08 | 2.90E+09 | 2.90E+10 | 2.90E+11 | 2.90E+12 | 2.90E+13 |
| 30,000 | 3.00E+06 | 3.00E+07 | 3.00E+08 | 3.00E+09 | 3.00E+10 | 3.00E+11 | 3.00E+12 | 3.00E+13 |
| 31,000 | 3.10E+06 | 3.10E+07 | 3.10E+08 | 3.10E+09 | 3.10E+10 | 3.10E+11 | 3.10E+12 | 3.10E+13 |
| 32,000 | 3.20E+06 | 3.20E+07 | 3.20E+08 | 3.20E+09 | 3.20E+10 | 3.20E+11 | 3.20E+12 | 3.20E+13 |
| 33,000 | 3.30E+06 | 3.30E+07 | 3.30E+08 | 3.30E+09 | 3.30E+10 | 3.30E+11 | 3.30E+12 | 3.30E+13 |
| 34,000 | 3.40E+06 | 3.40E+07 | 3.40E+08 | 3.40E+09 | 3.40E+10 | 3.40E+11 | 3.40E+12 | 3.40E+13 |
| 35,000 | 3.50E+06 | 3.50E+07 | 3.50E+08 | 3.50E+09 | 3.50E+10 | 3.50E+11 | 3.50E+12 | 3.50E+13 |
| 36,000 | 3.60E+06 | 3.60E+07 | 3.60E+08 | 3.60E+09 | 3.60E+10 | 3.60E+11 | 3.60E+12 | 3.60E+13 |
| 37,000 | 3.70E+06 | 3.70E+07 | 3.70E+08 | 3.70E+09 | 3.70E+10 | 3.70E+11 | 3.70E+12 | 3.70E+13 |
| 38,000 | 3.80E+06 | 3.80E+07 | 3.80E+08 | 3.80E+09 | 3.80E+10 | 3.80E+11 | 3.80E+12 | 3.80E+13 |
| 39,000 | 3.90E+06 | 3.90E+07 | 3.90E+08 | 3.90E+09 | 3.90E+10 | 3.90E+11 | 3.90E+12 | 3.90E+13 |
| 40,000 | 4.00E+06 | 4.00E+07 | 4.00E+08 | 4.00E+09 | 4.00E+10 | 4.00E+11 | 4.00E+12 | 4.00E+13 |
| 41,000 | 4.10E+06 | 4.10E+07 | 4.10E+08 | 4.10E+09 | 4.10E+10 | 4.10E+11 | 4.10E+12 | 4.10E+13 |

For in-furrow embodiments, in some embodiments, the dispersion of live microbes microbes is applied to provide microbes at a CFU concentration per acre of about 1E9-1E13 CFU/acre. In some embodiments, the dispersion of live microbes is applied to provide microbes at a CFU concentration per acre of about: 3E9, 1.5E10, 3E10, 1.5E11, 3E11, 8E11, 1.5E12, 3E12, or more.

In some embodiments, the dispersion of live microbes is applied in-furrow to provide microbes at a concentration of between about 3E9 to about 3E12 CFU per acre. In the dispersion of live microbes that is to be applied in furrow, the microbes can be present at a CFU concentration per milliliter of: 1×10¹, 1×10², 1×10³, 1×10⁴, 1-10⁵, 1×10⁶, 1×10⁷, 1×10⁸, 1×10⁹, 1×10¹⁰, 1×10¹¹, 1×10¹², 1×10¹³, or more. In certain aspects, the dispersion of live microbes that is to be applied in furrow comprise microbes at a concentration of about 1×10⁶ to about 1×10¹¹ CFU per milliliter. In other aspects, the dispersion of live microbes that is to be applied in furrow comprise microbes at a concentration of about 1×10⁷ to about 1×10¹⁰ CFU per milliliter. In other aspects, the dispersion of live microbes that is to be applied in furrow comprise microbes at a concentration of about 1×10⁸ to about 1×10⁹ CFU per milliliter. In other aspects, the liquid dispersion of live microbes that is to be applied in furrow comprise microbes at a concentration of up to about 1×10¹³ CFU per milliliter.

### Improvement of Plant Traits

The disclosure provides methods of using the water-soluble packages comprising dehydrated microbes disclosed herein for introducing or improving one or more of a variety of desirable traits in a plant through application of the dispersion of live microbes to a seed, seedling, plant, plant part, or the environs thereof prior to or during planting. Examples of traits that may be introduced or improved include: root biomass, root length, height, shoot length, leaf number, water use efficiency, overall biomass, yield, fruit size, grain size, photosynthesis rate, tolerance to drought, heat tolerance, salt tolerance, resistance to nematode stress, resistance to a fungal pathogen, resistance to a bacterial pathogen, resistance to a viral pathogen, level of a metabolite, and proteome expression. The desirable traits, including height, overall biomass, root and/or shoot biomass, seed germination, seedling survival, photosynthetic efficiency, transpiration rate, seed/fruit number or mass, plant grain or fruit yield, leaf chlorophyll content, photosynthetic rate, root length, or any combination thereof, can be used to measure growth, and compared with the growth rate of reference agricultural plants (e.g., plants without the improved traits) grown under identical conditions. In some embodiments, the methods described herein can improve plant traits, such as promoting plant growth, maintaining high chlorophyll content in leaves, increasing fruit or seed numbers, and increasing fruit or seed unit weight. In some embodiments, the plant, which has been applied a dispersion of live microbes described herein has improved health, yield, stress resistance, growth, or agronomic characteristics relative to a control plant.

Traits that may be improved by the methods disclosed herein include any observable characteristic of the seed or the plant resulting therefrom, including, for example, growth rate, height, weight, color, taste, smell, changes in the production of one or more compounds by the plant (including for example, metabolites, proteins, drugs, carbohydrates, oils, and any other compounds). In some embodiments, the methods disclosed herein may result in a change in genotypic information (for example, a change in the pattern of plant gene expression such as those associated with increased nitrogen fixation, in response to the microbes). In some embodiments, the plants show the absence, suppression or inhibition of a certain feature or trait (such as an undesirable feature or trait) as opposed to the presence of a certain feature or trait (such as a desirable feature or trait).

A preferred trait to be introduced or improved is nitrogen fixation, as described herein. A second preferred trait to be introduced or improved is colonization potential, as described herein. In some embodiments, a plant resulting from the methods described herein exhibits a difference in the trait that is at least about 5% greater, for example at least about 5%, at least about 8%, at least about 10%, at least about 15%, at least about 20%, at least about 25%, at least about 30%, at least about 40%, at least about 50%, at least about 60%, at least about 75%, at least about 80%, at least about 80%, at least about 90%, or at least 100%, at least about 200%, at least about 300%, at least about 400% or greater than a reference agricultural plant that is grown without the treatment/administration of the live dispersion of microbes. In additional examples, a plant resulting from the methods described herein exhibits a difference in the trait that is at least about 5% greater, for example at least about 5%, at least about 8%, at least about 10%, at least about 15%, at least about 20%, at least about 25%, at least about 30%, at least about 40%, at least about 50%, at least about 60%, at least about 75%, at least about 80%, at least about 80%, at least about 90%, or at least 100%, at least about 200%, at least about 300%, at least about 400% or greater than a reference agricultural plant grown under similar conditions in the soil.

The trait to be improved may be assessed under conditions including the application of one or more biotic or abiotic stressors. Examples of stressors include abiotic stresses (such as heat stress, salt stress, drought stress, cold stress, and low nutrient stress) and biotic stresses (such as nematode stress, insect herbivory stress, fungal pathogen stress, bacterial pathogen stress, and viral pathogen stress).

The trait improved may be nitrogen fixation, including in a plant not previously capable of nitrogen fixation. In some embodiments, enhanced levels of nitrogen fixation are achieved in the presence of fertilizer supplemented with glutamine, ammonia, or other chemical source of nitrogen. Methods for assessing degree of nitrogen fixation are known and may be employed to assess the methods described herein.

### Agricultural Plants

As described herein, the dispersion of live microbes produced by contacting the disclosed water-soluble packages with a liquid can be applied to plants or any plant part, such as seeds. In some embodiments, the plants are agricultural crops. In some embodiments the plants are monocots. In some embodiments, the plants are dicots.

In some embodiments, the plant belongs to the genera Hordeum, Oryza, Zea, and Triticeae. Other non-limiting examples of suitable plants include mosses, lichens, and algae. In some embodiments, the plants have economic, social and/or environmental value, such as food crops, fiber crops, oil crops, plants in the forestry or pulp and paper industries, feedstock for biofuel production and/or ornamental plants. In some embodiments, plants are used to produce economically valuable products such as a grain, a flour, a starch, a syrup, a meal, an oil, a film, a packaging, a nutraceutical product, a pulp, an animal feed, a fish fodder, a bulk material for industrial chemicals, a cereal product, a processed human-food product, a sugar, an alcohol, and/or a protein. Non-limiting examples of crop plants include maize, rice, wheat, barley, sorghum, millet, oats, rye triticale, buckwheat, sweet corn, sugar cane, onions, tomatoes, strawberries, asparagus, canola, soybean, potato, vegetables, cereals, and oilseeds. In some embodiments, the plant is a genetically modified organism (GMO), non-GMO, organic, or conventional plant. In some embodiments, the methods described herein are suitable for plant tissues from any of a variety of transgenic plants, non-transgenic plants, and hybrid plants thereof.

In some embodiments, the plants are important or interesting for agriculture, horticulture, biomass for the production of biofuel molecules and other chemicals, and/or forestry. Some examples of these plants may include pineapple, banana, coconut, lily, grasspeas and grass; and dicotyledonous plants, such as, for example, peas, alfalfa, tomatillo, melon, chickpea, chicory, clover, kale, lentil, soybean, tobacco, potato, sweet potato, radish, cabbage, rape, apple trees, grape, cotton, sunflower, thale cress, canola, citrus (including orange, mandarin, kumquat, lemon, lime, grapefruit, tangerine, tangelo, citron, and pomelo), pepper, bean, lettuce, Panicum virgatum (switch), Sorghum bicolor (sorghum, sudan), Miscanthus giganteus (miscanthus), Saccharum sp. (energycane), Populus balsamifera (poplar), Zea mays (corn), Glycine max (soybean), Brassica napus (canola), Triticum aestivum (wheat), Gossypium hirsutum (cotton), Oryza sativa (rice), Helianthus annuus (sunflower), Medicago sativa (alfalfa), Beta vulgaris (sugarbeet), Pennisetum glaucum (pearl millet), Panicum spp. Sorghum spp., Miscanthus spp., Saccharum spp., Erianthus spp., Populus spp., Secale cereale (rye), Salix spp. (willow), Eucalyptus spp. (eucalyptus), Triticosecale spp. (triticum—25 wheat X rye), bamboo, Carthamus tinctorius (safflower), Jatropha curcas (Jatropha), Ricinus communis (castor), Elaeis guineensis (oil palm), Phoenix dactylifera (date palm), Archontophoenix cunninghamiana (king palm), Syagrus romanzoffiana (queen palm), Linum usitatissimum (flax), Brassica juncea, Manihot esculenta (cassaya), Lycopersicon esculentum (tomato), Lactuca saliva (lettuce), Musa paradisiaca (banana), Solanum tuberosum (potato), Brassica oleracea (broccoli, cauliflower, brussel sprouts), Camellia sinensis (tea), Fragaria ananassa (strawberry), Theobroma cacao (cocoa), Coffea arabica (coffee), Vitis vinifera (grape), Ananas comosus (pineapple), Capsicum annum (hot & sweet pepper), Allium cepa (onion), Cucumis melo (melon), Cucumis sativus (cucumber), Cucurbita maxima (squash), Cucurbita moschata (squash), Spinacea oleracea (spinach), Citrullus lanatus (watermelon), Abelmoschus esculentus (okra), Solanum melongena (eggplant), Papaver somniferum (opium poppy), Papaver orientale, Taxus baccata, Taxus brevifolia, Artemisia annua, Cannabis saliva, Camptotheca acuminate, Catharanthus roseus, Vinca rosea, Cinchona officinalis, Coichicum autumnale, Veratrum californica, Digitalis lanata, Digitalis purpurea, Dioscorea 5 spp., Andrographis paniculata, Atropa belladonna, Datura stomonium, Berberis spp., Cephalotaxus spp., Ephedra sinica, Ephedra spp., Erythroxylum coca, Galanthus wornorii, Scopolia spp., Lycopodium serratum (Huperzia serrata), Lycopodium spp., Rauwolfia serpentina, Rauwolfia spp., Sanguinaria canadensis, Hyoscyamus spp., Calendula officinalis, Chrysanthemum parthenium, Coleus forskohlii, Tanacetum parthenium, Parthenium argentatum (guayule), Hevea spp. (rubber), Mentha spicata (mint), Mentha piperita (mint), Bixa orellana, Alstroemeria spp., Rosa spp. (rose), Dianthus caryophyllus (carnation), Petunia spp. (petunia), Poinsettia pulcherrima (poinsettia), Nicotiana tabacum (tobacco), Lupinus albus (lupin), Uniola paniculata (oats), Hordeum vulgare (barley), and Lolium spp. (rye).

In some embodiments, plant tissues or plant parts, e.g., seeds, from a monocotyledonous plant are treated. Monocotyledonous plants belong to the orders of the Alismatales, Arales, Arecales, Bromeliales, Commelinales, Cyclanthales, Cyperales, Eriocaulales, Hydrocharitales, Juncales, Lilliales, Najadales, Orchidales, Pandanales, Poales, Restionales, Triuridales, Typhales, and Zingiberales. Plants belonging to the class of the Gymnospermae are Cycadales, Ginkgoales, Gnetales, and Pinales. In some embodiments, the monocotyledonous plant can be selected from the group consisting of a maize, rice, wheat, barley, and sugarcane.

In some embodiments, plant tissues or plant parts, e.g., seeds, from a dicotyledonous plant are treated, including those belonging to the orders of the Aristochiales, Asterales, Batales, Campanulales, Capparales, Caryophyllales, Casuarinales, Celastrales, Cornales, Diapensales, Dilleniales, Dipsacales, Ebenales, Ericales, Eucomiales, Euphorbiales, Fabales, Fagales, Gentianales, Geraniales, Haloragales, Hamamelidales, Middles, Juglandales, Lamiales, Laurales, Lecythidales, Leitneriales, Magniolales, Malvales, Myricales, Myrtales, Nymphaeales, Papeverales, Piperales, Plantaginales, Plumb aginales, Podostemales, Polemoniales, Polygalales, Polygonales, Primulales, Proteales, Rafflesiales, Ranunculales, Rhamnales, Rosales, Rubiales, Salicales, Santales, Sapindales, Saraceniaceae, Scrophulariales, Theales, Trochodendrales, Umbellales, Urticales, and Violates. In some embodiments, the dicotyledonous plant can be selected from the group consisting of cotton, soybean, pepper, and tomato.

In some embodiments, the plant to be improved is not readily amenable to experimental conditions. For example, a crop plant may take too long to grow enough to practically assess an improved trait serially over multiple iterations. Accordingly, methods disclosed herein may be used on a model plant, such as a plant more amenable to evaluation under desired conditions. Non-limiting examples of model plants include Setaria, Brachypodium, and Arabidopsis. Additional plants and seeds acceptable for use within the methods and compositions of the present disclosure may be found in International Publication Nos. WO2020006246 and WO2020006064, the contents of each of which are herein incorporated by reference in their entirety.

The methods described herein are suitable for any of a variety of non-genetically modified maize plants or parts thereof. In some embodiments, the corn is organic. The methods described herein are suitable for any non-genetically modified hybrids, varieties, lineages, etc. In some embodiments, corn varieties generally fall under six categories: sweet corn, flint corn, popcorn, dent corn, pod corn, and flour corn.

The methods described herein are suitable for any of a hybrid, variety, lineage, etc. of genetically modified maize plants or part thereof. Furthermore, the methods described herein are suitable for any of the following genetically modified maize events, which have been approved in one or more countries, or any new genetically modified corn event, which may include Bt traits, glufosinate resistance, glyphosate resistance, etc.: 32138 (32138 SPT Maintainer), 3272 (ENOGEN), 3272×Bt11, 3272×bt11×GA21, 3272×Bt11×MIR604, 3272×Bt11×MIR604×GA21, 3272×Bt11×MIR604×TC1507×5307×GA21, 3272×GA21, 3272×MIR604, 3272×MIR604×GA21, 4114, 5307 (AGRISURE Duracade), 5307×GA21, 5307×MIR604×Bt11×TC1507×GA21 (AGRISURE Duracade 5122), 5307×MIR604×Bt11×TC1507×GA21×MIR162 (AGRISURE Duracade 5222), 59122 (Herculex RW), 59122×DAS40278, 59122×GA21, 59122×MIR604, 59122×MIR604×GA21, 59122×MIR604×TC1507, 59122×MIR604×TC1507×GA21, 59122×MON810, 59122×MON810×MIR604, 59122×MON810×NK603, 59122×MON810×NK603×MIR604, 59122×MON88017, 59122×MON88017×DAS40278, 59122×NK603 (Herculex RW ROUNDUP READY 2), 59122×NK603×MIR604, 59122×TC1507×GA21, 676, 678, 680, 3751 IR, 98140, 98140×59122, 98140×TC1507, 98140×TC1507×59122, Bt10 (Bt10), Bt11 [X4334CBR, X4734CBR](AGRISURE CB/LL), Bt11×5307, Bt11×5307×GA21, Bt11×59122×MIR604, Br11×59122×MIR604×GA21, Bt11×59122×MIR604×TC1507, M53, M56, DAS-59122-7, Bt11×59122×MIR604×TC1507×GA21, Bt11×59122×TC1507, TC1507×DAS-59122-7, Bt11×59122×TC1507×GA21, Bt11×GA21 (AGRISURE GT/CB/LL), Bt11×MIR162 (AGRISURE Viptera 2100), BT11×MIR162×5307, Bt11×MIR162×5307×GA21, Bt11×MIR162×GA21 (AGRISURE Viptera 3110), Bt11×MIR162×MIR604 (AGRISURE Viptera 3100), Bt11×MIR162×MIR604×5307, Bt11×MIR162×MIR604×5307×GA21, Bt11×MIR162×MIR604×GA21 (AGRISURE Viptera 3111/AGRISURE Viptera 4), Bt11, MIR162×MIR604×MON89034×5307×GA21, Bt11×MIR162×MIR604×TC1507, Bt11×MIR162×MIR604×TC1507×5307, Bt11×MIR162×MIR604×TC1507×GA21, Bt11×MIR162×MON89034, Bt11×MIR162×MON89034×GA21, Bt11×MIR162×TC1507, Bt11×MIR162×TC1507×5307, Bt11×MIR162×TC1507×5307×GA21, Bt11×MIR162×TC1507×GA21 (AGRISURE Viptera 3220), BT11×MIR604 (Agrisure BC/LL/RW), Bt11×MIR604×5307, Bt11×MIR604×5307×GA21, Bt11×MIR604×GA21, Bt11×MIR604×TC1507, Bt11×MIR604×TC1507×5307, Bt11×MIR604×TC1507×GA21, Bt11×MON89Ø34×GA21, Bt11×TC1507, Bt11×TC1507×5307, Bt11×TC1507×GA21, Bt176 [176](NaturGard KnockOut/Maximizer), BVLA430101, CBH-351 (STARLINK Maize), DAS40278 (ENLIST Maize), DAS40278×NK603, DBT418 (Bt Xtra Maize), DLL25 [B16], GA21 (ROUNDUP READY Maize/AGRISURE GT), GA21×MON810 (ROUNDUP READY Yieldgard Maize), GA21×T25, HCEM485, LY038 (MAVERA Maize), LY038×MON810 (MAVERA Yieldgard Maize), MIR162 (AGRISURE Viptera), MIR162×5307, MIR162×5307×GA21, MIR162×GA21, MIR162×MIR604, MIR162×MIR604×5307, MIR162×MIR604×5307×GA21, MIR162×MIR604×GA21, MIR162×MIR604×TC1507×5307, MIR162×MIR604×TC1507×5307×GA21, MIR162×MIR604×TC1507×GA21, MIR162×MON89034, MIR162×NK603, MIR162×TC1507, MIR162×TC1507×5307, MIR162×TC1507×5307×GA21, MIR162×TC1507×GA21, MIR604 (AGRISURE RW), MIR604×5307, MIR604×5307×GA21, MIR604×GA21 (AGRISURE GT/RW), MIR604×NK603, MIR604×TC1507, MIR604×TC1507×5307, MIR604×TC1507×5307×GA21, MIR604×TC1507×GA21, MON801 [MON80100], MON802, MON809, MON810 (YIELDGARD, MAIZEGARD), MON810×MIR162, MON810×MIR162×NK603, MON810×MIR604, MON810×MON88017 (YIELDGARD VT Triple), MON810×NK603×MIR604, MON832 (ROUNDUP READY Maize), MON863 (YIELDGARD Rootworm RW, MAXGARD), MON863×MON810 (YIELDGARD Plus), MON863×MON810×NK603 (YIELDGARD Plus with RR), MON863×NK603 (YIELDGARD RW+RR), MON87403, MON87411, MON87419, MON87427 (ROUNDUP READY Maize), MON87427×59122, MON87427×MON88017, MON87427×MON88017×59122, MON87427×MON89034, MON87427×MON89034×59122, MON87427×MON89034×MIR162×MON87411, MON87427×MON89034×MON88017, MON87427×MON89034×MON88017×59122, MON87427×MON89034×NK603, MON87427×MON89034×TC1507, MON87427×MON89034×TC1507×59122, MON87427×MON89034×TC1507×MON87411×59122, MON87427×MON89034×TC1507×MON87411×59122×DAS40278. MON87427×MON89034×TC1507×MON88017, MON87427×MON89Ø34×MIR162×NK603, MON87427×MON89Ø34×TC15Ø7×MON88Ø17×59122, MON87427×TC1507, MON87427×TC1507×59122, MON87427×TC1507×MON88017, MON87427×TC1507×MON88017×59122, MON87460 (GENUITY DROUGHTGARD), MON87460×MON88017, MON87460×MON89034×MON88017, MON87460×MON89034×NK603, MON87460×NK603, MON88017, MON88017×DAS40278, MON89034, MON89034×59122, MON89034×59122×DAS40278, MON89034×59122×MON88017, MON89034×59122×MON88017×DAS40278, MON89034×DAS40278, MON89034×MON87460, MON89034×MON88017 (GENUITY VT Triple Pro), MON89034×MON88017×DAS40278, MON89034×NK603 (GENUITY VT Double Pro), MON89034×NK603×DAS40278, MON89034×TC1507, MON89034×TC1507×59122, MON89034×TC1507×59122×DAS40278, MON89034×TC1507×DAS40278, MON89034×TC1507×MON88017, MON89034×TC1507×MON88017×59122 (GENUITY SMARTSTAX), MON89034×TC1507×MON88017×59122×DAS40278, MON89034×TC1507×MON88017×DAS40278, MON89034×TC1507×NK603 (POWER CORE), MON89034×TC1507×NK603×DAS40278, MON89034×TC1507×NK603×MIR162, MON89034×TC1507×NK603×MIR162×DAS40278, MON89034×GA21, MS3 (INVIGOR Maize), MS6 (INVIGOR Maize), MZHG0JG, MZIR098, NK603 (ROUNDUP READY 2 Maize), NK603×MON810×4114×MIR604, NK603×MON810 (YIELDGARD CB+RR), NK603×T25 (ROUNDUP READY LIBERTY LINK Maize), T14 (LIBERTY LINK Maize), T25 (LIBERTY LINK Maize), T25×MON810 (LIBERTY LINK YIELDGARD Maize), TC1507 (HERCULEX 1, HERCULEX CB), TC1507×59122×MON810×MIR604×NK603 (OPTIMUM INTRASECT XTREME), TC1507×MON810×MIR604×NK603, TC1507×5307, TC1507×5307×GA21, TC1507×59122 (HERCULEX XTRA), TC1507×59122×DAS40278, TC1507×59122×MON810, TC1507×59122×MON810×MIR604, TC1507×59122×MON810×NK603 (OPTIMUM INTRASECT XTRA), TC1507×59122×MON88017, TC1507×59122×MON88017×DAS40278, TC1507×59122×NK603 (HERCULEX XTRA RR), TC1507×59122×NK603×MIR604, TC1507×DAS40278, TC1507×GA21, TC1507×MIR162×NK603, TC1507×MIR604×NK603 (OPTIMUM TRISECT), TC1507×MON810, TC1507×MON810×MIR162, TC1507×MON810×MIR162×NK603, TC1507×MON810×MIR604, TC1507×MON810×NK603 (OPTIMUM INTRASECT), TC1507×MON810×NK603×MIR604, TC1507×MON88017, TC1507×MON88017×DAS40278, TC1507×NK603 (HERCULEX I RR), TC1507×NK603×DAS40278. TC6275, and VCO-01981-5.

### EXAMPLES

### Example 1: Encasement of Dry Plant Growth-Promoting Microbial Compositions in Water-Soluble Pouches Preserves Microbial Viability

The following experiment was performed to test whether two water-soluble packages—(1) paper pouch and (2) a clear pouch—may be used to package dry microbial compositions, such as those comprising: Klebsiella variicola 137-1036, sucrose and milk solids non-fat (MSNF). The paper pouch tested in this experiment was a SmartSolve® Blank Dissolving Pouch of 2″ width and 4″ Length. The clear pouch tested in this experiment was a pouch that is typically used in fishing. 5 g of dry microbial powder composition comprising Klebsiella variicola, sucrose and nonfat milk solids was sealed within each of the pouches.

The paper pouch and the clear pouch, each comprising the microbial powder composition, were added to 200 mL of PBS to produce a dispersion of live microbes referred to herein as the “paper pouch dispersion” and the “clear pouch dispersion” respectively. As a control, 5 g of the dry powder was also dispersed directly in 200 mL of PBS to produce a dispersion, referred to herein as “control dispersion”. The dispersion of the dry microbial powder was observed over time. For each of the samples, it took approximately 1-2 minutes with gentle shaking for the pouch to dissolve and allow the microbial powder to disperse and appear to dissolve. The paper pouch was not completely soluble since a fluffy sediment was noted in the bottle after storage overnight. Paper pouches showed faster dispersion of the dry microbial powder.

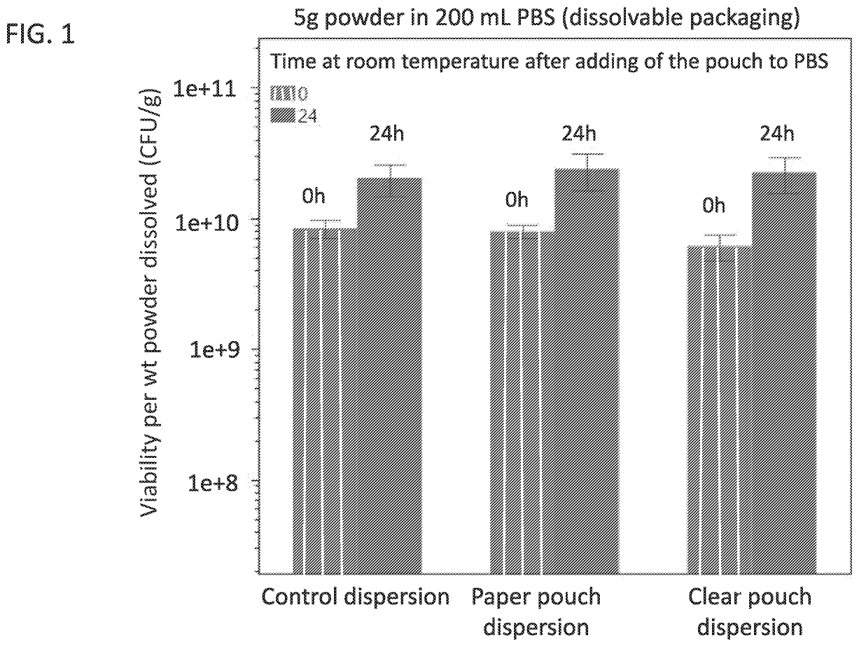

The viability of the microbes contained in the paper pouch dispersion, the clear pouch dispersion and the control dispersion was assessed by counting the number of colonies that were obtained upon spot plating the dispersion. The dispersion were spot plated either immediately after addition of the microbes to the PBS (time point “0 h”) or after 24 hours incubation at room temperature (time point “24h”). FIG. 1 shows the number of colony forming units (CFUs) obtained upon plating of each of the dispersions, relative to the weight of the microbial powder dispersed, for each of the time points. As a control, the powdered microbe was dissolved at the same concentration, and it was plated both initially and after the solution was incubated for 24 hours at room temperature.

FIG. 1 shows that the number of colonies obtained upon plating of paper pouch dispersion and clear pouch dispersion were comparable to the number of colonies obtained upon plating of the control dispersion after 0 hours and 24 hours time points. This indicates that packaging the microbes in the tested water dissolvable pouches does not adversely affect the viability of the microbes. FIG. 1 also shows that a higher number of colonies was obtained when the dispersion were plated after an incubation of 24 hours at room temperature. Without being bound by a theory, it is thought that, given the nonuniform particle size of this coarse microbial powder, a subpopulation of larger particles may have remained undissolved at 0 h, but were fully dissolved by 24 h. The microbial populations at 24h closely matched that measured three days earlier via a different protocol, in which the same microbial powder was completely dissolved at a lower concentration with more vigorous mixing (not depicted, but the titer was 3.2E10 CFU/g).

### Example 2: Biologically Relevant Concentrations of Microbes are Viable in the Presence of Commercial Grade Water-Soluble Films

To test whether the encasement of a nitrogen fixing-microbe in a water-soluble pouch would preserve the viability of the microbe at a concentration that is relevant for in-furrow applications, the following experiment was performed using two nitrogen fixing microbial strains: (i) Kosakonia sacchari designated with the strain ID 6-5687, represented by ATCC Accession No. PTA-126743, and (ii) Klebsiella variicola designated with the strain ID 137-2253, represented by ATCC Accession No. PTA-126740.

10% (w/v) of a freeze dried microbial powder of each of these strains was rehydrated, either in 1×PBS alone, or in 1×PBS in the presence of 2% (w/v) of a commercial grade, water-soluble MonoSol® film (either M7031, M8310 or M8534) by vortexing for 30 seconds to disperse the microbial cells. After rehydration by vortexing, the microbial suspensions were immediately diluted 1000× to a concentration of 0.01% w/v microbial powder. For microbial suspensions comprising one of the three tested water-soluble films, the concentration of the film upon 1000× dilution was 0.002% w/v in 1×PBS. The concentration of viable cells in the samples was measured after incubation at 21° C. for 1 hour by serially diluting the samples, and spot plating on media comprising tryptic soy agar.

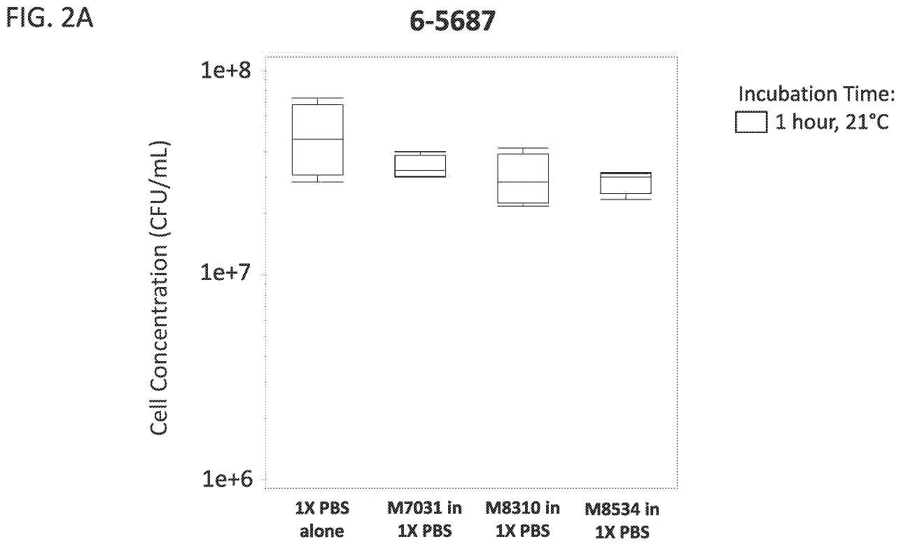

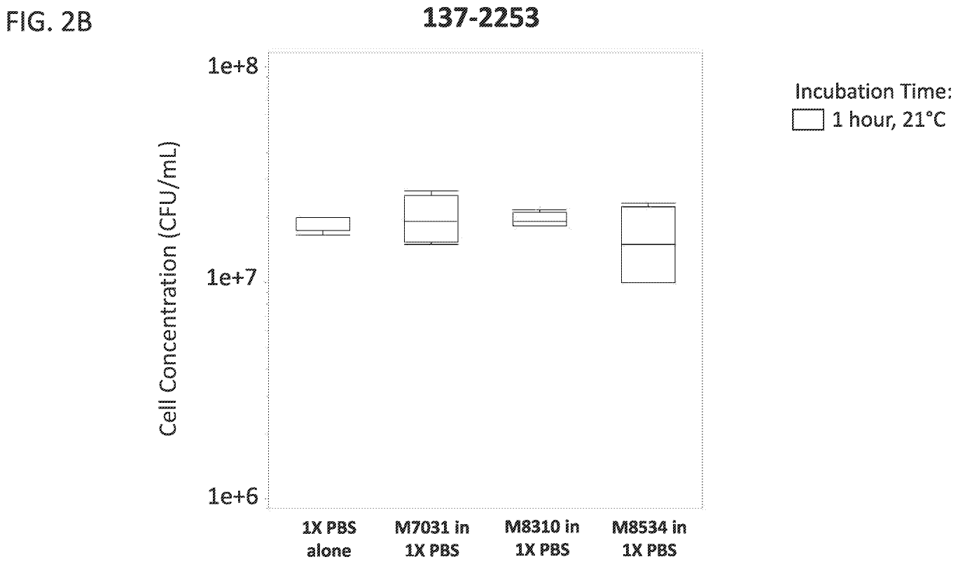

As shown in FIGS. 2A and 2B, the concentration of viable Kosakonia sacchari 6-5687 cells (FIG. 2A) or viable Klebsiella variicola 137-2253 cells (FIG. 2B) obtained after incubation of the rehydrated samples for 1 hour at 21° C. was comparable, irrespective of whether the microbe was rehydrated in 1×PBS alone, or in the presence of any of the tested water-soluble MonoSol® films.

These results are also remarkable because, the ratio of water-soluble film and microbial powder used in this experiment was more than 10-fold what would typically be used to encase the same amount of microbial powder, further highlighting the compatibility between the tested water-soluble films and the dry powder of nitrogen fixing microbes. Moreover, the concentration of microbes used in this experiment is comparable to the concentration of microbes used for in-furrow applications. This underlines that these results are pertinent to an agricultural setting and that water-soluble films may be used to encase and effectively deliver viable, plant growth-promoting microbes to plants and thus, improve plant growth and agricultural yield.

### Example 3: Plant Growth-Promoting Function is Maintained in the Presence of Water-Soluble Film

To test whether the encasement of a dried powder of a nitrogen fixing-microbe in a water-soluble pouch would retain the functional properties of the microbe, the following experiment was performed using two nitrogen fixing microbial strains: (i) Kosakonia sacchari designated with the strain ID 6-5687, represented by ATCC Accession No. PTA-126743, and (ii) Klebsiella variicola designated with the strain ID 137-2253, represented by ATCC Accession No. PTA-126740.

10% (w/v) of a dry microbial powder of each of these strains was rehydrated (approximately 1e10 CFU/mL), either in 1×PBS alone, or in 1×PBS in the presence of 2% (w/v) of a water-soluble film MonoSol® M8534, and incubated overnight at 4° C. Additionally, each of these strains was also grown overnight in a flask to obtain a microbial culture in suspension. The nitrogen fixation capability of each microbial strain—grown either in a liquid culture, or rehydrated in the presence or absence of the water-soluble film MonoSol® M8534—was measured using the acetylene reduction assay (ARA). The ARA assay uses the reduction of acetylene to ethylene as a readout for the nitrogen fixation capability of the microbe and was performed using the following method in the presence of 10 mM ammonium chloride (AmCl), which inhibits nitrogen fixation.

A modified version of the standard ASA assay described by Temme el al. PNAS, Apr. 16, 2012, 109 (18) 7085-7090 was used to measure nitrogenase activity in pure culture conditions. Strains were cultured from single colonies into 5 mL of SOB for 24 h (30° C., aerobic). The growth culture (1 mL) was then added to 25 mL of minimal media supplemented with 10 mM Glutamine and grown for 24 hours (30° C., aerobic). The growth culture (0.6 mL) was then added to 2.4 mL of minimal media supplemented with 10 mM ammonium chloride in airtight culture tubes prepared in an anaerobic chamber and grown for 4 h (30° C., anaerobic). A headspace of 10% was replaced by an equal volume of acetylene and incubation continued for an additional hour. A gas-tight syringe was used to remove 2 mL of headspace in preparation for ethylene production quantification using an Agilent 7890B gas chromatograph equipped with a flame ionization detector (FID). The initial culture biomass was compared to the end biomass by measuring OD590. Sterility is maintained throughout this experiment.

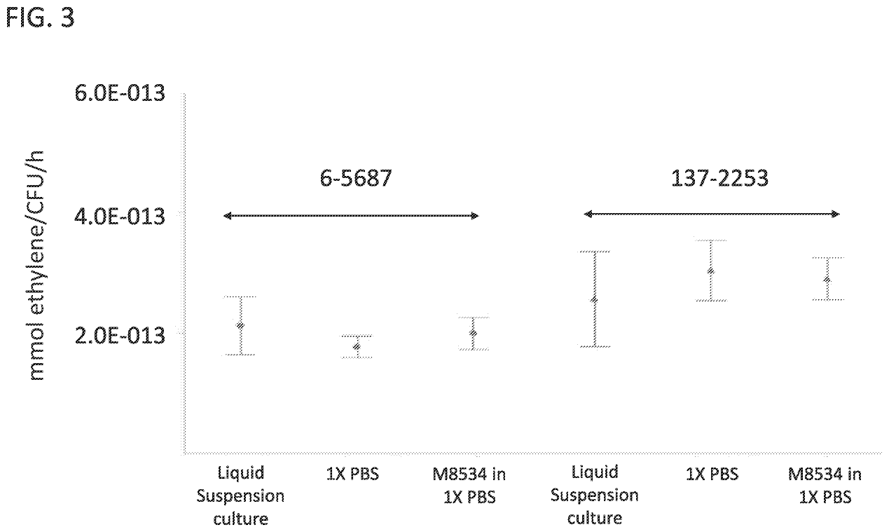

As shown in FIG. 3, in presence of ammonium chloride (AmCl), the amount of acetylene reduced to ethylene by either Kosakonia sacchari designated with the strain ID 6-5687 or Klebsiella variicola designated with the strain ID 137-2253 is surprisingly comparable, irrespective of whether the microbe was grown in a liquid suspension culture, or rehydrated in the presence or absence of the water-soluble film MonoSol® M8534. In other words, the nitrogen fixation capability of the microbe was unaffected by the differing formulations or growth conditions of the microbe and the presence of the water-soluble film. Notably, the ratio of water-soluble film and microbial powder used in this experiment was more than 10-fold of which would typically be used to encase the same amount of microbial powder, further highlighting the remarkable compatibility of the water-soluble film and functional dry microbial powders.

In sum, the results above indicate that the encasement of nitrogen fixing microbes in water-soluble pouches (such as, a water-soluble pouch comprising MonoSol® M8534) can effectively preserve the nitrogen fixing function of the microbes. These results further indicate that water-soluble pouches encasing agricultural formulations, such as dried powders of plant growth-promoting microbes can be used in a safe and consistent manner to increase plant productivity and agricultural yield.

### Example 4: Storage Conditions of Water-Soluble Pouches

The dry microbial powder compositions tested herein are added to water-dissolvable pouches and further encased in foil pouches along with an oxygen absorber. These foil pouches are stored at room temperature for different periods of time, for example, about 6 months, about 1 year, about 2 years or about 3 years. After storage for the fixed period of time, the viability of the microbes are tested as described in Example 1, and compared with viability of microbes in dry microbial powder that was not stored within the water-soluble pouch over this same time period. The physical properties of the dry microbial powder are also tested after storage. For instance, any changes in the appearance or dispersability of the microbial powder are noted.

### Example 5: Standardization of Water-Soluble Package Contents

The dry microbial powder compositions are blended with one or more different additives to measure how the presence of the additives affects the viability, shelf stability and dispersal of the compositions. For instance, the microbial compositions are blended with anticaking agents to test for improved dispersal. Further, the dispersion of the dry microbial powder upon contact of the water-soluble pouches with liquid are performed using the impeller mixers that are used for seed treatments.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

### Numbered Embodiments

Embodiment 1. A water-soluble film package composed of a water-soluble film, the package comprising: one or more compartment(s), wherein at least one of the one or more compartments comprises dehydrated microbes.

Embodiment 2. The water-soluble film package of embodiment 1, wherein the dehydrated microbes are nitrogen-fixing microbes.

Embodiment 3. The water-soluble film package of embodiment 2, wherein the nitrogen fixing microbes are diazotrophic bacteria.

Embodiment 4. The water-soluble film package of any one of embodiments 1-3, wherein the dehydrated microbes are in granular form.

Embodiment 5. The water-soluble film package of any one of embodiments 1-3, wherein the dehydrated microbes are powdered microbes.

Embodiment 6. The water-soluble film package of any one embodiments 1-5, wherein the one or more compartment(s) comprises an additive that enhances the shelf stability, uniformity, flow attributes, dissolution kinetics, or any combination thereof, of the dehydrated microbe.

Embodiment 7. The water-soluble film package of any one of embodiments 1-6, wherein the water-soluble film comprises a polymer selected from the group consisting of cellulose, a cellulose derivative, pullulan, polyvinyl pyrrolidone, polyvinyl alcohol, polyethylene glycol, carboxyvinyl copolymers, hydroxypropylmethyl cellulose, hydroxyethyl cellulose, hydroxypropyl cellulose, carboxymethyl cellulose, sodium alginate, xanthan gum, tragacanth gum, guar gum, acacia gum, arabic gum, polyacrylic acid, methylmethacrylate copolymer, carboxyvinyl copolymers, starch, gelatin, and combinations thereof.

Embodiment 8. The water-soluble film package of embodiment 7, wherein the water-soluble film further comprises a polyethylene oxide.

Embodiment 9. The water-soluble film package of any one of embodiments 1-8, wherein the polymer further comprises a water-insoluble polymer selected from the group consisting of ethylcellulose, hydroxypropyl ethyl cellulose, cellulose acetate phthalate, hydroxypropyl methyl cellulose phthalate, polyvinylacetatephthalates, phthalated gelatin, crosslinked gelatin, poly(lactic acid)/poly(glycolic acid)/polyethyleneglycol copolymers, polycaprolactone and combinations thereof.

Embodiment 10. The water-soluble film package of any one of embodiments 1-9 wherein the polymer further comprises a polymer selected from the group consisting of methylmethacrylate copolymer, polyacrylic acid polymer, poly(glycolic acid) (PGA), poly(lactic acid) (PLA), poly(lactic acid)/poly(glycolic acid)/polyethyleneglycol copolymers, polydioxanones, polyoxalates, poly(\acute{α}-esters), polyanhydrides, polyacetates, polycaprolactones, poly(orthoesters), polyamino acids, polyaminocarbonates, polyurethanes, polycarbonates, polyamides, poly(alkyl cyanoacrylates), and mixtures and copolymers thereof.

Embodiment 11. The water-soluble film package of any one of embodiments 1-10, wherein the water-soluble film comprises polyvinyl alcohol.

Embodiment 12. The water-soluble film package of any one of embodiments 1-11, wherein the dehydrated microbes comprise a controlled release composition coating.

Embodiment 13. The water-soluble film package of any one of embodiments 1-12, wherein the dehydrated microbes comprise a rapid dissolution formulation of the microbes.

Embodiment 14. The water-soluble film package of any one of embodiments 1-13, wherein the dehydrated microbes comprise a blend of dehydrated microbes with at least 25% of one or more hygroscopic salt(s).

Embodiment 15. The water-soluble film package of any one of embodiments 1-14, wherein the package comprises a first and a second compartment, and wherein the dehydrated microbes are in the first compartment.

Embodiment 16. The water-soluble film package of embodiment 15, wherein the first or second compartment comprises a carbon source capable of enhancing growth of the dehydrated microbes.

Embodiment 17. The water-soluble film package of any one of embodiments 1-14, wherein the one or more compartment(s) comprise a carbon source capable of enhancing growth of the dehydrated microbes.

Embodiment 18. The water-soluble film package of embodiment 15, wherein the first or second compartment comprises a dispersing agent.

Embodiment 19. The water-soluble film package of any one of embodiments 1-14, wherein the one or more compartment(s) comprise a dispersing agent.

Embodiment 20. The water-soluble film package of embodiment 15, wherein the first or second compartment comprises a fertilizer.

Embodiment 21. The water-soluble film package of any one of embodiments 1-14, wherein the one or more compartment(s) comprise a fertilizer.

Embodiment 21.1 The water-soluble film package of embodiment 15, wherein the first or second compartment comprises a plant growth hormone.

Embodiment 21.2 The water-soluble film package of any one of embodiments 1-14, wherein the one or more compartment(s) comprise a plant growth hormone.

Embodiment 21.3 The water-soluble film package of any one of embodiments 1-14, wherein the one or more compartment(s) comprise a bulking agent, an anticaking agent, a microbial stabilizer, a physical stabilizer, a dispersant, and/or milk solids nonfat.

Embodiment 21.4 The water-soluble film package of any one of embodiments 1-14, wherein the one or more compartment(s) comprise a bulking agent, an anticaking agent, a microbial stabilizer, a physical stabilizer, a dispersant, and/or milk solids nonfat,
wherein the microbial stabilizer selected from the list consisting of a monosaccharide, a disaccharide, a polysaccharide, a pentose, a hexose, an oligosaccharide, an oligofructose, a sugar alcohol, an amino acid, a protein or protein hydrolysate, a polymer, glucose, fructose, trehalose, sucrose, lactose, melibiose, inulin and lactulose, and
wherein the physical stabilizer selected from the list consisting of maltodextrin, polyethylene glycol (PEG), xanthan gum, pectin, alginates, microcrystalline cellulose, and dextran.

Embodiment 22. The water-soluble film package of embodiment 21.3 or embodiment 21.4, wherein the one or more compartment(s) comprise sucrose, milk solids nonfat, or a combination thereof.

Embodiment 23. The water-soluble film package of embodiment 15, wherein the first or second compartment comprises an agent selected from the group consisting of a bulking agent, an anticaking agent, a microbial stabilizer, a physical stabilizer, and a dispersant.

Embodiment 24. The water-soluble film package of any one of embodiments 1-14, wherein the one or more compartment(s) comprise an agent selected from the group consisting of a bulking agent, an anticaking agent, a microbial stabilizer, a physical stabilizer, and a dispersant.

Embodiment 25. The water-soluble film package of embodiment 15, wherein the first or second compartment comprises a microbial stabilizer selected from the list consisting of a monosaccharide, a disaccharide, a polysaccharide, a pentose, a hexose, an oligosaccharide, an oligofructose, a sugar alcohol, an amino acid, a protein or protein hydrolysate, and a polymer.

Embodiment 26. The water-soluble film package of any one of embodiments 1-14, wherein the one or more compartment(s) comprise a microbial stabilizer selected from the list consisting of a monosaccharide, a disaccharide, a polysaccharide, a pentose, a hexose, an oligosaccharide, an oligofructose, a sugar alcohol, an amino acid, a protein or protein hydrolysate, and a polymer.

Embodiment 27. The water-soluble film package of embodiment 15, wherein the first or second compartment comprises a microbial stabilizer selected from the list consisting of glucose, fructose, trehalose, sucrose, lactose, melibiose, inulin and lactulose.

Embodiment 28. The water-soluble film package of any one of embodiments 1-14, wherein the one or more compartment(s) comprise a microbial stabilizer selected from the list consisting of glucose, fructose, trehalose, sucrose, lactose, melibiose, inulin and lactulose.

Embodiment 29. The water-soluble film package of embodiment 15, wherein the first or second compartment comprises a physical stabilizer selected from the list consisting of maltodextrin, polyethylene glycol (PEG), xanthan gum, pectin, alginates, microcrystalline cellulose, and dextran.

Embodiment 30. The water-soluble film package of any one of embodiments 1-14, wherein the one or more compartment(s) comprise a physical stabilizer selected from the list consisting of maltodextrin, polyethylene glycol (PEG), xanthan gum, pectin, alginates, microcrystalline cellulose, and dextran.

Embodiment 31. The water-soluble film package of any one of embodiments 2-30, wherein the nitrogen fixing microbes are gram-negative.

Embodiment 32. The water-soluble film package of any one of embodiments 2-31, wherein the nitrogen fixing microbes are of a genus selected from the group consisting of: Acetobacter, Achromobacter, Aerobacter, Anabaena, Azoarcus, Azomonas, Azorhizobium, Azospirillum, Azotobacter, Beijernickia, Bradyrhizobium, Burkholderia, Citrobacter, Derxia, Enterobacter, Herbaspirillum, Klebsiella, Kluyvera, Kosakonia, Mesorhizobium, Metakosakonia, Paraburkholderia, Nostoc, Rahnella, Rhizobium, Rhodobacter, Rhodopseudomonas, Rhodospirillum, Serratia Sinorhizobium, Spirillum, Trichodesmium, Xanthomonas, and combinations thereof.

Embodiment 33. The water-soluble film package of any one of embodiments 2-32, wherein the nitrogen fixing microbes are of a species selected from the group consisting of: Achromobacter marplatensis, Achromobacter spiritinus, Azospirillum lipoferum, Enterobacter sacchari, Herbaspirillum aquaticum, Klebsiella variicola, Kluyvera intermedia, Kosakonia pseudosacchari, Kosakonia sacchari, Metakosakonia intestini, Paraburkholderia tropica, Rahnella aquatilis, and combinations thereof.

Embodiment 34. The water-soluble film package of any one of embodiments 2-26, wherein the nitrogen fixing microbes are gram-positive.

Embodiment 35. The water-soluble film package of any one of embodiments 2-30, and 34, wherein the nitrogen fixing microbes are of a genus selected from the group consisting of: Arthrobacter, Agromyces, Bacillus, Clostridium, Corynebacterium, Frankia, Heliobacillus, Heliobacterium, Heliophilum, Heliorestis Methanobacterium, Microbacterium, Micrococcus, Micromonospora, Mycobacterium, Paenibacillus, Propionibacterium, and Streptomyces.

Embodiment 36. The water-soluble film package of any one of embodiments 2-30, and 34-35, wherein the nitrogen fixing microbes are of a species selected from the group consisting of: Bacillus amyloliquefaciens, Bacillus macerans, Bacillus pumilus, Bacillus thuringiensis, Clostridium acetobutylicum, Corynebacterium autitrophicum Methanobacterium formicicum, Methanobacterium omelionski, Microbacterium murale, Mycobacterium flavum, Paenibacillus polymyxa, Paenibacillus riograndensis, Propionibacterium acidipropio, Propionibacterium freudenreichii, Streptococcus lactis, Streptomyces griseus, and combinations thereof.

Embodiment 37. The water-soluble film package of any one of embodiments 2-30, wherein the nitrogen fixing microbes are of the genus Klebsiella.

Embodiment 38. The water-soluble film package of any one of embodiments 2-30, wherein the nitrogen fixing microbes are of the species Klebsiella variicola.

Embodiment 39. The water-soluble film package of any one of embodiments 2-30, wherein the nitrogen fixing microbes comprise one or more strains identified by a strain deposit number disclosed herein.

Embodiment 40. The water-soluble film package of any one of embodiments 2-30, wherein the nitrogen fixing microbes comprise a strain deposited as NCMA 201712002, PTA-126740, PTA-126743, or any combination thereof.

Embodiment 40.1. The water-soluble film package of any one of claims 2-30, wherein the nitrogen fixing microbes comprise a strain deposited as PTA-126740.

Embodiment 40.2. The water-soluble film package of any one of claims 2-30, wherein the nitrogen fixing microbes comprise a strain deposited as PTA-126743.

Embodiment 41. The water-soluble film package of any one of embodiments 2-30, wherein the nitrogen fixing microbes are endophytic, epiphytic, or rhizospheric.

Embodiment 42. The water-soluble film package of any one of embodiments 2-38, and 41, wherein the nitrogen fixing microbes are wild type bacteria.

Embodiment 43. The water-soluble film package of any one of embodiments 2-41, wherein the nitrogen fixing microbes are engineered bacteria.

Embodiment 44. The water-soluble film package of any one of embodiments 2-41, and 43, wherein the nitrogen fixing microbes are transgenic bacteria.

Embodiment 45. The water-soluble film package of any one of embodiments 2-41, and 43, wherein the nitrogen fixing microbes are intragenic bacteria.

Embodiment 46. The water-soluble film package of any one of embodiments 2-41, and 43-45, wherein the nitrogen fixing microbes are remodeled bacteria.

Embodiment 47. The water-soluble film package of any one of embodiments 2-41, 43, and 46, wherein the nitrogen fixing microbes comprise a non-intergeneric genomic modification.

Embodiment 48. The water-soluble film package of any one of embodiments 2-41, and 43-47, wherein the nitrogen fixing microbes are engineered bacteria capable of fixing atmospheric nitrogen in the presence of exogenous nitrogen.

Embodiment 49. The water-soluble film package of any one of embodiments 2-41, and 43-48, wherein the nitrogen fixing microbes are engineered bacteria comprising at least one genetic variation introduced into at least one gene, or non-coding polynucleotide, of the nitrogen fixation or assimilation genetic regulatory network.

Embodiment 50. The water-soluble film package of any one of embodiments 2-41, and 43-49, wherein the nitrogen fixing microbes are engineered bacteria comprising an introduced control sequence operably linked to at least one gene of the nitrogen fixation or assimilation genetic regulatory network.

Embodiment 51. The water-soluble film package of any one of embodiments 2-41, and 43-50. wherein the nitrogen fixing microbes are engineered bacteria comprising a heterologous promoter operably linked to at least one gene of the nitrogen fixation or assimilation genetic regulatory network.

Embodiment 52. The water-soluble film package of any one of embodiments 2-41, and 43-51, wherein the nitrogen fixing microbes are engineered bacteria comprising at least one genetic variation selected from the group consisting of: nifA, nifL, ntrB, ntrC, polynucleotide encoding glutamine synthetase, glnA, glnB, glnK, drat, amtB, polynucleotide encoding glutaminase, glnD, glnE, nifJ, nifH, nifD, nifK, nifY, nifE, nifN, nifU, nifS, nifV, nifW, nifZ, nifM, nifF, nifB, nifQ, a gene associated with biosynthesis of a nitrogenase enzyme, and combinations thereof.

Embodiment 53. The water-soluble film package of any one of embodiments 2-41, and 43-52, wherein the nitrogen fixing microbes are engineered bacteria comprising at least one genetic variation introduced into at least one gene, or non-coding polynucleotide, of the nitrogen fixation or assimilation genetic regulatory network that results in one or more of: increased expression or activity of NifA or glutaminase; decreased expression or activity of NifL, NtrB, glutamine synthetase, GlnB, GlnK, DraT, AmtB; decreased adenylyl-removing activity of GlnE; or decreased expression or uridylyl-removing activity of GlnD.

Embodiment 54. The water-soluble film package of any one of embodiments 2-41, and 43-53, wherein the nitrogen fixing microbes are engineered bacteria comprising a mutated nifL gene that has been altered to comprise a heterologous promoter inserted into said mf gene.

Embodiment 55. The water-soluble film package of any one of embodiments 2-41, and 43-54, wherein the nitrogen fixing microbes are engineered bacteria comprising a mutated glnE gene that results in a truncated GlnE protein lacking an adenylyl-removing (AR) domain.

Embodiment 56. The water-soluble film package of any one of embodiments 2-41, and 43-55, wherein the nitrogen fixing microbes are engineered bacteria comprising a mutated glnD gene that results in the lack of expression of said glnD gene.

Embodiment 57. The water-soluble film package of any one of embodiments 2-41, and 43-56, wherein the nitrogen fixing microbes are engineered bacteria comprising a mutated amtB gene that results in the lack of expression of said amtB gene.

Embodiment 58. The water-soluble film package of any one of embodiments 2-41, and 43-57, wherein the nitrogen fixing microbes are engineered bacteria comprising at least one of: a mutated nifL gene that has been altered to comprise a heterologous promoter inserted into said nifL gene; a mutated glnE gene that results in a truncated GlnE protein lacking an adenylyl-removing (AR) domain; a mutated amtB gene that results in the lack of expression of said amtB gene; a mutated glnD gene that results in the lack of expression of said glnD gene; and combinations thereof.

Embodiment 59. A method for supplying nitrogen to a plant, the method comprising:
a) contacting the water-soluble film package of any one of embodiments 1-58 with a liquid to produce a dispersion of live microbes; and
b) applying the dispersion of live microbes to a locus comprising the plant, thereby colonizing the locus with the microbes;
   wherein the microbes fix atmospheric nitrogen, thereby supplying nitrogen to the plant.

Embodiment 60. A method for coating a seed with nitrogen-fixing microbes, the method comprising
a) contacting the water-soluble film package of any one of embodiments 1-58 with a liquid to produce a dispersion of live microbes; and
b) coating the seed with the dispersion of live microbes.

Embodiment 61 The method of embodiment 60, wherein the water-soluble film package comprises polyvinyl alcohols.

Embodiment 62. The method of any one of embodiments 59 to 61, wherein the liquid is water or an aqueous solution.

Embodiment 63. A method of producing a dispersible formulation of dehydrated microbes, comprising: encapsulating dehydrated microbes in a water-soluble film, thereby producing a water-soluble film package comprising one or more compartment(s).

Embodiment 64. The method of embodiment 63, comprising culturing a microbe in growth media to produce a microbial culture liquid, and dehydrating said microbial culture liquid to produce the dehydrated microbes.

Embodiment 65. The method of embodiment 64, comprising concentrating the microbial culture liquid to further increase cell density of the culture before the dehydrating step.

Embodiment 66. The method of embodiment 65, wherein the concentrating step comprises a technique selected from the group consisting of centrifugation, tangential flow filtration (TFF), and a combination thereof.

Embodiment 67. The method of any one of embodiments 63-66, wherein the microbial culture liquid comprises an excipient.

Embodiment 68. The method of embodiment 67, wherein the excipient comprises a bulking agent, an anticaking agent, a microbial stabilizer, a physical stabilizer, a dispersant, and/or milk solids nonfat.

Embodiment 68.1 The method of embodiment 67, wherein the excipient comprises a bulking agent, an anticaking agent, a microbial stabilizer, a physical stabilizer, a dispersant, and/or milk solids nonfat,
wherein the microbial stabilizer selected from the list consisting of a monosaccharide, a disaccharide, a polysaccharide, a pentose, a hexose, an oligosaccharide, an oligofructose, a sugar alcohol, an amino acid, a protein or protein hydrolysate, a polymer, glucose, fructose, trehalose, sucrose, lactose, melibiose, inulin and lactulose, and
wherein the physical stabilizer selected from the list consisting of maltodextrin, polyethylene glycol (PEG), xanthan gum, pectin, alginates, microcrystalline cellulose, and dextran.

Embodiment 69. The method of embodiment 68 or embodiment 68.1, wherein the excipient comprises sucrose, milk solids nonfat, or a combination thereof.

Embodiment 70. The method of any one of embodiments 63-69, wherein the dehydrating step comprises a technique selected from the group consisting of freeze drying, spray drying, fluidized bed drying, extrusion, drying, and a combination thereof.

Embodiment 71. The method of any one of embodiments 63-70, wherein the dehydrated microbes comprise a dry microbial powder.

Embodiment 72. The method of embodiment 71, wherein the dry microbial powder is produced by processing the dehydrated microbes using a technique selected from the group consisting of milling, sieving, and a combination thereof.

Embodiment 73. The method of embodiment 71 or 72, wherein the dehydrated microbes comprise granules of dehydrated microbes.

Embodiment 74. The method of any one of embodiments 63-73, wherein the dehydrated microbes comprise a dry excipient.

Embodiment 75. The method of embodiment 74, wherein the excipient comprises an ingredient selected from the group consisting of bulking agents, anticaking agents, dispersants, and any combination thereof.

Embodiment 76. The method of any one of embodiments 63-75, wherein the dehydrated microbes comprise at least 25% hygroscopic salt(s) by weight.

Embodiment 77. The method of any one of embodiments 63-76, wherein the water-soluble film package contains a unit dose of the dehydrated microbes for the treatment of an acre of a corn crop.

Embodiment 78. The method of any one of embodiments 63-77, wherein the water-soluble package is stored in a hermetically sealed vessel.

Embodiment 79. The method of any one of embodiments 63-78, wherein the one or more compartment(s) of the water-soluble film package comprise a first and a second compartment, and wherein the dehydrated microbes are in the first compartment.

Embodiment 80. The method of any one of embodiments 63 to 79, wherein the one or more compartment(s) of the water-soluble film package comprise an additive capable of enhancing a property of the dehydrated microbes selected from the group consisting of shelf stability, uniformity, flow attributes, dissolution kinetics, and any combination thereof.

Embodiment 81. The method of any one of embodiments 63-80, wherein the one or more compartment(s) of the water-soluble film package comprise a carbon source capable of enhancing growth of the dehydrated microbes.

Embodiment 82. The method of any one of embodiments 63 to 81, wherein the one or more compartment(s) of the water-soluble film package comprise a dispersing agent.

Embodiment 83. The method of any one of embodiments 63-82, wherein one or more compartment(s) of the water-soluble film package comprise a fertilizer.

Embodiment 84. The method of any one of embodiments 63-83, wherein the one or more compartment(s) of the water-soluble film package comprise a plant growth hormone.

Embodiment 85. The method of any one of embodiments 63 to 84, wherein the one or more compartment(s) of the water-soluble film package comprise an agent selected from the group consisting of a bulking agent, an anticaking agent, a microbial stabilizer, a physical stabilizer, and a dispersant.

Embodiment 86. The method of embodiment 85 wherein the microbial stabilizer is selected from the list consisting of a monosaccharide, a disaccharide, a polysaccharide, a pentose, a hexose, an oligosaccharide, an oligofructose, a sugar alcohol, an amino acid, a protein or protein hydrolysate, and a polymer.

Embodiment 87. The method of embodiment 85, wherein the microbial stabilizer is selected from the list consisting of glucose, fructose, trehalose, sucrose, lactose, melibiose, inulin and lactulose.

Embodiment 88. The method of embodiment 85, wherein the physical stabilizer selected from the list consisting of maltodextrin, polyethylene glycol (PEG), xanthan gum, pectin, alginates, microcrystalline cellulose, and dextran.

Embodiment 89. A method of producing a dispersible formulation of dehydrated microbes, comprising
a) providing microbes and a water-soluble film;
b) culturing the microbes in growth media to produce a microbial culture liquid;
c) admixing the microbial culture liquid with an ingredient selected from the group consisting of a bulking agent, an anticaking agent, a microbial stabilizer, a physical stabilizer, a dispersant, and/or milk solids nonfat;
d) dehydrating the microbial culture liquid to produce dried material, wherein the dried material comprises dehydrated microbes,
e) encapsulating the dried material in the water-soluble film, thereby producing a water-soluble film package.

Embodiment 89.1 A method of producing a dispersible formulation of dehydrated microbes, comprising.
a) providing microbes and a water-soluble film;
b) culturing the microbes in growth media to produce a microbial culture liquid;
c) admixing the microbial culture liquid with an ingredient selected from the group consisting of a bulking agent, an anticaking agent, a microbial stabilizer, a physical stabilizer, a dispersant, and/or milk solids nonfat;
d) dehydrating the microbial culture liquid to produce dried material, wherein the dried material comprises dehydrated microbes,
e) encapsulating the dried material in the water-soluble film, thereby producing a water-soluble film package,
   wherein the microbial stabilizer selected from the list consisting of a monosaccharide, a disaccharide, a polysaccharide, a pentose, a hexose, an oligosaccharide, an oligofructose, a sugar alcohol, an amino acid, a protein or protein hydrolysate, a polymer, glucose, fructose, trehalose, sucrose, lactose, melibiose, inulin and lactulose, and
   wherein the physical stabilizer selected from the list consisting of maltodextrin, polyethylene glycol (PEG), xanthan gum, pectin, alginates, microcrystalline cellulose, and dextran.

Embodiment 90. The method of embodiment 89 or embodiment 89.1, wherein step c) comprises admixing the microbial culture liquid with sucrose, milk solids nonfat, or a combination thereof.

Embodiment 91. The method of embodiment 89, wherein the method comprises concentrating the microbial culture liquid to further increase cell density prior to the dehydrating step.

Embodiment 92. The method of any one of embodiments 89 to 91, wherein the method comprises processing of the dried material to produce a dry microbial powder.

Embodiment 93. The method of embodiment 92, wherein the method comprises agglomerating the dry microbial powder to produce granules.

Embodiment 94. The method of embodiment 92 or 94, wherein the method comprises blending the dry microbial powder with a dry ingredient selected from the group consisting of bulking agents, anticaking agents, dispersants, and any combination thereof.

Embodiment 95. The method of any one of embodiments 63 to 94, wherein the water-soluble film comprises a polymer selected from the group consisting of cellulose, a cellulose derivative, pullulan, polyvinyl pyrrolidone, polyvinyl alcohol, polyethylene glycol, carboxyvinyl copolymers, hydroxypropylmethyl cellulose, hydroxyethyl cellulose, hydroxypropyl cellulose, carboxymethyl cellulose, sodium alginate, xanthan gum, tragacanth gum, guar gum, acacia gum, arabic gum, polyacrylic acid, methylmethacrylate copolymer, carboxyvinyl copolymers, starch, gelatin, and combinations thereof.

Embodiment 96. The method of embodiment 95, wherein the water-soluble film comprises a polyethylene oxide.

Embodiment 97. The method of embodiment 95 or 96, wherein the polymer further comprises a water-insoluble polymer selected from the group consisting of ethylcellulose, hydroxypropyl ethyl cellulose, cellulose acetate phthalate, hydroxypropyl methyl cellulose phthalate, polyvinylacetatephthalates, phthalated gelatin, crosslinked gelatin, poly(lactic acid)/poly(glycolic acid)/polyethyleneglycol copolymers, polycaprolactone and combinations thereof.

Embodiment 98. The method of any one of embodiments 95-97, wherein the polymer further comprises a polymer selected from the group consisting of methylmethacrylate copolymer, polyacrylic acid polymer, poly(glycolic acid) (PGA), poly(lactic acid) (PLA), poly(lactic acid)/poly(glycolic acid)/polyethyleneglycol copolymers, polydioxanones, polyoxalates, poly(\acute{α}-esters), polyanhydrides, polyacetates, polycaprolactones, poly(orthoesters), polyamino acids, polyaminocarbonates, polyurethanes, polycarbonates, polyamides, poly(alkyl cyanoacrylates), and mixtures and copolymers thereof.

Embodiment 99. The method of any one of embodiments 63-98, wherein the water-soluble film comprises polyvinyl alcohol.

Embodiment 100. The method of any one of embodiments 63 to 99, wherein the dehydrated microbes are nitrogen-fixing microbes.

Embodiment 101. The method of embodiment 100, wherein the nitrogen fixing microbes are diazotrophic bacteria.

Embodiment 102. The method of embodiment 100 or embodiment 101, wherein the nitrogen fixing microbes are gram-negative.

Embodiment 103. The method of any one of embodiments 100-102, wherein the nitrogen fixing microbes are of a genus selected from the group consisting of: Acetobacter, Achromobacter, Aerobacter, Anabaena, Azoarcus, Azomonas, Azorhizobium, Azospirillum, Azotobacter, Beijernickia, Bradyrhizobium, Burkholderia, Citrobacter, Derxia, Enterobacter, Herbaspirillum, Klebsiella, Kluyvera, Kosakonia, Mesorhizobium, Metakosakonia, Paraburkholderia, Nostoc, Rahnella, Rhizobium, Rhodobacter, Rhodopseudomonas, Rhodospirillum, Serratia Sinorhizobium, Spirillum, Trichodesmium, Xanthomonas, and combinations thereof.

Embodiment 104. The method of any one of embodiments 100-103, wherein the nitrogen fixing microbes are of a species selected from the group consisting of: Achromobacter marplatensis, Achromobacter spiritinus, Azospirillum lipoferum, Enterobacter sacchari, Herbaspirillum aquaticum, Klebsiella variicola, Kluyvera intermedia, Kosakonia pseudosacchari, Kosakonia sacchari, Metakosakonia intestini, Paraburkholderia tropica, Rahnella aquatilis, and combinations thereof.

Embodiment 105. The method of any one of embodiments 100 and 101, wherein the nitrogen fixing microbes are gram-positive.

Embodiment 106. The method of any one of embodiments 100, 101 and 105, wherein the nitrogen fixing microbes are of a genus selected from the group consisting of: Arthrobacter, Agromyces, Bacillus, Clostridium, Corynebacterium, Frankia, Heliobacillus, Heliobacterium, Heliophilum, Heliorestis Methanobacterium, Microbacterium, Micrococcus, Micromonospora, Mycobacterium, Paenibacillus, Propionibacterium, and Streptomyces.

Embodiment 107. The method of any one of embodiments 100 and 101, wherein the nitrogen fixing microbes are of a species selected from the group consisting of: Bacillus amyloliquefaciens, Bacillus macerans, Bacillus pumilus, Bacillus thuringiensis, Clostridium acetobutylicum, Corynebacterium autitrophicum Methanobacterium formicicum, Methanobacterium omelionski, Microbacterium murale, Mycobacterium flavum, Paenibacillus polymyxa, Paenibacillus riograidensis, Propionibacterium acidipropio, Propionibacterium freudenreichii, Streptococcus lactis, Streptomyces griseus, and combinations thereof.

Embodiment 108. The method of any one of embodiments 100-102, wherein the nitrogen fixing microbes comprise one or more strains identified by a strain deposit number disclosed herein.

Embodiment 109. The method of any one of embodiments 100-102, wherein the nitrogen fixing microbes comprise a strain deposited as NCMA 201712002, PTA-126740, PTA-126743, or any combination thereof.

Embodiment 110. The method of any one of embodiments 100-102, wherein the nitrogen fixing microbes are of the genus Klebsiella.

Embodiment 111. The method of embodiment 110, wherein the nitrogen fixing microbes are of the species Klebsiella variicola.

Embodiment 112. The method of embodiment 111, wherein the nitrogen fixing microbes are of the strain deposited as NCMA 201712002.

Embodiment 113. The method of embodiment 111, wherein the nitrogen fixing microbes are of the strain deposited as PTA-126740.

Embodiment 113.1 The method of claim 111, wherein the nitrogen fixing microbes comprise a strain deposited as PTA-126743.

Embodiment 114. The method of any one of embodiments 100-113.1, wherein the nitrogen fixing microbes are endophytic, epiphytic, or rhizospheric.

Embodiment 115. The method of any one of embodiments 100-114, wherein the nitrogen fixing microbes are wild type bacteria.

Embodiment 116. The method of any one of embodiments 100-111- and 114, wherein the nitrogen fixing microbes are engineered bacteria.

Embodiment 117. The method of any one of embodiments 100-114 and 116, wherein the nitrogen fixing microbes are transgenic bacteria.

Embodiment 118. The method of any one of embodiments 100-114 and 116-117, wherein the nitrogen fixing microbes are intragenic bacteria.

Embodiment 119. The method of any one of embodiments 100-114 and 116-118, wherein the nitrogen fixing microbes are remodeled bacteria.

Embodiment 120. The method of any one of embodiments 100-114 and 116-119, wherein the nitrogen fixing microbes comprise a non-intergeneric genomic modification.

Embodiment 121. The method of any one of embodiments 100-114 and 116-120, wherein the nitrogen fixing microbes are engineered bacteria capable of fixing atmospheric nitrogen in the presence of exogenous nitrogen.

Embodiment 122. The method of any one of embodiments 100-114 and 116-121, wherein the nitrogen fixing microbes are engineered bacteria comprising at least one genetic variation introduced into at least one gene, or non-coding polynucleotide, of the nitrogen fixation or assimilation genetic regulatory network.

Embodiment 123. The method of any one of embodiments 100-114 and 116-122, wherein the nitrogen fixing microbes are engineered bacteria comprising an introduced control sequence operably linked to at least one gene of the nitrogen fixation or assimilation genetic regulatory network.

Embodiment 124. The method of any one of embodiments 100-114 and 116-123, wherein the nitrogen fixing microbes are engineered bacteria comprising a heterologous promoter operably linked to at least one gene of the nitrogen fixation or assimilation genetic regulatory network.

Embodiment 125. The method of any one of embodiments 100-114 and 116-124, wherein the nitrogen fixing microbes are engineered bacteria comprising at least one genetic variation selected from the group consisting of: nifA, nifL, ntrB, ntrC, polynucleotide encoding glutamine synthetase, glnA, glnB, glnK, drat, amtB, polynucleotide encoding glutaminase, glnD, glnE, nifJ, nifH, nifD, nifK, nifY, nifE, nifN, nifU, nifS, nifV, nifW, nifZ, nifM, nifF, nifB, nifQ, a gene associated with biosynthesis of a nitrogenase enzyme, and combinations thereof.

Embodiment 126. The method of any one of embodiments 100-107 and 116-125, wherein the nitrogen fixing microbes are engineered bacteria comprising at least one genetic variation introduced into at least one gene, or non-coding polynucleotide, of the nitrogen fixation or assimilation genetic regulatory network that results in one or more of: increased expression or activity of NifA or glutaminase; decreased expression or activity of NifL, NtrB, glutamine synthetase, GlnB, GlnK, DraT, AmtB; decreased adenylyl-removing activity of GlnE; or decreased expression or uridylyl-removing activity of GlnD.

Embodiment 127. The method of any one of embodiments 100-114 and 116-126, wherein the nitrogen fixing microbes are engineered bacteria comprising a mutated nifL gene that has been altered to comprise a heterologous promoter inserted into said nifL gene.

Embodiment 128. The method of any one of embodiments 100-114 and 116-127, wherein the nitrogen fixing microbes are engineered bacteria comprising a mutated glnE gene that results in a truncated GlnE protein lacking an adenylyl-removing (AR) domain.

Embodiment 129. The method of any one of embodiments 100-114 and 116-128, wherein the nitrogen fixing microbes are engineered bacteria comprising a mutated glnD gene that results in the lack of expression of said glnD gene.

Embodiment 130. The method of any one of embodiments 100-114 and 116-129, wherein the nitrogen fixing microbes are engineered bacteria comprising a mutated amtB gene that results in the lack of expression of said amtB gene.

Embodiment 131. The method of any one of embodiments 100-114 and 116-130, wherein the nitrogen fixing microbes are engineered bacteria comprising at least one of: a mutated nifL gene that has been altered to comprise a heterologous promoter inserted into said nifL gene; a mutated glnE gene that results in a truncated GlnE protein lacking an adenylyl-removing (AR) domain; a mutated amtB gene that results in the lack of expression of said amtB gene; a mutated glnD gene that results in the lack of expression of said glnD gene; and combinations thereof.

1. A water-soluble film package composed of a water-soluble film, the package comprising: one or more compartment(s), wherein at least one of the one or more compartments comprises dehydrated microbes.

2. (canceled)

3. The water-soluble film package of claim 1, wherein the dehydrated microbes are diazotrophic bacteria.

4. The water-soluble film package of claim 1, wherein the dehydrated microbes are in granular form.

5. (canceled)

6. (canceled)

7. The water-soluble film package of claim 1, wherein the water-soluble film comprises a polymer selected from the group consisting of cellulose, a cellulose derivative, pullulan, polyvinyl pyrrolidone, polyvinyl alcohol, polyethylene glycol, carboxyvinyl copolymers, hydroxypropylmethyl cellulose, hydroxyethyl cellulose, hydroxypropyl cellulose, carboxymethyl cellulose, sodium alginate, xanthan gum, tragacanth gum, guar gum, acacia gum, arabic gum, polyacrylic acid, methylmethacrylate copolymer, carboxyvinyl copolymers, starch, gelatin, and combinations thereof.

8.-10. (canceled)

11. The water-soluble film package of claim 7, wherein the water-soluble film comprises polyvinyl alcohol.

12.-21. (canceled)

22. The water-soluble film package of claim 1, wherein the one or more compartment(s) comprise sucrose, milk solids nonfat, or a combination thereof.

23.-25. (canceled)

26. The water-soluble film package of claim 1, wherein the one or more compartment(s) comprise a microbial stabilizer selected from the list consisting of a monosaccharide, a disaccharide, a polysaccharide, a pentose, a hexose, an oligosaccharide, an oligofructose, a sugar alcohol, an amino acid, a protein or protein hydrolysate, and a polymer.

27.-29. (canceled)

30. The water-soluble film package of claim 1, wherein the one or more compartment(s) comprise a physical stabilizer selected from the list consisting of maltodextrin, polyethylene glycol (PEG), xanthan gum, pectin, alginates, microcrystalline cellulose, and dextran.

31. (canceled)

32. The water-soluble film package of claim 3, wherein the diazotrophic bacteria are of a genus selected from the group consisting of: Acetobacter, Achromobacter, Aerobacter, Anabaena, Azoarcus, Azomonas, Azorhizobium, Azospirillum, Azotobacter, Beijernickia, Bradyrhizobium, Burkholderia, Citrobacter, Derxia, Enterobacter, Herbaspirillum, Klebsiella, Kluyvera, Kosakonia, Mesorhizobium, Metakosakonia, Paraburkholderia, Nostoc, Rahnella, Rhizobium, Rhodobacter, Rhodopseudomonas, Rhodospirillum, Serratia Sinorhizobium, Spirillum, Trichodesmium, Xanthomonas, and combinations thereof.

33.-36. (canceled)

37. The water-soluble film package of claim 32, wherein the diazotrophic bacteria are of the genus Klebsiella.

38.-49. (canceled)

50. The water-soluble film package of claim 2, wherein the diazotrophic bacteria are engineered bacteria capable of fixing atmospheric nitrogen in the presence of exogenous nitrogen.

51.-61. (canceled)

62. A method for coating a seed with nitrogen-fixing microbes, the method comprising:
a) contacting the water-soluble film package of claim 1 with a liquid to produce a dispersion of live microbes; and
b) coating the seed with the dispersion of live microbes.

63. The method of claim 62, wherein the water-soluble film package comprises polyvinyl alcohol.

64.-134. (canceled)

135. A freeze dried composition comprising dehydrated microbes and a microbial stabilizer selected from a monosaccharide, a disaccharide, a polysaccharide, a pentose, a hexose, an oligosaccharide, an oligofructose, a sugar alcohol, an amino acid, a protein or protein hydrolysate, a polymer, and any combination thereof.

136. The freeze dried composition of claim 135, wherein the dehydrated microbes are in granular form.

137. The freeze dried composition of claim 135, wherein the dehydrated microbes are diazotrophic bacteria.

138. The freeze dried composition of claim 137, wherein the diazotrophic bacteria are of a genus selected from the group consisting of: Acetobacter, Achromobacter, Aerobacter, Anabaena, Azoarcus, Azomonas, Azorhizobium, Azospirillum, Azotobacter, Beijernickia, Bradyrhizobium, Burkholderia, Citrobacter, Derxia, Enterobacter, Herbaspirillum, Klebsiella, Kluyvera, Kosakonia, Mesorhizobium, Metakosakonia, Paraburkholderia, Nostoc, Rahnella, Rhizobium, Rhodobacter, Rhodopseudomonas, Rhodospirillum, Serratia Sinorhizobium, Spirillum, Trichodesmium, Xanthomonas, and any combination thereof.

139. The freeze dried composition of claim 138, wherein diazotrophic bacteria are of the genus Klebsiella.

140. The freeze dried composition of claim 135, wherein the microbial stabilizer is selected from glucose, fructose, trehalose, sucrose, lactose, melibiose, inulin, lactulose, and any combination thereof.

141. The freeze dried composition of claim 135, wherein the microbial stabilizer is a combination of sucrose and inulin.
